(19) United States
(12) Patent Application Publication
(10) Pub. No.: US 2023/0348911 A1
(43) Pub. Date: Nov. 2, 2023

(54) ANTISENSE OLIGONUCLEOTIDES FOR MODULATION OF LONG NONCODING RNAS

(71) Applicant: Aalborg Universitet, Aalborg Øst (DK)

(72) Inventors: Sakari Kauppinen, Holte (DK); Andreas Petri, Frederiksberg C (DK); Charlotte Albæk Thrue, Carcavelos (PT)

(21) Appl. No.: 16/307,695

(22) PCT Filed: Jun. 8, 2017
(86) PCT No.: PCT/EP2017/064047

§ 371 Date: Dec. 6, 2018

(30) Foreign Application Priority Data
Jun. 8, 2016 (DK) PA201670413
Feb. 7, 2017 (DK) PA201770073

(51) Int. Cl. C12N 15/113; C12N 15/88; A61P 35/00

(52) U.S. Cl. CPC C12N 15/1135; A61P 35/00; C12N 15/88; C12N 2310/11; C12N 2310/321; C12N 2310/322; C12N 2310/3231; C12N 2310/351

(57) ABSTRACT

The present invention relates to noncoding RNAs as novel disease targets, and methods of modulating the activity of such ncRNA targets in patients. In particular, the invention relates to modulation of long non-coding RNAs, such as circular RNAs (circRNAs) or large intergenic noncoding RNAs (lincRNAs) in cancer using antisense oligonucleotides.

### FIELD

The present invention relates to noncoding RNAs as novel disease targets, and methods of modulating the activity of such ncRNA targets in patients. In particular, the invention relates to modulation of long non-coding RNAs, such as circular RNAs (circRNAs) or large intergenic noncoding RNAs (lincRNAs) in cancer using antisense oligonucleotides.

### SEQUENCE LISTING

The present application is being filed along with a sequence listing in electronic format, and is provided as a file named seqListing_ST25_win.txt created on June 8th 2016, which is 1.1 MB (1146832 bytes) in size. The disclosure in the electronic format of the sequence listing is incorporated herein by reference in its entirety.

### BACKGROUND

One of the biggest surprises of the Human Genome Project was the finding that the human genome contains only about 21.000 protein-coding genes, comprising less than 2% of the total genomic sequence (Lander et al. 2001, Nature 409: 860-921; Venter et al. 2001, Science 291: 1304-51, Harrow et al. 2012, Genome Research 22: 1760-74). However, recent reports have shown that the human genome is pervasively transcribed, giving rise to tens of thousands of non-protein coding transcripts (ncRNAs) (Carninci et al. 2006, Science 309: 1559-1563; Djebali et al. 2012, Nature 489: 101-108; Derrien et al. 2012, Genome Research 22: 1775-1789). Indeed, the use of genetics, tiling arrays, RNA cloning and whole-transcriptome profiling by RNA sequencing (RNA-Seq) has uncovered multiple classes of ncRNAs, such as microRNAs (miRNAs), large intergenic noncoding RNAs (lincRNAs), and circular RNAs (circRNAs) (Ambros 2011, Curr Opin Genet Dev 21: 511-517; Bartel 2009, Cell 136: 215-233; Guttman and Rinn 2012, Nature 482: 339-346; Memczak, S, et al., Nature 495 (7441): 333-8; Mercer and Mattick 2013, Nat Struc Mol Biol 20: 300-307). Increasing evidence suggests that noncoding RNAs (ncRNAs) play key regulatory roles in many biological processes in the cell. Furthermore, ncRNA dysregulation is prevalent in human disease, suggesting that ncRNAs may represent a new class of targets for disease intervention (Ventura and Jacks 2009, Cell 136: 586-591.; Huarte and Rinn 2010, Human Mol Genet 19: R152-R161). Given that these findings can be translated from basic scientific discoveries to development of novel, ncRNA-targeted therapeutics, such therapies may provide life-changing treatments for a broad range of diseases.

The term long noncoding RNAs (IncRNAs) refers to an expanding inventory of ncRNAs whose defining characteristics are that they are longer than 200 nucleotides and that they lack a significant open reading frame. Recent technological advances in high-throughput sequencing have allowed rapid identification of IncRNAs. Various characteristics of IncRNAs are used to divide this growing list of molecules into subclasses, such as large intergenic ncRNAs (lincRNA), long intronic ncRNAs, antisense RNAs, pseudogene RNAs, circular RNAs (circRNA) and transcribed-ultraconserved regions.

While functional annotation of this class of RNAs is still limited, IncRNAs have emerged as important regulatory molecules in the pathogenesis of cancer. For example, the lincRNA HOX antisense intergenic RNA (HOTAIR) is part of the HOXC gene cluster on chromosome 12 and is an example of an IncRNA that functions as a scaffold and guides epigenetic regulators to genomic loci in trans. HOTAIR promotes silencing by acting as a scaffold to assemble the Polycomb Repressive Complex 2 (PRC2) and the Lysine-specific Demethylase 1 (LSD1) on the HOXD cluster, where these protein complexes specifically trimethylate histone H3 on lysine 27 and demethylate H3 on lysine 4, respectively, resulting in epigenetic silencing of HOXD genes (Rinn et al. 2007, Cell, 129:1311-1323; Tsai et al. Science 2010, 329:689-693). HOTAIR is highly expressed in primary as well as metastatic breast tumors and high level of expression in primary breast tumors is a powerful predictor of subsequent metastasis and death (Gupta et al. 2010, Nature, 464:1071-1076). CDKN2B-AS, also known as ANRIL (antisense non-coding RNA in the INK4 locus) exemplifies an antisense RNA transcript involved in cis regulation of the INK4b/ARF/INK4a tumor suppressor locus. The nascent ANRIL transcript directly interacts with PRC1 and PRC2 resulting in cis recruitment of gene silencing complexes to the INK4A-ARF-INK4B gene cluster and ANRIL has been shown to be up-regulated in prostate cancer cells (Yap et al. 2010, Mol Cell, 38:662-674, Kotake et al. 2011, Nature 448:943-946). The lincRNA Growth Arrest-Specific 5 (GAS5) is a negative regulator of gene expression exerting its function by acting as a decoy glucocorticoid response element (GRE) capable of binding the glucocorticoid receptor (GR) transcription factor. GAS5 transcripts can compete for binding to GR with GREs in promoter regions of GR target genes resulting in modulation of their expression (Kino et al. 2010, Sci Signal. 3:ra8). Reduced GAS5 transcript levels have been demonstrated in breast cancer relative to adjacent normal tissue; it hosts several snoRNAs in its introns, and plays an important role in controlling apoptosis and cell growth (Mourtada-Maarabouni et al. 2009, Oncogene, 28:195-208). The Malat1 lincRNA regulates alternative splicing (Tripathi et al. 2010, Mol Cell, 39:925-938). Malat1 is up-regulated in many solid tumors and associated with cancer metastasis and recurrence (Ji et al. 2003, Oncogene, 22:8031-8041; Yamada et al. 2006, Cancer Sci, 97:106-112; Lin et al. 2007, FEBS Lett, 585:671-676; Guffanti et al. 2009, BMC Genomics, 10:163; Lai et al., 2012, Med Oncol. 29:1810-1816). Taken together, IncRNAs comprise a class of RNAs that are highly interesting as biomarkers due to the fact that they often show tight spatio-temporal regulation, and as targets for novel anti-cancer therapeutic approaches due to their central role as regulators of many biological processes (Huarte and Rinn 2010, Human Mol Genet 19: R152-R161).

Recent studies combining RNA sequencing (RNAseq)-based transcriptome profiling with focused bioinformatic analyses have revealed large numbers of circRNAs that are stable and much more abundant than previously appreciated (Jeck et al., 2013, RNA 19: 141-57; Memczak et al., 2013, Nature 495: 333-8; Salzman et al., 2012, PLoS One 7: e30733). These molecules constitute the most recent addition to the continuously expanding list of long noncoding RNA (IncRNA) transcripts and tens of thousands have already been identified (Glažar et al., 2014, RNA 20: 1666-70). CircRNAs are formed by a backsplice event, in which a splice donor is joined to an upstream splice acceptor and the resulting RNA molecule can encompass exons (Hansen et al., 2013, Nature 495: 384-8; Memczak et al., 2013, Nature 495: 333-8), introns (Zhang et al., 2013, Mol. Cell 51: 792-806), or a combination of both (Li et al., 2015, Nat. Struct. Mol. Biol. 22: 256-64). Several pathways for biogenesis of circRNAs have been proposed, including circularization driven by inverted repeats in flanking introns (Zhang et al., 2014, Cell 159: 134-147), RNA binding proteins (Ashwal-Fluss et al., 2014, Mol. Cell 56: 55-66; Conn et al., 2015, Cell 160: 1125-34), and lariat formation following exon skipping (Jeck et al., 2013, RNA 19: 141-57). Recent data suggest that the canonical spliceosome machinery functions in the biogenesis of circular RNAs (Starke et al., 2015, Cell Rep. 10: 103-111), and circRNAs have been shown to exhibit cell type and developmental stage specific expression. Furthermore, the expression levels of circRNAs do not always correlate with the linear transcripts from which they are generated (Memczak et al., 2013, Nature 495: 333-8; Salzman et al., 2013, PLoS Genet. 9: e1003777), suggesting that the biogenesis of circRNAs is a regulated process.

Functional studies of specific circRNAs have shown that they can act as competitive endogenous RNAs (ceRNAs) (Salmena et al., 2011, Cell 146: 353-358), and they are involved in post-transcriptional regulation by functioning as miRNA sponges (Hansen et al., 2013, Nature 495: 384-8; Memczak et al., 2013, Nature 495: 333-8; Li et al., 2015, Oncotarget 6: 6001-6013), protein decoys (Ashwal-Fluss et al., 2014, Mol. Cell 56: 55-66), or modulators of transcription of their parent gene (Li et al., 2015, Nat. Struct. Mol. Biol. 22: 256-64). Four circRNAs shown to function as miRNA sponges include: (i) CDR1-AS/ciRS-7 acting as a decoy for the tumor suppressor miR-7 (Hansen et al., 2013, Nature 495: 384-8; Memczak et al., 2013, Nature 495: 333-8), (ii) the testis specific circRNA SRY capable of sequestering miR-138 (Hansen et al., 2013, Nature 495: 384-8), (iii) cir-ITCH, which acts as a sponge for miR-7, miR-17, and miR-214 (Li et al., 2015, Oncotarget 6: 6001-6013), and circHIPK3, which sponges multiple miRNAs, including miR-124 (Zheng et al., 2016, Nat. Commun. 7:11215). CircRNAs are well suited for their function as miRNA sponges, since they do not contain 5′ or 3′ ends and are therefore not subject to miRISC-mediated deadenylation and decapping, which in linear transcripts triggers target mRNA degradation. While ciRS-7 contains 74 miR-7 binding sites, genome-wide studies of circRNAs using a high-throughput sequencing technique called CircleSeq has shown that most exonic circRNAs only have a small number of putative miRNA binding sites (Jeck et al., 2014, Nat. Biotechnol. 32: 453-61). This implies that miRNA sponge activity might not be the prevalent mode of action for this class of molecules (Jeck et al., 2014, Nat. Biotechnol. 32: 453-61). Nonetheless, the identification of circular miRNA sponges, co-expressed with the cognate miRNA, as exemplified by miR-7/ciRS-7 (Hansen et al., 2013, Nature 495: 384-8; Memczak et al., 2013, Nature 495: 333-8), has revealed an increased complexity of miRNA regulatory networks (Salmena et al., 2011, Cell 146: 353-358).

A link between circRNAs and disease was first suggested by Burd et al., who showed that a circular variant of the IncRNA ANRIL correlates with increased risk of atherosclerosis (Burd et al., 2010, PLoS Genet. 6: e1001233). Furthermore, the effective ciRS-7 mediated regulation of miR-7 activity is highly interesting due to the role of miR-7 in suppressing cancer cell growth, proliferation, survival, migration and invasion, as well as increasing sensitivity of resistant tumor cells to therapeutics (Kalinowski et al., 2014, Int. J. Biochem. Cell Biol. 54: 312-7). Although the functions of most circRNAs in human disease are largely unknown, circRNAs are often found to be differentially expressed between cancer and normal tissues (Zheng et al., 2016, Nat. Commun. 7:11215), and many circRNAs are associated with human disease (Ghosal et al., 2013, Front Genet. 4:283), suggesting that circRNAs could represent a new class of targets for development of circRNA-based therapeutics for a wide range of human diseases.

### SUMMARY

The present invention provides novel antisense oligonucleotides (ASOs) and methods of using such ASOs for modulation of lincRNAs and circular RNAs (circRNAs) in cells. The antisense oligonucleotides and methods may in some embodiments be used for treatment of human disease, such as cancer.

### Targeting circRNAs

Specifically, an antisense oligonucleotide according to the invention is complementary to a circRNA, and is for use in knockdown of a circRNA. In one such embodiment, the antisense oligonucleotide is of 14-22 nucleotides in length, and is a gapmer comprising a stretch of DNA that varies in length from 6 to 16 nucleotides flanked at each end by wings comprising from 1 to 5 nucleotide analogues, and wherein the antisense oligonucleotide comprises from 1 to 21, such as from 6 to 21 phosphorothioate internucleotide linkages, and wherein all internucleotide linkages in the DNA stretch are phosphorothioate linkages. This allows the oligonucleotide to bind specifically to the target circRNA and cause degradation of the targeted circRNA, whereby the effect of the target circRNA in a disease is alleviated in whole or in part. In some embodiments, the nucleotide analogues in the antisense oligonucleotides of the invention are locked nucleic acids (LNA). In another embodiment, the antisense oligonucleotide is consisting of a sequence of 10-22 nucleobases in length that is a mixmer which does not comprise a region of more than anyone of 2, 3, 4 or 5 consecutive DNA nucleotides, and which comprises from 3 to 22 affinity-enhancing nucleotide analogues, and wherein the antisense oligonucleotide comprises 1 to 21 phosphorothioate internucleotide linkages, and wherein the oligonucleotide is complementary to an endogenous circRNA.

In some preferred embodiments, the antisense oligonucleotides of the invention are complementary to an endogenous circRNA. In some embodiments, the antisense oligonucleotide has a sequence, which is complementary to a circRNA back-splice junction. In a preferred embodiment, the antisense oligonucleotides of the invention are complementary to a circRNA sequence, which overlaps the back-splice junction by at least 3 nucleotides. This design provides the advantage of targeting the circRNA molecule and not its parental transcript. In some embodiments, the invention provides a siRNA that target a circRNA sequence which overlaps the back-splice juncion by at least 3 nuceotides.

In some embodiments, the antisense oligonucleotide of the invention is complementary to, and thereby targets a circRNA which is anyone of a circRNA selected from the list of ciRS-7, circPVT1, circHIPK3, circSRY, circSLC35E2B, circCDK11A, circUNKNOWN00000001, circARHGAP32, circSLC8A3, circHERC2, circZFAND6, circRP1-168P16.1, circAURKC, circAFTPH, circSCD, circSMC3, circSNORA23| IPO7.1, circZNF124.1, circSNX5 | OVOL2, circRALY, circTFPI, circAHSG.1, circAHSG.2, circAHSG.3, circUBXN7, circAFP, circHIST1H3A, circHIST1H3C.1, circANAPC2, circRMRPI RMRP, circCENPI, circFIRRE, circMBNL3, circGPC3.

In some embodiments, the antisense oligonucleotide of the invention is complementary to, and thereby targets a circRNA which is anyone of a circRNA selected from the list of ciRS-7, circPVT1, circHIPK3, circSRY, circSLC35E2B, circCDK11A, circUNKNOWN00000001, circARHGAP32, circSLC8A3, circHERC2, circZFAND6, circRP1-168P16.1, circAURKC, circAFTPH, circSCD, circSMC3, circSNORA23 | IPO7.1, circZNF124.1, circSNX5 | OVOL2, circRALY, circTFPI, circAHSG.1, circAHSG.2, circAHSG.3, circUBXN7, circAFP, circHIST1H3A, circHIST1H3C.1, circANAPC2, circRMRP | RMRP, circCENPI, circFIRRE, circMBNL3, circGPC3, circPROSER2, circMALRD1, circFAM208B, circMCU, circKIF20B, circABCC2, circEIF4G2|SNORD97.1, circEIF4G2|SNORD97.2, circEIF4G2|SNORD97.3, circEIF4G2|SNORD97.4, circEIF4G2|SNORD97.5, circEIF4G2|SNORD97.6, circEIF4G2|SNORD97.7, circEIF4G2|SNORD97.8, circEIF4G2|SNORD97.9, circEIF4G2|SNORD97.10, circlGF2, circQSER1, circUNKNOWN00000002, circCHD1L, circPRUNE, circSLC27A3, circGATAD2B, circKIAA0907, circCCT3, circPLEKHM2, circVWCE, circATF6, circMALAT1.1, circMALAT1.2, circMALAT1.3, circMALAT1.4, circMALAT1.5, circMALAT1.6, circMALAT1.7, circMALAT1.8, circMALAT1.9, circMALAT1.10, circMALAT1.11, circMALAT1.12, circMALAT1.13, circUNKNOWN00000003, circMALAT1.14, circMALAT1.15, circMALAT1.16, circMALAT1.17, circMALAT1.18, circMALAT1.19, circUCK2, circSUCO, circRAB6A, circRPS3|SNORD15B.1, circRPS3|SNORD15B.2, circRPS3|SNORD15B.3, circRSF1, circABL2, circGNB1, circRPLP2|SNORA52, circPICALM.1, circPICALM.2, circSNORA23|IPO7.2, circSNORA23|IPO7.3, circCFH, circSLC41A2.1, circSLC41A2.2, circCORO1C, circEIF4G3|RP11-487E1.2, circNAA25, circMED13L, circLPGAT1| RN7SL344P, circAACS, circTP53BP2, circSOX5, circDNAH14, circKDM1A|MIR3115, circTTC13, circEGLN1, circTCEA3, circTOMM20|SNORA14B, circSCCPDH, circZNF124.2, circGLS2, circR3HDM2, circDHDDS, circSNORA73AI RCC1| SNHG3.1, circSNORA73A|RCC1| SNHG3.2, circSNORA61|SNHG12, circCEP83|RBMS2P1, circFGD6, circPUM1, circTMCO3|RP11-230F18.6, circPTP4A2, circZMYM5, circN6AMT2, circRPL21|SNORA27, circGTF2F2, circZMYM4, circLINC00355, circUNKNOWN00000004, circFARP1, circDYNC1H1, circCDC42BPB, circCCNB1IP1|SNORA79|AL355075.1, circRPPH1|RPPH1.1, circRPPH1|RPPH1.2, circRPPH1|RPPH1.3, circRPPH1|RPPH1.4, circSNORD8|CHD8.1, circSNORD8|CHD8.2, circPPP1R3E, circCHMP4A|RP11-468E2.1|AL136419.6, circUNKNOWN00000005, circSEC23A, circSNORD46| RPS8, circSAMD4A, circPCNX, circPSEN1, circFCF1, circSCARNA13 |SNHG10.1, circSCARNA13|SNHG10.2, circSCARNA13|SNHG10.3, circUNKNOWN00000006, circTJP1, circRP11-632K20.7, circTTBK2, circPPIB, circUBE2Q2, circETFA, circSEC11A, circPDE8A, circDAB1|OMA1, circABHD2, circlQGAP1.1, circlQGAP1.2, circCHD2, circIGF1R, circNPRL3, circNDE1, circABCC1, circRPS2|SNORA64, circPOLR3E, circATXN2L, circMVP, circASPHD1, circITGAL, circRP5-857K21.6.1, circRP5-857K21.6.2, circRP5-857K21.6.3, circRP5-857K21.6.4, circZNF720, circLONP2, circCHD9, circSLC7A6, circCARHSP1, circFANCA, circRAD51D|RAD51L3-RFFL, circHDAC5, circUTP18, circSRSF1, circPPM1D, circBRIP1, circPRKCA.1, circPRKCA.2, circEIF4A1|SNORD10|RP11-186B7.4|SENP3-EIF4A1.1, circEIF4A1|SNORD10|RP11-186B7.4| SENP3-EIF4A1.2, circPGS1, circRPTOR, circRPL26|RP11-849F2.7, circRP11-206L10.8, circPIAS2, circTYMS, circPPP4R1, circZNF91, circWDR62, circADCK4, circARHGAP35, circNUCB1, circSNORD33| RPL13A.1, circSNORD33| RPL13A.2, circSNORD33| RPL13A.3, circMUC16, circLZIC, circSNX5|SNORD17|OVOL2.1, circSNX5|SNORD17 |OVOL2.2, circSNORA71A|SNHG17, circPLTP, circTMEM230, circCYP24A1, circZBTB46, circGART, circRAB3GAP1, circDYRK1A, circUNKNOWN00000007, circCOL18A1.1, circCOL18A1.2, circNBAS, circCH507-513H4.1.1, circCH507-513H4.1.2, circCH507-513H4.1.3, circANKAR, circGLS, circBMPR2, circRHBDD1, circATG16L1|SCARNA5, circDGKD, circPASK, circPPP6R2, circBIRC6, circPRKD3, circKIAA1841|RP11-493E12.3, circRTKN, circELMOD3, circREV1, circZBTB20, circTIMMDC1, circACAD9, circPLXND1, circHDAC11, circCEP70, circRNF13.1, circRNF13.2, circGOLIM4, circEIF4A2|SNORD2.1, circEIF4A2|SNORD2.2, circSDHAP1, circSETD2, circSCAP, circUSP4, circRPL29, circPHF7, circNEK4, circFLNB, circSLC25A26, circNFKB1, circFIP1L1|RP11-231C18.3, circTBC1D14, circALB.1, circALB.2, circALB.3, circNUP54, circAFF1, circSLC12A7|MIR4635, circMAN2A1.1, circMAN2A1.2, circAFF4, circUBE2D2, circANKHD1|ANKHD1-EIF4EBP3, circMAPK9, circGPBP1, circCEP72, circRP11-98J23.2, circFAM169A, circWDR41, circRASGRF2, circRHOBTB3, circCEP85L, circARID1B.1, circARID1B.2, circTULP4|RP11-732M18.4, circTULP4, circTMEM181, circHIST1H3B, circHIST1H3C.2, circUNKNOWN00000008, circC6orf136, circHLA-C|HLA-B|XXbac-BPG248L24.10|WASF5P|XXbac-BPG248L24.13.1, circHLA-C|HLA-B|XXbac-BPG248L24.10|WASF5P|XXbac-BPG248L24.13.2, circFKBP5, circCNPY3, circSRF, circRN7SK, circFARS2, circMLIP, circZNF292, circPNRC1, circUNKNOWN00000009, circNDUFB2, circKMT2C, circESYT2, circMPP6, circHERPUD2, circOGDH, circZNF680, circKDELR2|DAGLB, circZDHHC4, circCCZ1B, circPOM121, circBAZ1B, circGTF2I, circSNORA14A, circCDK14, circCCDC132, circTRRAP|MIR3609, circCYP3A7|CYP3A7-CYP3A51P, circCCAT1.1, circCCAT1.2, circCCAT1.3, circCCAT1.4, circCCAT1.5, circCCAT1.6, circCCAT1.7, circASAP1, circPTK2.1, circPTK2.2, circSLC45A4, circADGRB1, circRBPMS, circFGFR1, circHOOK3, circASPH, circTMEM245, circUNKNOWN00000010, circHSPA5, circGLE1, circFOCAD, circNFX1, circUBAP2, circKDM4C|RP11-146B14.1, circAGTPBP1, circFAM120A.1, circFAM120A.2, circHIATL1, circPPP2R3B, circATRX, or circTBL1X.

In some embodiments, the antisense oligonucleotide or the siRNA of the invention is complementary to, and thereby targets a circRNA selected from anyone of those listed in Table 1, such as targeting anyone of SEQ ID NOs: 1-359.

In some embodiments, the antisense oligonucleotides of the invention are complementary to a circRNA, which is expressed in cancer cells, or where its expression is upregulated in a cancer cell in comparison with normal liver cells. In some embodiments, the cancer cell is a hepatocellular carcinoma cell.

The oligonucleotides of the invention are for use as medicaments. In some embodiments, the antisense oligonucleotides of the invention are made for use in compositions for treatment of cancer, such as in non-limiting example, cancer that overexpresses a specific circRNA to which the antisense oligonucleotide is complemetary.

### Targeting of Long Noncoding RNAs

In one aspect, the antisense oligonucleotides of the invention are designed to target and downregulate expression of IncRNAs. Specifically, in such an aspect, the antisense oligonucleotide according to the invention is complementary to an IncRNA, and is for use in knockdown of an IncRNA. In such an embodiment, the antisense oligonucleotide is 14-20 nucleotides in length, and is a gapmer comprising a stretch of DNA that varies in length from 6 to 16 nucleotides flanked at each end by wings comprising from 1 to 5 nucleotide analogues, and wherein the antisense oligonucleotide comprises from 1 to 19, such as from 6 to 19 phosphorothioate internucleotide linkages, and wherein all internucleotide linkages in the DNA stretch are phosphorothioate linkages. This allows the oligonucleotide to bind specifically to the target IncRNA and cause degradation of the targeted IncRNA, whereby the effect of the target IncRNA in a disease is alleviated in whole or in part. In some embodiments, the nucleotide analogues in the antisense oligonucleotides of the invention are locked nucleic acids (LNA).

In some preferred embodiments, the antisense oligonucleotides of the invention are complementary to an endogenous IncRNA. In some more preferred embodiments, the antisense oligonucleotides of the invention are fully complementary to the endogenous IncRNA. In some preferred embodiments, the antisense oligonucleotides of the invention contain no DNA -or LNA mismatches to the endogenous IncRNA.

The antisense oligonucleotides of the present invention that target IncRNAs are designed to provide highly specific and efficient targeting of the IncRNA molecule and a minimum of off-target effects.

In some embodiments, the antisense oligonucleotide of the invention is complementary to, and thereby targets anyone of the long noncoding RNAs (IncRNAs) selected from the list of DANCR, H19, HOTAIR, HOTTIP, HULC, LINC-ROR, MALAT1, MVIH, NEAT1, PCBP2-OT1, PVT1, TUG1, UCA1, UFC1 and LINC01215.

In certain embodiments, the antisense oligonucleotides are designed to target IncRNAs and are compounds of anyone of SEQ ID NOs: 2149 to 2259, that target IncRNAs selected from the list of DANCR, H19, HOTAIR, HOTTIP, HULC, LINC-ROR, MALAT1, MVIH, NEAT1, PCBP2-OT1, PVT1, TUG1, UCA1, UFC1 and LINC01215, and their uses as medicaments.

In some embodiments, the antisense oligonucleotides of the invention, selected from the list of anyone of SEQ ID NOs: 2149 to 2259 are for use in the treatment of cancer.

In some embodiments, the antisense oligonucleotides of the invention, selected from the list of anyone of SEQ ID NOs: 2149 to 2259 comprise LNA in the wings, such as in non-limiting example, beta-D-Oxy LNA.

### FIGURE LEGENDS

FIG. 1. Knockdown of the PVT1 lincRNA in the lung cancer cell line A549 cells treated with antisense oligonucleotides (ASOs) targeting PVT1 at 25 nM, 5 nM, 1 nM concentration of the ASOs CRM0091 or CRM0092 (SEQ ID NOs 2233 and 2234 respectively) or mock. PVT1 expression was determined by quantitative real-time RT-PCR carried out on a Quantstudio 6 Flex Real-Time thermocycler (ABI) and results were normalized to GAPDH expression levels. PVT1 expression is shown as % of mock.

FIG. 2. Knockdown of the ciRS-7 circRNA in the lung cancer cell line A549 cells treated with antisense oligonucleotides targeting ciRS-7 RNA at 25 nM, 5 nM, 1 nM concentration of the antisense oligonucleotides CRM0106, CRM0107 or CRM0108 (SEQ ID NOs 360, 361 and 362 respectively) or mock. ciRS-7 expression was determined by quantitative real-time RT-PCR carried out on a Quantstudio 6 Flex Real-Time thermocycler (ABI) and results were normalized to GAPDH expression levels. ciRS-7 levels are shown as % of mock. Total amount of ciRS-7 transcript was measured using a Taqman assay designed with convergent PCR primers (conv or con) specific to the RNA, while the circularized form of ciRS-7 was measured using a Taqman assay designed with divergent PCR primers (div) specific to the ciRS-7 RNA.

FIG. 3. ciRS-7 levels in the lung cancer cell line A549 cells treated with antisense oligonucleotides targeting ciRS-7 RNA at 25 nM concentration of the oligonucleotides CRM0106, CRM0107 or CRM0108 (SEQ ID NOs 360, 361 and 362 respectively) or mock. ciRS-7 expression was determined after incubation for 24 hours, 48 hours or 72 hours by quantitative real-time RT-PCR carried out on a Quantstudio 6 Flex Real-Time thermocycler (ABI) and results were normalized to GAPDH expression levels. ciRS-7 expression is shown as % of mock.

FIG. 4. Knockdown of ciRS-7 in the human prostate cancer cell line PC3, after lipofectamine-assisted uptake of the antisense oligonucleotides CRM0106, or CRM0108 (SEQ ID NOs 360, and 362 respectively) at 1, 5 and 25 nM concentrations. The levels of ciRS-7 are marked “ciRS-7 div”.

FIG. 5. Knockdown of ciRS-7 in the multiple myeloma cell line MM.1S, after unassisted uptake of the antisense oligonucleotides CRM0106, CRM0107 or CRM0108 (SEQ ID NOs 360, 361 and 362 respectively) at 0.5 or 2.5 micromolar concentrations, respectively. The levels of ciRS-7 are marked “ciRS-7 div”.

FIG. 6. Effect of ciRS-7 knockdown on proliferation of A549 lung cancer cells after transfection at 1 nM, 5 nM or 25 nM concentrations of the ciRS-7 antisense oligonucleotides CRM0106, CRM0107 or CRM0108 (SEQ ID NOs 360, 361 and 362 respectively). Data are shown as cell density, measured as % of mock after 24 hours, 48 hours or 72 hours of incubation with the ciRS-7 antisense oligonucleotides.

FIG. 7. Knockdown of the MALAT1 lincRNA in the multiple myeloma cell line MM.1S after unassisted uptake of the antisense oligonucleotide (SEQ ID NO 2198) at 1 or 5 micromolar concentrations, respectively, for 72 hours, 96 hours or for 120 hours. MALAT1 levels were determined by quantitative real-time RT-PCR carried out on a Quantstudio 6 Flex RealTime thermocycler (ABI) and results were normalized to GAPDH expression levels. MALAT1 expression is shown as % of mock.

FIG. 8. Induction of apoptosis in A549 lung cancer cells by lipofectamine-mediated transfection of MALAT1 antisense oligonucleotide (SEQ ID NO: 2198). Antisense oligonucleotide concentration was 25 nM and incubation time 24 hours. Cells were harvested, stained and analyzed in a flow cytometer to detect apoptotic cells. Results were compared to mock. 8A shows mock-treated cells and 8B MALAT1 antisense oligonucleotide-treated cells, respectively.

FIG. 9. A) Knockdown of the ciRS-7 circRNA in the human liver adenocarcinoma cell line SK-Hep-1. Cells were treated with antisense oligonucleotides targeting ciRS-7 RNA at 25 nM or 5 nM concentration of the antisense oligonucleotides CRM0106, CRM0107 or CRM0108 (SEQ ID NOs 360, 361 and 362 respectively) or scrambled control (Scr. Control) or Mock. ciRS-7 expression was determined by quantitative real-time RT-PCR carried out on a Quantstudio 6 Flex Real-Time thermocycler (ABI) and results were normalized to GAPDH expression levels. ciRS-7 levels are shown as % of mock. B) Knockdown of the ciRS-7 circRNA in the human hepatoma cell line Hep3B. Same experimental setup as in 9A), using 25 nM or 5 nM concentration of the antisense oligonucleotides CRM0106, CRM0107 or CRM0108 (SEQ ID NOs 360, 361 and 362 respectively) or scrambled control (Scr. Control) or Mock. Results were normalized to GAPDH expression levels. ciRS-7 levels are shown as % of mock. Total amount of ciRS-7 transcript was measured using a Taqman assay designed with convergent PCR primers (conv or con) specific to the RNA, while the circularized form of ciRS-7 was measured using a Taqman assay designed with divergent PCR primers (div) specific to the ciRS-7 RNA.

FIG. 10. A549 Knockdown of the COROC1c circRNA in the human lung carcinoma cell line A549. Cells were treated with antisense oligonucleotides targeting COROC1c RNA at 25 nM or 5 nM concentration of the antisense oligonucleotides CRM00173, (SEQ ID NO 2266) or scrambled control (Scr. Control) or Mock. COROC1c expression was determined by quantitative real-time RT-PCR carried out on a Quantstudio 6 Flex Real-Time thermocycler (ABI) and results were normalized to GAPDH expression levels. COROC1c levels are shown as % of mock. B) Knockdown of the FAT1 circRNA in the human lung carcinoma cell line A549. Same experimental setup as in 10A), using 25 nM or 5 nM concentration of the antisense oligonucleotides CRM0168, (SEQ ID NO 2262) or scrambled control (Scr. Control) or Mock. Results were normalized to GAPDH expression levels. FAT1 levels are shown as % of mock. C) Knockdown of the HIPK3 circRNA in the human lung carcinoma cell line A549. Same experimental setup as in 10A), using 25 nM or 5 nM concentration of the antisense oligonucleotides CRM0171, (SEQ ID NO 374) or scrambled control (Scr. Control) or Mock. Results were normalized to GAPDH expression levels. HIPK3 levels are shown as % of mock. D) Knockdown of the PVT1 circRNA in the human lung carcinoma cell line A549. Same experimental setup as in 10A), using 25 nM or 5 nM concentration of the antisense oligonucleotides CRM0177, (SEQ ID NOs 2269) or scrambled control (Scr. Control) or Mock. Results were normalized to GAPDH expression levels. PVT1 levels are shown as % of mock. E) Knockdown of the FIRRE circRNA in the human lung carcinoma cell line A549. Same experimental setup as in 10A), using 25 nM or 5 nM concentration of the antisense oligonucleotides CRM0178, (SEQ ID NOs 2270) or scrambled control (Scr. Control) or Mock. Results were normalized to GAPDH expression levels. FIRRE levels are shown as % of mock. F) Knockdown of the CCT3 circRNA in the human lung carcinoma cell line A549. Same experimental setup as in 10A), using 25 nM or 5 nM concentration of the antisense oligonucleotides CRM0182, (SEQ ID NOs 2274 respectively) or scrambled control (Scr. Control) or Mock. Results were normalized to GAPDH expression levels. CCT3 levels are shown as % of mock. Total amount of circRNA transcript was measured using a Taqman assay designed with convergent PCR primers (conv or con) specific to the RNA, while the circularized form of the circRNA was measured using a Taqman assay designed with divergent PCR primers (div) specific to the circRNA.

FIG. 11. A) ) Knockdown of the CORO1C circRNA in the human liver adenocarcinoma cell line SK-Hep-1. Cells were treated with antisense oligonucleotides targeting COROC1c RNA at 25 nM or 5 nM concentration of the antisense oligonucleotides CRM00173, CRM0174 or CRM0175 (SEQ ID NOs 2269, 2270 and 2275 respectively) or scrambled control (Scr. Control) or Mock. COROC1c expression was determined by quantitative real-time RT-PCR carried out on a Quantstudio 6 Flex Real-Time thermocycler (ABI) and results were normalized to GAPDH expression levels. COROC1c levels are shown as % of mock. B) Knockdown of the FAT1 circRNA in the human liver adenocarcinoma cell line SK-Hep-1. Cells were treated with antisense oligonucleotides targeting FAT1 RNA at 25 nM or 5 nM concentration of the antisense oligonucleotides CRM00167, CRM0168 or CRM0169 (SEQ ID NOs 2285, 2286 and 2287 respectively) or scrambled control (Scr. Control) or Mock. FAT1 expression was determined by quantitative real-time RT-PCR carried out on a Quantstudio 6 Flex Real-Time thermocycler (ABI) and results were normalized to GAPDH expression levels. COROC1c levels are shown as % of mock. C) Knockdown of the HIPK3 circRNA in the human liver adenocarcinoma cell line SK-Hep-1. Cells were treated with antisense oligonucleotides targeting HIPK3 RNA at 25 nM or 5 nM concentration of the antisense oligonucleotides CRM0170, CRM0171 or CRM0172 (SEQ ID NOs 2264, 374 and 2265 respectively) or scrambled control (Scr. Control) or Mock. HIPK3 expression was determined by quantitative real-time RT-PCR carried out on a Quantstudio 6 Flex Real-Time thermocycler (ABI) and results were normalized to GAPDH expression levels. HIPK3 levels are shown as % of mock. D) Knockdown of the PVT1 circRNA in the human liver adenocarcinoma cell line SK-Hep-1. Cells were treated with antisense oligonucleotides targeting PVT1 RNA at 25 nM or 5 nM concentration of the antisense oligonucleotides CRM00176,or CRM0177 (SEQ ID NOs 2268 and 2269 respectively) or scrambled control (Scr. Control) or Mock. PVT1 expression was determined by quantitative real-time RT-PCR carried out on a Quantstudio 6 Flex Real-Time thermocycler (ABI) and results were normalized to GAPDH expression levels. PVT1 levels are shown as % of mock. E) Knockdown of the FIRRE circRNA in the human liver adenocarcinoma cell line SK-Hep-1. Cells were treated with antisense oligonucleotides targeting FIRRE RNA at 25 nM or 5 nM concentration of the antisense oligonucleotides CRM00178, CRM0179 or CRM0180 (SEQ ID NOs 2270, 2271 and 2272 respectively) or scrambled control (Scr. Control) or Mock. FIRRE expression was determined by quantitative real-time RT-PCR carried out on a Quantstudio 6 Flex Real-Time thermocycler (ABI) and results were normalized to GAPDH expression levels. FIRRE levels are shown as % of mock. F) Knockdown of the CCT3 circRNA in the human liver adenocarcinoma cell line SK-Hep-1. Cells were treated with antisense oligonucleotides targeting CCT3 RNA at 25 nM or 5 nM concentration of the antisense oligonucleotides CRM00181, or CRM0182 (SEQ ID NOs 2273 and 2274 respectively) or scrambled control (Scr. Control) or Mock. CCT3 expression was determined by quantitative real-time RT-PCR carried out on a Quantstudio 6 Flex Real-Time thermocycler (ABI) and results were normalized to GAPDH expression levels. CCT3 levels are shown as % of mock. Total amount of circRNA transcript was measured using a Taqman assay designed with convergent PCR primers (conv or con) specific to the RNA, while the circularized form of the circRNA was measured using a Taqman assay designed with divergent PCR primers (div) specific to the circRNA.

FIG. 12 A) Knockdown of the FAT1 circRNA in the human hepatoma cell line Hep3B. Same experimental setup as in 9A), using 25 nM or 5 nM concentration of the antisense oligonucleotides CRM0167, CRM0168 or CRM0169 (SEQ ID NOs 2285, 2286 and 2287 respectively) or scrambled control (Scr. Control) or Mock. Results were normalized to GAPDH expression levels. FAT1 levels are shown as % of mock. Total amount of FAT1 transcript was measured using a Taqman assay designed with convergent PCR primers (conv or con) specific to the RNA, while the circularized form of FAT1 was measured using a Taqman assay designed with divergent PCR primers (div) specific to the FAT1 RNA. B) Knockdown of the HIPK3 circRNA in the human hepatoma cell line Hep3B. Same experimental setup as in 12A), using 25 nM or 5 nM concentration of the antisense oligonucleotides CRM0170, CRM0171 or CRM0172 (SEQ ID NOs 2264, 374 and 2265 respectively) or scrambled control (Scr. Control) or Mock. Results were normalized to GAPDH expression levels. HIPK3 levels are shown as % of mock. C) Knockdown of the CORO1c circRNA in the human hepatoma cell line Hep3B. Same experimental setup as in 9A), using 25 nM or 5 nM concentration of the antisense oligonucleotides CRM0173, CRM0174 or CRM0175 (SEQ ID NOs 2267, 2275 and 2268 respectively) or scrambled control (Scr. Control) or Mock. Results were normalized to GAPDH expression levels. CORO1c levels are shown as % of mock. D) Knockdown of the PVT1 circRNA in the human hepatoma cell line Hep3B. Same experimental setup as in 12A), using 25 nM or 5 nM concentration of the antisense oligonucleotides CRM0176, or CRM0177 (SEQ ID NOs 2268 and 2269 respectively) or scrambled control (Scr. Control) or Mock. Results were normalized to GAPDH expression levels. PVT1 levels are shown as % of mock. E) Knockdown of the FIRRE circRNA in the human hepatoma cell line Hep3B. Same experimental setup as in 9A), using 25 nM or 5 nM concentration of the antisense oligonucleotides CRM0178, CRM01179 or CRM0180 (SEQ ID NOs 2270, 2271 and 2272 respectively) or scrambled control (Scr. Control) or Mock. Results were normalized to GAPDH expression levels. FIRRE levels are shown as % of mock. F) Knockdown of the CCT3 circRNA in the human hepatoma cell line Hep3B. Same experimental setup as in 9A), using 25 nM or 5 nM concentration of the antisense oligonucleotides CRM0181 or CRM0182 (SEQ ID NOs 2273 and 2274 respectively) or scrambled control (Scr. Control) or Mock. Results were normalized to GAPDH expression levels. CCT3 levels are shown as % of mock.

FIG. 13. Effect of circRNA knockdown by antisense oligonucleotides on cancer cell proliferation in A) the human lung carcinoma cell line A549, B) the human hepatoma cell line Hep3B and C) the human liver adenocarcinoma cell line SK-Hep-1. The effect of circRNA knockdown on cell proliferation in each cell line was tested using antisense oligonucleotides CRM0171 (circHIPK3), CRM0168 (circFAT1), CRM0173 (circCORO1C), CRM0177 (circPVT1), CRM0178 (circFIRRE), CRM0182 (circCCT3) (SEQ ID NOs 374, 2262, 2266, 2269, 2270, and 2274 respectively).

FIG. 14. RNase R treatment of total RNA from A549, Hep3B and SK-Hep-1 cells to validate the circular nature of the circRNAs identified. Experimental conditions are described in example 18. After RNase R treatment of the total RNA from each cell line, the amount of circular RNA and of linear RNA was quantified using QPCR. A) Shows results from the A549 cell line, B) shows results from the Hep3B cell line, and C) shows results from the SK-Hep-1 cell line.

FIG. 15 Shows the effect of ciRS-7 knockdown by antisense oligonucleotides on miR-7 target mRNAs in A549 cells. Results show effect after 48 hours and after 72 hours.

FIG. 16 Knockdown of ciRS-7 by perfect match gapmer antisense oligonucleotide CRM0106 (SEQ ID NO: 360) compared to different mismatched gapmer antisense oligonucleotides CRM220-227 in cultured SK-Hep1 cells. The scrambled sequence gapmer CRM0023 was used as a negative control in the experiment.

### DETAILED DESCRIPTION

### Definitions

“Back-splice junction” of a circRNA as referred to herein means the region of a circular RNA, where its 3′ and 5′ ends are joined covalently together to result in a circular form.

The term “therapeutically effective amount”, or “effective amount” or “effective dose”, refers to an amount of a therapeutic agent, which confers a desired therapeutic effect on an individual in need of the agent. The effective amount may vary among individuals depending on the health and physical condition of the individual to be treated, the taxonomic group of the individuals to be treated, the formulation of the composition, the method of administration, assessment of the individual’s medical condition, and other relevant factors.

The term “treatment” refers to any administration of a therapeutic medicament, herein comprising an antisense oligonucleotide that partially or completely cures or reduces one or more symptoms or features of a given disease.

The term “small interfering RNA (siRNA)” refers to are small pieces of double-stranded (ds) RNA, usually between 16 to 30 nucleotides long, with 3′ overhangs (2 nucleotides) at each end that can be used to “interfere” with the translation of proteins by binding to and promoting the degradation of messenger RNA (mRNA) at specific sequences.

“Antisense oligonucleotide” means a single-stranded oligonucleotide having a nucleobase sequence that permits hybridization to a corresponding region or segment of a target nucleic acid.

The antisense oligonucleotide of the present invention is preferably a gapmer.

A “gapmer” is a chimeric antisense compound, in which an internal region having a plurality of nucleosides (such as a region of at least 6 or 7 DNA nucleotides), which is capable of recruiting RNAse H activity, such as RNAseH, which region is positioned between external wings at each end, having one or more nucleosides, wherein the nucleosides comprising the internal region are chemically distinct from the nucleoside or nucleosides comprising the external wings.

The internal region of a gapmer may be referred to as the “gap”.

The external regions of a gapmer may be referred to as the “wings”.

A “mixmer” is an antisense compound, which in contrast to a gapmer does not have an internal region with a plurality of DNA nucleosides capable of recruiting RNase H activity. A mixmer is an antisense compound which has a mixture of stretches of affinity enhancing nucleotide analogues such as LNA nucleotides mixed with e.g. DNA nucleotides so that the antisense compound does not comprise a contiguous stretch of DNA that exceeds 3, 4 or 5 in length.

“Nucleoside analogues” are described by e.g. Freier & Altmann; Nucl. Acid. Res., 1997, 25, 4429 - 4443 and Uhlmann; Curr. Opinion in Drug Development, 2000, 3(2), 293-213, and examples of suitable and preferred nucleoside analogues are provided by WO2007031091, which are hereby incorporated by reference.

“5-methylcytosine” means a cytosine modified with a methyl group attached to the 5′ position. A 5-methylcytosine is a modified nucleobase.

“2′—O—methoxyethyl” (also 2′-MOE and 2′—O(CH^{~})^{~}-OCH3) refers to an O-methoxy-ethyl modification at the 2′ position of a furanose ring.

“2′-MOE nucleoside” (also 2′—O—methoxyethyl nucleoside) means a nucleoside comprising a 2′-MOE modified sugar moiety.

A “locked nucleic acid” or “LNA” is often referred to as inaccessible RNA, and is a modified RNA nucleobase. The ribose moiety of an LNA nucleobase is modified with an extra bridge connecting the 2′ oxygen and 4′ carbon. An LNA oligonucleotide offers substantially increased affinity for its complementary strand, compared to traditional DNA or RNA oligonucleotides. In some aspects bicyclic nucleoside analogues are LNA nucleotides, and these terms may therefore be used interchangeably, and is such embodiments, both are characterized by the presence of a linker group (such as a bridge) between C2′ and C4′ of the ribose sugar ring. When used in the present context, the terms “LNA unit”, “LNA monomer”, “LNA residue”, “locked nucleic acid unit”, “locked nucleic acid monomer” or “locked nucleic acid residue”, refer to a bicyclic nucleoside analogue. LNA units are described in inter alia WO 99/14226, WO 00/56746, WO 00/56748, WO 01/25248, WO 02/28875, WO 03/006475, WO2015071388, and WO 03/095467.

“Beta-D-Oxy LNA”, is a preferred LNA variant.

“Bicyclic nucleic acid” or “BNA” or “BNA nucleosides” means nucleic acid monomers having a bridge connecting two carbon atoms between the 4′ and 2′position of the nucleoside sugar unit, thereby forming a bicyclic sugar. Examples of such bicyclic sugar include, but are not limited to A) pt-L-methyleneoxy (4′—CH2—0—2′) LNA, (B) P-D-Methyleneoxy (4′—CH2—0-2′) LNA, (C) Ethyleneoxy (4′— (CH2)2—0-2′) LNA, (D) Aminooxy (4′-CH2-0-N(R)-2′) LNA and (E) Oxyamino (4′—CH2—N(R)—0—2′) LNA.

As used herein, LNA compounds include, but are not limited to, compounds having at least one bridge between the 4′ and the 2′ position of the sugar wherein each of the bridges independently comprises 1 or from 2 to 4 linked groups independently selected from —[C(R^{~})(R2)],,-, —C(R^{~})═C(R2)—, —C(R^{~})═N, —C(═NREM)—, —C(═O)—, —C(═S)—, —O—, —S(═O) -and -N(R&)-; wherein: x is 0, 1, or 2; n is 1, 2, 3, or 4; each R& and R2 is, independently, H, a protecting group, hydroxyl, C»C» alkyl, substituted C» (—CHz—) group connecting the 2′ oxygen atom and the 4′ carbon atom, for which the term methyleneoxy (4′—CH&-0-2′) LNA is used. Furthermore; in the case of the bicylic sugar moiety having an ethylene bridging group in this position, the ethyleneoxy (4′—CH&CH&-0-2′) LNA is used. n -L- methyleneoxy (4′—CH&-0-2′), an isomer of methyleneoxy (4′—CH&-0-2′) LNA is also encompassed within the definition of LNA, as used herein.

In some embodiments, the nucleoside unit is an LNA unit selected from the list of beta-D-oxy-LNA, alpha-L-oxy-LNA, beta-D-amino-LNA, alpha-L-amino-LNA, beta-D-thio-LNA, alpha-L-thio-LNA, 5′-methyl-LNA, beta-D-ENA and alpha-L-ENA.

“cEt″or “constrained ethyl” means a bicyclic sugar moiety comprising a bridge connecting the 4′-carbon and the 2′-carbon, wherein the bridge has the formula: 4′—CH(CHq)—0-2′.

“Constrained ethyl nucleoside” (also cEt nucleoside) means a nucleoside comprising a bicyclic sugar moiety comprising a 4′—CH(CH3)—0-2′ bridge. cEt and some of its properties is described in Pallan et al. Chem Commun (Camb). 2012, August 25; 48(66): 8195-8197.

“Tricyclo (tc)-DNA” belongs to the class of conformationally constrained DNA analogs that show enhanced binding properties to DNA and RNA. Structure and method of production may be seen in Renneberg et al. Nucleic Acids Res. 2002 Jul 1; 30(13): 2751-2757.

“2′-fluoro”, as referred to herein is a nucleoside comprising a fluoro group at the 2′ position of the sugar ring. 2′-fluorinated nucleotides are described in Peng et al. J Fluor Chem. 2008 September; 129(9): 743-766.

“2′—O—methyl”, as referred to herein, is a nucleoside comprising a sugar comprising an —OCH₃ group at the 2′ position of the sugar ring.

“Conformationally Restricted Nucleosides (CRN)” and methods for their synthesis, as referred to herein, are described in WO2013036868, which is hereby incorporated by reference. CRN are sugar-modified nucleosides, in which, similar to LNA, a chemical bridge connects the C2′ and C4′ carbons of the ribose. However, in a CRN, the C2′ - C4’ bridge is one carbon longer than in an LNA molecule. The chemical bridge in the ribose of a CRN locks the ribose in a fixed position, which in turn restricts the flexibility of the nucleobase and phosphate group. CRN substitution within an RNA- or DNA-based oligonucleotide has the advantages of increased hybridization affinity and enhanced resistance to nuclease degradation.

“Unlocked Nucleic Acid” or “UNA”, is as referred to herein unlocked nucleic acid typically where the C2 -C3 C-C bond of the ribose has been removed, forming an unlocked “sugar” residue (see Fluiter et al., Mol. Biosyst., 2009, 10, 1039, hereby incorporated by reference, and Snead et al. Molecular Therapy-Nucleic Acids (2013) 2, e103;).

“Cancer” is also known as malignant neoplasm, which is a term for diseases, in which abnormal cells divide without control, and can invade nearby tissues or spread to other parts of the body.

A “circRNA-positive cancer” is a cancer that expresses a particular circRNA. In example, “ciRS-7 positive cancer” is a cancer which expresses ciRS-7.

“Hepatocellular carcinoma” (HCC) is the most common type of liver cancer. Carcinoma means that it is a cancer found in tissues that cover or line the surfaces of the liver. This is the most common liver cancer type.

Internucleoside linkages are in preferred embodiments phosphorothioate linkages, however, it is recognized that the inclusion of phosphodiester linkages, such as one or two linkages, into an otherwise phosphorothioate oligonucleotide, particularly between or adjacent to nucleotide analogue units can modify the bioavailability and/or bio-distribution of an oligonucleotide as described in WO2008/053314, hereby incorporated by reference. In some embodiments, where suitable and not specifically indicated, all remaining linkage groups are either phosphodiester or phosphorothioate, or a mixture thereof.

The term” circRNA” (circular RNA) refers to a type of RNA, which forms a covalently closed continuous loop where the 3′ and the 5′ ends are joined together, unlike the linear RNA.

The term “unassisted uptake” refers to a transfection method in which cells are transfected with antisense oligonucleotides essentially as described in Soifer et al. (Methods Mol Biol. 2012; 815: 333-46).

The term “GaINAc” or “GaINAc Conjugate” Moieties as referred to herein is a galactose derivative, preferably an N-acetyl- galactosamine (GaINAc) conjugate moiety. More preferably a trivalent N-acetylgalactosamine moiety is used. GalNAc conjugation of antisense oligonucleotides is known previously as described in WO2015071388. Targeting to hepatocytes in the liver can be greatly enhanced by the addition of a conjugate moiety

“Target region” means a portion of a target nucleic acid to which one or more antisense compounds is targeted.

“Targeted delivery” as used herein means delivery, wherein the antisense oligonucleotide has either been formulated in a way that will facilitate efficient delivery in specific tissues or cells, or wherein the antisense oligonucleotide in other ways has been for example modified to comprise a targeting moiety, or in other way has been modified in order to facilitate uptake in specific target cells.

The term “siRNA as used herein is a single-stranded RNA molecule (usually from 21 to 25 nucleotides in length) produced by the cleavage and processing of double-stranded RNA; siRNAs bind to complementary sequences in mRNA and bring about the cleavage and degradation of the targeted mRNA. As used herein, an siRNA may be designed to target a circRNA backsplice junction, in a way to that the region of complementarity overlaps the junction by at least 3 nucleotides. The design and production of siRNAs is well known in the art.

### Compounds

Suitably the antisense oligonucleotides of the invention are capable of down-regulating their targets, i.e. a circRNA or a lincRNA selected from the lists below.

Preferred compounds according to the present invention are selected from the list of anyone of SEQ ID NO’s: 360-2148 and anyone of SEQ ID NO’s: 2285-2299.

### Modulation of circRNA

The present invention relates to chemically-modified antisense oligonucleotides (ASOs) designed to modulate ncRNAs for treatment of human disease, such as cancer. In one aspect, the present invention relates to chemically-modified antisense oligonucleotides designed to modulate circRNAs. The ASOs of the invention recruit RNase H activity for degradation of the target circRNA and comprise phosphorothioate internucleotide linkages, to enhance their pharmacokinetic properties in vivo. These features make the ASO compounds of the invention highly useful as novel medicaments, in particular as anti-cancer therapeutics.

The present invention provides novel methods for modulating the expression of circRNAs in cells. In one aspect of the invention, the invention provides an antisense oligonucleotide consisting of a sequence of 14-22 nucleobases in length that is complementary to an endogenous circRNA, and wherein the antisense oligonucleotide is a gapmer comprising a central region of 6 to 16 consecutive DNA nucleotides flanked in each end by wings each comprising 1 to 5 nucleotide analogues, and wherein the antisense oligonucletide comprises at least 1, or 2, or 3, or 4, or from 5 to 21, such as from 6 to 21, such as from 8 to 21, such as from 9 to 21 phosphorothioate internucleotide linkages, and wherein all internucleotide bonds in the DNA stretch are phosphorothioate linkages. These antisense oligonucleotides have surprisingly been found to be able to efficiently knockdown circRNAs in cells.

In some embodiments of the present invention, the antisense oligonucleotide according to the invention is designed to have a sequence of complementarity to a circRNA, which overlaps the circRNA back-splice junction by at least 3 nucleotides.

In preferred embodiments, the antisense oligonucleotide of the invention is complementary to, and thereby targets a circRNA which is anyone of a circRNA selected from the list of ciRS-7, circPVT1, circHIPK3, circSRY, circSLC35E2B, circCDK11A, circUNKNOWN00000001, circARHGAP32, circSLC8A3, circHERC2, circZFAND6, circRP1-168P16.1, circAURKC, circAFTPH, circSCD, circSMC3, circSNORA23|IPO7.1, circZNF124.1, circSNX5|OVOL2, circRALY, circTFPI, circAHSG.1, circAHSG.2, circAHSG.3, circUBXN7, circAFP, circHIST1H3A, circHIST1H3C.1, circANAPC2, circRMRP|RMRP, circCENPI, circFIRRE, circMBNL3, circGPC3 and circFAT1.

In another preferred embodiments, the antisense oligonucleotide of the invention is targeted to a circRNA which is selected from the list of anyone of ciRS-7, circFAT1, circPVT1, circHIPK3, circSRY, circSLC35E2B, circCDK11A, circUNKNOWN00000001, circARHGAP32, circSLC8A3, circHERC2, circZFAND6, circRP1-168P16.1, circAURKC, circAFTPH, circSCD, circSMC3, circSNORA23|IPO7.1, circZNF124.1, circSNX5|OVOL2, circRALY, circTFPI, circAHSG.1, circAHSG.2, circAHSG.3, circUBXN7, circAFP, circHIST1H3A, circHIST1H3C.1, circANAPC2, circRMRP|RMRP, circCENPI, circFIRRE, circMBNL3, circGPC3, circPROSER2, circMALRD1, circFAM208B, circMCU, circKIF20B, circABCC2, circEIF4G2|SNORD97.1, circEIF4G2|SNORD97.2, circEIF4G2|SNORD97.3, circEIF4G2|SNORD97.4, circEIF4G2 |SNORD97.5, circEIF4G2|SNORD97.6, circEIF4G2|SNORD97.7, circEIF4G2|SNORD97.8, circEIF4G2|SNORD97.9, circEIF4G2|SNORD97.10, circlGF2, circQSER1, circUNKNOWN00000002, circCHD1L, circPRUNE, circSLC27A3, circGATAD2B, circKIAA0907, circCCT3, circPLEKHM2, circVWCE, circATF6, circMALAT1.1, circMALAT1.2, circMALAT1.3, circMALAT1.4, circMALAT1.5, circMALAT1.6, circMALAT1.7, circMALAT1.8, circMALAT1.9, circMALAT1.10, circMALAT1.11, circMALAT1.12, circMALAT1.13, circUNKNOWN00000003, circMALAT1.14, circMALAT1.15, circMALAT1.16, circMALAT1.17, circMALAT1.18, circMALAT1.19, circUCK2, circSUCO, circRAB6A, circRPS3|SNORD15B.1, circRPS3|SNORD15B.2, circRPS3|SNORD15B.3, circRSF1, circABL2, circGNB1, circRPLP2|SNORA52, circPICALM.1, circPICALM.2, circSNORA23|IPO7.2, circSNORA23|IPO7.3, circCFH, circSLC41A2.1, circSLC41A2.2, circCORO1C, circEIF4G3|RP11-487E1.2, circNAA25, circMED13L, circLPGAT1|RN7SL344P, circAACS, circTP53BP2, circSOX5, circDNAH14, circKDM1A|MIR3115, circTTC13, circEGLN1, circTCEA3, circTOMM20|SNORA14B, circSCCPDH, circZNF124.2, circGLS2, circR3HDM2, circDHDDS, circSNORA73A|RCC1|SNHG3.1, circSNORA73AI RCC1|SNHG3.2, circSNORA61|SNHG12, circCEP83|RBMS2P1, circFGD6, circPUM1, circTMCO3|RP11-230F18.6, circPTP4A2, circZMYM5, circN6AMT2, circRPL21|SNORA27, circGTF2F2, circZMYM4, circLINC00355, circUNKNOWN00000004, circFARP1, circDYNC1H1, circCDC42BPB, circCCNB1IP1|SNORA79|AL355075.1, circRPPH1|RPPH1.1, circRPPH1|RPPH1.2, circRPPH1|RPPH1.3, circRPPH1|RPPH1.4, circSNORD8|CHD8.1, circSNORD8|CHD8.2, circPPP1R3E, circCHMP4A|RP11-468E2.1|AL136419.6, circUNKNOWN00000005, circSEC23A, circSNORD46|RPS8, circSAMD4A, circPCNX, circPSEN1, circFCF1, circSCARNA13|SNHG10.1, circSCARNA13|SNHG10.2, circSCARNA13|SNHG10.3, circUNKNOWN00000006, circTJP1, circRP11-632K20.7, circTTBK2, circPPIB, circUBE2Q2, circETFA, circSEC11A, circPDE8A, circDAB1|OMA1, circABHD2, circlQGAP1.1, circlQGAP1.2, circCHD2, circIGF1R, circNPRL3, circNDE1, circABCC1, circRPS2|SNORA64, circPOLR3E, circATXN2L, circMVP, circASPHD1, circITGAL, circRP5-857K21.6.1, circRP5-857K21.6.2, circRP5-857K21.6.3, circRP5-857K21.6.4, circZNF720, circLONP2, circCHD9, circSLC7A6, circCARHSP1, circFANCA, circRAD51D|RAD51L3-RFFL, circHDAC5, circUTP18, circSRSF1, circPPM1D, circBRIP1, circPRKCA.1, circPRKCA.2, circEIF4A1|SNORD10| RP11-186B7.4|SENP3-EIF4A1.1, circEIF4A1|SNORD10|RP11-186B7.4|SENP3-EIF4A1.2, circPGS1, circRPTOR, circRPL26|RP11-849F2.7, circRP11-206L10.8, circPIAS2, circTYMS, circPPP4R1, circZNF91, circWDR62, circADCK4, circARHGAP35, circNUCB1, circSNORD33| RPL13A.1, circSNORD33| RPL13A.2, circSNORD33| RPL13A.3, circMUC16, circLZIC, circSNX5|SNORD17|OVOL2.1, circSNX5|SNORD17|OVOL2.2, circSNORA71A|SNHG17, circPLTP, circTMEM230, circCYP24A1, circZBTB46, circGART, circRAB3GAP1, circDYRK1A, circUNKNOWN00000007, circCOL18A1.1, circCOL18A1.2, circNBAS, circCH507-513H4.1.1, circCH507-513H4.1.2, circCH507-513H4.1.3, circANKAR, circGLS, circBMPR2, circRHBDD1, circATG16L1|SCARNA5, circDGKD, circPASK, circPPP6R2, circBIRC6, circPRKD3, circKIAA1841|RP11-493E12.3, circRTKN, circELMOD3, circREV1, circZBTB20, circTIMMDC1, circACAD9, circPLXND1, circHDAC11, circCEP70, circRNF13.1, circRNF13.2, circGOLIM4, circEIF4A2|SNORD2.1, circEIF4A2|SNORD2.2, circSDHAP1, circSETD2, circSCAP, circUSP4, circRPL29, circPHF7, circNEK4, circFLNB, circSLC25A26, circNFKB1, circFIP1L1|RP11-231C18.3, circTBC1D14, circALB.1, circALB.2, circALB.3, circNUP54, circAFF1, circSLC12A7 | MIR4635, circMAN2A1.1, circMAN2A1.2, circAFF4, circUBE2D2, circANKHD1|ANKHD1-EIF4EBP3, circMAPK9, circGPBP1, circCEP72, circRP11-98J23.2, circFAM169A, circWDR41, circRASGRF2, circRHOBTB3, circCEP85L, circARID1B.1, circARID1B.2, circTULP4|RP11-732M18.4, circTULP4, circTMEM181, circHIST1H3B, circHIST1H3C.2, circUNKNOWN00000008, circC6orf136, circHLA-C|HLA-B|XXbac-BPG248L24.10|WASF5P|XXbac-BPG248L24.13.1, circHLA-C|HLA-B|XXbac-BPG248L24.10|WASF5P|XXbac-BPG248L24.13.2, circFKBP5, circCNPY3, circSRF, circRN7SK, circFARS2, circMLIP, circZNF292, circPNRC1, circUNKNOWN00000009, circNDUFB2, circKMT2C, circESYT2, circMPP6, circHERPUD2, circOGDH, circZNF680, circKDELR2|DAGLB, circZDHHC4, circCCZ1B, circPOM121, circBAZ1B, circGTF2I, circSNORA14A, circCDK14, circCCDC132, circTRRAP|MIR3609, circCYP3A7|CYP3A7-CYP3A51P, circCCAT1.1, circCCAT1.2, circCCAT1.3, circCCAT1.4, circCCAT1.5, circCCAT1.6, circCCAT1.7, circASAP1, circPTK2.1, circPTK2.2, circSLC45A4, circADGRB1, circRBPMS, circFGFR1, circHOOK3, circASPH, circTMEM245, circUNKNOWN00000010, circHSPA5, circGLE1, circFOCAD, circNFX1, circUBAP2, circKDM4C|RP11-146B14.1, circAGTPBP1, circFAM120A.1, circFAM120A.2, circHIATL1, circPPP2R3B, circATRX, or circTBL1X.

In such preferred embodiments, the antisense oligonucleotide of the invention is at least 80%, such as at least 85%, such as at least 90 %, such as at least 95%, such as at least 100% complementary to a sequence of between 14 and 22 nucleotides in length and which sequence is located within anyone of SEQ ID NOs: 1 - 359 and 2260, which are the sequences of the circRNA back-splice junctions in the above list of circRNAs. In a preferred embodiment, the antisense oligonucleotide that is complementary to a sequence within anyone of SEQ ID NOs: 1 - 359 and 2260, will be designed so that the region of complementarity overlaps the back-splice junction (see Tabel 1) by at least 3 nucleotides.

The back-splice junctions were identified as described in example nr. 4. Each back-splice junction was uniquely identified in the hg38 genome by the chromosome name (chrName), position of the donor and acceptor (posAcceptor and posDonor), and the strand of the chromosome (strand). A unique backsplice ID (bsID) was generated from this info ([chrName]:[posAcceptor]-[posDonor]|[strand], e.g. X:140783175-140784661|+).Vertical lines in sequences denote the back-splice junction in each circRNA.

TABLE 1

Back-splice junction-encompassing sequences identified in cancer-associated circRNAs.

| SEQ ID NO | bsID | Parental gene | Parent gene name | Back-splice junction sequence | circRNA name |
|---|---|---|---|---|---|
| 1 | X:140783175-140784661\|+ | ENSG00000281508 | CDR1-AS | GUGUCUGCAAUAUCCAGG\| GUUUCCGAUGGCACCUGU | ciRS-7 |
| 2 | 8:127890588-127890999\|+ | ENSG00000249859 | PVT1 | CGCUCAGCUGGGCUUGAG \| GCCUGAUCUUUUGGCCAG | circPVT1 |
| 3 | 11:33286412-33287512\|+ | ENSG00000110422 | HIPK3 | ACAAUCUCGGUACUACAG \| GUAUGGCCUCACAAGUCU | circHIPK3 |
| 4 | Y:2787700-2786854\|- | ENSG00000184895 | SRY | AAGGCCUUAUUCAUUUCA \| GUAACAAAGAAUCUGGUA | circSRY |
| 5 | 1:1719456-1655368\|- | ENSG00000189339 | SLC35E2B | AGACUCUAUGGAAGACAG \| AGGAGAAGAAGAUGAUUC | circSLC35E2B |
| 6 | 1:1734836-1669663\|- | ENSG00000008128 | CDK11A | GCCCGGGUUUUCUUUACG \| GACGUCCCAGUGAUCGGG | circCDK11A |
| 7 | 10:94588228-94594855\|+ | UNKNOWNOOOOOOO 1 | UNKNOWN00000001 | AGUUGAUCAUCCAUAAAA \| AUUUUAACACCUUUCUUA | circUNKNOWN00000001 |
| 8 | 11:129124895 -129123445\|- | ENSG00000134909 | ARHGAP32 | GAGUUCGGCAGCAUACAG \| GCAAGAGGCGCAGAUGUU | circARHGAP32 |
| 9 | 14:70168485-70166638\|- | ENSG00000100678 | SLC8A3 | CAAGAAUGAUGAAACUGU \| GUCUCUGGCCUAUCAGGA | circSLC8A3 |
| 10 | 15:28273005-28245880\|- | ENSG00000128731 | HERC2 | CCUGGCCUGGCAUAAUGG \| GGAUGUUCACGGAACGCC | circHERC2 |
| 11 | 15:80098415-80122801\|+ | ENSG00000086666 | ZFAND6 | AAACAGAAGAUGUGCAGG \| GAAUGAAUCUGAAGUCUG | circZFAND6 |
| 12 | 16:89431327-89418283\|- | ENSG00000261692 | RP1-168P16.1 | CUUUCAGCUAUGGAAUCG \| CCCGUGCAGCCUGCCAGG | circRP1-168P16.1 |
| 13 | 19:57211353-57248701\|+ | ENSG00000105146 | AURKC | CCCAAGGCACCUGUCCAG \| GCGACAAAGGAAAACCUA | circAURKC |
| 14 | 2:64551442-64553410\|+ | ENSG00000119844 | AFTPH | UCAGCCACUUCAGUUCAG \| GUGUAAAUUAAUUAUUUG | circAFTPH |
| 15 | 10:100361145 -100361314\|+ | ENSG00000099194 | SCD | UGCUCCAGAUAACUCUCU \| CUCUUCUAGGCCCAUUGU | circSCD |
| 16 | 10:110596397 -110598291\|+ | ENSG00000108055 | SMC3 | AAAACCUUCAUGCCUAAG \| GUGACCAAGUCAGCCAUC | circSMC3 |
| 17 | 11:9428767-9428954\|+ | ENSG00000201998 \|ENSG00000205339 | SNORA23\|IPO7 | UCCAAAUUCCCACACACA \| UGGCUGCUGUAAUGUGUG | circSNORA23\|IPO7.1 |
| 18 | 1:247159814-247138715\|- | ENSG00000196418 | ZNF124 | UUAUGGGUUUCUACUCUA \| AACUCGGUUGCCUUUGAG | circZNF124.1 |
| 19 | 20:17957038-17955364\|- | ENSG00000089006 \|ENSG00000125850 | SNX5\|OVOL2 | GACUAUGCUGGGCUUAUU \| CUGAGAUCUGUAUCUGUG | circSNX5\|OVOL2 |
| 20 | 20:34029768-34031605\|+ | ENSG00000125970 | RALY | AGCUCAUCAUUGUUACUG \| AGCUGCAGAGAUCCUUUG | circRALY |
| 21 | 2:187465961-187465763\|- | ENSG00000003436 | TFPI | GAAACAACCAUUUCAUUC \| UUGGGGAUUUAAUAUUUU | circTFPI |
| 22 | 3:186617235-186618631\|+ | ENSG00000145192 | AHSG | CAAGUGUAACCUGCUGGC \| CCCGCUGAACGACACCAG | circAHSG.1 |
| 23 | 3:186620981-186621179\|+ | ENSG00000145192 | AHSG | UUGACGGUAAGCCACCAU \| UUUGUCCAAGCCUGGGCA | circAHSG.2 |
| 24 | 3:186620982-186621220\|+ | ENSG00000145192 | AHSG | GACCUUACAAAAACCAUU \| UUGUCCAAGCCUGGGCAU | circAHSG.3 |
| 25 | 3:196352732-196352547\|- | ENSG00000163960 | UBXN7 | AAAUCUGAGAUCUAAUUC \| AUGAGCCACUGCGCCCGA | circUBXN7 |
| 26 | 4:73440693-73443441\|+ | ENSG00000081051 | AFP | GGACCCGAACUUUCCAAG \| ACAUAACUGUUUUCUUGC | circAFP |
| 27 | 6:26020635-26020938\|+ | ENSG00000275714 | HIST1H3A | GGUCCAAGCAAAGGCUCU \| GCGCGAGAUCCGCCGUUA | circHIST1H3A |
| 28 | 6:26045383-26045792\|+ | ENSG00000278272 | HIST1H3C | CCCAAAGAUAUCCAGCUG \| ACACUUUUGUGUGUGCUC | circHIST1H3C.1 |
| 29 | 9:137175708-137175499\|- | ENSG00000176248 | ANAPC2 | AGCCCGUGCCCAGGCUGC \| GUGAGCCCACCAGCCCCU | circANAPC2 |
| 30 | 9:35657983-35657774\|- | ENSG00000269900 \|ENSG00000277027 | RMRP\|RMRP | UCCGCGCACCAACCACAC \| UGAGGACUCUGUUCCUCC | circRMRP\|RMRP |
| 31 | X:101162107-101162293\|+ | ENSG00000102384 | CENPI | AGACCAUGCCUGUACUAA \| UUUUUGAUUUUUUAGUAG | circCENPI |
| 32 | X:131794467-131749305\|- | ENSG00000213468 | FIRRE | AGGAGAUACUUUAUGAGG \| AGACUAAGGUGUCAGUAU | circFIRRE |
| 33 | X:132378685-132378526\|- | ENSG00000076770 | MBNL3 | UUUACACAAAUGCCAUUU \| GUCAUCUUCAGCUGAGAA | circMBNL3 |
| 34 | X:133754177-133753481\|- | ENSG00000147257 | GPC3 | GGAAAGCUGACCACCACU \| AGGCCUUUGAAAUUGUUG | circGPC3 |
| 35 | 10:11851996-11852216\|+ | ENSG00000148426 | PROSER2 | UCCAGAAGCUUCACUUUG \| GAGUGAGCUGUUGCCGCA | circPROSER2 |
| 36 | 10:19257683-19283182\|+ | ENSG00000204740 | MALRD1 | AUCAGUGGAUCAUACACA \| AUAUUGGUGGAGGCUUCA | circMALRD1 |
| 37 | 10:5709529-5714208\|+ | ENSG00000108021 | FAM208B | UGGGUGAUCCAGCCAAAG \| CAUUUAUGACAUUAACAG | circFAM208B |
| 38 | 10:72715110-72715903\|+ | ENSG00000156026 | MCU | AAACUUGAACAUUCGCAG \| GUCUUUCACUACCAUUCC | circMCU |
| 39 | 10:89751345-89762836\|+ | ENSG00000138182 | KIF20B | AGUAAUGAAAUGGAGGAG \| AUCUAAAUGUUAAAGAGA | circKIF20B |
| 40 | 10:99834379-99834536\|+ | ENSG00000023839 | ABCC2 | AUUUAUGUAUCUGUUCAG \| GAUAUUUCCACAGUGGAU | circABCC2 |
| 41 | 11:10801577-10801475\|- | ENSG00000110321 \|ENSG00000238622 | EIF4G2\|SNORD97 | AGCGAAGAUUAUGAGAUA \| AGAGGACUUUAUGCUGGA | circEIF4G2\|SNORD 97.1 |
| 42 | 11:10801579-10801476\|- | ENSG00000110321 \|ENSG00000238622 | EIF4G2\|SNORD97 | GAGCGAAGAUUAUGAGAU \| UAAGAGGACUUUAUGCUG | circEIF4G2\|SNORD 97.2 |
| 43 | 11:10801593-10801475\|- | ENSG00000110321 \|ENSG00000238622 | EIF4G2\|SNORD97 | AGCGAAGAUUAUGAGAUA \| AAAAGACGCGUUAUUAAG | circEIF4G2\|SNORD 97.3 |
| 44 | 11:10801594-10801474\|- | ENSG00000110321 \|ENSG00000238622 | EIF4G2\|SNORD97 | GCGAAGAUUAUGAGAUAU \| AAAAAGACGCGUUAUUAA | circEIF4G2\|SNORD 97.4 |
| 45 | 11:10801594-10801481\|- | ENSG00000110321 \|ENSG00000238622 | EIF4G2\|SNORD97 | CCUGUGAGCGAAGAUUAU \| AAAAAGACGCGUUAUUAA | circEIF4G2\|SNORD 97.5 |
| 46 | 11:10801601-10801479\|- | ENSG00000110321 \|ENSG00000238622 | EIF4G2\|SNORD97 | UGUGAGCGAAGAUUAUGA \| UGAUUAUAAAAAGACGCG | circEIF4G2\|SNORD 97.6 |
| 47 | 11:10801601-10801488\|- | ENSG00000110321 \|ENSG00000238622 | EIF4G2\|SNORD97 | CCAGCGUCCUGUGAGCGA \| UGAUUAUAAAAAGACGCG | circEIF4G2\|SNORD 97.7 |
| 48 | 11:10801606-10801493\|- | ENSG00000110321 \|ENSG0000023862 2 | EIF4G2\|SNORD97 | AAUGUCCAGCGUCCUGUG \| CCCGAUGAUUAUAAAAAG | circEIF4G2\|SNORD 97.8 |
| 49 | 11:10801608-10801467\|- | ENSG00000110321 \|ENSG0000023862 2 | EIF4G2\|SNORD97 | UUAUGAGAUAUGAGGGCA \| UGCCCGAUGAUUAUAAAA | circEIF4G2\|SNORD 97.9 |
| 50 | 11:10801608-10801468\|- | ENSG00000110321 \|ENSG00000238622 | EIF4G2\|SNORD97 | AUUAUGAGAUAUGAGGGC \| UGCCCGAUGAUUAUAAAA | circEIF4G2\|SNORD 97.10 |
| 51 | 11:2132056-2131827\|- | ENSG00000167244 | IGF2 | ACAGCACACAAACGCACA \| CAGCACACACACAAACGC | circlGF2 |
| 52 | 11:32927156-32935436\|+ | ENSG00000060749 | QSER1 | AUAAAAAGAAGAAAACAG \| UUUAAGCUAUGAAGAAGG | circQSER1 |
| 53 | 11:33287512-33286412\|- | UNKNOWN00000002 | UNKNOWN00000002 | AGACUUGUGAGGCCAUAC \| CUGUAGUACCGAGAUUGU | circUNKNOWN00000002 |
| 54 | 1:147275353-147285488\|+ | ENSG00000131778 | CHD1L | GGCUGAACAUAAGAAAAA \| GUGGAGUUGGCAUGAACU | circCHD1L |
| 55 | 1:151024610-151025674\|+ | ENSG00000143363 | PRUNE | CAAAGUUUGAUGUAUCAG \| AAGUGACACAGCCCUAGA | circPRUNE |
| 56 | 1:153778949-153779098\|+ | ENSG00000143554 | SLC27A3 | UGACUCCCAGUUUCAGAU \| CUCAACUCUCUAAUCUGC | circSLC27A3 |
| 57 | 1:153813453-153811730\|- | ENSG00000143614 | GATAD2B | GCCUCCUUGGUAUGCCAG \| GCAAAAGCUGUGCCUCAC | circGATAD2B |
| 58 | 1:155921919-155921374\|- | ENSG00000132680 | KIAA0907 | CCAGCUAGCACUGGACAG \| GUUCAUGCUGAAUACUCU | circKIAA0907 |
| 59 | 1:156334919-156333546\|- | ENSG00000163468 | CCT3 | CAUCAGUAAUUAUUCUUG \| ACUAUUGCAGAUAUCAUC | circCCT3 |
| 60 | 1:15717892-15721389\|+ | ENSG00000116786 | PLEKHM2 | CCAGCACAGACUGGGAAG \| GCCGUGCCUGGCUGUACC | circPLEKHM2 |
| 61 | 11:61278477-61276592\|- | ENSG00000167992 | VWCE | GCUGUACUUGUGUUCCAG \| GCUGUUUUCACAGUGGUG | circVWCE |
| 62 | 1:161778243-161784097\|+ | ENSG00000118217 | ATF6 | UCAACUCAGCAUGUUCCU \| AUUCUGCUCUCUUUGCUG | circATF6 |
| 63 | 11:65499621-65499850\|+ | ENSG00000251562 | MALAT1 | AACUUAGAAGAAAAUUGG \| UAGGAUGAAACAAUUUGG | circMALAT1.1 |
| 64 | 11:65499631-65499877\|+ | ENSG00000251562 | MALAT1 | ACAAGAUAGAAAAUGAAA \| CAAUUUGGAGAAGAUAGA | circMALAT1.2 |
| 65 | 11:65499633-65499735\|+ | ENSG00000251562 | MALAT1 | AAUUAGAAGAUAAAAACA \| AUUUGGAGAAGAUAGAAG | circMALAT1.3 |
| 66 | 11:65499645-65499748\|+ | ENSG00000251562 | MALAT1 | AAACAUACUUUUAGAAGA \| UAGAAGUUUGAAGUGGAA | circMALAT1.4 |
| 67 | 11:65499647-65499757\|+ | ENSG00000251562 | MALAT1 | UUUAGAAGAAAAAAGAUA \| GAAGUUUGAAGUGGAAAA | circMALAT1.5 |
| 68 | 11:65499650-65499859\|+ | ENSG00000251562 | MALAT1 | GAAAAUUGGAAGAUAGAA \| GUUUGAAGUGGAAAACUG | circMALAT1.6 |
| 69 | 11:65499650-65499870\|+ | ENSG00000251562 | MALAT1 | GAUAGAAACAAGAUAGAA \| GUUUGAAGUGGAAAACUG | circMALAT1.7 |
| 70 | 11:65499665-65499846\|+ | ENSG00000251562 | MALAT1 | UACAAACUUAGAAGAAAA \| CUGGAAGACAGAAGUACG | circMALAT1.8 |
| 71 | 11:65499673-65499854\|+ | ENSG00000251562 | MALAT1 | UAGAAGAAAAUUGGAAGA \| CAGAAGUACGGGAAGGCG | circMALAT1.9 |
| 72 | 11:65499696-65499841\|+ | ENSG00000251562 | MALAT1 | AAAUGUACAAACUUAGAA \| AAGAAUAGAGAAGAUAGG | circMALAT1.10 |
| 73 | 11:65499698-65499846\|+ | ENSG00000251562 | MALAT1 | UACAAACUUAGAAGAAAA \| GAAUAGAGAAGAUAGGGA | circMALAT1.11 |
| 74 | 11:65499720-65499848\|+ | ENSG00000251562 | MALAT1 | CAAACUUAGAAGAAAAUU \| AGAAGAUAAAAACAUACU | circMALAT1.12 |
| 75 | 11:65499750-65499872\|+ | ENSG00000251562 | MALAT1 | UAGAAACAAGAUAGAAAA \| AAGAUAAAUUUAAACCUG | circMALAT1.13 |
| 76 | 11:65499751-65499641\|- | UNKNOWN0000003 | UNKNOWN00000003 | ACUUCAAACUUCUAUCUU \| UUUUCUUCUAAAAGUAUG | circUNKNOWN00000003 |
| 77 | 11:65499790-65499904\|+ | ENSG00000251562 | MALAT1 | AGAGUUUCAGAUAGAAAA \| AAGACAAGCUAGGAAACA | circMALAT1.14 |
| 78 | 11:65499798-65499925\|+ | ENSG00000251562 | MALAT1 | AAAACAAGCUAAGACAAG \| CUAGGAAACAAAAAGCUA | circMALAT1.15 |
| 79 | 11:65499936-65500058\|+ | ENSG00000251562 | MALAT1 | UUCUGGUGGUGCAGAAGU \| AUAGAAGAUAGAAAAAUA | circMALAT1.16 |
| 80 | 11:65500207-65500413 \|+ | ENSG00000251562 | MALAT1 | CCAGGUGCUACACAGAAG \| GCUUUUGGAAGAGUUAGA | circMALAT1.17 |
| 81 | 11:65500459-65500625\|+ | ENSG00000251562 | MALAT1 | GAAAAAGGAUUCCAGGAA\| CCAGUGUUUGAUGAAGCU | circMALAT1.18 |
| 82 | 11:65500517-65500646\|+ | ENSG00000251562 | MALAT1 | GCGAGUGCAAUUUGGUGA \| AGAUAGGAAAAGAGUCCA | circMALAT1.19 |
| 83 | 1:165890203-165891323\|+ | ENSG00000143179 | UCK2 | CUUUGUCUCCCAUUCCCG \| UCUUCCGUGUGUGCUAAG | circUCK2 |
| 84 | 1:172551511-172557795\|+ | ENSG00000094975 | SUCO | GAUAAUUUAAAAAAUGAG \| GCUUCCCAGCUGGCGUGU | circSUCO |
| 85 | 11:73718719-73707419\|- | ENSG00000175582 | RAB6A | GGAUACAAUGUAAAGCAG \| GUACGAUUGCAAUUAUGG | circRAB6A |
| 86 | 11:75404420-75404564\|+ | ENSG00000149273 \|ENSG00000207445 | RPS3\|SNORD15B | AGAGGCAUUUGUCUGAGA\| CUUCAGUGAUGACACGAU | circRPS3 \| SNORD15 B.1 |
| 87 | 11:75404420-75404566\|+ | ENSG00000149273 \|ENSG00000207445 | RPS3\|SNORD15B | AGGCAUUUGUCUGAGAAG \| CUUCAGUGAUGACACGAU | circRPS3 \| SNORD15 B.2 |
| 88 | 11:75404420-75404567\|+ | ENSG00000149273 \|ENSG00000207445 | RPS3\|SNORD15B | GGCAUUUGUCUGAGAAGG \| CUUCAGUGAUGACACGAU | circRPS3 \| SNORD15 B.3 |
| 89 | 11:77693612-77683709\|- | ENSG00000048649 | RSF1 | GAAAUGUAUAAGCUACAG \| AUUCUUCUGUGUGACUCU | circRSF1 |
| 90 | 1:179133375-179131310\|- | ENSG00000143322 | ABL2 | CACUCAGCAUCACUAAAG \| AUCACUUUGCCAGCUGUG | circABL2 |
| 91 | 1:1806539-1804418\|- | ENSG00000078369 | GNB1 | AGCUGGCAGGACACACAG \| GCUUCUCGUCAGUGCCUC | circGNB1 |
| 92 | 11:811681-811816\|+ | ENSG00000177600 \| ENSG00000199785 | RPLP2\|SNORA52 | GGCACUUCUAGACACUCU \| GGUCCAUCCUAAUCCCUG | circRPLP2 \| SNORA5 2 |
| 93 | 11:86003452-85996825\|- | ENSG00000073921 | PICALM | UAGCAAGUACAUGGGGAG \| GCCCCUAGCAGUCUUCUU | circPICALM.1 |
| 94 | 11:86031612-85996825\|- | ENSG00000073921 | PICALM | UAGCAAGUACAUGGGGAG \| ACUUAAUUCAGUGCACAA | circPICALM.2 |
| 95 | 11:9428765-9428954\|+ | ENSG00000201998 \|ENSG00000205339 | SNORA23\|IPO7 | UCCAAAUUCCCACACACA \| CAUGGCUGCUGUAAUGUG | circSNORA23\|IPO7. 2 |
| 96 | 11:9428767-9428952\|+ | ENSG00000201998 \|ENSG00000205339 | SNORA23\|IPO7 | CCUCCAAAUUCCCACACA \| UGGCUGCUGUAAUGUGUG | circSNORA23\|IPO7. 3 |
| 97 | 1:196725120-196728523\|+ | ENSG00000000971 | CFH | CAGAAGUGAACUGCUCAA\| AAAGAGAAUGCGAACUUC | circCFH |
| 98 | 12:104928695 -104909654\|- | ENSG00000136052 | SLC41A2 | UCCAGAUUAUCCACUGCA\| AUCCAGUCCUUCUGUGGA | circSLC41A2.1 |
| 99 | 12:104928695 -104927972\|- | ENSG00000136052 | SLC41A2 | GUACUGGAUAUAGUACAG \| AUCCAGUCCUUCUGUGGA | circSLC41A2.2 |
| 100 | 12:108654411 -108652271\|- | ENSG00000110880 | CORO1C | CAAAUGUGAGAUUGCCAG \| AAAAAUAUGCAGGAACCA | circCORO1C |
| 101 | 1:21089214-21050865\|- | ENSG00000075151 \| ENSG00000236073 | EIF4G3 \| RP11-487E1.2 | UCCCCUGGACUCUCUCAA\| AAUUCCUAGAGGACCUGU | circEIF4G3 \| RP11-487E1.2 |
| 102 | 12:112078742 -112075677\|- | ENSG00000111300 | NAA25 | GCGCCUCUUACUAAAAAA \| UCUAUAUCGGCACAGGAU | circNAA25 |
| 103 | 12:116237706 -116230532\|- | ENSG00000123066 | MED13L | GAUUUAUUUAUUUUUCAG \| GCUGAACUCACGGGAAUC | circMED13L |
| 104 | 1:211793191-211778917\|- | ENSG00000123684 \| ENSG00000241395 | LPGAT1\|RN75L344P | CACACAUGUACAUUACAG \| UGAUGGAAUGGGGAGAAG | circLPGAT1 \|RN7SL 344P |
| 105 | 12:125114476-125118766\|+ | ENSG00000081760 | AACS | CCUGGUUGACAGGAUAGG \|GGCACCCUCAUCCAGCAU | circAACS |
| 106 | 1:223806948-223804173\|- | ENSG00000143514 | TP53BP2 | UAAGCAAUGGGAAACUUG \| GUUAAUAGUCCUAGGAUG | circTP53BP2 |
| 107 | 12:23896025-23845982\|- | ENSG00000134532 | SOX5 | UCAAAAACGAGCCGGAAG \| GAUGUCUUCCAAGCGACC | circSOX5 |
| 108 | 1:224952669-224974154\|+ | ENSG00000185842 | DNAH14 | UAAGACAGAGAAGAGCAG \| CCAGUUCCUUUAUAGUUU | circDNAH14 |
| 109 | 1:23030468-23050521\|+ | ENSG00000004487 \|ENSG00000263793 | KDM1A\| MIR3115 | UUCAUUAGAAACCGCACA \| GUAGAGUACAGAGAGAUG | circKDM1A\| MIR31 15 |
| 110 | 1:230961304-230954332\|- | ENSG00000143643 | TTC13 | UUUUCAACAAUGCUUCAG \| AGUCAUCCUUUUUGAACU | circTTC13 |
| 111 | 1:231374100-231370561\|- | ENSG00000135766 | EGLN1 | ACCAGCAUAUGCUACAAG \| GCCAUGGUUGCUUGUUAU | circEGLN1 |
| 112 | 1:23419140-23408663\|- | ENSG00000204219 | TCEA3 | AAGACCAUCGGUGGAAAG \| GAAGGGGCCCUGGACCUU | circTCEA3 |
| 113 | 1:235127934-235127809\|- | ENSG00000173726 \| ENSG00000207181 | TOMM20\|SNORA14B | GUCUUAUUUCAUACCUGC \| AUUCUUAAACCCUCUUGG | circTOMM20\|SNO RA14B |
| 114 | 1:246726891-246740302\|+ | ENSG00000143653 | SCCPDH | CCAGAAAUAAAAUGAAUG \| GAAGACCAACACUGUCAU | circSCCPDH |
| 115 | 1:247159814-247155565\|- | ENSG00000196418 | ZNF124 | AGCUGAGGCAAGACUUCU \| AACUCGGUUGCCUUUGAG | circZNF124.2 |
| 116 | 12:56478263-56477659\|- | ENSG00000135423 | GLS2 | GUCAGCUCCCUGAUCAAG \| GUGGCAGCCUACAUCCCU | circGLS2 |
| 117 | 12:57398199-57395748\|- | ENSG00000179912 | R3HDM2 | GGCUGAGCAUUUAUUCAG \| AGUCUCGCUCUUUUGCCU | circR3HDM2 |
| 118 | 1:26446315-26447661\|+ | ENSG00000117682 | DHDDS | AGGCCUGUUGGAUCCCAG \| GGAGAAACUGCAGAAGCA | circDHDDS |
| 119 | 1:28507387-28507570\|+ | ENSG00000180198 \|ENSG00000242125\| ENSG00000274266 | SNORA73A\|RCC1\|SNHG3 | AAACCAUGCAGGAAACAG \| GUCACUCUCCCCGGGCUC | circSNORA73A \| RCC 1\|SNHG3.1 |
| 120 | 1:28507388-28507564\|+ | ENSG00000180198 \|ENSG00000242125 \|ENSG00000274266 | SNORA73A\|RCC1\|SNHG3 | GGAGACAAACCAUGCAGG \| UCACUCUCCCCGGGCUCU | circSNORA73A \| RCC 1\|SNHG3.2 |
| 121 | 1:28579894-28579763\|- | ENSG00000197989 \|ENSG00000278274 | SNORA61\|SNHG12 | GGCUAGUUUCAGACAGGU \| AUCCUCCUGAUCCCUUUC | circSNORA61 \| SNH G12 |
| 122 | 12:94424903-94424426\|- | ENSG00000173588 \| ENSG00000213250 | CEP83 \| RBMS2P1 | AGGUGUUGGCUUUGCAAG \| CCAUAUGUGUCAUUGGCU | circCEP83\| RBMS2P1 |
| 123 | 12:95211268-95208842\|- | ENSG00000180263 | FGD6 | CAGACAUCAGCAUUCCAG \| AGAUAAAGAAGCCACCAG | circFGD6 |
| 124 | 1:30981406-30980061\|- | ENSG00000134644 | PUM1 | CUGCAGCAGCGACACUAG \| CUGUUCCAAAGACCUAAU | circPUM1 |
| 125 | 13:113495501 -113520734\|+ | ENSG00000150403 \| ENSG00000276916 | TMCO3\| RP11-230F18.6 | CGCCAGUGCAUCUUCUAG \| CUGAAAAUGUGUGUCUGA | circTMCO3 \| RP11-230F18.6 |
| 126 | 1:31919659-31915894\|- | ENSG00000184007 \|ENSG00000269967 | PTP4A2 | GAAGGAAUCCACGUUCUA \| GUUUUUCGUUGGAAUAUA | circPTP4A2 |
| 127 | 13:19852191-19851354\|- | ENSG00000132950 | ZMYM5 | CAACUCAUCAUAGUCCUG \| GUUCAUUGGCAUGGAAAA | circZMYM5 |
| 128 | 13:20732122-20731840\|- | ENSG00000150456 | N6AMT2 | AGAUUCUGCUGUGCACAG \| AAUCGCAUGUGUGAGUGC | circN6AMT2 |
| 129 | 13:27255210-27255522\|+ | ENSG00000122026 \| ENSG00000207051 | RPL21\|SNORA27 | GUAUCCUGUCAGAGGAAA\| UGCUGGUAUAUAACAUUG | circRPL21\|SNORA2 7 |
| 130 | 13:45151686-45207506\|+ | ENSG00000188342 | GTF2F2 | CUACAUGCGAUUAAAAAG \| GUGUCAUUUACUUUGAAU | circGTF2F2 |
| 131 | 1:35358924-35361790\|+ | ENSG00000146463 | ZMYM4 | GAACCUGACAAUGCUCAA\| GUGGUGGUAUCAUGGAUA | circZMYM4 |
| 132 | 13:64034538-64002384\|- | ENSG00000227674 | LINC00355 | AGUCACUGAUGCUCAAAC\| GCCUCCAUUUGCUGUUUG | circLINC00355 |
| 133 | 13:64219224-64245479\|+ | UNKNOWN00000004 | UNKNOWN00000004 | ACGGUAGUGGUUGCUAAG \| GACAUUGUUGGUGAAUUU | circUNKNOWN000 00004 |
| 134 | 13:98278192-98278430\|+ | ENSG00000152767 | FARP1 | UGUGUGAGCAUUGUGUAU \| GUUCUGUGUGUCUUUGUG | circFARP1 |
| 135 | 14:102040235 -102040674 \| + | ENSG00000197102 | DYNC1H1 | GUGAAGAAACACCUGCAA \| GGAGCCCACCUACGAUGC | circDYNC1H1 |
| 136 | 14:103012189 -103003927\|- | ENSG00000198752 | CDC42BPB | CAGGACGAGAACCACCUG \| CUAAACCAUUUACACAGC | circCDC42BPB |
| 137 | 14:20323329-20323178\|- | ENSG00000100814 \|ENSG00000222489 \|ENSG00000281684 | ccNB1IP1\|SNORA79\| AL355075.1 | UUGCGGAGUCAAACAAUU \| UUUGAUGGCUGUUCCUCU | circCCNB1IP1\|SNO RA79\|AL355075.1 |
| 138 | 14:20343169-20343070\|- | ENSG00000259001 \|ENSG00000277209 | RPPH1\|RPPH1 | UACUCUCCUCCGCCCAUU \| GAACAGACUCACGGCCAG | circRPPH1\|RPPH1.1 |
| 139 | 14:20343196-20343073\|- | ENSG00000259001 \|ENSG00000277209 | RPPH1\|RPPH1 | GACUACUCUCCUCCGCCC \| GGCGGAUGCCUCCUUUGC | circRPPH1\|RPPH1.2 |
| 140 | 14:20343278-20343146\|- | ENSG00000259001 \|ENSG00000277209 | RPPH1\|RPPH1 | AGACUCACGGCCAGCGAA\| GUGAGUUCCCAGAGAACG | circRPPH1\|RPPH1.3 |
| 141 | 14:20343292-20343176\|- | ENSG00000259001 \|ENSG00000277209 | RPPH1\|RPPH1 | GCGGAUGCCUCCUUUGCC\| UGAGCUUCGGGGAGGUGA | circRPPH1\|RPPH1.4 |
| 142 | 14:21397402-21397291\|- | ENSG00000100888 \| ENSG00000200785 | SNORD8\|CHD8 | CAUGAAGAUCUGAGGGGC\| UCCCAAUGAUGAGUUGCC | circSNORD8 \| CHD8.1 |
| 143 | 14:21397403-21397291\|- | ENSG00000100888 \| ENSG00000200785 | SNORD8\|CHD8 | CAUGAAGAUCUGAGGGGC\| GUCCCAAUGAUGAGUUGC | circSNORD8 \| CHD8.2 |
| 144 | 14:23302160-23301878\|- | ENSG00000235194 | PPP1R3E | GCCCUGGGCUCCCUCCUC \| GUAGGCGGCGGGGGCACC | circPPP1R3E |
| 145 | 14:24211852-24209884\|- | ENSG00000254505 \|ENSG00000254692\| ENSG00000260669 | CHMP4A\|RP11-468E2.1\|AL136419.6 | AGCAGUUGGCUGAGUGGG \| CCUACUCCACUGGACUCC | circCHMP4A \| RP11-468E2.1\|AL136419 .6 |
| 146 | 14:34554913-34551713\|- | UNKNOWN00000005 | UNKNOWN00000005 | UCCAGAGUGCAGAUGAUG \| AGAAAUCACAGAGUUGCA | circUNKNOWN000 00005 |
| 147 | 14:39033640-39033513\|- | ENSG00000100934 | SEC23A | AUAAAGACACUCCUAUUA \| AAAGCACCAAGCCUCUAA | circSEC23A |
| 148 | 1:44776139-44776644 \|+ | ENSG00000142937 \| ENSG00000200913 | SNORD46\| RPS8 | UGCCUUGCUCUCCUUGGU \| GGUGGGUGCGAGCGUGGG | circSNORD46\| RPS8 |
| 149 | 14:54702061-54702581\|+ | ENSG00000020577 | SAMD4A | GAACCAGCACAAGUACAA \| GAAUCAUUAACCAAUGGC | circSAMD4A |
| 150 | 14:70988566-70995926\|+ | ENSG00000100731 | PCNX | GUUUGCACGAUGAACUUG \| CAGCACAAGCCAGCGAGG | circPCNX |
| 151 | 14:73147794-73148095\|+ | ENSG00000080815 | PSEN1 | AACCACCUGAGCAAUACU \| ACCUAAUCUGGGAGCCUG | circPSEN1 |
| 152 | 14:74735857-74736088\|+ | ENSG00000119616 | FCF1 | GAUGCUGUUUUGCCACUG \| UGCCUGGCCUGCUCACUU | circFCF1 |
| 153 | 14:95533539-95533354\|- | ENSG00000247092\| ENSG00000252481 | SCARNA13\|SNHG10 | CUAAGUACUGCCACAAGU \|GCCUGCUCGAGAGCCAGC | circSCARNA13 \| SNHG110.1 |
| 154 | 14:95533548-95533356\|- | ENSG00000247092 \|ENSG00000252481 | SCARNA13\|SNHG10 | CACUAAGUACUGCCACAA\| CUGAGGCAUGCCUGCUCG | circSCARNA13 \| SNHG10.2 |
| 155 | 14:95533605-95533408\|- | ENSG00000247092 \|ENSG00000252481 | SCARNA13\|SNHG10 | AGAAGCUUUGCAGUCGAG \| GGUUGGUGGUACCCUCGA | circSCARNA13 \| SNH G10.3 |
| 156 | 14:99458278-99465814\|+ | UNKNOWN00000006 | UNKNOWN00000006 | UCUUACCCAUUUUUCUGA \| CUGGAUGACUUUAUAGAA | circUNKNOWN00000006 |
| 157 | 15:29773358-29761138\|- | ENSG00000104067 | TJP1 | GCAGCCAAGCAAUGGCAG \| GCUCCUGGAUUUGGAUUU | circTJP1 |
| 158 | 15:32533369-32522447\|- | ENSG00000223509 | RP11-632K20.7 | ACUGCCCCUGGACCACAG \| GUUAUCCAAGGACAUGGA | circRP11-632K20.7 |
| 159 | 15:42878685-42872610\|- | ENSG00000128881 | TTBK2 | UUUUGAAAAAGCUGCAAG \| GUUAAAUGGAAACCACCC | circTTBK2 |
| 160 | 15:64156725-64156161\|- | ENSG00000166794 | PPIB | ACCAUGCCCUCCUGCUUC \| GUGAGUUGUGGAAGCAAG | circPPIB |
| 161 | 15:75859877-75873569\|+ | ENSG00000140367 | UBE2Q2 | AGGCAAGACCAUUUAAAU \| CUUCGUCAGCAAUUGAAG | circUBE2Q2 |
| 162 | 15:76292701-76287845\|- | ENSG00000140374 | ETFA | UGAGAACUAUUUAUGCAG \| GUGGCACAAGAUCUCUGU | circETFA |
| 163 | 15:84691645-84680712\|- | ENSG00000140612 | SEC11A | UGUGGGGAGAGCCAGGGG \| CUCUAUUAUCAAGUCCUA | circSEC11A |
| 164 | 15:85064369-85067205\|+ | ENSG00000073417 | PDE8A | UGCAGAGGCACUGUGCAG \| GUAGCAGUAGCUGAUGUG | circPDE8A |
| 165 | 1:58536742-58506059\|- | ENSG00000162600 \| ENSG00000173406 | DAB1\|OMA1 | GAUAGACUUAUACCUCAG \| GGGCAUAAGGAAAUGGUG | circDAB1\|OMA1 |
| 166 | 15:89113724-89116522\|+ | ENSG00000140526 | ABHD2 | UCCUCUUCUGACCAAAGA\| CUUUUAGGUUUGUUUGAA | circABHD2 |
| 167 | 15:90439331-90443479\|+ | ENSG00000140575 | IQGAP1 | AUAUAAACAAAGUCAAUA\| UUUGUACCUGUUCAAGCU | circlQGAP1.1 |
| 168 | 15:90441505-90443479\|+ | ENSG00000140575 | IQGAP1 | AUAUAAACAAAGUCAAUA\| UACAUGCUGCUGUUAUUG | circlQGAP1.2 |
| 169 | 15:92996956-93002318\|+ | ENSG00000173575 | CHD2 | AAAGAUAAGAAAGAGAAG \| GAAAAGGACCAGGGAAAA | circCHD2 |
| 170 | 15:98707561-98708108\|+ | ENSG00000140443 | IGF1R | CAAACCGCUGCCAGAAAA\| UCUGCGGGCCAGGCAUCG | circIGF1R |
| 171 | 16:100510-98144\|- | ENSG00000103148 | NPRL3 | GACCUGGCCUUGCUGCAG \| CCUGUGCACGUCGGGCGU | circNPRL3 |
| 172 | 16:15700735-15700927\|+ | ENSG00000072864 | NDE1 | GAACAGAGAACUUAACAG \| CCACCGUGCCUGGCCCCU | circNDE1 |
| 173 | 16:16007815-16016622\|+ | ENSG00000103222 | ABCC1 | GAAACCAUCCACGACCCU \| GACUGGAAUGUCACGUGG | circABCC1 |
| 174 | 16:1963149-1962930\|- | ENSG00000140988 \| ENSG00000207405 | RPS2\|SNORA64 | UGUAGGGCCACCAGCUGC \| GUACCCGGCUGUCCUGGA | circRPS2 \| SNORA64 |
| 175 | 16:22308147-22309511\|+ | ENSG00000058600 | POLR3E | CUGCACUCUACAGGCAAG \| UACCCUGUGCGUCCAGCC | circPOLR3E |
| 176 | 16:28835758-28835908\|+ | ENSG00000168488 | ATXN2L | CAGGAUUCUGUGGUCUUC\| GUAAGAGCCCAGCUGUCC | circATXN2L |
| 177 | 16:29833732-29834067\|+ | ENSG00000013364 | MVP | GCAAGGAGAGGGUGACAG \| GACAUCACACCCCUGCAG | circMVP |
| 178 | 16:29905787-29906128\|+ | ENSG00000174939 | ASPHD1 | CCUAGUACAACCAAACAG \| GCUCCCCCGAAGAUGGGC | circASPHD1 |
| 179 | 16:30483826-30484264\|+ | ENSG00000005844 | ITGAL | AGAUCUAUGUCAUUGAGG \| GACAGAGGUGUUCCGGGA | circITGAL |
| 180 | 1:631070- | ENSG00000237973 | RP5-857K21.6 | ACAGCUCUAAGCCUCCUU \| | circRP5-857K21.6.1 |
| | 631182\|+ | | | CUGAUGUUCGCCGACCGU | |
| 181 | 1:631070-631265\|+ | ENSG00000237973 | RP5-857K21.6 | CACAGCCCAUGCAUUUGU \| CUGAUGUUCGCCGACCGU | circRP5-857K21.6.2 |
| 182 | 1:631070-631364\|+ | ENSG00000237973 | RP5-857K21.6 | UAUGGCGUUUCCCCGCAU \| CUGAUGUUCGCCGACCGU | circRP5-857K21.6.3 |
| 183 | 1:631070-631373\|+ | ENSG00000237973 | RP5-857K21.6 | UCCCCGCAUAAACAACAU \| CUGAUGUUCGCCGACCGU | circRP5-857K21.6.4 |
| 184 | 16:31722625-31723354\|+ | ENSG00000197302 | ZNF720 | CAGUAGCCAUCCAGCCAG \| GGACUGUUGACAUUCAGG | circZNF720 |
| 185 | 16:48256609-48277480\|+ | ENSG00000102910 | LONP2 | GCAGCAGCUCUGCUUGAG \| UUGGUUGAAAUGUUGGAU | circLONP2 |
| 186 | 16:53141178-53157542\|+ | ENSG00000177200 | CHD9 | CUAUGUUUACAGCGACAG \| UCAUUUUUUGAGACCAGU | circCHD9 |
| 187 | 16:68266592-68275250\|+ | ENSG00000103064 | SLC7A6 | UGGCUGCUGCUUGCAUAU \| AGUUUAUGUGGCCGAGGC | circSLC7A6 |
| 188 | 16:8859336-8858349\|- | ENSG00000153048 | CARHSP1 | CUUCCUGCACAUCUCUGA\| GUCAGCCAUGUCAUCUGA | circCARHSP1 |
| 189 | 16:89783103-89782858\|- | ENSG00000187741 | FANCA | GCUGGGGACAUUACUGAG \| GUGCACAUUCUCCACCCA | circFANCA |
| 190 | 17:35103454-35103354\|- | ENSG00000185379 \|ENSG0000026761 8 | RAD51D \| RAD51L3-RFFL | UCCCUGGUUCCCCACUGC \| GUGAGUGAUGUGGCAGAG | circRAD51D \| RAD5 1L3-RFFL |
| 191 | 17:44083855-44083544\|- | ENSG00000108840 | HDAC5 | GCUGCAGGAGAGCUCAAG \| GCCCCAUCAGCCAGAAGA | circHDAC5 |
| 192 | 17:51263273-51268905\|+ | ENSG00000011260 | UTP18 | GGAAAACAUCUUCAGAUG \| GUUCAAGAACAUGAAGAC | circUTP18 |
| 193 | 17:58006412-58005869 \|- | ENSG00000136450 | SRSF1 | GGCACUGGUGUCGUGGAG \| GUGGCGGAGCUCCCCGAG | circSRSF1 |
| 194 | 17:60623520-60648083\|+ | ENSG00000170836 | PPM1D | GAGAAAAAAUACCUGAUG \| CGGAAUGGCCAAAGACUA | circPPM1D |
| 195 | 17:61780402-61776400\|- | ENSG00000136492 | BRIP1 | UUCUUGCCAUCUUACAAG \| GCCUUUUCAGAUAUUAAU | circBRIP1 |
| 196 | 17:66496200-66514467\|+ | ENSG00000154229 | PRKCA | UCAUCCUGGGAUUCAAAC\| UUUGCUGUUUUGUGGUCC | circPRKCA.1 |
| 197 | 17:66513335-66514467\|+ | ENSG00000154229 | PRKCA | UCAUCCUGGGAUUCAAAC \| UGAAGCCUGAGACCUGCC | circPRKCA.2 |
| 198 | 17:7576810-7576952 \|+ | ENSG00000161960\|ENSG00000238917\|ENSG00000264772\|ENSG00000277957 | EIF4A1\|SNORD10\|RP11-186B7.4 \| SENP3-EIF4A1 | GUCUUUGUACUCUGAGAG \| GCUCUGUGAUGGAGCCCA | circEIF4A1 \| SNORD 10\|RP11-186B7.4\| SENP3-EIF4A1.1 |
| 199 | 17:7576810-7576953 \|+ | ENSG00000161960\|ENSG00000238917\|ENSG00000264772\|ENSG00000277957 | EIF4A1\|SNORD10\|RP11-186B7.4 \| SENP3-EIF4A1 | UCUUUGUACUCUGAGAGC \| GCUCUGUGAUGGAGCCCA | circEIF4A1 \| SNORD 10\|RP11-186B7.4\| SENP3-EIF4A1.2 |
| 200 | 17:78400676-78400856\|+ | ENSG00000087157 | PGS1 | UGGUGCAUCCUUACAAAG \| UGCAAACCUGAGUGACUC | circPGS1 |
| 201 | 17:80619626-80643811\|+ | ENSG00000141564 | RPTOR | GAGAACUGGCAGCCAAGG \| CUCAUUUGGUGGCAACGC | circRPTOR |
| 202 | 17:8379519-8379345\|- | ENSG00000161970 \| ENSG00000263809 | RPL26\|RP11-849F2.7 | ACCUCAGGUGAUUUGCCU \| CACUGCAGCCUCCACCUC | circRPL26\| RP11-849F2.7 |
| 203 | 1:810171-805798\|- | ENSG00000230092 | RP11-206L10.8 | UUCAAUUAUGUGGUCAAA\| UAUCUUAAAUAGUGAAGA | circRP11-206L10.8 |
| 204 | 18:46821073-46815311\|- | ENSG00000078043 | PIAS2 | AUUCCAGUUGAUCCCCAG \| GGUUCUCAUGUAUCAGCC | circPIAS2 |
| 205 | 18:670691-671452\|+ | ENSG00000176890 | TYMS | GAGCCACUGAAAAUUCAG \| AUCUUCCUCUGAUGGCGC | circTYMS |
| 206 | 18:9595154-9583116\|- | ENSG00000154845 | PPP4R1 | AGUGAUCCUUCACGUUGG \| UUGGUGUGGAUGACUACA | circPPP4R1 |
| 207 | 19:23362726-23358429\|- | ENSG00000167232 | ZNF91 | GACCUUUUCUAUGGAAAG \| GUAUAUGUCCUCAUUUUC | circZNF91 |
| 208 | 19:36089037-36089307\|+ | ENSG00000075702 | WDR62 | CCAGGACCGCAAUGUGAG \| GCAACAGAGACAUCCAGA | circWDR62 |
| 209 | 19:40702600-40700472\|- | ENSG00000123815 | ADCK4 | AAGCACGAAGUCCCCUUC\| GUGAGCUGAGUGCCCUGA | circADCK4 |
| 210 | 19:46918487-46937409\|+ | ENSG00000160007 | ARHGAP35 | AGUACAUUGAAGCCACAG \| GAAGAAAUGUUGGCUAUU | circARHGAP35 |
| 211 | 19:48913010-48913565\|+ | ENSG00000104805 | NUCB1 | CCUUCUUCAUACUGCAUG \| GCCACCCGGGACCUUGCC | circNUCB1 |
| 212 | 19:49490618-49490707\|+ | ENSG00000142541 \| ENSG00000199631 | SNORD33\|RPL13A | UCUGAGGCCACCCCAUGG \| CCGGUGAUGAGAACUUCU | circSNORD33\| RPL1 3A.1 |
| 213 | 19:49490620-49490698\|+ | ENSG00000142541 \| ENSG0000019963 1 | SNORD33\|RPL13A | GCACUACCAUCUGAGGCC \| GGUGAUGAGAACUUCUCC | circSNORD33\| RPL1 3A.2 |
| 214 | 19:49490629-49490695\|+ | ENSG00000142541 \| ENSG00000199631 | SNORD33\|RPL13A | CAUGCACUACCAUCUGAG \| AACUUCUCCCACUCACAU | circSNORD33\| RPL1 3A.3 |
| 215 | 19:8876659-8876368\|- | ENSG00000181143 | MUC16 | CAGGGUCAUCGCACUAAG \| CCGGGGUGGUCAGCGAGG | circMUC16 |
| 216 | 1:9934861-9931890\|- | ENSG00000162441 | LZIC | AAGUCUCUACAGACCUUG \| GCUAUUCAGGCAGCUAUC | circLZIC |
| 217 | 20:17962878-17962711\|- | ENSG00000089006\|ENSG00000125850\|ENSG00000212232 | SNX5\|SNORD17\|OVOL2 | AGUGGUGAAAAUCUGAUC\| UGUUCUAGGAACUUGAGG | circSNX5 \| SNORD17 \|OVOL2.1 |
| 218 | 20:17962879-17962711\|- | ENSG00000089006 \|ENSG0000012585 0\|ENSG00000212232 | SNX5\|SNORD17\|OVOL2 | AGUGGUGAAAAUCUGAUC\| CUGUUCUAGGAACUUGAG | circSNX5 \| SNORD17 \|OVOL2.2 |
| 219 | 20:38427443-38427304\|- | ENSG00000196756 \|ENSG00000225091 | SNORA71A\|SNHG17 | GAAGCUUUCACACAACUC \| UCCUGCAUCCGAAAGUGA | circSNORA71A\|SN HG17 |
| 220 | 20:45902605-45902266\|- | ENSG00000100979 | PLTP | GCAGCUGGACCUGCGCAG \| AGCCCAGCAGUGAUUGAC | circPLTP |
| 221 | 20:5109446-5106187\|- | ENSG00000089063 | TMEM230 | GGCUACAUCAGCAAAGGG \| CUGUGUCAGCGUGUUAUG | circTMEM230 |
| 222 | 20:54171671-54157168\|- | ENSG00000019186 | CYP24A1 | AGCGAUAAUACGCCUCAG \| GGAAGGGGAAGACUGGCA | circCYP24A1 |
| 223 | 20:63790791-63775677\|- | ENSG00000130584 | ZBTB46 | GGGCCCACUCGCUGUCCC \| AGUCUGUAGAAGAGGCGA | circZBTB46 |
| 224 | 21:33535321-33534578\|- | ENSG00000159131 | GART | CUGCAGCUUCAUUUUGAG \| CCAUCUCAAUCAGUGACC | circGART |
| 225 | 2:135090997-135093694\|+ | ENSG00000115839 | RAB3GAP1 | ACAUUGCCUGGUAAGAUG \| GGUAUAUUUACUUCUGGC | circRAB3GAP1 |
| 226 | 21:37420298-37472881\|+ | ENSG00000157540 | DYRK1A | CAACCUCUAACUAACCAG \| UGUUAUAGUUUUGCCGCU | circDYRK1A |
| 227 | 21:43786514-43787130\|+ | UNKNOWN00000007 | UNKNOWN00000007 | CUGACAUACCCACUGUGC \| ACCUGACAUACCCACUGU | circUNKNOWN000 00007 |
| 228 | 21:45474703-45474804\|+ | ENSG00000182871 | COL18A1 | GGGCAGGAGACACCGUGG \| ACAUGGACCCACAGCCUC | circCOL18A1.1 |
| 229 | 21:45476519-45476868\|+ | ENSG00000182871 | COL18A1 | AGUGUGCGUAUUGUGUGU \| GUGUGUGUGGUGUGUAAC | circCOL18A1.2 |
| 230 | 2:15558635-15551492\|- | ENSG00000151779 | NBAS | UUAUUGGGAAAUGUCAAG \| CCUAGAGGCAACCAAAAA | circNBAS |
| 231 | 21:8213100-8213555\|+ | ENSG00000278996 | CH507-513H4.1 | GUCGUCCGCCGUCGCGCG \| UCGGCCCCGGCCGGGUGG | circCH507-513H4.1.1 |
| 232 | 21:8213118-8213463\|+ | ENSG00000278996 | CH507-513H4.1 | CGGGAGCCCGCCCCGCGG \| AAGGUCCCGUGCCCGUCG | circCH507-513H4.1.2 |
| 233 | 21:8213164-8213555\|+ | ENSG00000278996 | CH507-513H4.1 | GUCGUCCGCCGUCGCGCG \| GGGGCGUGUUGCGUGCGG | circCH507-513H4.1.3 |
| 234 | 2:189728659-189744789\|+ | ENSG00000151687 | ANKAR | UUCCUAUCUUUAAAAGAG \| GAGGUGAAGCUGUCAUAG | circANKAR |
| 235 | 2:190900563-190924594\|+ | ENSG00000115419 | GLS | UUAGACUUCUACUUCCAG \| AUGUGUUCAGAGCAACAU | circGLS |
| 236 | 2:202464808-202467690\|+ | ENSG00000204217 | BMPR2 | CUGACACAACACCACUCA \| CUUCGCAGAAUCAAGAAC | circBMPR2 |
| 237 | 2:226864603-226867319\|+ | ENSG00000144468 | RHBDD1 | UCAUUUAUUCUCACCAGG \| GUUCAGCCGUCUGUAUAU | circRHBDD1 |
| 238 | 2:233275831-233276005\|+ | ENSG00000085978 \| ENSG00000252010 | ATG16L1\|SCARNA5 | UUUUCAAUCUGAGACCUC\| AUGUGUAUGGGAUCAUGG | circATG16L1 \| SCAR NA5 |
| 239 | 2:233388256-233390484\|+ | ENSG00000077044 | DGKD | GUCAACAACAGUUUUACG \| ACCAUCAUCAAAGAGGGG | circDGKD |
| 240 | 2:241140754-241140520\|- | ENSG00000115687 | PASK | GAUGCCAAGACCACAGAG \| AAGACAGAUGGAGCUCCU | circPASK |
| 241 | 22:50372019-50394136\|+ | ENSG00000100239 | PPP6R2 | GGAGAAGGUCCGCUUCAA\| GUAAAGAGAUUAUAAAUC | circPPP6R2 |
| 242 | 2:32377587-32406558\|+ | ENSG00000115760 | BIRC6 | GAUCAGAUUCUGUGACAG \| CUAAACCAGGUGGACAGG | circBIRC6 |
| 243 | 2:37317180-37316236\|- | ENSG00000115825 | PRKD3 | UCCAUAGUUUAUCAAAAG \| GCUAACUAUAUGUCAGAA | circPRKD3 |
| 244 | 2:61112521-61118117\|+ | ENSG00000162929 \|ENSG00000274769 | KIAA1841\| RP11-493E12.3 | UCAAAGGAAGCAAAAGAA\| AGUCUUGUUCUGUCAUCC | circKIAA1841\| RP11 -493E12.3 |
| 245 | 2:74428943-74428847\|- | ENSG00000114993 | RTKN | UCACCCUUGCCAGUCAUG \| UGGUCCUCGUUACCACCU | circRTKN |
| 246 | 2:85368685-85369831\|+ | ENSG00000115459 | ELMOD3 | GACCUUUCCCCCUUCAAG \| UUGUGAGUACAGAGGUGG | circELMOD3 |
| 247 | 2:99464986-99449335\|- | ENSG00000135945 | REV1 | AGAAUGGAUUGUGGAAAG \| AAGCUCCACCAUGAGGCG | circREV1 |
| 248 | 3:114351879-114350273\|- | ENSG00000181722 | ZBTB20 | ACAUGUUCGUACACACAG \| GUGACAUCAGUUGCAAGG | circZBTB20 |
| 249 | 3:119503531-119517316\|+ | ENSG00000113845 | TIMMDC1 | GCUAAAACUGGAAGAGUG \| CAAUCUGCACAUCGUGCU | circTIMMDC1 |
| 250 | 3:128901349-128902506\|+ | ENSG00000177646 | ACAD9 | UUUCGUUGCGGCCUCCUC \| GUGAGUUGCCAGCCACAG | circACAD9 |
| 251 | 3:129586720-129585951\|- | ENSG00000004399 | PLXND1 | GUGCGCCAGGAGUACCCA \| AUCAACCUGAACGAGAGC | circPLXND1 |
| 252 | 3:13500712-13502981\|+ | ENSG00000163517 | HDAC11 | GGGACCGCUUUGCCAAGC \| GGGGUGGCUUCCACCACU | circHDAC11 |
| 253 | 3:138572933-138570317\|- | ENSG00000114107 | CEP70 | GAGCAGAAAACUUUACAG \| UAACUAUGUUUCCGGUAG | circCEP70 |
| 254 | 3:149846010-149912084\|+ | ENSG00000082996 | RNF13 | UUGAUAGUCAUUUUCAUG \| GUGAUUUUACAACGAGAU | circRNF13.1 |
| 255 | 3:149846010-149921228\|+ | ENSG00000082996 | RNF13 | UACAUAAAUUCAAGAAAG \| GUGAUUUUACAACGAGAU | circRNF13.2 |
| 256 | 3:168041475-168036835\|- | ENSG00000173905 | GOLIM4 | ACCCGAGAGGUGCAGGAG \| AGACUGUAUACAAUUUGA | circGOLIM4 |
| 257 | 3:186784800-186784930\|+ | ENSG00000156976 \|ENSG00000238942 | EIF4A2 \| SNORD2 | AAGUGACAAUUUGAUGUG \| AAAUGAUGGCAAUCAUCU | circEIF4A2 \| SNORD 2.1 |
| 258 | 3:186784800-186784945\|+ | ENSG00000156976 \|ENSG0000023894 2 | EIF4A2 \| SNORD2 | GUGGGAUCGGUAAAUGUG \| AAAUGAUGGCAAUCAUCU | circEIF4A2 \| SNORD 2.2 |
| 259 | 3:195960087-1959591821- | ENSG00000185485 | SDHAP1 | UAUCUGGACUUUGAAAUU \| UGCACAGCCAGAGAACAA | circSDHAP1 |
| 260 | 3:47106121-47097954\|- | ENSG00000181555 | SETD2 | UCUCGUAAGAAGGAUUCA \| GAACACCUUUGUCCUAGA | circSETD2 |
| 261 | 3:47428671-47425484\|- | ENSG00000114650 | SCAP | GCCCACCCUCAAUGGCGG \| UAUGUGGGUGCCCCGGUG | circSCAP |
| 262 | 3:49335597-49335019\|- | ENSG00000114316 | USP4 | UGUGAGGAGAGGUUGAAG \| GUAUCUUAUUGACAGCCG | circUSP4 |
| 263 | 3:51995042-51994151\|- | ENSG00000162244 | RPL29 | UGGGGACCCACAUGCUGC \| GUGAGUGCACUGAAUCAC | circRPL29 |
| 264 | 3:52412810-52414588\|+ | ENSG00000010318 | PHF7 | GUGCAUUAUUUCUGUCUU \| CUGGAAGAGCCUGUAUUG | circPHF7 |
| 265 | 3:52741500-52737585\|- | ENSG00000114904 | NEK4 | GAUGAAUUUGAUAGAGAG \| GAAAGGAAACAGAUUCAU | circNEK4 |
| 266 | 3:58125580-58126763\|+ | ENSG00000136068 | FLNB | GAGGAGCCCACAUCCCCG \| GUCUCCAUGUAGUGGAGG | circFLNB |
| 267 | 3:66243202-66263380\|+ | ENSG00000144741 | SLC25A26 | AGCACAGUUUUAAGAGAG \| CUGCUGCAUUUUUUAUCA | circSLC25A26 |
| 268 | 4:102525511-102537957\|+ | ENSG00000109320 | NFKB1 | UCUUACCCUCAGGUCAAA \| CUUCAGAAUGGCAGAAGA | circNFKB1 |
| 269 | 4:53414614-53428184\|+ | ENSG00000145216 \|ENSG00000282278 | FIP1L1 \| RP11-231C18.3 | CUCUGAUUCCACCACCGG \| AAACAGCACUUCUUCUCA | circFIP1L1 \| RP11-231C18.3 |
| 270 | 4:6923372-6924112\|+ | ENSG00000132405 | TBC1D14 | UAGUAACUUCUUUGCAAG \| UUUCUCCUUGGACCAAGA | circTBC1D14 |
| 271 | 4:73404341-73406704\|+ | ENSG00000163631 | ALB | AUUAGUGAAUGAAGUAAC \| CUUUAUUUCCCUUCUUUU | circALB.1 |
| 272 | 4:73413492-73413603\|+ | ENSG00000163631 | ALB | AAGGAUGUUUGCAAAAAC \| CUCUGUUGGAAAAAUCCC | circALB.2 |
| 273 | 4:73415067-73417639\|+ | ENSG00000163631 | ALB | AUGUUGUAAACAUCCUGA \| UUACUCUGUCGUGCUGCU | circALB.3 |
| 274 | 4:76144474-76134174\|- | ENSG00000138750 | NUP54 | AACAUUGCCAGAUGAUCA \| GUGGGUUUGGAGGAUUUG | circNUP54 |
| 275 | 4:87046165-87047595\|+ | ENSG00000172493 | AFF1 | CCUUCUCAGUCAGUUGAG \| UUUGUACAAUGACGACAG | circAFF1 |
| 276 | 5:1065479-1057470\|- | ENSG00000113504 \| ENSG00000263834 | SLC12A7\| MIR4635 | CCUCUUCAGCAUGAAGCC \| AACAUACGGUCCCUAAUG | circSLC12A7 \| MIR4 635 |
| 277 | 5:109713519-109716265\|+ | ENSG00000112893 | MAN2A1 | AUUCCCAUAACGACCCAG \| GGCCAGCUCUCAAUGUUG | circMAN2A1.1 |
| 278 | 5:109713519-109729514\|+ | ENSG00000112893 | MAN2A1 | GAAGGAUGCUGUUAAAAG \| GGCCAGCUCUCAAUGUUG | circMAN2A1.2 |
| 279 | 5:132893119-1328921631- | ENSG00000072364 | AFF4 | AGCUCCAAGGAGGUUAAG \| AAUGAAGAUGAUAACCGA | circAFF4 |
| 280 | 5:139600371-139614967\|+ | ENSG00000131508 | UBE2D2 | CACUAACUAUUUCAAAAG \| GAAUUGAAUGAUCUGGCA | circUBE2D2 |
| 281 | 5:140440118-140445976\|+ | ENSG00000131503 \| ENSG00000254996 | ANKHD1\|ANKHD1-EIF4EBP3 | CACUUGCUUGCUACAAAG \| UCGCAGUCUAGCAGAAGC | circANKHD1 \| ANKH D1-EIF4EBP3 |
| 282 | 5:180280609-180261683\|- | ENSG00000050748 | MAPK9 | GCUGGUAUAAUUCAUAGA \| GGAUCUGAAACUUGCCCA | circMAPK9 |
| 283 | 5:57246299-57251142\|+ | ENSG00000062194 | GPBP1 | ACUUGAGGCAGAACACAG \| AAUAUCCUCCGAAUCCUA | circGPBP1 |
| 284 | 5:618989-655585\|+ | ENSG00000112877 | CEP72 | GUUAGCACCUUCACUCCG \| CUGAGCUUCAGUCAUUGU | circCEP72 |
| 285 | 5:69914157-69916256\|+ | ENSG00000198237 | RP11-98J23.2 | CUUGUAAGGCUUCACCAG \| GAUGGUGAUUGCUCACAC | circRP11-98J23.2 |
| 286 | 5:74841680-74834425\|- | ENSG00000198780 | FAM169A | AUUCAGUUAAGCCUACAG \| AGGAUGGCAUUCCCUGUG | circFAM169A |
| 287 | 5:77464810-77463094\|- | ENSG00000164253 | WDR41 | GCUGAUAGAACAGUUAUU \| AUUUGCAUCUGCUGGUGA | circWDR41 |
| 288 | 5:81123641-81127164\|+ | ENSG00000113319 | RASGRF2 | GACAUGGGAGUCCACCAG \| CAGUCCUAGAGUCUGCAC | circRASGRF2 |
| 289 | 5:95755395-95763621\|+ | ENSG00000164292 | RHOBTB3 | UUAAAAACACCAGGAAAG \| AAAAAAUGCCUGUCUUAA | circRHOBTB3 |
| 290 | 6:118566317-118511297\|- | ENSG00000111860 | CEP85L | UAUGUGGCUAGUUUGCAG \| AUCAUUCAACUUCAAGUG | circCEP85L |
| 291 | 6:157036834-157084906\|+ | ENSG00000049618 | ARID1B | CUCCUGCAAGUAUCCCAG \| GUUGAAGUCUUGGCCUCG | circARID1B.1 |
| 292 | 6:157084661-157110562\|+ | ENSG00000049618 | ARID1B | CAAUGCCACAGGAAAGAG \| GAUCUGUCUGGCUCCAUU | circARID1B.2 |
| 293 | 6:158312050-158314269\|+ | ENSG00000130338 \|ENSG0000027402 3 | TU LP4\| RP11-732M18.4 | CGGGGCCACAAUAGCGAG \| AUUUGUAAGACUCCAGGG | circTULP4\| RP11-732M18.4 |
| 294 | 6:158448995-158452269\|+ | ENSG00000130338 | TULP4 | UCCGCUCAGGGCUGAAAG \| GUGCUGUUUGGCACGGCC | circTULP4 |
| 295 | 6:158583953-158585426\|+ | ENSG00000146433 | TMEM181 | ACCCUCACAUGUGCAGGG \| CUAAAGCCAAUUCAAAUA | circTMEM181 |
| 296 | 6:26031841-26031708\|- | ENSG00000274267 | HIST1H3B | CCAUCCAUGCUAAGCGAG \| AAAUCGCCCAAGACUUCA | circHIST1H3B |
| 297 | 6:26045383-26045863\|+ | ENSG00000278272 | HIST1H3C | AAAAGGCUCUUUUCAGAG \| ACACUUUUGUGUGUGCUC | circHIST1H3C.2 |
| 298 | 6:27293887-27293974\|+ | UNKNOWN0000000 8 | UNKNOWN00000008 | GUACGGGCCUUUGGCUUU \| GGUCGGCCGGUUAGCUCA | circUNKNOWN00000008 |
| 299 | 6:30650993-30651467\|+ | ENSG00000204564 | C6orf136 | CAAAGACGAGCAUUACCG \| CUUCCCAAGCUCUUCCUU | circC6orf136 |
| 300 | 6:31354061-31268939\|- | ENSG00000204525 \| ENSG00000229836 \| ENSG00000231402 \| ENSG00000234745 \| ENSG00000256166 | HLA-C\| HLA-B \| XXbac-BPG248L24.10\|WASF5P \| XXbac-BPG248L24.13 | CUGUUCCAGAGAGGUGGG \| CUGGAUGUCUCCAUCUCU | circHLA-C \|HLA-B \| XXbac-BPG248L24.10\|WA SF5P\|XXbac-BPG248L24.13.1 |
| 301 | 6:31354666-31269965\|- | ENSG00000204525 \| ENSG00000229836 \| ENSG00000231402 \| ENSG00000234745 \| ENSG00000256166 | HLA-C\| HLA-B \| XXbac-BPG248L24.10\|WASF5P \| XXbac-BPG248L24.13 | GUAGGAGGAAGAGCUCAG \| GUGGAAAAGGAGGGAGCU | circHLA-C \|HLA-B \| XXbac-BPG248L24.10\|WA SF5P\|XXbac-BPG248L24.13.2 |
| 302 | 6:35587095-35580159\|- | ENSG00000096060 | FKBP5 | GUCCUGGUUAGAGAUGGA \| UACCAAAGAAAAAUUGGA | circFKBP5 |
| 303 | 6:42934474-42935671\|+ | ENSG00000137161 | CNPY3 | AGCAAUCGAUUUGCCAAG \| UGUGUAAAUAUGUUGCUG | circCNPY3 |
| 304 | 6:43178882-43179067\|+ | ENSG00000112658 | SRF | UGGCUGGCCAGUCCCUGC \| GUAGGUAGGGAUAUCUUU | circSRF |
| 305 | 6:52995710-52995833\|+ | ENSG00000202198 | RN7SK | GAGUAGCUGCGCUCCCCU \| UCCUCCCUCACCGCUCCA | circRN7SK |
| 306 | 6:5404541-5431173\|+ | ENSG00000145982 | FARS2 | AACAACUGGUCAAUUCAG \| UUAUUUGCUGGUAUAAAG | circFARS2 |
| 307 | 6:54121446-54169573\|+ | ENSG00000146147 | MLIP | GGUCGAGAAACCAAAUAU \| GUCUCUGCUGGUGGUUCU | circMLIP |
| 308 | 6:87215902-87218732\|+ | ENSG00000188994 | ZNF292 | CAUUGGAUAAGGAUAAAG \| ACACUCCUAGAAUAUGCA | circZNF292 |
| 309 | 6:89083752-89084644\|+ | ENSG00000146278 | PNRC1 | AUCUGUGGCACAAUCAAU \| GUUUUAAAAUCAAAGAUG | circPNRC1 |
| 310 | 7:100024308-100023418\|- | UNKNOWN00000009 | UNKNOWN00000009 | UGUGUUUCUUUACUAUUC \| CUCGUGACUGUAAGAGGC | circUNKNOWN00000009 |
| 311 | 7:140702865-140704964\|+ | ENSG00000090266 | NDUFB2 | CAGCCUCACUCUGUACAA \| UGCCGGUGGUGGUGUGCA | circNDUFB2 |
| 312 | 7:152250967-152249875\|- | ENSG00000055609 | KMT2C | AUAGUCUUCUUAUUGCUG \| AUUAUAACAAUGAAAUGG | circKMT2C |
| 313 | 7:158764854-158759485\|- | ENSG00000117868 | ESYT2 | UCAGCAAGGAACCUUCCG \| GGUGUUCUAAGGAUACAU | circESYT2 |
| 314 | 7:24620051-24668661 \|+ | ENSG00000105926 | MPP6 | UUGGAACUACGGUGCCAU \| GACUAAAUCAUUUUGCUA | circMPP6 |
| 315 | 7:35673279-35667433\|- | ENSG00000122557 | HERPUD2 | UCCAUAUGUAAUGCAAGG \| UUGACGAAGGAUCAGAGA | circHERPUD2 |
| 316 | 7:44674410-44675269\|+ | ENSG00000105953 | OGDH | CUGGAGGCAGCUGAUGAG \| GUUUGAGGAGUUCCUACA | circOGDH |
| 317 | 7:64544433-64543706\|- | ENSG00000173041 | ZNF680 | UGGUAGCCAAACCCCCAG \| GGACCACUGACAUUUAGG | circZNF680 |
| 318 | 7:6466243-6466115\|- | ENSG00000136240 \| ENSG00000164535 | KDELR2\| DAGLB | UGGUGGCCGGCGUAGUCC \| GAGACCAUCACCACCCAC | circKDELR2\| DAGLB |
| 319 | 7:6585015-6585261\|+ | ENSG00000136247 | ZDHHC4 | ACGGUCUUUCUUAUUCAG \| GUGUGUGUAACUGGUGUG | circZDHHC4 |
| 320 | 7:6822365-6814763\|- | ENSG00000146574 | CCZ1B | AGGCACAUCGAACCUGAG \| CUUUUUAAUGGUACAUUU | circCCZ1B |
| 321 | 7:72890626-72891111\|+ | ENSG00000196313 | POM121 | CAUGUCAACAUAGUCCAG \| GGGCCAGUGUCAUUCAAA | circPOM121 |
| 322 | 7:73470484-73469516\|- | ENSG00000009954 | BAZ1B | AGGAUUACUGUCCUCGCA \| UGAAACUGGAACGCCAAG | circBAZ1B |
| 323 | 7:74705163-74716951\|+ | ENSG00000263001 | GTF2l | UGGAAGUACCAGCAGAAG \| GUGGUCGUGUGAUGGUAA | circGTF2l |
| 324 | 7:75943780-75943917\|+ | ENSG00000127948 \| ENSG00000201643 | SNORA14A | UUCAUACCUGCAACAUCU \| AUUGCAUUCUUAAACCCU | circSNORA14A |
| 325 | 7:90726566-90726813\|+ | ENSG00000058091 | CDK14 | CACUCCAGCCCCAGCUCG \| AUAUGUGUCACAAAGAUG | circCDK14 |
| 326 | 7:93291702-93297244\|+ | ENSG00000004766 | CCDC132 | UUUCAAGAAUUACCAUAG \| CAUGUUACACCAGACAGC | circCCDC132 |
| 327 | 7:98881699-98881886\|+ | ENSG00000196367 \| ENSG00000266019 | TRRAP\| MIR3609 | CUGAUAAGAUCUGAUUGC \| CAAAGUGAUGAGUAAUAC | circTRRAP\|MIR360 9 |
| 328 | 7:99722019-99721866\|- | ENSG00000160870 \|ENSG00000282301 | CYP3A7\|CYP3A7-CYP3A51P | GUUUUGCCAAGUAUUUUG \| AAAUAGUUCUCCUUCACU | circCYP3A7 \| CYP3A 7-CYP3A51P |
| 329 | 8:127218967-127218809\|- | ENSG00000247844 | CCAT1 | UGCCACUUACCAGGUUGG \| GCCAGGCACUACUCUGUC | circCCAT1.1 |
| 330 | 8:127218967-127218814\|- | ENSG00000247844 | CCAT1 | AGCCCUGCCACUUACCAG \| GCCAGGCACUACUCUGUC | circCCAT1.2 |
| 331 | 8:127220406-127218809\|- | ENSG00000247844 | CCAT1 | UGCCACUUACCAGGUUGG \| GAUUUAAUGGCAAGAUGC | circCCAT1.3 |
| 332 | 8:127223645-127218809\|- | ENSG00000247844 | CCAT1 | UGCCACUUACCAGGUUGG \| GAGCCUGACAUCAUGGUG | circCCAT1.4 |
| 333 | 8:127223667-127218809\|- | ENSG00000247844 | CCAT1 | UGCCACUUACCAGGUUGG \| UUAUUAAUGGCUCUCCUA | circCCAT1.5 |
| 334 | 8:127224685-127218809\|- | ENSG00000247844 | CCAT1 | UGCCACUUACCAGGUUGG \| GACUGUAUUGUCUGCUAG | circCCAT1.6 |
| 335 | 8:127227542-127218809\|- | ENSG00000247844 | CCAT1 | UGCCACUUACCAGGUUGG \| AAAGAGACAGAACAAUGU | circCCAT1.7 |
| 336 | 8:130180881-130152735\|- | ENSG00000153317 | ASAP1 | ACCAUCUCACAUGCCACA \| UACAAAAAUUGAGAAAGA | circASAP1 |
| 337 | 8:140706206-140700890\|- | ENSG00000169398 | PTK2 | AAAGAGGAAAGAUUUCUG \| CCCAGCAGACCGGGUUAU | circPTK2.1 |
| 338 | 8:140890770-140864311\|- | ENSG00000169398 | PTK2 | AAUUUCUUCUAUCAACAG \| AAUAUGACAGAUACCUAG | circPTK2.2 |
| 339 | 8:141254630-141253988\|- | ENSG00000022567 | SLC45A4 | CAAUACUGUUGCAGAUUG \| AUCAUCCAGUCACUCGAU | circSLC45A4 |
| 340 | 8:142501136-142501376\|+ | ENSG00000181790 | ADGRB1 | AUGGUGGUGGUAGUGAUG \| UGCUUAUAGUGGAGGUGA | circADGRB1 |
| 341 | 8:30474778-30479378\|+ | ENSG00000157110 | RBPMS | GCAAAGAAUGCUUUGAAU \| GUCCGGACCCUAUUUGUC | circRBPMS |
| 342 | 8:38457535-38457355\|- | ENSG00000077782 | FGFR1 | CGACCUUGCCUGAACAAG \| GGUCAGUUUGAAAAGGAG | circFGFR1 |
| 343 | 8:42957093-42964475\|+ | ENSG00000168172 | HOOK3 | CCAAGAAGAAACAUUCAG \| CUGAUGAGUAAAGAAUCU | circHOOK3 |
| 344 | 8:61684189-61680967\|- | ENSG00000198363 | ASPH | AUGCCAAAGUUUUAUUAG \| AGACAAAGCAUGGAGGAC | circASPH |
| 345 | 9:109108571-109106507\|- | ENSG00000106771 | TMEM245 | AUGGAAAAGAGUCUUCUG \| AUCUGGACGUUGGUGGUU | circTMEM245 |
| 346 | 9:114074050-114078172\|+ | UNKNOWN00000010 | UNKNOWN00000010 | GCGCUCACCGCCAGGCAG \| UAAUGGUGGGUCCAUGAU | circUNKNOWN00000010 |
| 347 | 9:125238678-125238150\|- | ENSG00000044574 | HSPA5 | CCAACUGUUACAAUCAAG \| GAACCAUCCCGUGGCAUA | circHSPA5 |
| 348 | 9:128508875-128515640\|+ | ENSG00000119392 | GLE1 | CACAGAAUGAAAGGAACA \| GAUGUUUUAGAAGAAUGU | circGLE1 |
| 349 | 9:20881870-20933104\|+ | ENSG00000188352 | FOCAD | CACUAGUCUUGAAUACAA \| CUUUGGAGGAAUUUUUUA | circFOCAD |
| 350 | 9:33351559-33352720\|+ | ENSG00000086102 | NFX1 | AUCUAGUACUUAUCAAAG \| UUUCGGAGCAACAUCCCC | circNFX1 |
| 351 | 9:33948588-33948373\|- | ENSG00000137073 | UBAP2 | CAGUCCUCAGUCAUCUUG \| UCAUCCGUCCUUGGCUCA | circUBAP2 |
| 352 | 9:6880011-6893233\|+ | ENSG00000107077 \|ENSG00000274527 | KDM4C \| RP11-146B14.1 | GGAAAAGUUGCCAAAUUG \| GUAUGCUAUACCUCCGGA | circKDM4C\|RP11-146B14.1 |
| 353 | 9:85633375-85596361\|- | ENSG00000135049 | AGTPBP1 | UGACAUUUGUUACUAUAA \| GACUAUGAUUUAAUCUCC | circAGTPBP1 |
| 354 | 9:93471140-93476339\|+ | ENSG00000048828 | FAM120A | CCACUAGCCUCACUAAAG \| GUUGCACAGAGCAUUGAG | circFAM120A.1 |
| 355 | 9:93471140-93497600\|+ | ENSG00000048828 | FAM120A | GUUUUCCAGCAUUCACAG \| GUUGCACAGAGCAUUGAG | circFAM120A.2 |
| 356 | 9:94429188-94438529\|+ | ENSG00000148110 | HIATL1 | GAUGAGGAUCAGCCCAUG \| GUUCUACAUGAAACAUUU | circHIATL1 |
| 357 | X:362333-361404\|- | ENSG00000167393 | PPP2R3B | AUGGGCCUGGUGGCCAAG \| AUGGUGUUUUAACAUCAC | circPPP2R3B |
| 358 | X:77656654-77652113\|- | ENSG00000085224 | ATRX | CGAGAAAAAUUGAGAGAG \| UGAGCAGUGAAGAUUCAG | circATRX |
| 359 | X:9568616-9569271\|+ | ENSG00000101849 | TBL1X | UGGUGUGCUGUGUGUGUG \| GUGUGCUGUAUGUCUGUC | circTBL1X |
| 2260 | 4:186709846-186706562\|- | ENSG00000083857 | FAT1 | AAATAGGTGAAGAGACAG\|AT TCCCGACAGTTAAGCA | circFAT1 |

In some embodiments, the antisense oligonucleotide of the invention and according to the above embodiments, comprises in total at least three sugar-modified nucleobases that enhance the binding affinity of the antisense oligonucleotide to the circRNA. In one such embodiment, the antisense oligonucleotide according to the invention, comprises a total of at least three sugar-modified nucleobases that enhance the binding affinity of the antisense oligonucleotide to the circRNA, and wherein the antisense oligonucleotide comprises a gap of at least 7, 8, 9, 10, 11, 12, 13 or 14 DNA units, flanked in each end by wings comprising at least one sugar-modified nucleobase.

In some embodiments, the antisense oligonucleotide according to anyone of the above embodiments, comprises sugar-modified nucleobase units selected from the list of LNA (Locked nucleic acid), beta-D-oxy LNA, alpha-L-oxy-LNA, beta-D-amino-LNA, alpha-L-amino-LNA, beta-D-thio-LNA, alpha-L-thio-LNA, 5′-methyl-LNA, beta-D-ENA and alpha-L-ENA, 2′Fluoro, 2′-O-methyl, 2′-methoxyethyl (2′-MOE), 2′cyclic ethyl (cET), UNA and Conformationally Restricted Nucleoside (CRN). In some embodiments, the antisense oligonucleotide comprises only LNA nucleobases in the wings, and in some embodiments, the antisense oligonucleotide of the invention comprises a mixture of LNA and one or more other nucleobase units, such as a mixture of LNA and one or more of tricyclo-DNA, 2′-fluoro, 2′-O-methyl, 2′methoxyethyl (2′-MOE), 2′cyclic ethyl (cET), UNA and Conformationally Restricted Nucleoside (CRN) nucleobase units. In some preferred embodiments, the antisense oligonucleotide comprises a 5′ wing of 2, 3 or 4 LNA nucleobase units, such as in a non-limiting example Beta-D-Oxy LNA units, a central region of 6 to 16 consecutive DNA nucleotides and a 3′ end wing of 2, 3 or 4 LNA nucleobase units, such as in a non-limiting example Beta-D-Oxy LNA units. Where X represents the central region of 6-16 DNA nucleotides, and 2, 3 or 4 represent number of LNA in the wings, an antisense oligonucleotide of the invention may be designed to be complementary to a region overlapping the back-splice junction of anyone of SEQ ID NOs: 1 - 359, and wherein the antisense oligonucleotide is a gapmer that is designed as a 2 × 2, or a 2 × 3, or a 2 × 4, or a 3 × 2, or a 3 × 3, or a 3 × 4, or a 4 × 2, or a 4 × 3, or a 4 × 4 oligonucleotide.

In a preferred embodiment the DNA region X is anyone of 6, 7, 8, 9, 10, 11, 12, 13, 14, 15 or 16 nucleotides in length, such as anyone of 10, 11, 12, 13, 14, 15 or 16 nucleotides in length. In some embodiments, the gap region “X” may comprise one or more gap shortening LNA nucleotides in order to decrease off target effects (as described in Rukov et al. 2015, Nucleic Acids Res. 2015 Sep 30;43(17):8476-87). In some embodiments, one or more LNA nucleotides are inserted in the DNA gap in order to decrease gapsize to be a maximum of 4 DNA, or 5 DNA, or 6 DNA or 7 DNA, or 8 DNA or 9 DNA or 10 DNA or 11 DNA or 12 DNA in length. In some preferred embodiments according to the invention as described throughout the application, each cytosine is a 5-methylcytosine. In some preferred embodiments, the LNA units in the wings of the antisense oligonucleotide of the invention are Beta-D-Oxy LNA and the target region is anyone of SEQ ID NOs: 1- 359 and 2260. In some embodiments, the nucleoside analogues in the wings are not LNA, but tricyclo-DNA and the target region is anyone of SEQ ID NOs: 1- 359 and 2260. In some embodiments, the nucleoside analogues in the wings are not LNA but 2′-Fluoro and the target region is anyone of SEQ ID NOs: 1- 359 and 2260. In some embodiments, the nucleoside analogues in the wings are not LNA but 2′-O-methyl and the target region is anyone of SEQ ID NOs: 1- 359 and 2260. In some embodiments, the nucleoside analogues in the wings are not LNA but 2′-MOE and the target region is anyone of SEQ ID NOs: 1 - 359 and 2260. In some embodiments, the nucleoside analogues in the wings are not LNA but 2′cyclic ethyl (cET) and the target region is anyone of SEQ ID NOs: 1 - 359 and 2260. In some embodiments, the nucleoside analogues in the wings are not LNA but UNA and the target region is anyone of SEQ ID NOs: 1 -359 and 2260. In some embodiments, the nucleoside analogues in the wings are not LNA but CRN and the target region is anyone of SEQ ID NOs: 1 - 359 and 2260. In some embodiments, the nucleoside analogues in the wings are partly LNA but mixed with another nucleotide analogue selected from the list of tricyclo-DNA, 2′-Fluoro, 2′-O-methyl, 2′-methoxyethyl (2′-MOE), 2′cyclic ethyl (cET), UNA, and Conformationally Restricted Nucleoside (CRN) and the target region is anyone of SEQ ID NOs: 1 - 359 and 2260.

In some embodiments, all internucleoside linkages of the antisense oligonucleotide according to the invention are phosphorothioate linkages. In some embodiments, the antisense oligonucleotide of the invention comprises at least one phosphorothioate internucleoside linkage. In some embodiments, the antisense oligonucleotide of the invention comprises at least two phosphorothioate internucleoside linkages, which are the 5′ most linkage and the 3′ most linkage of the antisense oligonucleotide. In some embodiments, the antisense oligonucleotide of the invention comprises at least two phosphorothioate internucleoside linkages, which are the 5′ most linkage and the 3′ most linkage, and wherein all the internucleoside linkages in the DNA gap are phosphorothioate linkages. In certain embodiments, the oligonucleotide comprises at least a total of 6 phosphorothioate internucleoside linkages. In certain embodiments, the oligonucleotide comprises at least a total of 8 phosphorothioate internucleoside linkages. In certain embodiments, the oligonucleotide comprises at least a total of 10 phosphorothioate internucleoside linkages.

In certain embodiments, the antisense oligonucleotide of the present invention, are designed to comprise wings that comprise 1, 2, 3, 4, 5, or 6 sugar modified nucleobase units, such as 2 to 5 modified nucleobase units, such as 2-4 sugar modified nucleobase units.

In certain preferred embodiments, the antisense oligonucleotide according to the present invention is anyone of the antisense oligonucleotides presented in Table 2, corresponding to anyone of SEQ ID NOs: 360 - 2148 and 2285-2299.

The antisense oligonucleotides (ASOs) of the present invention are listed in Table 2 (LNA, such as in a non-limiting example Beta-D-Oxy LNA units = uppercase, DNA = lowercase, complete phosphorothioate backbone, LNA cytosine units are LNA 5-methylcytosines).

TABLE 2

Gapmer antisense oligonucleotides targeting back-splice junction-encompassing sequences for modulation of cancer-associated circRNAs (target sequences are shown in Table 1 for the individual circRNAs)

| SEQ ID NO | Oligonucleotide (5′-3′) | Target name |
|---|---|---|
| 360 | GTgccatcggaaaccCT | ciRS-7 |
| 361 | CATcggaaaccctggatAT | ciRS-7 |
| 362 | TCggaaaccctggatatTG | ciRS-7 |
| 363 | ATcggaaaccctGGA | ciRS-7 |
| 364 | AAccctggatattGCA | ciRS-7 |
| 365 | GAaaccctggatattGC | ciRS-7 |
| 366 | CAtcggaaaccctggaTA | ciRS-7 |
| 367 | ATcggaaaccctggataTT | ciRS-7 |
| 368 | AAagatcaggcCTCA | circPVT1 |
| 369 | AGatcaggcctcaaGC | circPVT1 |
| 370 | AAagatcaggcctCAAG | circPVT1 |
| 371 | CAaaagatcaggcctCAA | circPVT1 |
| 372 | AAagatcaggcctcaagCC | circPVT1 |
| 373 | AGgccatacctgtAG | circHIPK3 |
| 374 | GCcatacctgtagtAC | circHIPK3 |
| 375 | GGccatacctgtagtAC | circHIPK3 |
| 376 | GAggccatacctgtagTA | circHIPK3 |
| 377 | ATacctgtagtaccgagAT | circHIPK3 |
| 378 | TTGTtactgaaATGA | circSRY |
| 379 | ATTCtttgttacTGAA | circSRY |
| 380 | AGattctttgttaCTGA | circSRY |
| 381 | TCtttgttactgaaATGA | circSRY |
| 382 | GATtctttgttactgAAAT | circSRY |
| 383 | CTtcttctcctctGT | circSLC35E2B |
| 384 | TCctctgtcttccaTA | circSLC35E2B |
| 385 | TCttcttctcctctgTC | circSLC35E2B |
| 386 | TCtcctctgtcttccaTA | circSLC35E2B |
| 387 | CTtctcctctgtcttcAT | circSLC35E2B |
| 388 | ACtgggacgtccgTA | circCDK11A |
| 389 | ACtgggacgtccgtAA | circCDK11A |
| 390 | ACtgggacgtccgtaAA | circCDK11A |
| 391 | ACtgggacgtccgtaaAG | circCDK11A |
| 392 | ACtgggacgtccgtaaaGA | circCDK11A |
| 393 | AGGTgttaaaattTT | circUNKNOWN00000001 |
| 394 | AGGTgttaaaatTTTT | circUNKNOWN00000001 |
| 395 | AATTtttatggatGATC | circUNKNOWN00000001 |
| 396 | GTTAaaatttttatGGAT | circUNKNOWN00000001 |
| 397 | AAAGgtgttaaaattTTTA | circUNKNOWN00000001 |
| 398 | TGcgcctctgccTG | circARHGAP32 |
| 399 | CTtgcctgtatgctGC | circARHGAP32 |
| 400 | CGcctcttgcctgtaTG | circARHGAP32 |
| 401 | CGcctcttgcctgtatGC | circARHGAP32 |
| 402 | ATctgcgcctcttgcctGT | circARHGAP32 |
| 403 | GCcagagacacagTT | circSLC8A3 |
| 404 | CCagagacacagtTTC | circSLC8A3 |
| 405 | TAggccagagacacaGT | circSLC8A3 |
| 406 | AGgccagagacacagTT | circSLC8A3 |
| 407 | GAcacagtttcatcattCT | circSLC8A3 |
| 408 | GAacatccccaTTAT | circHERC2 |
| 409 | CGtgaacatccccaTT | circHERC2 |
| 410 | CGtgaacatccccatTA | circHERC2 |
| 411 | TGaacatccccattatGC | circHERC2 |
| 412 | GAacatccccattatgcCA | circHERC2 |
| 413 | ATtccctgcacatCT | circZFAND6 |
| 414 | TCattccctgcacaTC | circZFAND6 |
| 415 | TCagattcattccctGC | circZFAND6 |
| 416 | AGattcattccctgcaCA | circZFAND6 |
| 417 | GActtcagattcattccCT | circZFAND6 |
| 418 | AGgctgcacgggcGA | circRP1-168P16.1 |
| 419 | AGgctgcacgggcgAT | circRP1-168P16.1 |
| 420 | AGgctgcacgggcgaTT | circRP1-168P16.1 |
| 421 | AGgctgcacgggcgatTC | circRP1-168P16.1 |
| 422 | AGgctgcacgggcgattCC | circRP1-168P16.1 |
| 423 | CTttgtcgcctggAC | circAURKC |
| 424 | TCctttgtcgcctgGA | circAURKC |
| 425 | TTtcctttgtcgcctGG | circAURKC |
| 426 | TTcctttgtcgcctggAC | circAURKC |
| 427 | TTcctttgtcgcctggaCA | circAURKC |
| 428 | ATTAatttacaCCTG | circAFTPH |
| 429 | ACacctgaactgAAGT | circAFTPH |
| 430 | TTAcacctgaactGAAG | circAFTPH |
| 431 | ATTAatttacacctGAAC | circAFTPH |
| 432 | TAatttacacctgaacTGA | circAFTPH |
| 433 | AGAgagagttaTCTG | circSCD |
| 434 | GAgagagttatcTGGA | circSCD |
| 435 | CCtagaagagagagaGT | circSCD |
| 436 | AGagagagagttatcTGG | circSCD |
| 437 | TAgaagagagagagTATC | circSCD |
| 438 | ACttggtcacctTAG | circSMC3 |
| 439 | ACttggtcaccttaGG | circSMC3 |
| 440 | TCaccttaggcatgaAG | circSMC3 |
| 441 | TTggtcaccttaggcaTG | circSMC3 |
| 442 | CTtggtcaccttaggcaTG | circSMC3 |
| 443 | ACagcagccatgtGT | circSMC3 |
| 444 | AGccatgtgtgtggGA | circSNORA23\|IPO7.1 |
| 445 | CCatgtgtgtgggaaTT | circSNORA23\|IPO7.1 |
| 446 | CAttacagcagccatgTG | circSNORA23\|IPO7.1 |
| 447 | CAcattacagcagccatGT | circSNORA23\|IPO7.1 |
| 448 | CAACcgagtttaGAG | circZNF124.1 |
| 449 | CAACcgagtttagaGT | circZNF124.1 |
| 450 | AAggcaaccgagttTAG | circZNF124.1 |
| 451 | AGtttagagtagaaacCC | circZNF124.1 |
| 452 | AACcgagtttagagtaGAA | circZNF124.1 |
| 453 | GATCtcagaataAGC | circSNX5 \| OVOL2 |
| 454 | ATCtcagaataagcCC | circSNX5 \| OVOL2 |
| 455 | GATCtcagaataagcCC | circSNX5 \| OVOL2 |
| 456 | ACagatctcagaataAGC | circSNX5 \| OVOL2 |
| 457 | AGatacagatctcagAATA | circSNX5 \| OVOL2 |
| 458 | TGCagctcagtaaCA | circRALY |
| 459 | AGCtcagtaacaaTGA | circRALY |
| 460 | TGcagctcagtaaCAAT | circRALY |
| 461 | GCagctcagtaacaatGA | circRALY |
| 462 | CTgcagctcagtaacaaTG | circRALY |
| 463 | AATccccaagaATGA | circTFPI |
| 464 | AATCcccaagaaTGAA | circTFPI |
| 465 | CCccaagaatgaaatGG | circTFPI |
| 466 | AATCcccaagaatgaAAT | circTFPI |
| 467 | CAAgaatgaaatggtTGTT | circTFPI |
| 468 | CGttcagcggggcCA | circAHSG.1 |
| 469 | CGttcagcggggcCAG | circAHSG.1 |
| 470 | TGtcgttcagcggggCC | circAHSG.1 |
| 471 | CAgcggggccagcaggTT | circAHSG.1 |
| 472 | TGtcgttcagcggggcCAG | circAHSG.1 |
| 473 | GCTTggacaaaaTGG | circAHSG.2 |
| 474 | TGgacaaaatggtGGC | circAHSG.2 |
| 475 | TTggacaaaatggtGGC | circAHSG.2 |
| 476 | GGcttggacaaaatggTG | circAHSG.2 |
| 477 | TTggacaaaatggtggcTT | circAHSG.2 |
| 478 | CTTggacaaaaTGGT | circAHSG.3 |
| 479 | TTGGacaaaatgGTTT | circAHSG.3 |
| 480 | GCttggacaaaatgGTT | circAHSG.3 |
| 481 | CAggcttggacaaaatGG | circAHSG.3 |
| 482 | CAAaatggtttttgtaAGG | circAHSG.3 |
| 483 | GTGgctcatgaaTTA | circUBXN7 |
| 484 | CAgtggctcatgAATT | circUBXN7 |
| 485 | CTCatgaattagaTCTC | circUBXN7 |
| 486 | GGctcatgaattagatCT | circUBXN7 |
| 487 | TCatgaattagatctcAGA | circUBXN7 |
| 488 | CAgttatgtctTGGA | circAFP |
| 489 | AACAgttatgtctTGG | circAFP |
| 490 | TATgtcttggaaaGTTC | circAFP |
| 491 | CAgttatgtcttggAAAG | circAFP |
| 492 | GTtatgtcttggaaagTTC | circAFP |
| 493 | CGcgcagagccttTG | circHIST1H3A |
| 494 | CTcgcgcagagcctTT | circHIST1H3A |
| 495 | TCgcgcagagcctttGC | circHIST1H3A |
| 496 | CGcgagagcctttgcTT | circHIST1H3A |
| 497 | ATctcgcgcagagccttTG | circHIST1H3A |
| 498 | GTgtcagctggaTAT | circHIST1H3C.1 |
| 499 | ACacaaaagtgtCAGC | circHIST1H3C.1 |
| 500 | TGtcagctggatatcTT | circHIST1H3C.1 |
| 501 | TGtcagctggatatctTT | circHIST1H3C.1 |
| 502 | ACaaaagtgtcagctggAT | circHIST1H3C.1 |
| 503 | GTgggctcacgcaGC | circANAPC2 |
| 504 | CTcacgcagcctggGC | circANAPC2 |
| 505 | CTggtgggctcacgcAG | circANAPC2 |
| 506 | GGctggtgggctcacgCA | circANAPC2 |
| 507 | TCacgcagcctgggcacGG | circANAPC2 |
| 508 | TCagtgtggttgGTG | circRMRP\|RMRP |
| 509 | CCtcagtgtggttgGT | circRMRP\|RMRP |
| 510 | GAgtcctcagtgtggTT | circRMRP\|RMRP |
| 511 | TCctcagtgtggttggTG | circRMRP\|RMRP |
| 512 | ACagagtcctcagtgtgGT | circRMRP\|RMRP |
| 513 | AAATtagtacaGGCA | circCENPI |
| 514 | AAtcaaaaattagtAC | circCENPI |
| 515 | AAATtagtacaggCATG | circCENPI |
| 516 | TCaaaaattagtacAGGC | circCENPI |
| 517 | AAattagtacaggcatGGT | circCENPI |
| 518 | ACaccttagtctcCT | circFIRRE |
| 519 | GTctcctcataaaGTA | circFIRRE |
| 520 | CCttagtctcctcatAA | circFIRRE |
| 521 | TAgtctcctcataaagTA | circFIRRE |
| 522 | TCtcctcataaagtatcTC | circFIRRE |
| 523 | ATGAcaaatggCATT | circMBNL3 |
| 524 | AGatgacaaatgGCAT | circMBNL3 |
| 525 | GAagatgacaaatGGCA | circMBNL3 |
| 526 | CTgaagatgacaaatGGC | circMBNL3 |
| 527 | GAagatgacaaatggCATT | circMBNL3 |
| 528 | GGcctagtggtggTC | circGPC3 |
| 529 | CCtagtggtggtcaGC | circGPC3 |
| 530 | TTcaaaggcctagtgGT | circGPC3 |
| 531 | AGgcctagtggtggtcAG | circGPC3 |
| 532 | CCtagtggtggtcagctTT | circGPC3 |
| 533 | ACTccaaagtgaAGC | circPROSER2 |
| 534 | AGctcactccaaagTG | circPROSER2 |
| 535 | TCactccaaagtgaaGC | circPROSER2 |
| 536 | ACagctcactccaaagTG | circPROSER2 |
| 537 | GCtcactccaaagtgaaGC | circPROSER2 |
| 538 | CTccaccaataTTGT | circMALRD1 |
| 539 | CACcaatattgtGTAT | circMALRD1 |
| 540 | AATattgtgtatgATCC | circMALRD1 |
| 541 | CAatattgtgtatgaTCC | circMALRD1 |
| 542 | GCctccaccaatattgtGT | circMALRD1 |
| 543 | CAtaaatgcttTGGC | circFAM208B |
| 544 | CAtaaatgctttgGCT | circFAM208B |
| 545 | TGtcataaatgctTTGG | circFAM208B |
| 546 | TTAAtgtcataaatGCTT | circFAM208B |
| 547 | GTcataaatgctttggcTG | circFAM208B |
| 548 | AGtgaaagaccTGCG | circMCU |
| 549 | AGtgaaagacctgCGA | circMCU |
| 550 | AAagacctgcgaaTGTT | circMCU |
| 551 | GTagtgaaagacctgcGA | circMCU |
| 552 | GAaagacctgcgaatgtTC | circMCU |
| 553 | TAgatctcctcCATT | circKIF20B |
| 554 | GAtctcctccatttCA | circKIF20B |
| 555 | AAcatttagatctCCTC | circKIF20B |
| 556 | TTagatctcctccattTC | circKIF20B |
| 557 | TTtaacatttagatctCCT | circKIF20B |
| 558 | AATAtcctgaaCAGA | circABCC2 |
| 559 | TATCctgaacagATAC | circABCC2 |
| 560 | AATatcctgaacaGATA | circABCC2 |
| 561 | GAaatatcctgaacAGAT | circABCC2 |
| 562 | ATatcctgaacagataCAT | circABCC2 |
| 563 | GTcctcttatctCAT | circEIF4GC\|SNORD 97.1 |
| 564 | TCCtcttatctcaTAA | circEIF4GC\|SNORD 97.1 |
| 565 | AGtcctcttatctcaTA | circEIF4GC\|SNORD 97.1 |
| 566 | ATaaagtcctcttatCTC | circEIF4G2\|SNORD 97.1 |
| 567 | GTcctcttatctcataaTC | circEIF4G2\|SNORD 97.1 |
| 568 | AGtcctcttaaTCTC | circEIF4G2\|SNORD 97.2 |
| 569 | CTTAatctcataATCT | circEIF4G2\|SNORD 97.2 |
| 570 | GTcctcttaatctcaTA | circEIF4G2\|SNORD 97.2 |
| 571 | GTcctcttaatctcaTAA | circEIF4G2\|SNORD 97.2 |
| 572 | TTaatctcataatcttCGC | circEIF4G2\|SNORD 97.2 |
| 573 | CGcgtctttttATCT | circEIF4G2\|SNORD 97.3 |
| 574 | GCgtctttttatctCA | circEIF4G2\|SNORD 97.3 |
| 575 | CGtctttttatctCATA | circEIF4G2\|SNORD 97.3 |
| 576 | CGtctttttatctcATAA | circEIF4G2\|SNORD 97.3 |
| 577 | GTctttttatctcatAATC | circEIF4G2\|SNORD 97.3 |
| 578 | CGCgtctttttaTAT | circEIF4G2\|SNORD 97.4 |
| 579 | GCgtctttttataTCT | circEIF4G2\|SNORD 97.4 |
| 580 | ACgcgtctttttaTATC | circEIF4G2\|SNORD 97.4 |
| 581 | GCgtctttttatatctCA | circEIF4G2\|SNORD 97.4 |
| 582 | CGtctttttatatctcATA | circEIF4G2\|SNORD 97.4 |
| 583 | CGCGtctttttaTAA | circEIF4G2\|SNORD 97.5 |
| 584 | CGTCtttttataaTCT | circEIF4G2\|SNORD 97.5 |
| 585 | CGTCtttttataatCTT | circEIF4G2\|SNORD 97.5 |
| 586 | CTttttcataatcttcGCT | circEIF4G2\|SNORD 97.5 |
| 587 | TTtataatcttcgctcaCA | circEIF4G2\|SNORD 97.5 |
| 588 | TCATcataatcTTCG | circEIF4G2\|SNORD 97.6 |
| 589 | AAtcatcataatcTTC | circEIF4G2\|SNORD 97.6 |
| 590 | AATCatcataatcTTCG | circEIF4G2\|SNORD 97.6 |
| 591 | TCatcataatcttcgcTC | circEIF4G2\|SNORD 97.6 |
| 592 | TATaatcatcataatCTTC | circEIF4G2\|SNORD 97.6 |
| 593 | TCatcgctcacagGA | circEIF4G2\|SNORD 97.7 |
| 594 | AAtcatcgctcacAGG | circEIF4G2\|SNORD 97.7 |
| 595 | ATaatcatcgctcaCAG | circEIF4G2\|SNORD 97.7 |
| 596 | TTataatcatcgctCACA | circEIF4G2\|SNORD 97.7 |
| 597 | CTttttataatcatcgCTC | circEIF4G2\|SNORD 97.7 |
| 598 | ATcgggcacaggaCG | circEIF4G2\|SNORD 97.8 |
| 599 | TCgggcacaggacgCT | circEIF4G2\|SNORD 97.8 |
| 600 | ATcatcgggcacaggAC | circEIF4G2\|SNORD 97.8 |
| 601 | ATcatcgggcacaggaCG | circEIF4G2\|SNORD 97.8 |
| 602 | TTataatcatcgggcacAG | circEIF4G2\|SNORD 97.8 |
| 603 | GCatgccctcataTC | circEIF4G2\|SNORD 97.9 |
| 604 | GCatgccctcatatCT | circEIF4G2\|SNORD 97.9 |
| 605 | ATaatcatcgggcatGC | circEIF4G2\|SNORD 97.9 |
| 606 | GCatgccctcatatctCA | circEIF4G2\|SNORD 97.9 |
| 607 | GCatgccctcatatctcAT | circEIF4G2\|SNORD 97.9 |
| 608 | GCagccctcatatCT | circEIF4G2\|SNORD 97.10 |
| 609 | GCagccctcatatcTC | circEIF4G2\|SNORD 97.10 |
| 610 | TAatcatcgggcagcCC | circEIF4G2\|SNORD 97.10 |
| 611 | GCagccctcatatctcAT | circEIF4G2\|SNORD 97.10 |
| 612 | GCagccctcatatctcaTA | circEIF4G2\|SNORD 97.10 |
| 613 | GCtgtgtgcgtttGT | circlGF2 |
| 614 | GTgctgtgtgcgttTG | circlGF2 |
| 615 | GTgtgctgtgtgcgtTT | circlGF2 |
| 616 | TGtgctgtgtgcgtttGT | circlGF2 |
| 617 | TGtgtgtgctgtgtgcgTT | circlGF2 |
| 618 | TCATagcttaaACTG | circQSER1 |
| 619 | GCttaaactgttTTCT | circQSER1 |
| 620 | CATagcttaaactGTTT | circQSER1 |
| 621 | TTCatagcttaaacTGTT | circQSER1 |
| 622 | AGcttaaactgttttcTTC | circQSER1 |
| 623 | GTACtacaggtatGG | circUNKNOWN00000002 |
| 624 | GTactacaggtatgGC | circUNKNOWN00000002 |
| 625 | CTcggtactacaggtAT | circUNKNOWN00000002 |
| 626 | ATctcggtactacaggTA | circUNKNOWN00000002 |
| 627 | CTcggtactacaggtatGG | circUNKNOWN00000002 |
| 628 | CCActttttctTATG | circCHD1L |
| 629 | GCcaactccactttTT | circCHD1L |
| 630 | ATgccaactccacttTT | circCHD1L |
| 631 | CAactccactttttctTA | circCHD1L |
| 632 | CActttttcttatgttcAG | circCHD1L |
| 633 | TGTcacttctgATAC | circPRUNE |
| 634 | TCActtctgataCATC | circPRUNE |
| 635 | TGtcacttctgataCAT | circPRUNE |
| 636 | TGtcacttctgatacATC | circPRUNE |
| 637 | CTtctgatacatcaaaCTT | circPRUNE |
| 638 | AGAgagttgagATCT | circSLC27A3 |
| 639 | GTTGagatctgaAACT | circSLC27A3 |
| 640 | ATTagagagttgaGATC | circSLC27A3 |
| 641 | TTagagagttgagaTCTG | circSLC27A3 |
| 642 | AGAgagttgagatctgAAA | circSLC27A3 |
| 643 | ACagcttttgcctGG | circGATAD2B |
| 644 | TTgcctggcataccAA | circGATAD2B |
| 645 | TTttgcctggcatacCA | circGATAD2B |
| 646 | TTttgcctggcataccAA | circGATAD2B |
| 647 | CAGcttttgcctggcatAC | circGATAD2B |
| 648 | CAtgaacctgtcCAG | circKIAA0907 |
| 649 | CAgcatgaacctgtCC | circKIAA0907 |
| 650 | AAcctgtccagtgctAG | circKIAA0907 |
| 651 | TCagcatgaacctgtcCA | circKIAA0907 |
| 652 | CAgcatgaacctgtccaGT | circKIAA0907 |
| 653 | GCAAtagtcaaGAAT | circCCT3 |
| 654 | ATCTgcaatagtCAAG | circCCT3 |
| 655 | TCTgcaatagtcaAGAA | circCCT3 |
| 656 | GCAAtagtcaagaaTAAT | circCCT3 |
| 657 | GCAAtagtcaagaatAATT | circCCT3 |
| 658 | ACggccttcccagTC | circPLEKHM2 |
| 659 | ACggccttcccagtCT | circPLEKHM2 |
| 660 | CAcggccttcccagtCT | circPLEKHM2 |
| 661 | GGcacggccttcccagTC | circPLEKHM2 |
| 662 | CGgccttcccagtctgtGC | circPLEKHM2 |
| 663 | TGTgaaaacagcCTG | circVWCE |
| 664 | CAgcctggaacacaAG | circVWCE |
| 665 | AGcctggaacacaagTA | circVWCE |
| 666 | AAcagcctggaacacAAG | circVWCE |
| 667 | AGcctggaacacaagtaCA | circVWCE |
| 668 | GCAgaataggaACAT | circATF6 |
| 669 | AATaggaacatgCTGA | circATF6 |
| 670 | AATaggaacatgcTGAG | circATF6 |
| 671 | GAgcagaataggaaCATG | circATF6 |
| 672 | AGagagcagaataggaaCA | circATF6 |
| 673 | TTTCatcctacCAAT | circMALAT1.1 |
| 674 | TGtttcatcctacCAA | circMALAT1.1 |
| 675 | TGtttcatcctaccaAT | circMALAT1.1 |
| 676 | ATtgtttcatccatcCAA | circMALAT1.1 |
| 677 | ATtgtttcatcctaccaAT | circMALAT1.1 |
| 678 | TTGTttcatttTCTA | circMALAT1.2 |
| 679 | CTTCtccaaattgTTT | circMALAT1.2 |
| 680 | CTccaaattgtttCATT | circMALAT1.2 |
| 681 | TTctccaaattgtttCAT | circMALAT1.2 |
| 682 | TCttctccaaattgtttCA | circMALAT1.2 |
| 683 | TTCTccaaattGTTT | circMALAT1.3 |
| 684 | CCAAattgttttTATC | circMALAT1.3 |
| 685 | CCAaattgtttttaTCT | circMALAT1.3 |
| 686 | TAtcttctccaaatTGTT | circMALAT1.3 |
| 687 | CTtctccaaattgttttTA | circMALAT1.3 |
| 688 | ACTTctatcttCTAA | circMALAT1.4 |
| 689 | TCAaacttctatCTTC | circMALAT1.4 |
| 690 | CTTcaaacttctaTCTT | circMALAT1.4 |
| 691 | TCaaacttctatctTCA | circMALAT1.4 |
| 692 | TTCtatcttctaaaGTAT | circMALAT1.4 |
| 693 | TCAAacttctaTCTT | circMALAT1.5 |
| 694 | CTTCaaacttctATCT | circMALAT1.5 |
| 695 | CACttcaaacttcTATC | circMALAT1.5 |
| 696 | TCcacttcaaacttcTAT | circMALAT1.005 |
| 697 | CActtcaaacttctatCTT | circMALAT1.5 |
| 698 | CCACttcaaacTTCT | circMALAT1.6 |
| 699 | CACttcaaacttCTAT | circMALAT1.6 |
| 700 | TCcacttcaaacttCTA | circMALAT1.6 |
| 701 | CCacttcaaacttctaTC | circMALAT1.6 |
| 702 | AAACttctatcttccaATT | circMALAT1.6 |
| 703 | CCACttcaaacTTCT | circMALAT1.7 |
| 704 | CACttcaaacttCTAT | circMALAT1.7 |
| 705 | TCAaacttctatctTGT | circMALAT1.7 |
| 706 | CCacttcaaacttctaTC | circMALAT1.7 |
| 707 | AActtctatcttgtttCTA | circMALAT1.7 |
| 708 | TGTcttccagttTTC | circMALAT1.8 |
| 709 | GTcttccagttttcTT | circMALAT1.8 |
| 710 | TCcagttttcttcTAAG | circMALAT1.8 |
| 711 | TCtgtcttccagttttCT | circMALAT1.8 |
| 712 | TGtcttccagttttcttCT | circMALAT1.8 |
| 713 | CCcgtacttctgtCT | circMALAT1.9 |
| 714 | CCgtacttctgtctTC | circMALAT1.9 |
| 715 | TCccgtacttctgtcTT | circMALAT1.9 |
| 716 | CGtacttctgtcttccAA | circMALAT1.9 |
| 717 | ACttctgtcttccaattTT | circMALAT1.9 |
| 718 | TCTCtattctttTCT | circMALAT1.10 |
| 719 | TCTtttctaagtTTGT | circMALAT1.10 |
| 720 | ATTCttttctaagTTTG | circMALAT1.10 |
| 721 | TTCtctattcttttCTAA | circMALAT1.10 |
| 722 | CTctattcttttctaaGTT | circMALAT1.10 |
| 723 | TCTCtattctttTCT | circMALAT1.11 |
| 724 | CTAttcttttcttCTA | circMALAT1.11 |
| 725 | TCtctattcttttCTTC | circMALAT1.11 |
| 726 | CTatcttctctattcTTT | circMALAT1.11 |
| 727 | CTctattcttttcttcTAA | circMALAT1.11 |
| 728 | TTtatcttctaatTT | circMALAT1.12 |
| 729 | TCTAattttcttCTAA | circMALAT1.12 |
| 730 | CTTctaattttctTCTA | circMALAT1.12 |
| 731 | TCTaattttcttctaAGT | circMALAT1.12 |
| 732 | TCtaattttcttctaAGTT | circMALAT1.12 |
| 733 | TAaatttatctttTT | circMALAT1.13 |
| 734 | TTaaatttatctttTT | circMALAT1.13 |
| 735 | TTtaaatttatctttTT | circMALAT1.13 |
| 736 | GTttaaatttatctttTT | circMALAT1.13 |
| 737 | GGTTtaaatttatctTTTT | circMALAT1.13 |
| 738 | AAaaagatagaagTT | circUNKNOWN00000003 |
| 739 | AAaaagatagaagtTT | circUNKNOWN00000003 |
| 740 | AAaaagatagaagttTG | circUNKNOWN00000003 |
| 741 | AAaaagatagaagtttGA | circUNKNOWN00000003 |
| 742 | AAaaagatagaagtttGAA | circUNKNOWN00000003 |
| 743 | CTAgcttgtctTTTT | circMALAT1.14 |
| 744 | CCtagcttgtctttTT | circMALAT1.14 |
| 745 | TCctagcttgtctttTT | circMALAT1.14 |
| 746 | TTcctagcttgtcttTT | circMALAT1.14 |
| 747 | TTtcctagcttgtctttTT | circMALAT1.14 |
| 748 | CTagcttgtcttaGC | circMALAT1.15 |
| 749 | TCctagcttgtctTAG | circMALAT1.15 |
| 750 | TTtcctagcttgtctTA | circMALAT1.15 |
| 751 | TTcctagcttgtcttaGC | circMALAT1.15 |
| 752 | TTgtttcctagcttgtcTT | circMALAT1.15 |
| 753 | CTatacttctgcACC | circMALAT1.16 |
| 754 | TCttctatacttcTGC | circMALAT1.16 |
| 755 | TTctatacttctgcaCC | circMALAT1.16 |
| 756 | TAtcttctatacttctGC | circMALAT1.16 |
| 757 | ATcttctatacttctgcAC | circMALAT1.16 |
| 758 | AAagccttctgTGTA | circMALAT1.17 |
| 759 | AAagccttctgtGTAG | circMALAT1.17 |
| 760 | TTccaaaagccttctGT | circMALAT1.17 |
| 761 | CAaaagccttctgtgtAG | circMALAT1.17 |
| 762 | CTtccaaaagccttctgTG | circMALAT1.17 |
| 763 | ACactggttcctgGA | circMALAT1.18 |
| 764 | TCaaacactggttCCT | circMALAT1.18 |
| 765 | CAtcaaacactggttCC | circMALAT1.18 |
| 766 | TTcatcaaacactgGTTC | circMALAT1.18 |
| 767 | TCaaacactggttcctgGA | circMALAT1.18 |
| 768 | TCctatcttcaCCAA | circMALAT1.19 |
| 769 | CCtatcttcaccAAAT | circMALAT1.19 |
| 770 | TTcctatcttcacCAAA | circMALAT1.19 |
| 771 | TTtcctatcttcacCAAA | circMALAT1.19 |
| 772 | CTtttcctatcttcaccAA | circMALAT1.19 |
| 773 | ACacggaagacgGGA | circUCK2 |
| 774 | ACGgaagacgggaATG | circUCK2 |
| 775 | ACacggaagacggGAAT | circUCK2 |
| 776 | CGgaagacgggaatggGA | circUCK2 |
| 777 | AAgacgggaatgggagaCA | circUCK2 |
| 778 | TGggaagcctcaTTT | circSUCO |
| 779 | GCtgggaagcctcaTT | circSUCO |
| 780 | GCtgggaagcctcatTT | circSUCO |
| 781 | CAgctgggaagcctcaTT | circSUCO |
| 782 | CAgctgggaagcctcatTT | circSUCO |
| 783 | TTgcaatcgtaCCTG | circRAB6A |
| 784 | CAatcgtacctgcTTT | circRAB6A |
| 785 | CAatcgtacctgcttTA | circRAB6A |
| 786 | GCaatcgtacctgcttTA | circRAB6A |
| 787 | ATtgcaatcgtacctgcTT | circRAB6A |
| 788 | ATCactgaagtCTCA | circRPS3\|SNORD15 B.1 |
| 789 | TCatcactgaagTCTC | circRPS3\|SNORD15 B.1 |
| 790 | TGtcatcactgaagTCT | circRPS3\|SNORD15 B.1 |
| 791 | GTcatcactgaagtctCA | circRPS3\|SNORD15 B.1 |
| 792 | CAtcactgaagtctcagAC | circRPS3\|SNORD15 B.1 |
| 793 | AAGCttctcagaCAA | circRPS3\|SNORD15 B.2 |
| 794 | AAGCttctcagaCAAA | circRPS3\|SNORD15 B.2 |
| 795 | AAGCttctcagacaaAT | circRPS3\|SNORD15 B.2 |
| 796 | AAGcttctcagacaaATG | circRPS3\|SNORD15 B.2 |
| 797 | AAgcttctcagacaaatGC | circRPS3\|SNORD15 B.2 |
| 798 | ACtgaagccttcTCA | circRPS3\|SNORD15 B.3 |
| 799 | ACtgaagccttctCAG | circRPS3\|SNORD15 B.3 |
| 800 | GTcatcactgaagccTT | circRPS3\|SNORD15 B.3 |
| 801 | GTcatcactgaagcctTC | circRPS3\|SNORD15 B.3 |
| 802 | AAgccttctcagacaaaTG | circRPS3\|SNORD15 B.3 |
| 803 | AGAagaatctgTAGC | circRSF1 |
| 804 | AGAatctgtagcTTAT | circRSF1 |
| 805 | GAagaatctgtagCTTA | circRSF1 |
| 806 | CAgaagaatctgtagCTT | circRSF1 |
| 807 | AGaagaatctgtagctTAT | circRSF1 |
| 808 | GAtctttagtgATGC | circABL2 |
| 809 | CTggcaaagtgaTCTT | circABL2 |
| 810 | AAGtgatctttagTGAT | circABL2 |
| 811 | CTggcaaagtgatcttTA | circABL2 |
| 812 | CTggcaaagtgatctttAG | circABL2 |
| 813 | CGagaagcctgtGTG | circGNB1 |
| 814 | TGacgagaagcctgTG | circGNB1 |
| 815 | ACtgacgagaagcctGT | circGNB1 |
| 816 | TGacgagaagcctgtgTG | circGNB1 |
| 817 | CActgacgagaagcctgTG | circGNB1 |
| 818 | GAccagagtgtcTAG | circRPLP2 \| SNORA52 |
| 819 | ATtaggatggacCAGA | circRPLP2 \| SNORA52 |
| 820 | GGatggaccagagtgTC | circRPLP2 \| SNORA52 |
| 821 | ACcagagtgtctagaaGT | circRPLP2 \| SNORA52 |
| 822 | TTaggatggaccagagtGT | circRPLP2 \| SNORA52 |
| 823 | GGgcctccccatgTA | circPICALM.1 |
| 824 | GGgcctccccatgtAC | circPICALM.1 |
| 825 | GGcctccccatgtacttGC | circPICALM.1 |
| 826 | ACTGaattaagTCTC | circPICALM.2 |
| 827 | AAttaagtctccCAT | circPICALM.2 |
| 828 | GAattaagtctccccAT | circPICALM.2 |
| 829 | TGcactgaattaagtCTC | circPICALM.2 |
| 830 | ACtgaattaagtctcccCA | circPICALM.2 |
| 831 | ACagcagccatgtGT | circSNORA23\|IP07.2 |
| 832 | CAtgtgtgtgggGAA | circSNORA23\|IP07.2 |
| 833 | CCatgtgtgtgtgggAA | circSNORA23\|IP07.2 |
| 834 | AGccatgtgtgtgtggGA | circSNORA23\|IP07.2 |
| 835 | ATgtgtgtgtgggaattTG | circSNORA23\|IP07.2 |
| 836 | ACagcagccatgtGT | circSNORA23\|IP07.3 |
| 837 | TTacagcagccatgTG | circSNORA23\|IP07.3 |
| 838 | ACagcagccatgttGG | circSNORA23\|IP07.3 |
| 839 | ACattacagcagccatGT | circSNORA23\|IP07.3 |
| 840 | CAttacagcagccatgtGT | circSNORA23\|IPO7.3 |
| 841 | CTTtttgagcaGTTC | circCFH |
| 842 | GCattctctttttGAG | circCFH |
| 843 | TCgcattctctttttGA | circCFH |
| 844 | GTtcgcattctcttttTG | circCFH |
| 845 | ATtctctttttgagcagTT | circCFH |
| 846 | GATTgcagtggATAA | circSLC41A2.1 |
| 847 | GAaggactggattGCA | circSLC41A2.1 |
| 848 | TGgattgcagtggaTAA | circSLC41A2.1 |
| 849 | CAgaaggactggattgCA | circSLC41A2.1 |
| 850 | TGgattgcagtggataaTC | circSLC41A2.1 |
| 851 | GActggatctgTACT | circSLC41A2.2 |
| 852 | GAtctgtactatATCC | circSLC41A2.2 |
| 853 | TGgatctgtactaTATC | circSLC41A2.2 |
| 854 | GAtctgtactatatcCAG | circSLC41A2.2 |
| 855 | AGaaggactggatctgtAC | circSLC41A2.2 |
| 856 | GCatatttttcTGGC | circCORO1C |
| 857 | GCatatttttctggCA | circCORO1C |
| 858 | ATttttctggcaatCTC | circCORO1C |
| 859 | ATttttctggcaatctCA | circCORO1C |
| 860 | CAtatttttctggcaatCT | circCORO1C |
| 861 | CCTCtaggaatttTG | circEIF4G3\| RP11-487E1.2 |
| 862 | CTAggaattttgAGAG | circEIF4G3\| RP11-487E1.2 |
| 863 | TAGgaattttgagaGAG | circEIF4G3\| RP11-487E1.2 |
| 864 | GAattttgagagagtcCA | circEIF4G3\| RP11-487E1.2 |
| 865 | AGgaattttgagagagtCC | circEIF4G3\| RP11-487E1.2 |
| 866 | CCgatatagatttTT | circNAA25 |
| 867 | TGccgatatagattTT | circNAA25 |
| 868 | TGccgatatagatttTT | circNAA25 |
| 869 | TGtgccgatatagattTT | circNAA25 |
| 870 | TGtgccgatatagatttTT | circNAA25 |
| 871 | TGagttcagccTGAA | circMED13L |
| 872 | CCgtgagttcagccTG | circMED13L |
| 873 | CGtgagttcagcctgAA | circMED13L |
| 874 | CGtgagttcagcctgaAA | circMED13L |
| 875 | TGagttcagcctgaaaAAT | circMED13L |
| 876 | ATCActgtaatGTAC | circLPGAT1\|RN7SL344P |
| 877 | CCatcactgtaatGTA | circLPGAT1\|RN7SL344P |
| 878 | TTccatcactgtaaTGT | circLPGAT1\|RN7SL344P |
| 879 | TCcatcactgtaatgTAC | circLPGAT1\|RN7SL344P |
| 880 | TCcatcactgtaatgtaCA | circLPGAT1\|RN7SL344P |
| 881 | GCccctatcctgtCA | circAACS |
| 882 | GTgcccctatcctgTC | circAACS |
| 883 | GTgcccctatcctgtCA | circAACS |
| 884 | GGgtgcccctatcctgTC | circAACS |
| 885 | GCccctatcctgtcaacCA | circAACS |
| 886 | AGGactattaaCCAA | circTP53BP2 |
| 887 | TTaaccaagtttcCCA | circTP53BP2 |
| 888 | ACTAttaaccaagTTTC | circTP53BP2 |
| 889 | TAaccaagtttcccatTG | circTP53BP2 |
| 890 | CTattaaccaagtttccCA | circTP53BP2 |
| 891 | AAgacatccttcCGG | circSOX5 |
| 892 | AAgacatccttccgGC | circSOX5 |
| 893 | ACatccttccggctcGT | circSOX5 |
| 894 | AAgacatccttccggcTC | circSOX5 |
| 895 | CGcttggaagacatcctTC | circSOX5 |
| 896 | GGaactggctgctCT | circDNAH14 |
| 897 | AAggaactggctgCTC | circDNAH14 |
| 898 | TAaaggaactggctgCT | circDNAH14 |
| 899 | AAaggaactggctgctCT | circDNAH14 |
| 900 | ATaaaggaactggctgcTC | circDNAH14 |
| 901 | TGtactctactgTGC | circKDM1A\|MIR3115 |
| 902 | TGtactctactgtgCG | circKDM1A\|MIR3115 |
| 903 | TGtactctactgtgcGG | circKDM1A\|MIR3115 |
| 904 | CTctgtactctactgtGC | circKDM1A\|MIR3115 |
| 905 | TCtgtactctactgtgcGG | circKDM1A\|MIR3115 |
| 906 | AAAGgatgactCTGA | circTTC13 |
| 907 | AGgatgactctgaaGC | circTTC13 |
| 908 | AAAGgatgactctGAAG | circTTC13 |
| 909 | ACtctgaagcattgttGA | circTTC13 |
| 910 | ATgactctgaagcattgTT | circTTC13 |
| 911 | CAtggccttgtagCA | circEGLN1 |
| 912 | TGgccttgtagcatAT | circEGLN1 |
| 913 | CAtggccttgtagcaTA | circEGLN1 |
| 914 | CAtggccttgtagcatAT | circEGLN1 |
| 915 | AAcaagcaaccatggccTT | circEGLN1 |
| 916 | CCttcctttccacCG | circEGLN3 |
| 917 | CTtcctttccaccgAT | circEGLN3 |
| 918 | CCttcctttccaccAT | circEGLN3 |
| 919 | TTcctttccaccgatgGT | circEGLN3 |
| 920 | TTcctttccaccgatggTC | circEGLN3 |
| 921 | GTTTaagaatgCAGG | circTOMM20\|SNORA14B |
| 922 | AAGAatgcaggtaTGA | circTOMM20\|SNORA14B |
| 923 | TTTaagaatgcagGTAT | circTOMM20\|SNORA14B |
| 924 | GGtttaagaatgcaggTA | circTOMM20\|SNORA14B |
| 925 | TAAGaatgcaggtatGAAA | circTOMM20\|SNORA14B |
| 926 | GTCttccattcATTT | circSCCPDH |
| 927 | AGtgttggtcttccAT | circSCCPDH |
| 928 | GTtggtcttccattcAT | circSCCPDH |
| 929 | GTcttccattcattttAT | circSCCPDH |
| 930 | CTtccattcattttattTC | circSCCPDH |
| 931 | AACCgagttagaAGT | circZNF124.2 |
| 932 | AACcgagttagaAGTC | circZNF124.2 |
| 933 | CCgagttagaagtcTTG | circZNF124.2 |
| 934 | AAaggcaaccgagttAGA | circZNF124.2 |
| 935 | AAggcaaccgagttagaAG | circZNF124.2 |
| 936 | CCaccttgatcagGG | circGLS2 |
| 937 | TGccaccttgatcaGG | circGLS2 |
| 938 | ATgtaggctgccaccTT | circGLS2 |
| 939 | AGgctgccaccttgatCA | circGLS2 |
| 940 | ATgtaggctgccaccttGA | circGLS2 |
| 941 | GCGagactctgaATA | circR3HDM2 |
| 942 | GACTctgaataaaTGC | circR3HDM2 |
| 943 | GActctgaataaaTGCT | circR3HDM2 |
| 944 | GAgcgagactctgaaTAA | circR3HDM2 |
| 945 | CGagactctgaataaaTGC | circR3HDM2 |
| 946 | TGcagtttctcccTG | circDHDDS |
| 947 | TCcctgggatccaaCA | circDHDDS |
| 948 | AGtttctccctgggaTC | circDHDDS |
| 949 | TGcagtttctccctggGA | circDHDDS |
| 950 | TTtctccctgggatccaAC | circDHDDS |
| 951 | GAgagtgacctGTTT | circSNORA73A\|RCC1\|SNHG3.1 |
| 952 | TGacctgtttcctgCA | circSNORA73A\|RCC1\|SNHG3.1 |
| 953 | AGagtgacctgtttcCT | circSNORA73A\|RCC1\|SNHG3.1 |
| 954 | GGagagtgacctgtttCC | circSNORA73A\|RCC1\|SNHG3.1 |
| 955 | TGacctgtttcctgcatGG | circSNORA73A\|RCC1\|SNHG3.1 |
| 956 | AGAgtgacctgcaTG | circSNORA73A\|RCC1\|SNHG3.2 |
| 957 | GGagagtgacctgcAT | circSNORA73A\|RCC1\|SNHG3.2 |
| 958 | GGagagtgacctgcaTG | circSNORA73A\|RCC1\|SNHG3.2 |
| 959 | GAgagtgacctgcatgGT | circSNORA73A\|RCC1\|SNHG3.2 |
| 960 | ATcaggaggataCCT | circSNORA61\|SNHG12 |
| 961 | GGatcaggaggataCC | circSNORA61\|SNHG12 |
| 962 | GAggatacctgtctgAA | circSNORA61\|SNHG12 |
| 963 | GAggatacctgtctgaAA | circSNORA61\|SNHG12 |
| 964 | GAtacctgtctgaaactAG | circSNORA61\|SNHG12 |
| 965 | ACacatatggcTTGC | circCEP83\|RBMS2P1 |
| 966 | TGacacatatggCTTG | circCEP83\|RBMS2P1 |
| 967 | GAcacatatggcttgCA | circCEP83\|RBMS2P1 |
| 968 | ATgacacatatggcttGC | circCEP83\|RBMS2P1 |
| 969 | ATacatatggcttgcaaAG | circCEP83\|RBMS2P1 |
| 970 | GCttctttatctCTG | circFGD6 |
| 971 | TTatctctggaaTGCT | circFGD6 |
| 972 | GGcttctttatctctGG | circFGD6 |
| 973 | CTttatctctggaatgCT | circFGD6 |
| 974 | GTggcttctttatctctGG | circFGD6 |
| 975 | AAcagctagtgtCGC | circPUM1 |
| 976 | GGaacagctagtgtCG | circPUM1 |
| 977 | TCtttggaacagctaGT | circPUM1 |
| 978 | TTtggaacagctagtgTC | circPUM1 |
| 979 | TTggaacagctagtgtcGC | circPUM1 |
| 980 | CAgctagaagaTGCA | circTMCO3\| RP11-230F18.6 |
| 981 | TTcagctagaagATGC | circTMCO3\| RP11-230F18.6 |
| 982 | TTcagctagaagatGCA | circTMCO3\| RP11-230F18.6 |
| 983 | CAcattttcagctaGAAG | circTMCO3\| RP11-230F18.6 |
| 984 | GAcacacattttcagcTAG | circTMCO3\| RP11-230F18.6 |
| 985 | AAACtagaacgTGGA | circPTP4A2 |
| 986 | AAACtagaacgtGGAT | circPTP4A2 |
| 987 | CCAAcgaaaaactAGAA | circPTP4A2 |
| 988 | CCAAcgaaaaactaGAAC | circPTP4A2 |
| 989 | CCAacgaaaaactagaACG | circPTP4A2 |
| 990 | AACCaggactaTGAT | circZMYM5 |
| 991 | CAtgccaatgaacCAG | circZMYM5 |
| 992 | ATgccaatgaaccagGA | circZMYM5 |
| 993 | CCaatgaaccaggactAT | circZMYM5 |
| 994 | CAatgaaccaggactatGA | circZMYM5 |
| 995 | TGcgattctgtgCAC | circN6AMT2 |
| 996 | ACatgcgattctgtGC | circN6AMT2 |
| 997 | ACatgcgattctgtgCA | circN6AMT2 |
| 998 | CAcacatgcgattctgTG | circN6AMT2 |
| 999 | CAcatgcgattctgtgcAC | circN6AMT2 |
| 1000 | ATATaccagcaTTTC | circRPL21\|SNORA27 |
| 1001 | CAgcatttcctctgAC | circRPL21\|SNORA27 |
| 1002 | ATaccagcatttcctCT | circRPL21\|SNORA27 |
| 1003 | ATataccagcatttccTC | circRPL21\|SNORA27 |
| 1004 | TTatataccagcatttcCT | circRPL21\|SNORA27 |
| 1005 | CACCttttaatcGC | circGTF2F2 |
| 1006 | GACacctttttaATCG | circGTF2F2 |
| 1007 | GAcaccttttaatcGC | circGTF2F2 |
| 1008 | ATgacacctttttaaTCG | circGTF2F2 |
| 1009 | CAAagtaaatgacacCTTT | circGTF2F2 |
| 1010 | TTAccaccacttgaGC | circZMYM4 |
| 1011 | CCacttgagcattgTC | circZMYM4 |
| 1012 | CAccacttgagcattGT | circZMYM4 |
| 1013 | ACcaccacttgagacatTG | circZMYM4 |
| 1014 | ATaccaaccacttgagcaTT | circZMYM4 |
| 1015 | AATggaggcgttTGA | circLINC00355 |
| 1016 | ACcaaatggaggcGTT | circLINC00355 |
| 1017 | AAtggaggcgtttgaGC | circLINC00355 |
| 1018 | GCaaatggaggcgtttGA | circLINC00355 |
| 1019 | AAcagcaaatggaggcgTT | circLINC00355 |
| 1020 | GTccttagcaacCAC | circUNKNOWN00000004 |
| 1021 | ATgtccttagcaacCA | circUNKNOWN00000004 |
| 1022 | CAatgtccttagcaaCC | circUNKNOWN00000004 |
| 1023 | AAtgtccttagcaaccAC | circUNKNOWN00000004 |
| 1024 | ATtcaccaacaatgtccTT | circUNKNOWN00000004 |
| 1025 | GACAcacagaaCATA | circFARP1 |
| 1026 | GACAcacagaacATAC | circFARP1 |
| 1027 | AGaacatacacaaTGCT | circFARP1 |
| 1028 | CACacagaacatacACAA | circFARP1 |
| 1029 | CAcagaacatacacaatGC | circFARP1 |
| 1030 | GTgggctccttgcAG | circDYNC1H1 |
| 1031 | GTgggctccttgcaGG | circDYNC1H1 |
| 1032 | CGtaggtgggctcctTG | circDYNC1H1 |
| 1033 | TAggtgggctccttgcAG | circDYNC1H1 |
| 1034 | TCcttgcaggtgtttctTC | circDYNC1H1 |
| 1035 | TGgtttagcaggtGG | circCDC42BPB |
| 1036 | AATggtttagcagGTG | circCDC42BPB |
| 1037 | AATggtttagcaggtGG | circCDC42BPB |
| 1038 | TAaatggtttagcagGTG | circCDC42BPB |
| 1039 | AAtggtttagcaggtggTT | circCDC42BPB |
| 1040 | CCATcaaaaatTGTT | circCCNB1IP1\|SNORA79\|AL355075.1 |
| 1041 | AGCcatcaaaaatTGT | circCCNB1IP1\|SNORA79\|AL355075.1 |
| 1042 | ACAgccatcaaaaATTG | circCCNB1IP1\|SNORA79\|AL355075.1 |
| 1043 | GCcatcaaaaattgTTTG | circCCNB1IP1\|SNORA79\|AL355075.1 |
| 1044 | AGgaacagccatcaaaAAT | circCCNB1IP1\|SNORA79\|AL355075.1 |
| 1045 | TGttcaatgggcgGA | circRPPH1\|RPPH1.1 |
| 1046 | TTcaatgggcggagGA | circRPPH1\|RPPH1.1 |
| 1047 | TTcaatgggcggaggAG | circRPPH1\|RPPH1.1 |
| 1048 | CGtgagtctgttcaatGG | circRPPH1\|RPPH1.1 |
| 1049 | CGtgagtctgttcaatgGG | circRPPH1\|RPPH1.1 |
| 1050 | GCatccgccgggcGG | circRPPH1\|RPPH1.2 |
| 1051 | CGccgggcggaggaGA | circRPPH1\|RPPH1.2 |
| 1052 | CGccgggcggaggagAG | circRPPH1\|RPPH1.2 |
| 1053 | GGcatccgccgggcggAG | circRPPH1\|RPPH1.2 |
| 1054 | GCcgggcggaggagagtAG | circRPPH1\|RPPH1.2 |
| 1055 | AActcacttcgCTGG | circRPPH1\|RPPH1.3 |
| 1056 | GGgaactcacttcgCT | circRPPH1\|RPPH1.3 |
| 1057 | GGaactcacttcgctGG | circRPPH1\|RPPH1.3 |
| 1058 | CTgggaactcacttcgCT | circRPPH1\|RPPH1.3 |
| 1059 | TGggaactcacttcgctGG | circRPPH1\|RPPH1.3 |
| 1060 | TCaggcaaaggagGC | circRPPH1\|RPPH1.4 |
| 1061 | CCgaagctcaggcaAA | circRPPH1\|RPPH1.4 |
| 1062 | CGaagctcaggcaaaGG | circRPPH1\|RPPH1.4 |
| 1063 | CGaagctcaggcaaagGA | circRPPH1\|RPPH1.4 |
| 1064 | CGaagctcaggcaaaggAG | circRPPH1\|RPPH1.4 |
| 1065 | CAttgggagccccTC | circSNORD8\|CHD8.2 |
| 1066 | CTcatcattgggagCC | circSNORD8\|CHD8.2 |
| 1067 | GGagcccctcagatcTT | circSNORD8\|CHD8.2 |
| 1068 | GGagcccctcagatctTC | circSNORD8\|CHD8.2 |
| 1069 | TCatcattgggagccccTC | circSNORD8\|CHD8.2 |
| 1070 | GAcgcccctcagaTC | circSNORD8\|CHD8.2 |
| 1071 | TCatcattgggacgCC | circSNORD8\|CHD8.2 |
| 1072 | CAtcattgggacgccCC | circSNORD8\|CHD8.2 |
| 1073 | GAcgcccctcagatctTC | circSNORD8\|CHD8.2 |
| 1074 | GAcgcccctcagatcttCA | circSNORD8\|CHD8.2 |
| 1075 | CGccgcctacgagGA | circPPP1R3E |
| 1076 | TAcgaggagggagcCC | circPPP1R3E |
| 1077 | CCtacgaggagggagCC | circPPP1R3E |
| 1078 | CCcgccgcctacgaggAG | circPPP1R3E |
| 1079 | GCccccgccgcctacgaGG | circPPP1R3E |
| 1080 | CAgtggagtaggcCC | circCHMP4A\|RP11-468E2.1\|AL136419.6 |
| 1081 | CAgtggagtaggccCA | circCHMP4A\|RP11-468E2.1\|AL136419.6 |
| 1082 | CAgtggagtaggcccAC | circCHMP4A\|RP11-468E2.1\|AL136419.6 |
| 1083 | GGagtaggcccactcaGC | circCHMP4A\|RP11-468E2.1\|AL136419.6 |
| 1084 | AGgcccactcagccaacTG | circCHMP4A\|RP11-468E2.001\|AL136419.6 |
| 1085 | GATttctcatcATCT | circUNKNOWN00000005 |
| 1086 | ATttctcatcatcTGC | circUNKNOWN00000005 |
| 1087 | TTtctcatcatctgCAC | circUNKNOWN00000005 |
| 1088 | GAtttctcatcatctgCA | circUNKNOWN00000005 |
| 1089 | AActctgtgatttctcATC | circUNKNOWN00000005 |
| 1090 | GTGcttttaataGGA | circSEC23A |
| 1091 | CTTGgtgcttttaaTA | circSEC23A |
| 1092 | TTggtgcttttaaTAGG | circSEC23A |
| 1093 | TTggtgcttttaataGGA | circSEC23A |
| 1094 | CTtttaataggagtgtCTT | circSEC23A |
| 1095 | CAccaccaaggaGAG | circSNORD46\|RPS8 |
| 1096 | CTcgcacccaccacCA | circSNORD46\|RPS8 |
| 1097 | TCgcacccaccaccaAG | circSNORD46\|RPS8 |
| 1098 | CCcaccaccaaggagaGC | circSNORD46\|RPS8 |
| 1099 | ACcaccaaggagagcaaGG | circSNORD46\|RPS8 |
| 1100 | TGATtcttgtaCTTG | circSAMD4A |
| 1101 | GTTAatgattctTGTA | circSAMD4A |
| 1102 | GTTaatgattcttGTAC | circSAMD4A |
| 1103 | ATgattcttgtacttGTG | circSAMD4A |
| 1104 | GTtaatgattcttgtaCTT | circSAMD4A |
| 1105 | CTgcaagttcatCGT | circPCNX |
| 1106 | TGctgcaagttcatCG | circPCNX |
| 1107 | TTGtgctgcaagttcAT | circPCNX |
| 1108 | TTgtgctgcaagttcaTC | circPCNX |
| 1109 | CTgcaagttcatcgtgcAA | circPCNX |
| 1110 | CCcagattaggtaGT | circPSEN1 |
| 1111 | CCcagattaggtagTA | circPSEN1 |
| 1112 | TCccagattaggtagTA | circPSEN1 |
| 1113 | GCtcccagattaggtaGT | circPSEN1 |
| 1114 | GCtcccagattaggtagTA | circPSEN1 |
| 1115 | CAggcacagtggCAA | circFCF1 |
| 1116 | GGcacagtggcaAAAC | circFCF1 |
| 1117 | CCaggcacagtggcaAA | circFCF1 |
| 1118 | GCacagtggcaaaacaGC | circFCF1 |
| 1119 | CCaggcacagtggcaaaAC | circFCF1 |
| 1120 | TCgagcaggcacTTG | circSCARNA13\|SNHG10.1 |
| 1121 | TCtcgagcaggcacTT | circSCARNA13\|SNHG10.1 |
| 1122 | TCtcgagcaggcacTTG | circSCARNA13\|SNHG10.1 |
| 1123 | CGagcaggcacttgtgGC | circSCARNA13\|SNHG10.1 |
| 1124 | CAggcacttgtggcagtAC | circSCARNA13\|SNHG10.1 |
| 1125 | CAgttgtggcagTAC | circSCARNA13\|SNHG10.2 |
| 1126 | GCatgcctcagttgTG | circSCARNA13\|SNHG10.2 |
| 1127 | CTcagttgtggcagtAC | circSCARNA13\|SNHG10.2 |
| 1128 | AGgcatgcctcagttgTG | circSCARNA13\|SNHG10.2 |
| 1129 | CAgttgtggcagtacttAG | circSCARNA13\|SNHG10.2 |
| 1130 | ACcctcgactgCAAA | circSCARNA13\|SNHG10.3 |
| 1131 | CAaccctcgactgcAA | circSCARNA13\|SNHG10.3 |
| 1132 | TAccaccaaccctcgAC | circSCARNA13\|SNHG10.3 |
| 1133 | AAccctcgactgcaaaGC | circSCARNA13\|SNHG10.3 |
| 1134 | AAccctcgactgcaaagCT | circSCARNA13\|SNHG10.3 |
| 1135 | GTcatccagtcAGAA | circUNKNOWN00000006 |
| 1136 | GTcatccagtcaGAAA | circUNKNOWN00000006 |
| 1137 | TAaagtcatccagtCAG | circUNKNOWN00000006 |
| 1138 | AAgtcatccagtcAGAA | circUNKNOWN00000006 |
| 1139 | TAtaaagtcatccagtcAG | circUNKNOWN00000006 |
| 1140 | AGgagcctgccatTG | circTJP1 |
| 1141 | AGgagcctgccattGC | circTJP1 |
| 1142 | AAatccaggagcctgCC | circTJP1 |
| 1143 | AGgagcctgccattgcTT | circTJP1 |
| 1144 | AAatccaggagcctgccAT | circTJP1 |
| 1145 | GAtaacctgtggtCC | circRP11-632K20.7 |
| 1146 | GAtaacctgtggtcCA | circRP11-632K20.7 |
| 1147 | CTtggataacctgtgGT | circRP11-632K20.7 |
| 1148 | TTggataacctgtggtCC | circRP11-632K20.7 |
| 1149 | ATgtccttggataacctGT | circRP11-632K20.7 |
| 1150 | TAaccttgcagCTTT | circTTBK2 |
| 1151 | GTTtccatttaacCTT | circTTBK2 |
| 1152 | TTtccatttaaccttGC | circTTBK2 |
| 1153 | ATttaaccttgcagctTT | circTTBK2 |
| 1154 | GGtttccatttaaccttGC | circTTBK2 |
| 1155 | CAACtcacgaagCAG | circPPIB |
| 1156 | CCacaactcacgaaGC | circPPIB |
| 1157 | TTccacaactcacGAAG | circPPIB |
| 1158 | GCttccacaactcacgAA | circPPIB |
| 1159 | TTccacaactcacgaagCA | circPPIB |
| 1160 | GCTgacgaagaTTTA | circUBE2Q2 |
| 1161 | GCTgacgaagatTTAA | circUBE2Q2 |
| 1162 | GCTGacgaagattTAAA | circUBE2Q2 |
| 1163 | CAattgctgacgaaGATT | circUBE2Q2 |
| 1164 | GCTgacgaagatttaaaTG | circUBE2Q2 |
| 1165 | ATcttgtgccaccTG | circETFA |
| 1166 | GCcacctgcataaaTA | circETFA |
| 1167 | TGccacctgcataaaTA | circETFA |
| 1168 | TGtgccacctgcataaAT | circETFA |
| 1169 | CCacctgcataaatagtTC | circETFA |
| 1170 | GAtaatagagcCCCT | circSEC11A |
| 1171 | TTgataatagagcCCC | circSEC11A |
| 1172 | ACTTgataatagagcCC | circSEC11A |
| 1173 | CTtgataatagagcccCT | circSEC11A |
| 1174 | GActtgataatagagccCC | circSEC11A |
| 1175 | GCtacctgcacagTG | circPDE8A |
| 1176 | CTactgctacctgcAC | circPDE8A |
| 1177 | CTgctacctgcacagTG | circPDE8A |
| 1178 | AGctactgctacctgcAC | circPDE8A |
| 1179 | TCagctactgctacctgCA | circPDE8A |
| 1180 | TTccttatgccccTG | circDAB1\|OMA1 |
| 1181 | TTatgcccctgaggTA | circDAB1\|OMA1 |
| 1182 | TTccttatgcccctgAG | circDAB1\|OMA1 |
| 1183 | ATttccttatgccctGA | circDAB1\|OMA1 |
| 1184 | TAtgccctgaggtataAG | circDAB1\|OMA1 |
| 1185 | CTAAaagtcttTGGT | circABHD2 |
| 1186 | AAAAgtctttggTCAG | circABHD2 |
| 1187 | AAacctaaagtcttTG | circABHD2 |
| 1188 | AACAaacctaaaagTCTT | circABHD2 |
| 1189 | AACAaacctaaaagtCTTT | circABHD2 |
| 1190 | CAGGtacaaatATTG | circlQGAP1.1 |
| 1191 | GGTacaaatattGACT | circlQGAP1.1 |
| 1192 | CAGgtacaaatatTGAC | circlQGAP1.1 |
| 1193 | AGGtacaaatattgaCTT | circlQGAP1.1 |
| 1194 | GAacaggtacaaataTTGA | circlQGAP1.1 |
| 1195 | AGCatgtatatTGAC | circlQGAP1.2 |
| 1196 | AGCatgtatattgACT | circlQGAP1.2 |
| 1197 | GTATattgactttgTTT | circlQGAP1.2 |
| 1198 | CAgcagcatgtatattGA | circlQGAP1.2 |
| 1199 | GTatattgatttgtTTAT | circlQGAP1.2 |
| 1200 | GTccttttccttcTC | circCHD2 |
| 1201 | CTggtccttttccTC | circCHD2 |
| 1202 | TTccttctctttctTAT | circCHD2 |
| 1203 | CTtttccttctctttcTT | circCHD2 |
| 1204 | TTccttctctttcttatVT | circCHD2 |
| 1205 | TGgcccgcagattTT | circlGF1R |
| 1206 | TGgcccgcagatttTC | circlGF1R |
| 1207 | GGcccgcagattttcTG | circlGF1R |
| 1208 | CTggcccgcagattttCT | circlGF1R |
| 1209 | AGattttctggcagcggTT | circlGF1R |
| 1210 | CGtgcacaggctgCA | circNPRL3 |
| 1211 | GAcgtgcacaggctGC | circNPRL3 |
| 1212 | CGtgcacaggctgcaGC | circNPRL3 |
| 1213 | CCcgacgtgcacaggcTG | circNPRL3 |
| 1214 | CGtgcacaggctgcagcAA | circNPRL3 |
| 1215 | GGtggctgttaaGTT | circNDE1 |
| 1216 | GCacggtggctgttAA | circNDE1 |
| 1217 | CAcggtggctgttaaGT | circNDE1 |
| 1218 | CGgtggctgttaagttCT | circNDE1 |
| 1219 | TGgctgttaagttctctGT | circNDE1 |
| 1220 | GTcagggtcgtGGAT | circABCC1 |
| 1221 | TCcagtcagggtcgTG | circABCC1 |
| 1222 | CAttccagtcagggtCG | circABCC1 |
| 1223 | CAgtcagggtcgtggaTG | circABCC1 |
| 1224 | ATtccagtcagggtcgtGG | circABCC1 |
| 1225 | CCgggtacgcagcTG | circRPS2 \|SNORA64 |
| 1226 | ACagccgggtacgcAG | circRPS2 \|SNORA64 |
| 1227 | ACagccgggtacgcaGC | circRPS2 \|SNORA64 |
| 1228 | AGgacagccgggtacgCA | circRPS2 \|SNORA64 |
| 1229 | ACagccgggtacgcagcTG | circRPS2 \|SNORA64 |
| 1230 | CGcacagggtacTTG | circPOLR3E |
| 1231 | GAcgcacagggtacTT | circPOLR3E |
| 1232 | GTacttgcctgtagaGT | circPOLR3E |
| 1233 | GGacgcacagggtactTG | circPOLR3E |
| 1234 | GGacgcacagggtacttGC | circPOLR3E |
| 1235 | CTcttacgaagacCA | circATXN2L |
| 1236 | CTcttacgaagaccAC | circATXN2L |
| 1237 | CTcttacgaagacca\|CA | circATXN2L |
| 1238 | CAgctgggctcttacgAA | circATXN2L |
| 1239 | AGctgggctcttacgaaGA | circATXN2L |
| 1240 | GTgtgatgtcctgTC | circMVP |
| 1241 | TGatgtcctgtcacCC | circMVP |
| 1242 | GGtgtgatgtcctgtCA | circMVP |
| 1243 | GAtgtvvtgtcaccctCT | circMVP |
| 1244 | GTcctgtcaccctctccTT | circMVP |
| 1245 | GAgcctgtttggtTG | circASPHD1 |
| 1246 | GAgcctgtttggttGT | circASPHD1 |
| 1247 | AAGcctgtttggttgtacTA | circASPHD1 |
| 1248 | TCtgtccctcaaTGA | circlTGAL |
| 1249 | CCtctgtccctcaaTG | circlTGAL |
| 1250 | AAcacctctgtccctCA | circlTGAL |
| 1251 | CGgaacacctctgtccCT | circlTGAL |
| 1252 | GTccctcaatgacatagAT | circlTGAL |
| 1253 | ACatcagaaggaGGC | circRP5-857K21.6.1 |
| 1254 | CGgcgaacatcaGAAG | circRP5-857K21.6.1 |
| 1255 | CGgcgaacatcagaaGG | circRP5-857K21.6.1 |
| 1256 | GGcgaacatcagaaggAG | circRP5-857K21.6.1 |
| 1257 | GAacatcagaaggaggcTT | circRP5-857K21.6.1 |
| 1258 | GCGaacatcagACAA | circRP5-857K21.6.2 |
| 1259 | CGgcgaacatcagaCA | circRP5-857K21.6.2 |
| 1260 | CGgcgaacatcagaCAA | circRP5-857K21.6.2 |
| 1261 | CGaacatcagacaaaTGC | circRP5-857K21.6.2 |
| 1262 | CGaacatcagacaaatgCA | circRP5-857K21.6.2 |
| 1263 | GAacatcagatGCGG | circRP5-857K21.6.3 |
| 1264 | GCgaacatcagatgCG | circRP5-857K21.6.3 |
| 1265 | CGaacatcagatgcgGG | circRP5-857K21.6.3 |
| 1266 | GGcgaacatcagatgcGG | circRP5-857K21.6.3 |
| 1267 | TCggcgaacatcagatgCG | circRP5-857K21.6.3 |
| 1268 | GCgaacatcagATGT | circRP5-857K21.6.4 |
| 1269 | ATCAgatgttgtTTAT | circRP5-857K21.6.4 |
| 1270 | ATCagatgttgttTATG | circRP5-857K21.6.4 |
| 1271 | GTcggcgaacatcagaTG | circRP5-857K21.6.4 |
| 1272 | CAgatgttgtttatgcgGG | circRP5-857K21.6.4 |
| 1273 | CAacagtccctggCT | circZNF720 |
| 1274 | GTcaacagtccctgGC | circZNF720 |
| 1275 | ATgtcaacagtccctGG | circZNF720 |
| 1276 | GAatgtcaacagtcccTG | circZNF720 |
| 1277 | AAcagtccctggctggaTG | circZNF720 |
| 1278 | ACcaactcaagCAGA | circLONP2 |
| 1279 | ACAtttcaaccaACTC | circLONP2 |
| 1280 | TCaaccaactcaagCAG | circLONP2 |
| 1281 | ATttcaaccaactcaaGC | circLONP2 |
| 1282 | AAcatttcaaccaacTCAA | circLONP2 |
| 1283 | AAATgactgtcGCTG | circCHD9 |
| 1284 | AAatgactgtcgCTGT | circCHD9 |
| 1285 | AAaatgactgtcgCTGT | circCHD9 |
| 1286 | TGactgtcgctgtaaaCA | circCHD9 |
| 1287 | TGactgtcgctgtaaacAT | circCHD9 |
| 1288 | GCCacataaacTATA | circSLC7A6 |
| 1289 | GCCacataaactaTAT | circSLC7A6 |
| 1290 | AAActatatgcaaGCAG | circSLC7A6 |
| 1291 | GCcacataaactatatGC | circSLC7A6 |
| 1292 | CACataaactatatgCAAG | circSLC7A6 |
| 1293 | GAcatggctgacTCA | circCARHSP1 |
| 1294 | CTgactcagagaTGTG | circCARHSP1 |
| 1295 | CTgactcagagatgtGC | circCARHSP1 |
| 1296 | ATggctgactcagagaTG | circCARHSP1 |
| 1297 | GAcatggctgactcagaGA | circCARHSP1 |
| 1298 | CACCtcagtaaGTC | circFANCA |
| 1299 | TGcacctcagtaaTGT | circFANCA |
| 1300 | CAcctcagtaatgtcCC | circFANCA |
| 1301 | AAtgtcacctcagTAAT | circFANCA |
| 1302 | AAtgtgcacctcagtaaTG | circFANCA |
| 1303 | TCACtcacgcagtGG | circRAD51D\|RAD51L3–RFFL |
| 1304 | ACatcactcacgcaGT | circRAD51D\|RAD51L3–RFFL |
| 1305 | CCacatcactcacgcAG | circRAD51D\|RAD51L3–RFFL |
| 1306 | AVatcactcacgcagtGG | circRAD51D\|RAD51L3–RFFL |
| 1307 | GCcacatcactcacgcaGT | circRAD51D\|RAD51L3–RFFL |
| 1308 | GGgccttgagctcTC | circHDAC5 |
| 1309 | GGgccttgagctctCC | circHDAC5 |
| 1310 | GGccttgagctctcctgCA | circHDAC5 |
| 1311 | TCttgaaccatCTGA | circUTP18 |
| 1312 | ATgttcttgaacCATC | circUTP18 |
| 1313 | TTgaaccatctgaAGAT | circUTP18 |
| 1314 | TTcttgaaccatctGAAG | circUTP18 |
| 1315 | TGttcttgaaccatctgAA | circUTP18 |
| 1316 | CAcctccacgacaCC | circSRSF1 |
| 1317 | CAcctccacgacacCA | circSRSF1 |
| 1318 | TCcgccacctccacgAC | circSRSF1 |
| 1319 | CCgccacctccacgacAC | circSRSF1 |
| 1320 | CTccgccacctccacgaCA | circSRSF1 |
| 1321 | CCattccgcatcaGG | circPPM1D |
| 1322 | TCcgcatcaggtaTTT | circPPM1D |
| 1323 | ATtccgcatcaggtaTT | circPPM1D |
| 1324 | CCattccgcatcaggtAT | circPPM1D |
| 1325 | GGccattccgcatcaggTA | circPPM1D |
| 1326 | GGccttgtaagaTGG | circBRIP1 |
| 1327 | AAggccttgtaaGATG | circBRIP1 |
| 1328 | AGgccttgtaagatgGC | circBRIP1 |
| 1329 | AAaggccttgtaagaTGG | circBRIP1 |
| 1330 | AAtatctgaaaaggcCTTG | circBRIP1 |
| 1331 | CAGCaaagtttGAAT | circPRKCA.1 |
| 1332 | CAGCaaagtttgaATC | circPRKCA.1 |
| 1333 | AGcaaagtttgaatcCC | circPRKCA.1 |
| 1334 | AAagtttgaatcccaGGA | circPRKCA.1 |
| 1335 | CACaaaacagcaaagTTTG | circPRKCA.1 |
| 1336 | AGGCttcagtttGAA | circPRKCA.2 |
| 1337 | AGGCttcagtttGAAT | circPRKCA.2 |
| 1338 | GCttcagtttgaatcCC | circPRKCA.2 |
| 1339 | GGcttcagtttgaatcCC | circPRKCA.2 |
| 1340 | TCagtttgaatcccaggAT | circPRKCA.2 |
| 1341 | GAgcctctcagagTA | circEIF4A1\|SNORD1 0\|RP11- 186B7.4\|SENP3- EIF4A1.1 |
| 1342 | CAtcacagagcctcTC | circEIF4A1\|SNORD1 0\|RP11- 186B7.4\|SENP3- EIF4A1.1 |
| 1343 | CTccatcacagagccTC | circEIF4A1\|SNORD1 0\|RP11- 186B7.4\|SENP3- EIF4A1.1 |
| 1344 | CAtcacagagcctctcAG | circEIF4A1\|SNORD1 0\|RP11- 186B7.4\|SENP3- EIF4A1.1 |
| 1345 | AGcctctcagagtacaaAG | circEIF4A1\|SNORD1 0\|RP11- 186B7.4\|SENP3- EIF4A1.1 |
| 1346 | CAtcacagagagcgCTC | circEIF4A1\|SNORD1 0\|RP11- 186B7.4\|SENP3- EIF4A1.2 |
| 1347 | TCcatcacagagcgCT | circEIF4A1\|SNORD1 0\|RP11- 186B7.4\|SENP3- EIF4A1.2 |
| 1348 | AGcgctctcagagtaCA | circEIF4A1\|SNORD1 0\|RP11- 186B7.4\|SENP3- EIF4A1.2 |
| 1349 | CAtcacagagcgctctCA | circEIF4A1\|SNORD1 0\|RP11- 186B7.4\|SENP3- EIF4A1.2 |
| 1350 | ATcacagagcgctctcaGA | circEIF4A1\|SNORD1 0\|RP11- 186B7.4\|SENP3- EIF4A1.2 |
| 1351 | CAggtttgcacTTTG | circPGS1 |
| 1352 | ACtcaggtttgcaCTT | circPGS1 |
| 1353 | CTcaggtttgcacttTG | circPGS1 |
| 1354 | TCactcaggtttgcacTT | circPGS1 |
| 1355 | TCaggtttgcactttgtAA | circPGS1 |
| 1356 | ACCaaatgagccTTG | circRPTOR |
| 1357 | CCaccaaatgagccTT | circRPTOR |
| 1358 | CCaccaaatgagcctTG | circRPTOR |
| 1359 | TGccaccaaatgagccTT | circRPTOR |
| 1360 | TGccaccaaatgagcctTG | circRPTOR |
| 1361 | GTgaggcaaatcACC | circRPL26\|RP11-849F2.7 |
| 1362 | TGcagtgaggcaAATC | circRPL26\|RP11-849F2.7 |
| 1363 | GCagtgaggcaaatcAC | circRPL26\|RP11-849F2.7 |
| 1364 | CTgcagtgaggcaaatCA | circRPL26\|RP11-849F2.7 |
| 1365 | GTgaggcaaatcacctgAG | circRPL26\|RP11-849F2.7 |
| 1366 | AAGAtatttgaCCAC | circRP11-206L10.8 |
| 1367 | GATatttgaccaCATA | circRP11-206L10.8 |
| 1368 | GATAtttgaccacaTAA | circRP11-206L10.8 |
| 1369 | TTaagatatttgacCACA | circRP11-206L10.8 |
| 1370 | TAagatatttgaccaCATA | circRP11-206L10.8 |
| 1371 | ACatgagaacccTGG | circPIAS2 |
| 1372 | ATAcatgagaaccCTG | circPIAS2 |
| 1373 | ATacatgagaaccctGG | circPIAS2 |
| 1374 | TGatacatgagaacccTG | circPIAS2 |
| 1375 | TGatacatgagaaccctGG | circPIAS2 |
| 1376 | CAGaggaagatCTGA | circTYMS |
| 1377 | GAGGaagatctgAATT | circTYMS |
| 1378 | GAAGatctgaattTTCA | circTYMS |
| 1379 | TCAgaggaagatctgAAT | circTYMS |
| 1380 | CAtcagaggaagatcTGAA | circTYMS |
| 1381 | CAACcaacgtgaAGG | circPPP4R1 |
| 1382 | AVvaavvaavgtGAAG | circPPP4R1 |
| 1383 | CAcaccaaccaacgtGA | circPPP4R1 |
| 1384 | ATccacaccaaccaacGT | circPPP4R1 |
| 1385 | AVvaavvaavgtgaaggAT | circPPP4R1 |
| 1386 | GAggacatataCCTT | circZNF91 |
| 1387 | AGGacatatacctTTC | circZNF91 |
| 1388 | GAcatatacctttcCAT | circZNF91 |
| 1389 | GAcatatacctttccaTA | circZNF91 |
| 1390 | GAcatatacctttccatAG | circZNF91 |
| 1391 | TGcctcacattgcGG | circWDR62 |
| 1392 | ATgtctctgttgccTC | circWDR62 |
| 1393 | TGttgcctcacattgCG | circWDR62 |
| 1394 | TGtctctgttgcctcaCA | circWDR62 |
| 1395 | GAtgtctctgttgcctcAC | circWDR62 |
| 1396 | ACTcagctcacGAAG | circADCK4 |
| 1397 | GCactcagctcacgAA | circADCK4 |
| 1398 | GCactcagctcacgaAG | circADCK4 |
| 1399 | GGcactcagctcacgAA | circADCK4 |
| 1400 | GGgcactcagctcacgaAG | circADCK4 |
| 1401 | ATttcttcctgtgGC | circARHGAP35 |
| 1402 | AAcatttcttccTGTG | circARHGAP35 |
| 1403 | CCaacatttcttcctGT | circARHGAP35 |
| 1404 | AGccaacatttcttccTG | circARHGAP35 |
| 1405 | AAcatttcttcctgtggCT | circARHGAP35 |
| 1406 | CGggtggccatgcAG | circNUCB1 |
| 1407 | GTggccatgcagtaTG | circNUCB1 |
| 1408 | GTggccatgcagtatGA | circNUCB1 |
| 1409 | CGggtggccatgcagtAT | circNUCB1 |
| 1410 | GGccatgcagtatgaagAA | circNUCB1 |
| 1411 | TCatcaccggccaTG | circSNORD33\|RPL1 3A.1 |
| 1412 | TCtcatcaccggccAT | circSNORD33\|RPL1 3A.1 |
| 1413 | TCtcatcaccggccaTG | circSNORD33\|RPL1 3A.1 |
| 1414 | GTtctcatcaccggccAT | circSNORD33\|RPL1 3A.1 |
| 1415 | GTtctcatcaccggccaTG | circSNORD33\|RPL1 3A.1 |
| 1416 | ATcaccggcctcaGA | circSNORD33\|RPL1 3A.2 |
| 1417 | TCtcatcaccggccTC | circSNORD33\|RPL1 3A.2 |
| 1418 | ATcaccggcctcagaTG | circSNORD33\|RPL1 3A.2 |
| 1419 | ATcaccggcctcagatGG | circSNORD33\|RPL1 3A.2 |
| 1420 | GTtctcatcaccggcctCA | circSNORD33\|RPL1 3A.2 |
| 1421 | AGttctcagatGGTA | circSNORD33\|RPL1 3A.3 |
| 1422 | AGttctcagatggTAG | circSNORD33\|RPL1 3A.3 |
| 1423 | AAgttctcagatgGTAG | circSNORD33\|RPL1 3A.3 |
| 1424 | AGaagttctcagatggTA | circSNORD33\|RPL1 3A.3 |
| 1425 | AGaagttctcagatggtAG | circSNORD33\|RPL1 3A.3 |
| 1426 | CGgcttagtgcgaTG | circMUC16 |
| 1427 | ACcccggcttagtgCG | circMUC16 |
| 1428 | ACcaccccggcttagTG | circMUC16 |
| 1429 | ACcccggcttagtgcgAT | circMUC16 |
| 1430 | CCaccccggcttagtgcGA | circMUC16 |
| 1431 | GAatagccaagGTCT | circLZIC |
| 1432 | CTgaatagccaagGTC | circLZIC |
| 1433 | CTgaatagccaaggtCT | circLZIC |
| 1434 | GCctgaatagccaaggTC | circLZIC |
| 1435 | AAtagccaaggtctgtaGA | circLZIC |
| 1436 | TTCCtagaacaGATC | circSNX5\|SNORD17 \|OVOL2.1 |
| 1437 | CCtagaacagatcAGA | circSNX5\|SNORD17 \|OVOL2.1 |
| 1438 | TTcctagaacagaTCAG | circSNX5\|SNORD17 \|OVOL2.1 |
| 1439 | TTcctagaacagatcAGA | circSNX5\|SNORD17 \|OVOL2.1 |
| 1440 | AGaacagatcagatttTCA | circSNX5\|SNORD17 \|OVOL2.1 |
| 1441 | CTAgaacaggaTCAG | circSNX5\|SNORD17 \|OVOL2.2 |
| 1442 | TAGaacaggatcAGAT | circSNX5\|SNORD17 \|OVOL2.2 |
| 1443 | CCtagaacaggatcaGA | circSNX5\|SNORD17 \|OVOL2.2 |
| 1444 | CCtagaacaggatcagAT | circSNX5\|SNORD17 \|OVOL2.2 |
| 1445 | TTcctagaacaggatcaGA | circSNX5\|SNORD17 \|OVOL2.2 |
| 1446 | TCggatgcaggaGAG | circSNORA71A\|SNH G17 |
| 1447 | CGgatgcaggagagTT | circSNORA71A\|SNH G17 |
| 1448 | CGgatgcaggagagtTG | circSNORA71A\|SNH G17 |
| 1449 | CGgatgcaggagagttGT | circSNORA71A\|SNH G17 |
| 1450 | AGgagagttgtgtgaaaGC | circSNORA71A\|SNH G17 |
| 1451 | GCtctgcgcaggtCC | circPLTP |
| 1452 | TGctgggctctgcgCA | circPLTP |
| 1453 | TGctgggctctgcgcAG | circPLTP |
| 1454 | AAtcactgctgggctcTG | circPLTP |
| 1455 | GCtgggctctgcgcaggTC | circPLTP |
| 1456 | GAcacagccctttGC | circTMEM230 |
| 1457 | CTgacacagcccttTG | circTMEM230 |
| 1458 | CTgacacagccctttGC | circTMEM230 |
| 1459 | TGacacagccctttgcTG | circTMEM230 |
| 1460 | AAcacgctgacacagccCT | circTMEM230 |
| 1461 | TCcctgaggcgtaTT | circCYP24A1 |
| 1462 | CTtccctgaggcgtAT | circCYP24A1 |
| 1463 | CTtccctgaggcgtaTT | circCYP24A1 |
| 1464 | CCcttccctgaggcgtAT | circCYP24A1 |
| 1465 | TCcctgaggcgtattatCG | circCYP24A1 |
| 1466 | GACTgggacagcgAG | circZBTB46 |
| 1467 | TCttctacagactgGG | circZBTB46 |
| 1468 | AGactggacagcgaGT | circZBTB46 |
| 1469 | TCttctacagactgggAV | circZBTB46 |
| 1470 | CTacagactgggacagcGA | circZBTB46 |
| 1471 | GAttgagatggCTCA | circGART |
| 1472 | CTgattgagatggCTC | circGART |
| 1473 | CTgattgagatggctCA | circGART |
| 1474 | AGAtggctcaaatGAAG | circGART |
| 1475 | TCactgattgagatggcTC | circGART |
| 1476 | ACccatcttaccaGG | circRAB3GAP1 |
| 1477 | ATacccatcttaccAG | circRAB3GAP1 |
| 1478 | AAtatacccatctTACC | circRAB3GAP1 |
| 1479 | GAagtaaatataccCATC | circRAB3GAP1 |
| 1480 | ATatacccatcttaccaGG | circRAB3GAP1 |
| 1481 | AVActggttagTTAG | circDYRK1A |
| 1482 | TATaacactggtTAGT | circDYRK1A |
| 1483 | AACTataacactgGTTA | circDYRK1A |
| 1484 | CAaaactataacacTGGT | circDYRK1A |
| 1485 | TAtaacactggttagtTAG | circDYRK1A |
| 1486 | TAtgtcaggtgCACA | circUNKNOWN0000 0007 |
| 1487 | TAtgtcaggtgcaCAG | circUNKNOWN0000 0007 |
| 1488 | GGgtatgtcaggtgcAV | circUNKNOWN0000 0007 |
| 1489 | TAtgtcaggtgcacagTG | circUNKNOWN0000 0007 |
| 1490 | TAtgtcaggtgcacagtGG | circUNKNOWN0000 0007 |
| 1491 | GTccatgtccacgGT | circCOL18A1.1 |
| 1492 | TGggtccatgtccaCG | circCOL18A1.1 |
| 1493 | CAtgtccacggtgtcTC | circCOL18A1.1 |
| 1494 | ATgtccacggtgtctcCT | circCOL18A1.1 |
| 1495 | GTccatgtccacggtgtCT | circCOL18A1.1 |
| 1496 | ACAcacaatacGCAC | circCOL18A1.2 |
| 1497 | ACacacacaataCGCA | circCOL18A1.2 |
| 1498 | ACacacacacaataCGC | circCOL18A1.2 |
| 1499 | CACacacacaatacgcAC | circCOL18A1.2 |
| 1500 | CAcacacacacaatacgCA | circCOL18A1.2 |
| 1501 | CCtctaggcttgaCA | circNBAS |
| 1502 | CCtctaggcttgacAT | circNBAS |
| 1503 | GCctctaggcttgacAT | circNBAS |
| 1504 | TAggcttgacatttccCA | circNBAS |
| 1505 | AGgcttgacatttcccaAT | circNBAS |
| 1506 | GGgccgacgcgcgAC | circCH507-513H4.1.1 |
| 1507 | GGGCcgacgcgcgaCG | circCH507-513H4.1.1 |
| 1508 | GCcgacgcgcgacggCG | circCH507-513H4.1.1 |
| 1509 | CGacgcgcgacggcggaCG | circCH507-513H4.1.1 |
| 1510 | CAcgggaccttccGC | circCH507-513H4.1.2 |
| 1511 | CAcgggaccttccgCG | circCH507-513H4.1.2 |
| 1512 | GCacgggaccttccgCG | circCH507-513H4.1.2 |
| 1513 | GGgcacgggaccttccGC | circCH507-513H4.1.2 |
| 1514 | GGgcacgggaccttccgCG | circCH507-513H4.1.2 |
| 1515 | CAacacgcccccgCG | circCH507- 513H4.1.3 |
| 1516 | CAacacgcccccgcGC | circCH507- 513H4.1.3 |
| 1517 | ACgcaacacgccccGC | circCH507- 513H4.1.3 |
| 1518 | ACgcaacacgcccccgCG | circCH507- 513H4.1.3 |
| 1519 | GCacgcaacacgcccccGC | circCH507- 513H4.1.3 |
| 1520 | CAcctcctcttTTAA | circANKAR |
| 1521 | AGcttcacctcctcTT | circANKAR |
| 1522 | CTtcacctcctctttTA | circANKAR |
| 1523 | CAgcttacacctcctctTT | circANKAR |
| 1524 | GCttcacctcctcttttAA | circANKAR |
| 1525 | CTCTgaacacatCTG | circGL5 |
| 1526 | TCTgaacacatcTGGA | circGL5 |
| 1527 | TCtgaacacatctGGAA | circGL5 |
| 1528 | TGctctgaacacatctGG | circGL5 |
| 1529 | TCtgaacacatctggaaGT | circGL5 |
| 1530 | TGcgaagtgagtGGT | circBMOPR2 |
| 1531 | TGcgaagtgagtggTG | circBMOPR2 |
| 1532 | CGaagtgagtggtgTTG | circBMOPR2 |
| 1533 | CGaagtgagtggtgttGT | circBMOPR2 |
| 1534 | CGaagtgagtggtgttgTG | circBMOPR2 |
| 1535 | AGacggctgaaccCT | circRHBDD1 |
| 1536 | CGgctgaaccctggTG | circRHBDD1 |
| 1537 | GAcggctgaaccctgGT | circRHBDD1 |
| 1538 | TGaaccctggtgagaaTA | circRHBDD1 |
| 1539 | AACcctggtgagaataAAT | circRHBDD1 |
| 1540 | CCatacacatgaGGT | circATG16L1\|SCARNA5 |
| 1541 | TCccatacacatgaGG | circATG16L1\|SCARNA5 |
| 1542 | GAtcccatacacatgAG | circATG16L1\|SCARNA5 |
| 1543 | ATcccatacacatgagGT | circATG16L1\|SCARNA5 |
| 1544 | TGatcccatacacatgaGG | circATG16L1\|SCARNA5 |
| 1545 | GATGgtcgtaaAACT | circDGKD |
| 1546 | GATggtcgtaaaACTG | circDGKD |
| 1547 | ATggtcgtaaaacTGTT | circDGKD |
| 1548 | TGATggtcgtaaaacTGT | circDGKD |
| 1549 | TGgtcgtaaaactgttgTT | circDGKD |
| 1550 | CTtctctgtggtCTT | circPASK |
| 1551 | GTcttctctgtggtCT | circPASK |
| 1552 | CTgtcttctctgtggTC | circPASK |
| 1553 | CTccatctgtcttctcTG | circPASK |
| 1554 | TCcatctgtcttctctgTG | circPASK |
| 1555 | CTttacttgaaGCGG | circPPP6R2 |
| 1556 | TTTacttgaagcGGAC | circPPP6R2 |
| 1557 | TCtttacttgaagcgGA | circPPP6R2 |
| 1558 | TCtttacttgaagcggAC | circPPP6R2 |
| 1559 | ATAatctctttacttGAAG | circPPP6R2 |
| 1560 | GTttagctgtcACAG | circBIRC6 |
| 1561 | GTttagctgtcacaGA | circBIRC6 |
| 1562 | ACctggtttagctgtCA | circBIRC6 |
| 1563 | ACctggtttagctgtcAC | circBIRC6 |
| 1564 | GTttagctgtcacagaaTC | circBIRC6 |
| 1565 | AGttagcctttTGAT | circPRKD3 |
| 1566 | AGttagccttttGATA | circPRKD3 |
| 1567 | ACatatagttagcCTTT | circPRKD3 |
| 1568 | TGacatatagttagcCTT | circPRKD3 |
| 1569 | TTagccttttgataaaCTA | circPRKD3 |
| 1570 | AGactttctttTGCT | circKIAA1841\|RP11- 493E12.3 |
| 1571 | GActttcttttgCTTC | circKIAA1841\|RP11- 493E12.3 |
| 1572 | AAgactttcttttGCTT | circKIAA1841\|RP11- 493E12.3 |
| 1573 | AGactttcttttgcttCC | circKIAA1841\|RP11- 493E12.3 |
| 1574 | ATGacagaacaagacTTTC | circKIAA1841\|RP11- 493E12.3 |
| 1575 | CGaggaccacaTGAC | circRTKN |
| 1576 | TAacgaggaccaCATG | circRTKN |
| 1577 | TAacgaggaccacaTGA | circRTKN |
| 1578 | TGTaacgaggaccacAT | circRTKN |
| 1579 | GTaacgaggaccactgAC | circRTKN |
| 1580 | TCAcaacttgaAGGG | circELMOD3 |
| 1581 | ACTcacaacttgAAGG | circELMOD3 |
| 1582 | ACtcacaacttgaaGGG | circELMOD3 |
| 1583 | TCtgtactcacaacttGA | circELMOD3 |
| 1584 | TGtactcacaacttaaGG | circELMOD3 |
| 1585 | TGgtggagcttcTTT | circREV1 |
| 1586 | GAgcttctttccaCAA | circREV1 |
| 1587 | GAgcttctttccacaAT | circREV1 |
| 1588 | GAgcttctttccacaaTC | circREV1 |
| 1589 | CTtctttccacaatccaTT | circREV1 |
| 1590 | ACtgatgtcaccTGT | circZBTB20 |
| 1591 | TCacctgtgtgtacGA | circZBTB20 |
| 1592 | TGtcacctgtgtgtaCG | circZBTB20 |
| 1593 | GAtgtcacctgtgtgtAC | circZBTB20 |
| 1594 | ATgtcacctgtgtgtacGA | circZBTB20 |
| 1595 | GCagattgcactCTT | circTIMMDC1 |
| 1596 | AGattgcactcttcCA | circTIMMDC1 |
| 1597 | GTgcagattgcactcTT | circTIMMDC1 |
| 1598 | GCagattgcactcttcCA | circTIMMDC1 |
| 1599 | ATgtgcagattgcactcTT | circTIMMDC1 |
| 1600 | CTggcaactcacGAG | circACAD9 |
| 1601 | CTggcaactcacgaGG | circACAD9 |
| 1602 | TGgcaactcacgaggAG | circACAD9 |
| 1603 | CTggcaactcacgaggAG | circACAD9 |
| 1604 | CAACtcacgaggaggcCGC | circACAD9 |
| 1605 | TCaggttgattgGGT | circPLXND1 |
| 1606 | GTtcaggttgattgGG | circPLXND1 |
| 1607 | TCgttcaggttgattGG | circPLXND1 |
| 1608 | CGttcaggttgattggGT | circPLXND1 |
| 1609 | CTcgttcaggttgattgGG | circPLXND1 |
| 1610 | CCccgcttggcaaAG | circHDAC11 |
| 1611 | CAccccgcttggcaAA | circHDAC11 |
| 1612 | TGgaagccaccccgcTT | circGDAC11 |
| 1613 | TGgaagccaccccgctTG | circGDAC11 |
| 1614 | TGgaagccacccgcttGG | circGDAC11 |
| 1615 | TAGTtactgtaAAGT | circCEP70 |
| 1616 | TAGTtactgtaaAGTT | circCEP70 |
| 1617 | GAAAcatagttacTGTA | circCEP70 |
| 1618 | CCggaaacatagttacTG | circCEP70 |
| 1619 | ACatagttactgtaaAGTT | circCEP70 |
| 1620 | TCACcatgaaaATGA | circRNF13.1 |
| 1621 | CACCatgaaaatgACT | circRNF13.1 |
| 1622 | CACcatgaaaatgACTA | circRNF13.1 |
| 1623 | GTtgtaaaatcaccaTGA | circRNF13.1 |
| 1624 | TCgttgtaaaatcaccATG | circRNF13.1 |
| 1625 | CACCtttcttgaATT | circRNF13.2 |
| 1626 | ATCacctttcttGAAT | circRNF13.2 |
| 1627 | CACctttcttgaatTTA | circRNF13.2 |
| 1628 | GTaaatcacctttCTTG | circRNF13.2 |
| 1629 | TAaaatcacctttctTGAA | circRNF13.2 |
| 1630 | GTctctcctgcacCT | circGOLIM4 |
| 1631 | TGtatacagtctctCC | circGOLIM4 |
| 1632 | CAgtctctcctgcacCT | circGOLIM4 |
| 1633 | TCtctcctgcacctctCG | circGOLIM4 |
| 1634 | AAttgtatacagtctctCC | circGOLIM4 |
| 1635 | GCcatcatttcaCAT | circEIF4A2\|SNORD2.1 |
| 1636 | CCatcatttcacatCA | circEIF4A2\|SNORD2.1 |
| 1637 | TGccatctttcacaTC | circEIF4A2\|SNORD2.1 |
| 1638 | TTgccatcatttcacaTC | circEIF4A2\|SNORD2.1 |
| 1639 | CCatcatttcacatcaaAT | circEIF4A2\|SNORD2.1 |
| 1640 | TTTcacatttaCCGA | circEIF4A2\|SNORD2.2 |
| 1641 | CAtttcacatttACCG | circEIF4A2\|SNORD2.2 |
| 1642 | ATcatttcacattTACC | circEIF4A2\|SNORD2.2 |
| 1643 | TCatttcacatttacCGA | circEIF4A2\|SNORD2.2 |
| 1644 | GCcatcatttcacatttAC | circEIF4A2\|SNORD2.2 |
| 1645 | TCTggctgtgcaAAT | circSDHAP1 |
| 1646 | GTGCaaatttcaaAGT | circSDHAP1 |
| 1647 | GTgcaaatttcaaAGTC | circSDHAP1 |
| 1648 | TGgctgtgcaaatttCAA | circSDHAP1 |
| 1649 | GCaaatttcaaagtccaGA | circSDHAP1 |
| 1650 | GGTgttctgaatcCT | circSETD2 |
| 1651 | GTgttctgaatccTTC | circSETD2 |
| 1652 | GTgttctgaatccttCT | circSETD2 |
| 1653 | TTctgaatccttcttACG | circSETD2 |
| 1654 | GAcaaaggtgttctgaATC | circSETD2 |
| 1655 | CAtaccgccattGAG | circSCAP |
| 1656 | CAcataccgccattGA | circSCAP |
| 1657 | CCcacataccgccatTG | circSCAP |
| 1658 | CAcataccgccattgaGG | circSCAP |
| 1659 | CCcacataccgccattgAG | circSCAP |
| 1660 | AGataccttcaaCCT | circUSP4 |
| 1661 | GTCaataagataCCTT | circUSP4 |
| 1662 | AATAagataccttCAAC | circUSP4 |
| 1663 | GTcaataagatacctTCA | circUSP4 |
| 1664 | ATaagataccttcaaccTC | circUSP4 |
| 1665 | ACtcacgcagcatGT | circRPL29 |
| 1666 | GCactcacgcagcaTG | circRPL29 |
| 1667 | GCactcacgcagcatGT | circRPL29 |
| 1668 | GTgcactcacgcagcaTG | circRPL29 |
| 1669 | TCagtgcactcacgcagCA | circRPL29 |
| 1670 | GCtcttccagaaGAC | circPHF7 |
| 1671 | GCtcttccagaagaCA | circPHF7 |
| 1672 | TTCCagaagacagAAAT | circPHF7 |
| 1673 | TAcaggctcttccagaAG | circPHF7 |
| 1674 | CCAgaagacagaaataATG | circPHF7 |
| 1675 | GTttcctttcctcTC | circNEK4 |
| 1676 | CTttcctctctaTCAA | circNEK4 |
| 1677 | GTttcctttcctctcTA | circNEK4 |
| 1678 | CCtttcctctctatcaAA | circNEK4 |
| 1679 | TTcctctctatcaaattCA | circNEK4 |
| 1680 | TAcatggagaccgGG | circFLNB |
| 1681 | ACtacatggagaccGG | circFLNB |
| 1682 | ACtacatggagaccgGG | circFLNB |
| 1683 | CCactacatggagaccGG | circFLNB |
| 1684 | CCactacatggagaccgGG | circFLNB |
| 1685 | AGCagctctctTAAA | circSLC25A26 |
| 1686 | GCAgctctcttaaAAC | circSLC25A26 |
| 1687 | TGcagcagctctcttAA | circSLC25A26 |
| 1688 | TGcagcagctctcttaAA | circSLC25A26 |
| 1689 | AGcagctctcttaaaacTG | circSLC25A26 |
| 1690 | TCtgaagtttgACCT | circNFKB1 |
| 1691 | ATtctgaagtttGACC | circNFKB1 |
| 1692 | ATtctgaagtttgaCCT | circNFKB1 |
| 1693 | CCattctgaagtttgaCC | circNFKB1 |
| 1694 | CCattctgaagtttgacCT | circNFKB1 |
| 1695 | TGtttccggtggtGG | circFIP1L1\|RP11-231C18.3 |
| 1696 | AAgtgctgtttccgGT | circFIP1L1\|RP11-231C18.3 |
| 1697 | AGaagtgctgtttccGG | circFIP1L1\|RP11-231C18.3 |
| 1698 | TGctgtttccggtggtGG | circFIP1L1\|RP11-231C18.3 |
| 1699 | AAgtgctgtttccggtgGT | circFIP1L1\|RP11-231C18.3 |
| 1700 | CCAaggagaaaCTTG | circTBC1D14 |
| 1701 | AAGGagaaacttGCAA | circTBC1D14 |
| 1702 | CCaaggagaaacttgCA | circTBC1D14 |
| 1703 | GAGaaacttgcaaaGAAG | circTBC1D14 |
| 1704 | TTggtccaaggagaaacTT | circTBC1D14 |
| 1705 | AAGGttacttcATTC | circALB.1 |
| 1706 | GGGAaataaaggTTAC | circALB.1 |
| 1707 | ATAaaggttacttCATT | circALB.1 |
| 1708 | GGAaataaaggttaCTTC | circALB.1 |
| 1709 | AGggaaataaaggttACTT | circALB.1 |
| 1710 | TTCcaacagagGTTT | circALB.2 |
| 1711 | CAacagaggttttTGC | circALB.2 |
| 1712 | CAacagaggtttttgCA | circALB.2 |
| 1713 | CAacagaggtttttgCAA | circALB.2 |
| 1714 | TTttccaacagaggttTTT | circALB.2 |
| 1715 | ACGAcagagtaATCA | circALB.3 |
| 1716 | AGTaatcaggatGTTT | circALB.3 |
| 1717 | CAgagtaatcaggaTGT | circALB.3 |
| 1718 | AGagtaatcaggatGTTT | circALB.3 |
| 1719 | GAcagagtaatcaggatGT | circALB.3 |
| 1720 | CCactgatcatctGG | circNUP54 |
| 1721 | CCactgatcatctgGC | circNUP54 |
| 1722 | CActgatcatctggCAA | circNUP54 |
| 1723 | CActgatcatctggcaAT | circNUP54 |
| 1724 | AAtcctccaaacccactGA | circNUP54 |
| 1725 | TGTAcaaactcaACT | circAFF1 |
| 1726 | GTCAttgtacaaaCTC | circAFF1 |
| 1727 | GTAcaaactcaacTGAC | circAFF1 |
| 1728 | GTacaactcaactgACT | circAFF1 |
| 1729 | TGtacaaactcaactgACT | circAFF1 |
| 1730 | CGtatgttggctTCA | circSLC12A7\|MIR46 35 |
| 1731 | ACcgtatgttggctTC | circSLC12A7\|MIR46 35 |
| 1732 | GGgaccgtatgttggCT | circSLC12A7\|MIR46 35 |
| 1733 | GAccgtatgttggcttCA | circSLC12A7\|MIR46 35 |
| 1734 | TTagggaccgtatgttgGC | circSLC12A7\|MIR46 35 |
| 1735 | TGgccctgggtcgTT | circMAN2A1.1 |
| 1736 | GCcctgggtcgttaTG | circMAN2A1.1 |
| 1737 | GCcctgggtcgttatGG | circMAN2A1.1 |
| 1738 | CTggccctgggtcgttAT | circMAN2A1.1 |
| 1739 | TGagagctggccctgggTC | circMAN2A1.1 |
| 1740 | GCccttttaacagCA | circMAN2A1.2 |
| 1741 | GCtggcccttttaaCA | circMAN2A1.2 |
| 1742 | AGagctggcccttttAA | circMAN2A1.2 |
| 1743 | ATtgagagctggccctTT | circMAN2A1.2 |
| 1744 | GCccttttaacagcatcCT | circMAN2A1.2 |
| 1745 | TCattcttaacCTCC | circAFF4 |
| 1746 | TCAttcttaacctcCT | circAFF4 |
| 1747 | ATcttcattcttaaCCT | circAFF4 |
| 1748 | TCttcattcttaacctCC | circAFF4 |
| 1749 | TCatcttcattcttaacCT | circAFF4 |
| 1750 | ATCattcaattCCTT | circUBE2D2 |
| 1751 | ATTCaattcctttTGA | circUBE2D2 |
| 1752 | GAtcattcaattcCTTT | circUBE2D2 |
| 1753 | CAATtccttttgaaATAG | circUBE2D2 |
| 1754 | ATtccttttgaaataGTTA | circUBE2D2 |
| 1755 | AGactgcgactttGT | circANKHD1\|ANKHD1-EIF4EBP3 |
| 1756 | AGactgcgactttgTA | circANKHD1\|ANKHD1-EIF4EBP3 |
| 1757 | AGactgcgactttgtAG | circANKHD1\|ANKHD1-EIF4EBP3 |
| 1758 | ACtgcgactttgtagcAA | circANKHD1\|ANKHD1-EIF4EBP3 |
| 1759 | ACtgcgactttgtagcaAG | circANKHD1\|ANKHD1-EIF4EBP3 |
| 1760 | AGATcctctatGAAT | circMAPK9 |
| 1761 | GTttcagatcctcTAT | circMAPK9 |
| 1762 | GATcctctatgaaTTAT | circMAPK9 |
| 1763 | CAgatcctctatgaaTTA | circMAPK9 |
| 1764 | TTcagatcctctatgaATT | circMAPK9 |
| 1765 | ATtctgtgttctgCC | circGPBP1 |
| 1766 | CGgaggatattcTGTG | circGPBP1 |
| 1767 | GAtattctgtgttctGC | circGPBP1 |
| 1768 | ATtctgtgttctgcctCA | circGPBP1 |
| 1769 | GAtattctgtgttctgcCT | circGPBP1 |
| 1770 | CAgcggagtgaagGT | circCEP72 |
| 1771 | CTcagcggagtgaaGG | circCEP72 |
| 1772 | AGctcagcggagtgaAG | circCEP72 |
| 1773 | GCtcagcggagtgaagGT | circCEP72 |
| 1774 | AAtgactgaagctcagcGG | circCEP72 |
| 1775 | AAtcaccatccatGGT | circRP11-98J23.2 |
| 1776 | GCaatcaccatcctGG | circRP11-98J23.2 |
| 1777 | GCaatcaccatcctgGT | circRP11-98J23.2 |
| 1778 | AAtcaccatcctggtgAA | circRP11-98J23.2 |
| 1779 | CAtcctggtgaagccttAC | circRP11-98J23.2 |
| 1780 | CCtctgtaggctTAA | circFAM169A |
| 1781 | ATcctctgtaggctTA | circFAM169A |
| 1782 | CCatcctctgtaggcTT | circFAM169A |
| 1783 | AAtgccatcctctgtaGG | circFAM169A |
| 1784 | ATcctctgtaggcttaaCT | circFAM169A |
| 1785 | CAaataataacTGTT | circWDR41 |
| 1786 | TGCAaataataaCTGT | circWDR41 |
| 1787 | CAGcagatgcaaaTAAT | circWDR41 |
| 1788 | GCAaataataactgtTCT | circWDR41 |
| 1789 | GCaaataataactgtTCTA | circWDR41 |
| 1790 | ACtgctggtggacTC | circRASGRF2 |
| 1791 | GGactgctggtggaCT | circRASGRF2 |
| 1792 | AGactctaggactgcTG | circRASGRF2 |
| 1793 | AGgactgctggtggacTC | circRASGRF2 |
| 1794 | CTaggactgctggtggaCT | circRASGRF2 |
| 1795 | TTtctttcctggTGT | circRHOBTB3 |
| 1796 | TTtttctttcctgGTG | circRHOBTB3 |
| 1797 | TTtttctttcctggtGT | circRHOBTB3 |
| 1798 | TTtctttcctggtgtttTT | circRHOBTB3 |
| 1799 | AGttgaatgatCTGC | circCEP85L |
| 1800 | TGatctgcaaacTAGC | circCEP85L |
| 1801 | GAtctgcaaactagcCA | circCEP85L |
| 1802 | GAtctgcaaactagccAC | circCEP85L |
| 1803 | GAatgatctgcaaactaGC | circCEP85L |
| 1804 | CAagacttcaaCCTG | circARID1B.1 |
| 1805 | TTcaacctgggaTACT | circARID1B.1 |
| 1806 | CCaagacttcaacctGG | circARID1B.1 |
| 1807 | TTcaacctgggatactTG | circARID1B.1 |
| 1808 | CAagacttcaacctgggAT | circARID1B.1 |
| 1809 | CAgacagatcctCTT | circARID1B.2 |
| 1810 | GCcagacagatcctCT | circARID1B.2 |
| 1811 | AGatcctctttcctgTG | circARID1B.2 |
| 1812 | GGagccagacagatccTC | circARID1B.2 |
| 1813 | GAgccagacagatcctcTT | circARID1B.2 |
| 1814 | CTtacaaatctCGCT | circTULP4\|RP11- 732M18.4 |
| 1815 | GTCttacaaatcTCGC | circTULP4\|RP11- 732M18.4 |
| 1816 | GAgtcttacaaatCTCG | circTULP4\|RP11- 732M18.4 |
| 1817 | AGtcttacaaatctcgCT | circTULP4\|RP11- 732M18.4 |
| 1818 | AGtcttacaaatctcgcTA | circTULP4\|RP11- 732M18.4 |
| 1819 | CAgcacctttcagCC | circTULP4 |
| 1820 | CCaaacagcaccttTC | circTULP4 |
| 1821 | AAacagcacctttcaGC | circTULP4 |
| 1822 | GTgccaaacagcacctTT | circTULP4 |
| 1823 | TGccaaacagcacctttCA | circTULP4 |
| 1824 | TAgccctgcacatGT | circTMEM181 |
| 1825 | CTttagccctgcacAT | circTMEM181 |
| 1826 | TTtagccctgcacatGT | circTMEM181 |
| 1827 | GGctttagccctgcacAT | circTMEM181 |
| 1828 | TTtgaattggctttagcCC | circTMEM181 |
| 1829 | CTTgggcgatttcTC | circHIST1H3B |
| 1830 | CGatttctcgttaGC | circHIST1H3B |
| 1831 | CGatttctcgcttagCA | circHIST1H3B |
| 1832 | GGgcgatttctcgcttAG | circHIST1H3B |
| 1833 | ATttctcgcttagcatgGA | circHIST1H3B |
| 1834 | GTGTctctgaaaAGA | circHIST1H3C.2 |
| 1835 | AAGTgtctctgaaAAG | circHIST1H3C.2 |
| 1836 | CAcaaaagtgtctCTGA | circHIST1H3C.2 |
| 1837 | CAcacaaaagtgtctCTG | circHIST1H3C.2 |
| 1838 | CAcacacaaaagtgtctCT | circHIST1H3C.2 |
| 1839 | GCcgaccaaagccAA | circUNKNOWN00000008 |
| 1840 | CCgaccaaagccaaAG | circUNKNOWN00000008 |
| 1841 | GGccgaccaaagccaAA | circUNKNOWN00000008 |
| 1842 | CCgaccaaagccaaagGC | circUNKNOWN00000008 |
| 1843 | AGctaaccggccgaccaAA | circUNKNOWN00000008 |
| 1844 | CTTgggaagcggTAA | circC6orf136 |
| 1845 | TTGggaagcggtaATG | circC6orf136 |
| 1846 | GCttgggaagcggtaAT | circC6orf136 |
| 1847 | GCttgggaagcggtaaTG | circC6orf136 |
| 1848 | TGggaagcggtaatgctCG | circC6orf136 |
| 1849 | GAgacatccagccCA | circHLA-C\|HLA-B\|XXbac-BPG248L24.10 \|WASF5P \|XXbac-BPG248L24.13.1 |
| 1850 | GAgacatccagcccAC | circHLA-C\|HLA-B \|XXbac-BPG248L24.10 \|WASF5P\|XXbac-BPG248L24.13.1 |
| 1851 | GAgacatccagcccaCC | circHLA-C\|HLA-B \|XXbac-BPG248L24.10 \|WASF5P\|XXbac-BPG248L24.13.1 |
| 1852 | CAgcccacctctctggAA | circHLA-C\|HLA-B \|XXbac-BPG248L24.10\|WASF5P\|XXbac-BPG248L24.13.1 |
| 1853 | ATggagacatccagcccAC | circHLA-C\|HLA- B\|XXbac- BPG248L24.10\|WA SF5P\|XXbax-BPG248L24.13.1 |
| 1854 | CTtttccacctgaGC | circHLA-C\|HLA- B\|XXbac- BPG248L24.10\|WA SF5P\|XXbax-BPG248L24.13.2 |
| 1855 | CTccttttccacctGA | circHLA-C\|HLA- B\|XXbac- BPG248L24.10\|WA SF5P\|XXbax-BPG248L24.13.2 |
| 1856 | CTccttttccacctgAG | circHLA-C\|HLA- B\|XXbac- BPG248L24.10\|WA SF5P\|XXbax-BPG248L24.13.2 |
| 1857 | CTccttttccacctgaGC | circHLA-C\|HLA- B\|XXbac- BPG248L24.10\|WA SF5P\|XXbax-BPG248L24.13.2 |
| 1858 | TCcttttccacctgagcTC | circHLA-C\|HLA- B\|XXbac- BPG248L24.10\|WA SF5P\|XXbax-BPG248L24.13.2 |
| 1859 | GTAtccatctcTAAC | circFKBP5 |
| 1860 | GTatccatctctaaCC | circFKBP5 |
| 1861 | GTatccatctctaacCA | circFKBP5 |
| 1862 | GTatccatctctaaccAG | circFKBP5 |
| 1863 | TCtttggtatccatctcTA | circFKBP5 |
| 1864 | ACACttggcaaaTCG | circCNPY3 |
| 1865 | TTtacacacttgGCAA | circCNPY3 |
| 1866 | ACacacttggcaaATCG | circCNPY3 |
| 1867 | TAtttacacacttggCAA | circCNPY3 |
| 1868 | TTacacacttggcaaatCG | circCNPY3 |
| 1869 | TACCtacgcagggAC | circSRF |
| 1870 | ATccctacctacgcAG | circSRF |
| 1871 | CTacctacgcagggaCT | circSRF |
| 1872 | CCctacctacgcagggAC | circSRF |
| 1873 | CTacctacgcagggactGG | circSRF |
| 1874 | GTgagggaggaagGG | circRN7SK |
| 1875 | CGgtgagggaggaaGG | circRN7SK |
| 1876 | CGgtgagggaggaagGG | circRN7SK |
| 1877 | AGcggtgagggaggaaGG | circRN7SK |
| 1878 | AGcggtgagggaggaagGG | circRN7SK |
| 1879 | TAACtgaattgACCA | circFARS2 |
| 1880 | ACCagcaaataacTGA | circFARS2 |
| 1881 | AGCAaataactgaATTG | circFARS2 |
| 1882 | TACcagcaaataacTGAA | circFARS2 |
| 1883 | TTataccagcaaataACTG | circFARS2 |
| 1884 | GCAGagacatatTTG | circMLIP |
| 1885 | CACcagcagagacaTA | circMLIP |
| 1886 | CAgcagagacataTTTG | circMLIP |
| 1887 | ACcaccagcagagacaTA | circMLIP |
| 1888 | AGacatatttggtttctCG | circMLIP |
| 1889 | CTAggagtgtcTTTA | circZNF292 |
| 1890 | CTaggagtgtctTTAT | circZNF292 |
| 1891 | CTaggagtgtctttATC | circZNF292 |
| 1892 | CTaggagtgtctttatCC | circZNF292 |
| 1893 | AGtgtctttatccttatCC | circZNF292 |
| 1894 | AAcattgattgTGCC | circPNRC1 |
| 1895 | AAAAcattgattGTGC | circPNRC1 |
| 1896 | AAaacattgattgTGCC | circPNRC1 |
| 1897 | TTAaaacattgattGTGC | circPNRC1 |
| 1898 | TTGAttttaaaacatTGAT | circPNRC1 |
| 1899 | GTcacgaggaaTAGT | circUNKNOWN0000 0009 |
| 1900 | TCACgaggaataGTAAA | circUNKNOWN0000 0009 |
| 1901 | AGtcacgagaggaataGTA | circUNKNOWN0000 0009 |
| 1902 | CTtacagtcacgaggAAT | circUNKNOWN0000 0009 |
| 1903 | AGtcacgaggaatagTAAA | circUNKNOWN0000 0009 |
| 1904 | ACcaccggcattgTA | circNDUFB2 |
| 1905 | CGgcattgtacagaGT | circNDUFB2 |
| 1906 | ACcggcattgtacagAG | circNDUFB2 |
| 1907 | CCaccggcattgtacaGA | circNDUFB2 |
| 1908 | ACcggcattgtacagagTG | circNDUFB2 |
| 1909 | GTTAtaatcagCAAT | circKMT2C |
| 1910 | TTGTtataatcaGCAA | circKMT2C |
| 1911 | TTCAttgttataaTCAG | circKMT2C |
| 1912 | TGTTataatcagcaATAA | circKMT2C |
| 1913 | GTTataatcagcaataAGA | circKMT2C |
| 1914 | TTAgaacacccgGAA | circESYT2 |
| 1915 | CCttagaacacccgGA | circESYT2 |
| 1916 | ATccttagaacacccGG | circESYT2 |
| 1917 | TCcttagaacacccggAA | circESYT2 |
| 1918 | TTagaacacccggaaggTT | circESYT2 |
| 1919 | GTcatggcaccgtAG | circMPP6 |
| 1920 | TAgtcatggcaccgTA | circMPP6 |
| 1921 | TTtagtcatggcaccGT | circMPP6 |
| 1922 | GTcatggcaccgtagtTC | circMPP6 |
| 1923 | GCaaaatgatttagtCATG | circMPP6 |
| 1924 | TTcgtcaaccttGCA | circHERPUD2 |
| 1925 | CCttcgtcaaccttGC | circHERPUD2 |
| 1926 | ATccttcgtcaacctTG | circHERPUD2 |
| 1927 | TGatccttcgtcaaccTT | circHERPUD2 |
| 1928 | CAaccttgcattacataTG | circHERPUD2 |
| 1929 | TCaaacctcatCAGC | circOGDH |
| 1930 | AActcctcaaacCTCA | circOGDH |
| 1931 | CCtcaaacctcatcaGC | circOGDH |
| 1932 | GGaactcctcaaacctCA | circOGDH |
| 1933 | AActcctcaaacctcatCA | circOGDH |
| 1934 | TCagtggtccctgGG | circZNF680 |
| 1935 | TGtcagtggtccctGG | circZNF680 |
| 1936 | AAtgtcagtggtcccTG | circZNF680 |
| 1937 | ATgtcagtggtccctgGG | circZNF680 |
| 1938 | AAatgtcagtggtccctGG | circZNF680 |
| 1939 | GTctcggactacgCC | circKDELR2\|DAGLB |
| 1940 | TGgtgatggtctcgGA | circKDELR2\|DAGLB |
| 1941 | GGtctcggactacgcCG | circKDELR2\|DAGLB |
| 1942 | GGtgatggtctcggacTA | circKDELR2\|DAGLB |
| 1943 | TGatggtctcggactacGC | circKDELR2\|DAGLB |
| 1944 | ACACacctgaaTAAG | circZDHHC4 |
| 1945 | GTtacacacaccTGAA | circZDHHC4 |
| 1946 | CAgttacacacacCTGA | circZDHHC4 |
| 1947 | TAcacacacctgaaTAAG | circZDHHC4 |
| 1948 | ACAcaccctgaataagAAAG | circZDHHC4 |
| 1949 | AAGCtcaggttcgAT | circCCZ1B |
| 1950 | AAAagctcaggtTCGA | circCCZ1B |
| 1951 | AAGCtcaggttcgatGT | circCCZ1B |
| 1952 | AAaaagctcaggttCGAT | circCCZ1B |
| 1953 | GTaccattaaaaagctCAG | circCCZ1B |
| 1954 | GCccctggactatGT | circPOM121 |
| 1955 | GCccctggactatgTT | circPOM121 |
| 1956 | ACactggcccctggaCT | circPOM121 |
| 1957 | TGavavtggcccctggAV | circPOM121 |
| 1958 | CTggcccctggactatgTT | circPOM121 |
| 1959 | TCatgcgaggacAGT | circBAZ1B |
| 1960 | AGtttcatgcgagGAC | circBAZ1B |
| 1961 | GTtccagtttcatgcGA | circBAZ1B |
| 1962 | TTtcatgcgaggacagTA | circBAZ1B |
| 1963 | TCatgcgaggacagtaaTC | circBAZ1B |
| 1964 | ACgaccaccttcGC | circGTF2I |
| 1965 | TCacacgaccacctTC | circGTF2I |
| 1966 | CAcacgaccaccttcTG | circGTF2I |
| 1967 | ACacgaccaccttctgCT | circGTF2I |
| 1968 | ACacgaccaccttctgcTG | circGTF2I |
| 1969 | GAATgcaatagATGT | circSNORA14A |
| 1970 | AATGcaatagatGTTG | circSNORA14A |
| 1971 | GCaatagatgttgcaGG | circSNORA14A |
| 1972 | AAtagatgttgcagGTAT | circSNORA14A |
| 1973 | GTTtaagaatgcaatAGAT | circSNORA14A |
| 1974 | CAcatatcgagcTGG | circCDK14 |
| 1975 | GAcacatatcgagCTG | circCDK14 |
| 1976 | GTgacacatatcgagCT | circCDK14 |
| 1977 | TGacacatatcgagcTGG | circCDK14 |
| 1978 | TGTgacacatatcgagcTG | circCDK14 |
| 1979 | TGgtgtaacatGCTA | circCCDC132 |
| 1980 | GTaacatgctatgGTA | circCCDC132 |
| 1981 | GGtgtaacatgctatGG | circCCDC132 |
| 1982 | CTggtgtaacatgctaTG | circCCDC132 |
| 1983 | ATgctatggtaattcttGA | circCCDC132 |
| 1984 | TTGgcaatcagATCT | circTRRAP \|MIR3609 |
| 1985 | CTTTggcaatcagATC | circTRRAP \|MIR3609 |
| 1986 | CActttggcaatcaGAT | circTRRAP \|MIR3609 |
| 1987 | ACtttggcaatcagatCT | circTRRAP \|MIR3609 |
| 1988 | TTggcaatcagatcttaTC | circTRRAP \|MIR3609 |
| 1989 | ACtatttcaaaatAC | circCYP3A7 \|CYP3A7-CYP3A51P |
| 1990 | AGGAgaactatttCAA | circCYP3A7 \|CYP3A7-CYP3A51P |
| 1991 | TTtcaaaatacttGGCA | circCYP3A7 \|CYP3A7-CYP3A51P |
| 1992 | TTTcaaaatacttgGCAA | circCYP3A7 \|CYP3A7-CYP3A51P |
| 1993 | GAACtatttcaaaatACTT | circCYP3A7 \|CYP3A7-CYP3A51P |
| 1994 | TGgcccaacctggTA | circCCAT1.1 |
| 1995 | GTagtgcctggcccAA | circCCAT1.1 |
| 1996 | CTggcccaacctggtAA | circCCAT1.1 |
| 1997 | GGcccaacctggtaagTG | circCCAT1.1 |
| 1998 | GCctggcccaacctggtAA | circCCAT1.1 |
| 1999 | TGcctggtaagtGG | circCCAT1.2 |
| 2000 | CCtggcctggtaagTG | circCCAT1.2 |
| 2001 | GAgtagtgcctggccTG | circCCAT1.2 |
| 2002 | AGtgcctggcctggtaAG | circCCAT1.2 |
| 2003 | GAgtagtgcctggcctgGT | circCCAT1.2 |
| 2004 | AAtcccaacctgGTA | circCCAT1.3 |
| 2005 | TAaatcccaacctgGT | circCCAT1.3 |
| 2006 | TTgccattaaatccCAA | circCCAT1.3 |
| 2007 | TAaatcccaacctggTAA | circCCAT1.3 |
| 2008 | ATcttgccattaaatccCA | circCCAT1.3 |
| 2009 | TGtcaggctcccaAC | circCCAT1.4 |
| 2010 | CTcccaacctggtaAG | circCCAT1.4 |
| 2011 | TGatgtcaggctcccAA | circCCAT1.4 |
| 2012 | GAtgtcaggctcccaaCC | circCCAT1.4 |
| 2013 | CTcccaacctggtaagtGG | circCCAT1.4 |
| 2014 | TAATaaccaacCTGG | circCCAT1.5 |
| 2015 | GCCattaataacCAAC | circCCAT1.5 |
| 2016 | CCAttaataaccaacCT | circCCAT1.5 |
| 2017 | CCattaataaccaaccTG | circCCAT1.5 |
| 2018 | GAgagccattaataacCAA | circCCAT1.5 |
| 2019 | ATacagtcccaacCT | circCCAT1.6 |
| 2020 | ACAatacagtccCAAC | circCCAT1.6 |
| 2021 | CAatacagtcccaacCT | circCCAT1.6 |
| 2022 | GCagacaatacagtccCA | circCCAT1.6 |
| 2023 | GCagacaatacagtcccAA | circCCAT1.6 |
| 2024 | GTctctttccaacCT | circCCAT1.7 |
| 2025 | CTgtctctttccaaCC | circCCAT1.7 |
| 2026 | CTgtctctttccaacCT | circCCAT1.7 |
| 2027 | TTtccaacctggtaagTG | circCCAT1.7 |
| 2028 | ATtgttctgtctctttcCA | circCCAT1.7 |
| 2029 | TTTgtatgtggCATG | circASAP1 |
| 2030 | ATttttgtatgtgGCA | circASAP1 |
| 2031 | CAatttttgtatgtGGC | circASAP1 |
| 2032 | ATttttgtatgtggcATG | circASAP1 |
| 2033 | TTctcaatttttgtaTGTG | circASAP1 |
| 2034 | TGCtgggcagaaaTC | circPTK2.1 |
| 2035 | TGGgcagaaatcTTTC | circPTK2.1 |
| 2036 | GCTgggcagaaatcTTT | circPTK2.1 |
| 2037 | GCtgggcagaaatcttTC | circPTK2.1 |
| 2038 | TGggcagaaatctttccTC | circPTK2.1 |
| 2039 | ATCTgtcatattCTG | circPTK2.2 |
| 2040 | TCATattctgttGATA | circPTK2.2 |
| 2041 | TGtcatattctgtTGAT | circPTK2.2 |
| 2042 | TCtgtcatattctgttGA | circPTK2.2 |
| 2043 | TAtctgtcatattctgtTG | circPTK2.2 |
| 2044 | CTGgatgatcaATCT | circSLC45A4 |
| 2045 | TGATcaatctgcaaCA | circSLC45A4 |
| 2046 | GATgatcaatctgCAAC | circSLC45A4 |
| 2047 | ATgatcaatctgcaaCAG | circSLC45A4 |
| 2048 | ATgatcaatctgcaacaGT | circSLC45A4 |
| 2049 | AGcacatcactaCCA | circADGRB1 |
| 2050 | ATAagcacatcaCTAC | circADGRB1 |
| 2051 | TAagccatcactacCA | circADGRB1 |
| 2052 | CACtataagcacatcaCT | circADGRB1 |
| 2053 | ACtataagcacatcacTAC | circADGRB1 |
| 2054 | TCCGgacattcaaAG | circRBPMS |
| 2055 | TAgggtccggacatTC | circRBPMS |
| 2056 | CGgacattcaaagCATT | circRBPMS |
| 2057 | CGgacattcaaagcaTTC | circRBPMS |
| 2058 | CGgacattcaaagcattCT | circRBPMS |
| 2059 | TGacccttgttcaGG | circFGFR1 |
| 2060 | ACtgacccttgttcAG | circFGFR1 |
| 2061 | TGacccttgttcaggCA | circFGFR1 |
| 2062 | ACtgacccttgttcagGC | circFGFR1 |
| 2063 | AAactgacccttgttcaGG | circFGFR1 |
| 2064 | TTACtcatcagcTGA | circHOOK3 |
| 2065 | TACtcatcagctGAAT | circHOOK3 |
| 2066 | TCtttactcatcagcTG | circHOOK3 |
| 2067 | TCatcagctgaatgtTTC | circHOOK3 |
| 2068 | CAgctgaatgtttcttcTT | circHOOK3 |
| 2069 | CAtgctttgtcTCTA | circASPH |
| 2070 | TGCtttgtctctaaTA | circASPH |
| 2071 | ATGctttgtctctaATA | circASPH |
| 2072 | CTccatgctttgtctcTA | circASPH |
| 2073 | ATGctttgtctctaaTAAA | circASPH |
| 2074 | CCagatcagaaGACT | circTMEM245 |
| 2075 | AACgtccagatcAGAA | circTMEM245 |
| 2076 | ACcaacgtccagatcAG | circTMEM245 |
| 2077 | CGtccagatcagaagaCT | circTMEM245 |
| 2078 | CCaccaacgtccagatcAG | circTMEM245 |
| 2079 | CAttactgcctggCG | circUNKNOWN00000010 |
| 2080 | GAcccaccattactGC | circUNKNOWN00000010 |
| 2081 | TGgacccaccattacTG | circUNKNOWN00000010 |
| 2082 | CAccattactgcctggCG | circUNKNOWN00000010 |
| 2083 | ATggacccaccattactGC | circUNKNOWN00000010 |
| 2084 | TTCCttgattgTAAC | circHSPAS |
| 2085 | TTCCttgattgtaaCA | circHSPAS |
| 2086 | ATggttccttgattgTA | circHSPAS |
| 2087 | ATggttccttgattgTAA | circHSPAS |
| 2088 | CGggatggttccttgatTG | circHSPAS |
| 2089 | CAtctgttccttTCA | circGLE1 |
| 2090 | CAtctgttcctttCAT | circGLE1 |
| 2091 | AAACatctgttccTTTC | circGLE1 |
| 2092 | CTaaaacatctgttcCTT | circGLE1 |
| 2093 | AAacatctgttcctttCAT | circGLE1 |
| 2094 | TCctccaaagtTGTA | circFOCAD |
| 2095 | ATtcctccaaagtTGT | circFOCAD |
| 2096 | CCtccaaagttgtatTC | circFOCAD |
| 2097 | CCaaagttgtattcaAGA | circFOCAD |
| 2098 | CCtccaaagttgtattcAA | circFOCAD |
| 2099 | GTtgctccgaaACTT | circNFX1 |
| 2100 | GTtgctccgaaaCTTT | circNFX1 |
| 2101 | TGctccgaaactttGAT | circNFX1 |
| 2102 | TGttgctccgaacttTG | circNFX1 |
| 2103 | GCtccgaaactttgataAG | circNFX1 |
| 2104 | GATGacaagatGACT | circUBAP2 |
| 2105 | CAAggacggatgACAA | circUBAP2 |
| 2106 | GAcggatgacaagaTGA | circUBAP2 |
| 2107 | AAggacggatgacaaGAT | circUBAP2 |
| 2108 | CGgatgacaagatgactGA | circUBAP2 |
| 2109 | GTATagcatacCAAT | circKDM4C\|RP11- 146B14.1 |
| 2110 | GCataccaatttggCA | circKDM4C\|RP11- 146B14.1 |
| 2111 | TAgcataccaatttgGC | circKDM4C\|RP11- 146B14.1 |
| 2112 | GTatacataccaatTTG | circKDM4C\|RP11- 146B14.1 |
| 2113 | AGcataccaatttggcaAC | circKDM4C\|RP11- 146B14.1 |
| 2114 | AGTCttatagtaACA | circAGTPBP1 |
| 2115 | AGTCttatagtaACAA | circAGTPBP1 |
| 2116 | AGTCttatagtaaCAAA | circAGTPBP1 |
| 2117 | ATTAaatcatagtcTTAT | circAGTPBP1 |
| 2118 | GTCttatagtaacaaaTGT | circAGTPBP1 |
| 2119 | CTgtgcaaccttTAG | circFAM120A.1 |
| 2120 | TGtgcaacctttaGTG | circFAM120A.1 |
| 2121 | CTgtgcaacctttagTG | circFAM120A.1 |
| 2122 | GCtctgtgcaacctttAG | circFAM120A.1 |
| 2123 | CTctgtgcaacctttagTG | circFAM120A.1 |
| 2124 | AACctgtgaatGCTG | circFAM120A.2 |
| 2125 | TGcaacctgtgaatGC | circFAM120A.2 |
| 2126 | CTgtgcaacctgtgaAT | circFAM120A.2 |
| 2127 | CTctgtgcaacctgtgAA | circFAM120A.2 |
| 2128 | AAcctgtgaatgctgGAAA | circFAM120A.2 |
| 2129 | GTagaaccatgggCT | circHIATL1 |
| 2130 | GAaccatgggctgaTC | circHIATL1 |
| 2131 | GTttcatgtagaacCAT | circHIATL1 |
| 2132 | TAgaaccatgggctgaTC | circHIATL1 |
| 2133 | TGtagaaccatgggctgAT | circHIATL1 |
| 2134 | AAcaccatcttggCC | circPPP2R3B |
| 2135 | AAaacaccatctTGGC | circPPP2R3B |
| 2136 | AAaacaccatcttggCC | circPPP2R3B |
| 2137 | TTaaaacaccatcttgGC | circPPP2R3B |
| 2138 | TTaaaacaccatcttggCC | circPPP2R3B |
| 2139 | TCACtctctcaATTT | circATRX |
| 2140 | GCtcactctctcaaTT | circATRX |
| 2141 | CTgctcactctctcaAT | circATRX |
| 2142 | AAtcttcactgctcacTC | circATRX |
| 2143 | TCactgctcactctctcAA | circATRX |
| 2144 | TAcagcacaccaCAC | circTBL1X |
| 2145 | CAtacagcacacCACA | circTBL1X |
| 2146 | ATacagcacaccacaCA | circTBL1X |
| 2147 | ATacagcacaccacacAC | circTBL1X |
| 2148 | CAgacatacagcacaccAC | circTBL1X |
| 2285 | AATctgtctcttcACC | circFAT1 |
| 2286 | CGggaatctgtctcTTC | circFAT1 |
| 2287 | TGTcgggaatctgtCT | circFAT1 |
| 2288 | ATAcctgtagtacCGA | circHIPK3 |
| 2289 | TGaggccatacctgtAG | circHIPK3 |
| 2290 | ATATttttctggcAATC | circCORO1C |
| 2291 | ATTtttctggcaatCTC | circCORO1C |
| 2292 | AGgcctcaagcccaGC | circPVT1 |
| 2293 | GGcctcaagcccagCT | circPVT1 |
| 2294 | CTTagtctcctcATAA | circFIRRE |
| 2295 | GACaccttagtctcCT | circFIRRE |
| 2296 | TCTCctcataaaGTAT | circFIRRE |
| 2297 | TATCtgcaatagTCAA | circCCT3 |
| 2298 | TGCAatagtcaaGAAT | circCCT3 |
| 2299 | CTGcatatttttctgGC | circCORO1C |

In the examples and figures, compounds named CRM0167-CRM170 and CRM0172-CRM0174 and CRM0175-CRM0182 corresponds to SEQ ID NO’s: 2285-2288 and SEQ ID NO’s: 2289-2291 and SEQ ID NO’s 2292-2298 respectively. CRM0171 correspond to SEQ ID NO: 374. CRM0175 corresponds to SEQ ID NO 2299.

Each compound listed in Table 2 is to be viewed as single embodiments. In some preferred embodiments, the LNA units in the wings of the antisense oligonucleotide of the invention are Beta-D-Oxy LNA and the antisense oligonucleotide is anyone of SEQ ID NOs: 360 - 2148 or anyone of SEQ ID NO’s: 2285-2299. In some preferred embodiments, the LNA units in the wings of the antisense oligonucleotide of the invention are alpha-L-oxy-LNA and the antisense oligonucleotide is anyone of SEQ ID NOs: 360 - 2148 or anyone of SEQ ID NO’s: 2285-2299. In some preferred embodiments, the LNA units in the wings of the antisense oligonucleotide of the invention are beta-D-amino-LNA and the antisense oligonucleotide is anyone of SEQ ID NOs: 360 - 2148 or anyone of SEQ ID NO’s: 2285-2299. In some preferred embodiments, the LNA units in the wings of the antisense oligonucleotide of the invention are alpha-L-amino-LNA and the antisense oligonucleotide is anyone of SEQ ID NOs: 360 - 2148 or anyone of SEQ ID NO’s: 2285-2299. In some preferred embodiments, the LNA units in the wings of the antisense oligonucleotide of the invention are beta-D-thio-LNA and the antisense oligonucleotide is anyone of SEQ ID NOs: 360 - 2148 or anyone of SEQ ID NO’s: 2285-2299. In some preferred embodiments, the LNA units in the wings of the antisense oligonucleotide of the invention are alpha-L-thio-LNA and the antisense oligonucleotide is anyone of SEQ ID NOs: 360 - 2148 or anyone of SEQ ID NO’s: 2285-2299. In some preferred embodiments, the LNA units in the wings of the antisense oligonucleotide of the invention are 5′-methyl-LNA and the antisense oligonucleotide is anyone of SEQ ID NOs: 360 - 2148 or anyone of SEQ ID NO’s: 2285-2299. In some preferred embodiments, the LNA units in the wings of the antisense oligonucleotide of the invention are beta-D-ENA and the antisense oligonucleotide is anyone of SEQ ID NOs: 360 - 2148 or anyone of SEQ ID NO’s: 2285-2299. In some preferred embodiments, the LNA units in the wings of the antisense oligonucleotide of the invention are alpha-L-ENA and the antisense oligonucleotide is anyone of SEQ ID NOs: 360 - 2148 or anyone of SEQ ID NO’s: 2285-2299. In some embodiments, the nucleoside analogues in the wings are not LNA, but tricyclo-DNA and the antisense oligonucleotide is anyone of SEQ ID NOs: 360 -2148 or anyone of SEQ ID NO’s: 228561-2299. In some embodiments, the nucleoside analogues in the wings are not LNA, but 2′-Fluoro and the antisense oligonucleotide is anyone of SEQ ID NOs: 360 - 2148 or anyone of SEQ ID NO’s: 2285-2299. In some embodiments, the nucleoside analogues in the wings are not LNA, but 2′-O-methyl and the antisense oligonucleotide is anyone of SEQ ID NOs: 360 - 2148 or anyone of SEQ ID NO’s: 2285-2299. In some embodiments, the nucleoside analogues in the wings are not LNA, but 2′-MOE and antisense oligonucleotide is anyone of SEQ ID NOs: 360 - 2148 or anyone of SEQ ID NO’s: 2285-2299. In some embodiments, the nucleoside analogues in the wings are not LNA, but 2′cyclic ethyl (cET) and the antisense oligonucleotide is anyone of SEQ ID NOs: 360 - 2148 or anyone of SEQ ID NO’s: 2285-2299. In some embodiments, the nucleoside analogues in the wings are not LNA, but UNA and the antisense oligonucleotide is anyone of SEQ ID NOs: 360 - 2148 or anyone of SEQ ID NO’s: 2285-2299. In some embodiments, the nucleoside analogues in the wings are not LNA, but CRN and the antisense oligonucleotide is anyone of SEQ ID NOs: 360 - 2148 or anyone of SEQ ID NO’s: 2285-2299. In some embodiments, the nucleoside analogues in the wings are partly LNA, but mixed with another nucleotide analogue selected from the list of tricyclo-DNA, 2′Fluoro, 2′-O-methyl, 2′methoxyethyl (2′-MOE), 2′cyclic ethyl (cET), UNA and Conformationally Restricted Nucleoside (CRN) and the antisense oligonucleotide is anyone of SEQ ID NOs: 360 - 2148 or anyone of SEQ ID NO’s: 2285-2299.

For most pharmaceuticals, efficient delivery is important, in order to ensure cost efficient, effective treatment without adverse effects.

In certain embodiments, the antisense oligonucleotide according to the invention is conjugated with a ligand for targeted delivery, to ensure that the compound is directed to the right target cells for uptake in an efficient manner. In some embodiments, this is achieved by conjugation with folic acid or N-acetylgalactosamine (GalNAc). Folic acid conjugation will ensure uptake in, for example folate receptor-positive cancer cells. Likewise, conjugation with GalNac markedly improves delivery to hepatocytes in the liver.

In some instances, it is preferred to deliver the antisense oligonucleotide in an unconjugated form in a pharmaceutical composition. This approach may be used in order to ensure delivery to the right cellular compartment in the target cell.

In some instances, the antisense oligonucleotide according to the invention is formulated in lipid nanoparticles for delivery. It is well known that lipid nanoparticle formulations of e.g. siRNA may be an effective way of delivery to e.g. hepatocytes in vivo. Further, particle size seems to be important for potency, so that if particle size is above 30 mm, the formulation is more potent than for smaller particle sizes (Chen et al., J Control Release, 2016 May 26, pii: 50168-3659 (16)30349-2).

It is well known that long noncoding RNAs, including circRNAs, may be implicated in disease pathogenesis, why the antisense oligonucleotides according to the invention in preferred embodiments are for use as a medicament.

In particular, the antisense oligonucleotide according to the invention is for use as a medicament in the treatment of cancer, such as hepatocellular carcinoma.

In some embodiments, the antisense oligonucleotides of the invention are formulated in a composition comprising the antisense oligonucleotide and a pharmaceutically acceptable diluent, carrier, salt or adjuvant.

The antisense oligonucleotides of the invention are also useful in compositions for pharmaceutical use or in methods of treatment. In some embodiments, the compositions of the present invention may comprise more than one of the antisense oligonucleotides according to the invention. In one such embodiment, one or more antisense oligonucleotides comprised in the compositions target different circRNAs or different IncRNAs, such as lincRNAs.

When the compositions according to the invention comprise more than one antisense oligonucleotide, such antisense oligonucleotides may be selected from the list of anyone of SEQ ID NOs: 360 - 2148 or anyone of SEQ ID NO’s: 2285-2299.

The invention also provides compositions that comprise the antisense oligonucleotides of the invention, for the treatment of cancer, such as for treatment of hepatocellular carcinoma, breast cancer, CNS tumors, leukemias, melanoma, non-small cell lung cancer, prostate cancer or renal cancer.

In some embodiments, the antisense oligonucleotide according to the invention, or composition, is for treatment of human subjects.

In some embodiments, the antisense oligonucleotide or composition according to the invention is for treatment of a cell ex vivo.

In some embodiments, the invention provides methods of downregulating an endogenous circRNA in a cell, by the administration of a composition comprising an effective amount of an antisense oligonucleotide according to the invention to a cell.

In some embodiments, the invention provides a method for the treatment of cancer, comprising the administration of an effective dosage of an antisense oligonucleotide or a composition according to the invention to a human.

In some embodiments, the invention provides a method of treatment of cancer, wherein the cancer is selected from the list of cancers such as hepatocellular carcinoma or prostate cancer, and wherein the antisense oligonucleotides of the invention are administered to a cancer patient in an effective dosage.

In some embodiments, the invention provides, the antisense oligonucleotides of the invention, or the compositions or methods of treatment according to the invention, wherein the antisense oligonucleotide, or compositions or methods of treatment are for use in combination with another compound, composition or method of treatment, by which combination a synergic or additive effect may be achieved, or treatment of different symptoms of the disease may be achieved.

A method of treating cancer, characterized by the following steps:
a) Isolate cancer cells from a patient.
b) Testing the presence of circRNA in the cancer cells.
c) If the cancer cell is tested positive for a circRNA in step b, for one or more circRNAs, a composition comprising an antisense oligonucleotide according to anyone of claims 1-14 is selected, wherein the antisense oligonucleotide is antisense to the circRNA that is expressed according to the test in step b.

The cancer is treated with the composition of step c, by administering an efficient amount of the composition to the patient having the cancer.

The method according to the previous embodiment, wherein the circRNA level measured in step b is anyone of a circRNA that is expressed in a cancer cell.

The method according to the previous embodiment, wherein the circRNA level measured in step b is anyone selected from the list of ciRS-7, circFAT1, circPVT1, circHIPK3, circSRY, circSLC35E2B, circCDK11A, circUNKNOWN00000001, circARHGAP32, circSLC8A3, circHERC2, circZFAND6, circRP1-168P16.1, circAURKC, circAFTPH, circSCD, circSMC3, circSNORA23|IP07.1, circZNF124.1, circSNX5|OVOL2, circRALY, circTFPI, circAHSG.1, circAHSG.2, circAHSG.3, circUBXN7, circAFP, circHIST1H3A, circHIST1H3C.1, circANAPC2, circRMRP |RMRP, circCENPI, circFIRRE, circMBNL3, circGPC3, circPROSER2, circMALRD1, circFAM208B, circMCU, circKIF20B, circABCC2, circEIF4G2|SNORD97.1, circEIF4G2 |SNORD97.2, circEIF4G2|SNORD97.3, circEIF4G2|SNORD97.4, circEIF4G2|SNORD97.5, circEIF4G2|SNORD97.6, circEIF4G2|SNORD97.7, circEIF4G2|SNORD97.8, circEIF4G2|SNORD97.9, circEIF4G2|SNORD97.10, circIGF2, circQSER1, circUNKNOWN00000002, circCHD1L, circPRUNE, circSLC27A3, circGATAD2B, circKIAA0907, circCCT3, circPLEKHM2, circVWCE, circATF6, circMALAT1.1, circMALAT1.2, circMALAT1.3, circMALAT1.4, circMALAT1.5, circMALAT1.6, circMALAT1.7, circMALAT1.8, circMALAT1.9, circMALAT1.10, circMALAT1.11, circMALAT1.12, circMALAT1.13, circUNKNOWN00000003, circMALAT1.14, circMALAT1.15, circMALAT1.16, circMALAT1.17, circMALAT1.18, circMALAT1.19, circUCK2, circSUCO, circRAB6A, circRPS3 |SNORD15B.1, circRPS3|SNORD15B.2, circRPS3|SNORD15B.3, circRSF1, circABL2, circGNB1, circRPLP2|SNORA52, circPICALM.1, circPICALM.2, circSNORA23|IPO7.2, circSNORA23 |IPO7.3, circCFH, circSLC41A2.1, circSLC41A2.2, circCORO1C, circEIF4G3|RP11-487E1.2, circNAA25, circMED13L, circLPGAT1|RN7SL344P, circAACS, circTP53BP2, circSOX5, circDNAH14, circKDM1A|MIR3115, circTTC13, circEGLN1, circTCEA3, circTOMM20|SNORA14B, circSCCPDH, circZNF124.2, circGLS2, circR3HDM2, circDHDDS, circSNORA73A|RCC1|SNHG3.1, circSNORA73A|RCC1|SNHG3.2, circSNORA61|SNHG12, circCEP831 RBMS2P1, circFGD6, circPUM1, circTMCO3|RP11-230F18.6, circPTP4A2, circZMYM5, circN6AMT2, circRPL21|SNORA27, circGTF2F2, circZMYM4, circLINC00355, circUNKNOWN00000004, circFARP1, circDYNC1H1, circCDC42BPB, circCCNB1IP1|SNORA79AL355075.1, circRPPH1 |RPPH1.1, circRPPH1|RPPH1.2, circRPPH1|RPPH1.3, circRPPH1|RPPH1.4, circSNORD8|CHD8.1, circSNORD8|CHD8.2, circPPP1R3E, circCHMP4A|RP11-468E2.1|AL136419.6, circUNKNOWN00000005, circSEC23A, circSNORD46| RPS8, circSAMD4A, circPCNX, circPSEN1, circFCF1, circSCARNA13|SNHG10.1, circSCARNA13|SNHG10.2, circSCARNA13 |SNHG10.3, circUNKNOWN00000006, circTJP1, circRP11-632K20.7, circTTBK2, circPPIB, circUBE2Q2, circETFA, circSEC11A, circPDE8A, circDAB1|OMA1, circABHD2, circlQGAP1.1, circlQGAP1.2, circCHD2, circlGF1R, circNPRL3, circNDE1, circABCC1, circRPS2 |SNORA64, circPOLR3E, circATXN2L, circMVP, circASPHD1, circITGAL, circRP5-857K21.6.1, circRP5-857K21.6.2, circRP5-857K21.6.3, circRP5-857K21.6.4, circZNF720, circLONP2, circCHD9, circSLC7A6, circCARHSP1, circFANCA, circRAD51D|RAD51L3-RFFL, circHDAC5, circUTP18, circSRSF1, circPPM1D, circBRIP1, circPRKCA.1, circPRKCA.2, circEIF4A1|SNORD10|RP11-186B7.4|SENP3-EIF4A1.1, circEIF4A1|SNORD10|RP11-186B7.4|SENP3-EIF4A1.2, circPGS1, circRPTOR, circRPL26|RP11-849F2.7, circRP11-206L10.8, circPIAS2, circTYMS, circPPP4R1, circZNF91, circWDR62, circADCK4, circARHGAP35, circNUCB1, circSNORD33|RPL13A.1, circSNORD33|RPL13A.2, circSNORD33|RPL13A.3, circMUC16, circLZIC, circSNX5|SNORD17|OVOL2.1, circSNX5 |SNORD17|OVOL2.2, circSNORA71A|SNHG17, circPLTP, circTMEM230, circCYP24A1, circZBTB46, circGART, circRAB3GAP1, circDYRK1A, circUNKNOWN00000007, circCOL18A1.1, circCOL18A1.2, circNBAS, circCH507-513H4.1.1, circCH507-513H4.1.2, circCH507-513H4.1.3, circANKAR, circGLS, circBMPR2, circRHBDD1, circATG16L1|SCARNA5, circDGKD, circPASK, circPPP6R2, circBIRC6, circPRKD3, circKIAA1841|RP11-493E12.3, circRTKN, circELMOD3, circREV1, circZBTB20, circTIMMDC1, circACAD9, circPLXND1, circHDAC11, circCEP70, circRNF13.1, circRNF13.2, circGOLIM4, circEIF4A2|SNORD2.1, circEIF4A2|SNORD2.2, circSDHAP1, circSETD2, circSCAP, circUSP4, circRPL29, circPHF7, circNEK4, circFLNB, circSLC25A26, circNFKB1, circFIP1L1|RP11-231C18.3, circTBC1D14, circALB.1, circALB.2, circALB.3, circNUP54, circAFF1, circSLC12A7| MIR4635, circMAN2A1.1, circMAN2A1.2, circAFF4, circUBE2D2, circANKHD1|ANKHD1-EIF4EBP3, circMAPK9, circGPBP1, circCEP72, circRP11-98J23.2, circFAM169A, circWDR41, circRASGRF2, circRHOBTB3, circCEP85L, circARID1B.1, circARID1B.2, circTULP4|RP11-732M18.4, circTULP4, circTMEM181, circHIST1H3B, circHIST1H3C.2, circUNKNOWN00000008, circC6orf136, circHLA-C|HLA-B|XXbac-BPG248L24.10|WASF5P|XXbac-BPG248L24.13.1, circHLA-C|HLA-B |XXbac-BPG248L24.10|WASF5P|XXbac-BPG248L24.13.2, circFKBP5, circCNPY3, circSRF, circRN7SK, circFARS2, circMLIP, circZNF292, circPNRC1, circUNKNOWN00000009, circNDUFB2, circKMT2C, circESYT2, circMPP6, circHERPUD2, circOGDH, circZNF680, circKDELR2|DAGLB, circZDHHC4, circCCZ1B, circPOM121, circBAZ1B, circGTF2I, circSNORA14A, circCDK14, circCCDC132, circTRRAP|MIR3609, circCYP3A7|CYP3A7-CYP3A51P, circCCAT1.1, circCCAT1.2, circCCAT1.3, circCCAT1.4, circCCAT1.5, circCCAT1.6, circCCAT1.7, circASAP1, circPTK2.1, circPTK2.2, circSLC45A4, circADGRB1, circRBPMS, circFGFR1, circHOOK3, circASPH, circTMEM245, circUNKNOWN00000010, circHSPA5, circGLE1, circFOCAD, circNFX1, circUBAP2, circKDM4C|RP11-146B14.1, circAGTPBP1, circFAM120A.1, circFAM120A.2, circHIATL1, circPPP2R3B, circATRX, or circTBL1X.

In some embodiments, the present invention provides antisense oligonucleotides suitable for the manufacture of a medicament for the treatment of a disease as referred to herein.

In one embodiment, the invention comprises a method for treating a disease as referred to herein, said method comprising administering an antisense oligonucleotide as disclosed herein, and/or a conjugate, and/or a pharmaceutical composition to a patient in need thereof.

One or more embodiment provided herein relates to methods of treating or preventing a cancer disease by modulating the activity of specific targets in cancer patients.

In one embodiment, the invention comprises a method of treating cancer in humans comprising administering to the human a therapeutically effective amount of the compound or composition according to the invention, thereby treating the cancer. In such embodiments, the skilled artisan will know how to determine what an effective dosage for the individual patient will be.

The present invention further provides pharmaceutical compositions, comprising therapeutically active antisense oligonucleotides in accordance with the invention, together with one or more pharmaceutically acceptable excipients. In some preferred embodiments, the invention provides compositions, such as pharmaceutical compositions comprising antisense oligonucleotides according to the invention, which are in single embodiments complementary to anyone of a circRNA selected from the list of ciRS-7, circFAT1, circPVT1, circHIPK3, circSRY, circSLC35E2B, circCDK11A, circUNKNOWN00000001, circARHGAP32, circSLC8A3, circHERC2, circZFAND6, circRP1-168P16.1, circAURKC, circAFTPH, circSCD, circSMC3, circSNORA23|IP07.1, circZNF124.1, circSNX5|OVOL2, circRALY, circTFPI, circAHSG.1, circAHSG.2, circAHSG.3, circUBXN7, circAFP, circHIST1H3A, circHIST1H3C.1, circANAPC2, circRMRP|RMRP, circCENPI, circFIRRE, circMBNL3, circGPC3.

In other preferred embodiments, the invention provides compositions, such as pharmaceutical compositions comprising antisense oligonucleotides according to the invention which are in single embodiments complementary to anyone of the circRNAs selected from the list of ciRS-7, circFAT1, circPVT1, circHIPK3, circSRY, circSLC35E2B, circCDK11A, circUNKNOWN00000001, circARHGAP32, circSLC8A3, circHERC2, circZFAND6, circRP1-168P16.1, circAURKC, circAFTPH, circSCD, circSMC3, circSNORA23|IP07.1, circZNF124.1, circSNX5|OVOL2, circRALY, circTFPI, circAHSG.1, circAHSG.2, circAHSG.3, circUBXN7, circAFP, circHIST1H3A, circHIST1H3C.1, circANAPC2, circRMRP|RMRP, circCENPI, circFIRRE, circMBNL3, circGPC3, circPROSER2, circMALRD1, circFAM208B, circMCU, circKIF20B, circABCC2, circEIF4G2|SNORD97.1, circEIF4G2|SNORD97.2, circEIF4G2|SNORD97.3, circEIF4G2|SNORD97.4, circEIF4G2|SNORD97.5, circEIF4G2|SNORD97.6, circEIF4G2|SNORD97.7, circEIF4G2|SNORD97.8, circEIF4G2|SNORD97.9, circEIF4G2|SNORD97.10, circlGF2, circQSER1, circUNKNOWN00000002, circCHD1L, circPRUNE, circSLC27A3, circGATAD2B, circKIAA0907, circCCT3, circPLEKHM2, circVWCE, circATF6, circMALAT1.1, circMALAT1.2, circMALAT1.3, circMALAT1.4, circMALAT1.5, circMALAT1.6, circMALAT1.7, circMALAT1.8, circMALAT1.9, circMALAT1.10, circMALAT1.11, circMALAT1.12, circMALAT1.13, circUNKNOWN00000003, circMALAT1.14, circMALAT1.15, circMALAT1.16, circMALAT1.17, circMALAT1.18, circMALAT1.19, circUCK2, circSUCO, circRAB6A, circRPS3|SNORD15B.1, circRPS3|SNORD15B.2, circRPS3|SNORD15B.3, circRSF1, circABL2, circGNB1, circRPLP2|SNORA52, circPICALM.1, circPICALM.2, circSNORA23|IPO7.2, circSNORA23|IPO7.3, circCFH, circSLC41A2.1, circSLC41A2.2, circCORO1C, circEIF4G3|RP11-487E1.2, circNAA25, circMED13L, circLPGAT1|RN7SL344P, circAACS, circTP53BP2, circSOX5, circDNAH14, circKDM1A|MIR3115, circTTC13, circEGLN1, circTCEA3, circTOMM20|SNORA14B, circSCCPDH, circZNF124.2, circGLS2, circR3HDM2, circDHDDS, circSNORA73A|RCC1|SNHG3.1, circSNORA73A|RCC1|SNHG3.2, circSNORA61|SNHG12, circCEP83|RBMS2P1, circFGD6, circPUM1, circTMCO3|RP11-230F18.6, circPTP4A2, circZMYM5, circN6AMT2, circRPL21|SNORA27, circGTF2F2, circZMYM4, circLINC00355, circUNKNOWN00000004, circFARP1, circDYNC1H1, circCDC42BPB, circCCNB1IP1|SNORA79|AL355075.1, circRPPH1|RPPH1.1, circRPPH1|RPPH1.2, circRPPH1|RPPH1.3, circRPPH1|RPPH1.4, circSNORD8|CHD8.1, circSNORD8|CHD8.2, circPPP1R3E, circCHMP4A|RP11-468E2.1|AL136419.6, circUNKNOWN00000005, circSEC23A, circSNORD46| RPS8, circSAMD4A, circPCNX, circPSEN1, circFCF1, circSCARNA13|SNHG10.1, circSCARNA13|SNHG10.2, circSCARNA13|SNHG10.3, circUNKNOWN00000006, circTJP1, circRP11-632K20.7, circTTBK2, circPPIB, circUBE2Q2, circETFA, circSEC11A, circPDE8A, circDAB1|OMA1, circABHD2, circlQGAP1.1, circlQGAP1.2, circCHD2, circIGF1R, circNPRL3, circNDE1, circABCC1, circRPS2|SNORA64, circPOLR3E, circATXN2L, circMVP, circASPHD1, circITGAL, circRP5-857K21.6.1, circRP5-857K21.6.2, circRP5-857K21.6.3, circRP5-857K21.6.4, circZNF720, circLONP2, circCHD9, circSLC7A6, circCARHSP1, circFANCA, circRAD51D|RAD51L3-RFFL, circHDAC5, circUTP18, circSRSF1, circPPM1D, circBRIP1, circPRKCA.1, circPRKCA.2, circEIF4A1|SNORD10|RP11-186B7.4|SENP3-EIF4A1.1, circEIF4A1|SNORD10|RP11-186B7.4|SENP3-EIF4A1.2, circPGS1, circRPTOR, circRPL26|RP11-849F2.7, circRP11-206L10.8, circPIAS2, circTYMS, circPPP4R1, circZNF91, circWDR62, circADCK4, circARHGAP35, circNUCB1, circSNORD33|RPL13A.1, circSNORD33|RPL13A.2, circSNORD33|RPL13A.3, circMUC16, circLZIC, circSNX5|SNORD17|OVOL2.1, circSNX5|SNORD17|OVOL2.2, circSNORA71A|SNHG17, circPLTP, circTMEM230, circCYP24A1, circZBTB46, circGART, circRAB3GAP1, circDYRK1A, circUNKNOWN00000007, circCOL18A1.1, circCOL18A1.2, circNBAS, circCH507-513H4.1.1, circCH507-513H4.1.2, circCH507-513H4.1.3, circANKAR, circGLS, circBMPR2, circRHBDD1, circATG16L1|SCARNA5, circDGKD, circPASK, circPPP6R2, circBIRC6, circPRKD3, circKIAA184|RP11-493E12.3, circRTKN, circELMOD3, circREV1, circZBTB20, circTIMMDC1, circACAD9, circPLXND1, circHDAC11, circCEP70, circRNF13.1, circRNF13.2, circGOLIM4, circEIF4A2|SNORD2.1, circEIF4A2|SNORD2.2, circSDHAP1, circSETD2, circSCAP, circUSP4, circRPL29, circPHF7, circNEK4, circFLNB, circSLC25A26, circNFKB1, circFIP1L1|RP11-231C18.3, circTBC1D14, circALB.1, circALB.2, circALB.3, circNUP54, circAFF1, circSLC12A7| MIR4635, circMAN2A1.1, circMAN2A1.2, circAFF4, circUBE2D2, circANKHD1|ANKHD1-EIF4EBP3, circMAPK9, circGPBP1, circCEP72, circRP11-98J23.2, circFAM169A, circWDR41, circRASGRF2, circRHOBTB3, circCEP85L, circARID1B.1, circARID1B.2, circTULP4|RP11-732M18.4, circTULP4, circTMEM181, circHIST1H3B, circHIST1H3C.2, circUNKNOWN00000008, circC6orf136, circHLA-C|HLA-B|XXbac-BPG248L24.10|WASF5P|XXbac-BPG248L24.13.1, circHLA-C|HLA-B|XXbac-BPG248L24.10|WASF5P|XXbac-BPG248L24.13.2, circFKBP5, circCNPY3, circSRF, circRN7SK, circFARS2, circMLIP, circZNF292, circPNRC1, circUNKNOWN00000009, circNDUFB2, circKMT2C, circESYT2, circMPP6, circHERPUD2, circOGDH, circZNF680, circKDELR2|DAGLB, circZDHHC4, circCCZ1B, circPOM121, circBAZ1B, circGTF2I, circSNORA14A, circCDK14, circCCDC132, circTRRAP|MIR3609, circCYP3A7|CYP3A7-CYP3A51P, circCCAT1.1, circCCAT1.2, circCCAT1.3, circCCAT1.4, circCCAT1.5, circCCAT1.6, circCCAT1.7, circASAP1, circPTK2.1, circPTK2.2, circSLC45A4, circADGRB1, circRBPMS, circFGFR1, circHOOK3, circASPH, circTMEM245, circUNKNOWN00000010, circHSPA5, circGLE1, circFOCAD, circNFX1, circUBAP2, circKDM4C|RP11-146B14.1, circAGTPBP1, circFAM120A.1, circFAM120A.2, circHIATL1, circPPP2R3B, circATRX, or circTBL1X.

The diseases to be treated with the compositions or with the antisense oligonucleotides of the invention may in non-limiting example be anyone selected from the list of: hepatocellular carcinoma, ciRS-7 positive cancer, circFAT1 positive cancer, circPVT1 positive cancer, circHIPK3 positive cancer, circSRY positive cancer, circSLC35E2B positive cancer, circCDK11A positive cancer, circUNKNOWN00000001 positive cancer, circARHGAP32 positive cancer, circSLC8A3 positive cancer, circHERC2 positive cancer, circZFAND6 positive cancer, circRP1-168P16.1 positive cancer, circAURKC positive cancer, circAFTPH positive cancer, circSCD positive cancer, circSMC3 positive cancer, circSNORA23|IPO7.1 positive cancer, circZNF124.1 positive cancer, circSNX5|OVOL2 positive cancer, circRALY positive cancer, circTFPI positive cancer, circAHSG.1 positive cancer, circAHSG.2 positive cancer, circAHSG.3 positive cancer, circUBXN7 positive cancer, circAFP positive cancer, circHIST1H3A positive cancer, circHIST1H3C.1 positive cancer, circANAPC2 positive cancer, circRMRP|RMRP positive cancer, circCENPI positive cancer, circFIRRE positive cancer, circMBNL3 positive cancer, circGPC3 positive cancer, circPROSER2 positive cancer, circMALRD1 positive cancer, circFAM208B positive cancer, circMCU positive cancer, circKIF20B positive cancer, circABCC2 positive cancer, circEIF4G2|SNORD97.1 positive cancer, circEIF4G2|SNORD97.2 positive cancer, circEIF4G2|SNORD97.3 positive cancer, circEIF4G2|SNORD97.4 positive cancer, circEIF4G2|SNORD97.5 positive cancer, circEIF4G2|SNORD97.6 positive cancer, circEIF4G2|SNORD97.7 positive cancer, circEIF4G2|SNORD97.8 positive cancer, circEIF4G2|SNORD97.9 positive cancer, circEIF4G2|SNORD97.10 positive cancer, circlGF2 positive cancer, circQSER1 positive cancer, circUNKNOWN00000002 positive cancer, circCHD1L positive cancer, circPRUNE positive cancer, circSLC27A3 positive cancer, circGATAD2B positive cancer, circKIAA0907 positive cancer, circCCT3 positive cancer, circPLEKHM2 positive cancer, circVWCE positive cancer, circATF6 positive cancer, circMALAT1.1 positive cancer, circMALAT1.2 positive cancer, circMALAT1.3 positive cancer, circMALAT1.4 positive cancer, circMALAT1.5 positive cancer, circMALAT1.6 positive cancer, circMALAT1.7 positive cancer, circMALAT1.8 positive cancer, circMALAT1.9 positive cancer, circMALAT1.10 positive cancer, circMALAT1.11 positive cancer, circMALAT1.12 positive cancer, circMALAT1.13 positive cancer, circUNKNOWN00000003 positive cancer, circMALAT1.14 positive cancer, circMALAT1.15 positive cancer, circMALAT1.16 positive cancer, circMALAT1.17 positive cancer, circMALAT1.18 positive cancer, circMALAT1.19 positive cancer, circUCK2 positive cancer, circSUCO positive cancer, circRAB6A positive cancer, circRPS3|SNORD15B.1 positive cancer, circRPS3|SNORD15B.2 positive cancer, circRPS3|SNORD15B.3 positive cancer, circRSF1 positive cancer, circABL2 positive cancer, circGNB1 positive cancer, circRPLP2|SNORA52 positive cancer, circPICALM.1 positive cancer, circPICALM.2 positive cancer, circSNORA23|IPO7.2 positive cancer, circSNORA23|IPO7.3 positive cancer, circCFH positive cancer, circSLC41A2.1 positive cancer, circSLC41A2.2 positive cancer, circCORO1C positive cancer, circEIF4G31 RP11-487E1.2 positive cancer, circNAA25 positive cancer, circMED13L positive cancer, circLPGAT1|RN7SL344P positive cancer, circAACS positive cancer, circTP53BP2 positive cancer, circSOX5 positive cancer, circDNAH14 positive cancer, circKDM1A|MIR3115 positive cancer, circTTC13 positive cancer, circEGLN1 positive cancer, circTCEA3 positive cancer, circTOMM20|SNORA14B positive cancer, circSCCPDH positive cancer, circZNF124.2 positive cancer, circGLS2 positive cancer, circR3HDM2 positive cancer, circDHDDS positive cancer, circSNORA73A|RCC1|SNHG3.1 positive cancer, circSNORA73A|RCC1|SNHG3.2 positive cancer, circSNORA61|SNHG12 positive cancer, circCEP83|RBMS2P1 positive cancer, circFGD6 positive cancer, circPUM1 positive cancer, circTMCO3|RP11-230F18.6 positive cancer, circPTP4A2 positive cancer, circZMYM5 positive cancer, circN6AMT2 positive cancer, circRPL21|SNORA27 positive cancer, circGTF2F2 positive cancer, circZMYM4 positive cancer, circLINC00355 positive cancer, circUNKNOWN00000004 positive cancer, circFARP1 positive cancer, circDYNC1H1 positive cancer, circCDC42BPB positive cancer, circCCNB1IP1|SNORA79|AL355075.1 positive cancer, circRPPH1|RPPH1.1 positive cancer, circRPPH1|RPPH1.2 positive cancer, circRPPH1|RPPH1.3 positive cancer, circRPPH1|RPPH1.4 positive cancer, circSNORD8|CHD8.1 positive cancer, circSNORD8|CHD8.2 positive cancer, circPPP1R3E positive cancer, circCHMP4A|RP11-468E2.1|AL136419.6 positive cancer, circUNKNOWN00000005 positive cancer, circSEC23A positive cancer, circSNORD46|RPS8 positive cancer, circSAMD4A positive cancer, circPCNX positive cancer, circPSEN1 positive cancer, circFCF1 positive cancer, circSCARNA13|SNHG10.1 positive cancer, circSCARNA13|SNHG10.2 positive cancer, circSCARNA13|SNHG10.3 positive cancer, circUNKNOWN00000006 positive cancer, circTJP1 positive cancer, circRP11-632K20.7 positive cancer, circTTBK2 positive cancer, circPPIB positive cancer, circUBE2Q2 positive cancer, circETFA positive cancer, circSEC11A positive cancer, circPDE8A positive cancer, circDAB1|OMA1 positive cancer, circABHD2 positive cancer, circlQGAP1.1 positive cancer, circlQGAP1.2 positive cancer, circCHD2 positive cancer, circlGF1R positive cancer, circNPRL3 positive cancer, circNDE1 positive cancer, circABCC1 positive cancer, circRPS2|SNORA64 positive cancer, circPOLR3E positive cancer, circATXN2L positive cancer, circMVP positive cancer, circASPHD1 positive cancer, circITGAL positive cancer, circRP5-857K21.6.1 positive cancer, circRP5-857K21.6.2 positive cancer, circRP5-857K21.6.3 positive cancer, circRP5-857K21.6.4 positive cancer, circZNF720 positive cancer, circLONP2 positive cancer, circCHD9 positive cancer, circSLC7A6 positive cancer, circCARHSP1 positive cancer, circFANCA positive cancer, circRAD51D|RAD51L3-RFFL positive cancer, circHDAC5 positive cancer, circUTP18 positive cancer, circSRSF1 positive cancer, circPPM1D positive cancer, circBRIP1 positive cancer, circPRKCA.1 positive cancer, circPRKCA.2 positive cancer, circEIF4A1|SNORD10|RP11-186B7.4|SENP3-EIF4A1.1 positive cancer, circEIF4A1|SNORD10|RP11-186B7.4|SENP3-EIF4A1.2 positive cancer, circPGS1 positive cancer, circRPTOR positive cancer, circRPL26|RP11-849F2.7 positive cancer, circRP11-206L10.8 positive cancer, circPIAS2 positive cancer, circTYMS positive cancer, circPPP4R1 positive cancer, circZNF91 positive cancer, circWDR62 positive cancer, circADCK4 positive cancer, circARHGAP35 positive cancer, circNUCB1 positive cancer, circSNORD33 | RPL13A.1 positive cancer, circSNORD33|RPL13A.2 positive cancer, circSNORD33|RPL13A.3 positive cancer, circMUC16 positive cancer, circLZIC positive cancer, circSNX5|SNORD17|OVOL2.1 positive cancer, circSNX5|SNORD17|OVOL2.2 positive cancer, circSNORA71A|SNHG17 positive cancer, circPLTP positive cancer, circTMEM230 positive cancer, circCYP24A1 positive cancer, circZBTB46 positive cancer, circGART positive cancer, circRAB3GAP1 positive cancer, circDYRK1A positive cancer, circUNKNOWN00000007 positive cancer, circCOL18A1.1 positive cancer, circCOL18A1.2 positive cancer, circNBAS positive cancer, circCH507-513H4.1.1 positive cancer, circCH507-513H4.1.2 positive cancer, circCH507-513H4.1.3 positive cancer, circANKAR positive cancer, circGLS positive cancer, circBMPR2 positive cancer, circRHBDD1 positive cancer, circATG16L1|SCARNA5 positive cancer, circDGKD positive cancer, circPASK positive cancer, circPPP6R2 positive cancer, circBIRC6 positive cancer, circPRKD3 positive cancer, circKIAA184|RP11-493E12.3 positive cancer, circRTKN positive cancer, circELMOD3 positive cancer, circREV1 positive cancer, circZBTB20 positive cancer, circTIMMDC1 positive cancer, circACAD9 positive cancer, circPLXND1 positive cancer, circHDAC11 positive cancer, circCEP70 positive cancer, circRNF13.1 positive cancer, circRNF13.2 positive cancer, circGOLIM4 positive cancer, circEIF4A2|SNORD2.1 positive cancer, circEIF4A2|SNORD2.2 positive cancer, circSDHAP1 positive cancer, circSETD2 positive cancer, circSCAP positive cancer, circUSP4 positive cancer, circRPL29 positive cancer, circPHF7 positive cancer, circNEK4 positive cancer, circFLNB positive cancer, circSLC25A26 positive cancer, circNFKB1 positive cancer, circFIP1L1|RP11-231C18.3 positive cancer, circTBC1D14 positive cancer, circALB.1 positive cancer, circALB.2 positive cancer, circALB.3 positive cancer, circNUP54 positive cancer, circAFF1 positive cancer, circSLC12A7| MIR4635 positive cancer, circMAN2A1.1 positive cancer, circMAN2A1.2 positive cancer, circAFF4 positive cancer, circUBE2D2 positive cancer, circANKHD1|ANKHD1-EIF4EBP3 positive cancer, circMAPK9 positive cancer, circGPBP1 positive cancer, circCEP72 positive cancer, circRP11-98J23.2 positive cancer, circFAM169A positive cancer, circWDR41 positive cancer, circRASGRF2 positive cancer, circRHOBTB3 positive cancer, circCEP85L positive cancer, circARID1B.1 positive cancer, circARID1B.2 positive cancer, circTULP41 RP11-732M18.4 positive cancer, circTULP4 positive cancer, circTMEM181 positive cancer, circHIST1H3B positive cancer, circHIST1H3C.2 positive cancer, circUNKNOWN00000008 positive cancer, circC6orf136 positive cancer, circHLA-C|HLA-B |XXbac-BPG248L24.10|WASF5P|XXbac-BPG248L24.13.1 positive cancer, circHLA-C|HLA-B |XXbac-BPG248L24.10|WASF5P|XXbac-BPG248L24.13.2 positive cancer, circFKBP5 positive cancer, circCNPY3 positive cancer, circSRF positive cancer, circRN7SK positive cancer, circFARS2 positive cancer, circMLIP positive cancer, circZNF292 positive cancer, circPNRC1 positive cancer, circUNKNOWN00000009 positive cancer, circNDUFB2 positive cancer, circKMT2C positive cancer, circESYT2 positive cancer, circMPP6 positive cancer, circHERPUD2 positive cancer, circOGDH positive cancer, circZNF680 positive cancer, circKDELR2| DAGLB positive cancer, circZDHHC4 positive cancer, circCCZ1B positive cancer, circPOM121 positive cancer, circBAZ1B positive cancer, circGTF2l positive cancer, circSNORA14A positive cancer, circCDK14 positive cancer, circCCDC132 positive cancer, circTRRAP|MIR3609 positive cancer, circCYP3A7 |CYP3A7-CYP3A51P positive cancer, circCCAT1.1 positive cancer, circCCAT1.2 positive cancer, circCCAT1.3 positive cancer, circCCAT1.4 positive cancer, circCCAT1.5 positive cancer, circCCAT1.6 positive cancer, circCCAT1.7 positive cancer, circASAP1 positive cancer, circPTK2.1 positive cancer, circPTK2.2 positive cancer, circSLC45A4 positive cancer, circADGRB1 positive cancer, circRBPMS positive cancer, circFGFR1 positive cancer, circHOOK3 positive cancer, circASPH positive cancer, circTMEM245 positive cancer, circUNKNOWN00000010 positive cancer, circHSPA5 positive cancer, circGLE1 positive cancer, circFOCAD positive cancer, circNFX1 positive cancer, circUBAP2 positive cancer, circKDM4C|RP11-146B14.1 positive cancer, circAGTPBP1 positive cancer, circFAM120A.1 positive cancer, circFAM120A.2 positive cancer, circHIATL1 positive cancer, circPPP2R3B positive cancer, circATRX positive cancer, or circTBL1X positive cancer. A circ positive cancer is a cancer characterized in that the cancer cells express that particuolar circRNA, or where the cancer cells expresses abnormal amounts of the particular circRNA.

### Methods of Treatment

The antisense oligonucleotides of the invention are for use in a method of treatment. The antisense oligonucleotides of the present invention may be used in methods of treatment of many diseases, in example cancer. In some embodiments, the antisense oligonucleotides of the invention are for use in a method of treating cancer, wherein the antisense oligonucleotide is provided in an effective dosage.

### ANTISENSE OLIGONUCLEOTIDE-MEDIATED MODULATION OF INCRNAS

One aspect of the invention is to provide antisense oligonucleotides that are effective in modulating IncRNAs, such as large intergenic noncoding RNAs (lincRNAs).

In some embodiments the antisense oligonucleotides of the invention targets a IncRNA selected from the list of DANCR, H19, HOTAIR, HOTTIP, HULC, LINC-ROR, MALAT1, MVIH, NEAT1, PCBP2-OT1, PVT1, TUG1, UCA1, UFC1 and LINC01215 (for review, see Parasramka et al., 2016, Pharmacol. Ther. 161: 67-78). These IncRNAs have all been implicated in the pathogenesis of cancer, such as hepatocellular carcinoma. The expression of some of these have been linked to poor prognosis, increased tumor-driven angiogenesis or metastasis formation, and there are indications that they are important for various cancers, including, but not limited to hepatocellular carcinoma, glioma, osteosarcoma, esophageal squamous cell carcinoma, pancreatic cancer, gastric cancer, breast cancer, non-small cell lung cancer, prostate cancer, ovarian cancer, B-cell lymphoma, colorectal cancer, cutaneous squamous cell carcinoma, multiple myeloma, and tongue squamous cell carcinoma. In some embodiments, the antisense oligonucleotides of the invention having any one of SEQ ID NOs: 2149 - 2259 are for use as medicaments. In some embodiments, the antisense oligonucleotides of the invention having any one of SEQ ID NOs: 2149 - 2259 are for use as medicaments in the treatment of cancer, such as any one of hepatocellular carcinoma, glioma, osteosarcoma, esophageal squamous cell carcinoma, pancreatic cancer, gastric cancer, breast cancer, non-small cell lung cancer, prostate cancer, ovary cancer, B-cell lymphoma, colorectal cancer, cutaneous squamous cell carcinoma, multiple myeloma, and tongue squamous cell carcinoma.

In specific embodiments 1-27 below, the IncRNA-targeting antisense oligonucleotides of the invention, their design, delivery, and uses are described.

1) A compound comprising the modified antisense oligonucleotide consisting of any one of SEQ ID NOs: 2149 - 2259.

2) A compound according to embodiment 1, wherein the nucleotide analogues of the wings are selected from the list of beta-D-oxy LNA, alpha-L-oxy-LNA, beta-D-amino-LNA, alpha-L-amino-LNA, beta-D-thio-LNA, alpha-L-thio-LNA, 5′-methyl-LNA, beta-D-ENA and alpha-L-ENA.

3) A compound according to embodiment 2, wherein the modified antisense oligonucleotide is 100% complementary to the target nucleic acid.

4) A compound according to embodiment 2, wherein the nucleotide analogues of the wings are Beta-D-Oxy LNA.

5) A compound according to embodiment 1, wherein the nucleoside analogues of the wings are not LNA, but anyone of tricyclo-DNA, 2′-Fluoro, 2′-O-methyl, 2′-methoxyethyl (2′-MOE), 2′cyclic ethyl (cET), UNA and Conformationally Restricted Nucleoside (CRN).

6) A compound according to embodiment 1, wherein the nucleoside analogues of the wings are a mixture of LNA and anyone of tricyclo-DNA, 2′Fluoro, 2′-O-methyl, 2′-methoxyethyl (2′-MOE), 2′cyclic ethyl (cET), UNA, and Conformationally Restricted Nucleoside (CRN).

7) A compound according to embodiment 6, wherein the nucleoside analogues of the wings are a mixture of LNA and 2′-Fluoro.

8) The compound according to anyone of embodiments 1-7, wherein the antisense oligonucleotide is conjugated with a ligand for targeted delivery

9) The antisense oligonucleotide according to embodiment 8, wherein the antisense oligonucleotide is conjugated with folic acid or N-acetylgalactosamine (GalNAc).

10) The antisense oligonucleotide according to anyone of embodiments 1-9, wherein the antisense oligonucleotide is unconjugated in a pharmaceutical composition for delivery.

11) The antisense oligonucleotide according to anyone of embodiments 1-9, wherein the antisense oligonucleotide is formulated in lipid nanoparticles for delivery.

12) The antisense oligonucleotide according to any one of the preceding embodiments, for use as a medicament.

13) The antisense oligonucleotide according to embodiment 12, wherein the antisense oligonucleotide is for use as a medicament in the treatment of cancer.

14) The antisense oligonucleotide according to embodiment 13, wherein the cancer is hepatocellular carcinoma.

15) A composition comprising an antisense oligonucleotide according to anyone of the preceding embodiments and a carrier.

16) A composition comprising an antisense oligonucleotide according to any one of embodiments 1-11, for use as a pharmaceutical or in a method of treatment.

17) A composition according to embodiments 15-16, wherein the composition comprises more than one antisense oligonucleotide according to anyone of embodiments 1-14.

18) A composition according to embodiment 17, wherein the two or more antisense oligonucleotides are selected from the list of anyone of SEQ ID NOs: 2149 - 2259.

19) The composition according to anyone of embodiments 15-18, wherein the antisense oligonucleotide or composition is for treatment of cancer.

20) The composition according to embodiment 19, wherein the cancer is selected from the list of cancers such as hepatocellular carcinoma, or prostate cancer.

21) The antisense oligonucleotide according to anyone of embodiments 1-14, or composition according embodiments 15 to 20, wherein the antisense oligonucleotide or composition is for treatment of a human subject.

22) The antisense oligonucleotide or composition according to anyone of the preceding embodiments, wherein the antisense oligonucleotide or composition is for treatment of a cell ex vivo.

23) A method of downregulating an endogenous IncRNA selected from the list of DANCR, H19, HOTAIR, HOTTIP, HULC, LINC-ROR, MALAT1, MVIH, NEAT1, PCBP2-OT1, PVT1, TUG1, UCA1, UFC1 and LINC01215 in a cell, by administration of an effective amount of an antisense oligonucleotide that is complementary to the target and selected from the list according to anyone of embodiments 1-14, or a composition according to anyone of embodiments 15-20 to a cell.

24) The method of embodiment 23, wherein the cell is in a human body.

25) The method of embodiment 24, wherein the cell is a cancer cell in a human body.

26) A method of treatment of cancer, comprising the administration of an effective dosage of an antisense oligonucleotide or a composition according to anyone of embodiments 1-22 to a human subject.

27) The method according to embodiment 26, wherein the cancer is selected from the list of cancers such as hepatocellular carcinoma or prostate cancer.

28) The antisense oligonucleotides, or compositions or methods of treatment according to any one of embodiments 1-27, wherein the antisense oligonucleotide, or compositions or methods of treatment are for use in combination with another compound, composition or method of treatment.

Table 3 shows a list of specific antisense oligonucleotides (SEQ ID NOs: 2149 - 2259) targeting the IncRNAs; DANCR, H19, HOTAIR, HOTTIP, HULC, LINC-ROR, MALAT1, MVIH, NEAT1, PCBP2-OT1, PVT1, TUG1, UCA1, UFC1 and LINC01215. (LNA, such as in non-limiting example Beta-D-Oxy LNA = uppercase, DNA lowercase, complete phosphorothioate backbone, LNA cytosine units are LNA 5-methylcytosines).

TABLE 3

Gapmer antisense oligonucleotides for modulation of long noncoding RNAs.

| SEQ.ID.NO | Oligonucleotide (5′-3′) | Gene symbol | Ensembl gene id |
|---|---|---|---|
| 2149 | CAAaccagagaggcgGC | DANCR | ENSG00000226950 |
| 2150 | CCGcagacgtaagagAC | DANCR | ENSG00000226950 |
| 2151 | CGAaagccgaagacTGG | DANCR | ENSG00000226950 |
| 2152 | TGgcgacaaacaGACG | DANCR | ENSG00000226950 |
| 2153 | TTAgtacgcatattTGG | DANCR | ENSG00000226950 |
| 2154 | TAcgcatatttggCCT | DANCR | ENSG00000226950 |
| 2155 | CActcaccgcgcaACT | DANCR | ENSG00000226950 |
| 2156 | TGtattgcttgttcTAT | DANCR | ENSG00000226950 |
| 2157 | TTacagggttcactaCTAT | DANCR | ENSG00000226950 |
| 2158 | CTGcattgagttaGCG | DANCR | ENSG00000226950 |
| 2159 | CTatagcgcctagataACG | DANCR | ENSG00000226950 |
| 2160 | ACATagtggcgcgtcAG | DANCR | ENSG00000226950 |
| 2161 | GAGAccgaaagccgAAG | DANCR | ENSG00000226950 |
| 2162 | AGAcctgcgccgggAA | DANCR | ENSG00000226950 |
| 2163 | GTTGtcaacctataGAA | DANCR | ENSG00000226950 |
| 2164 | ACgtgttggttgtgtGG | H19 | ENSG00000130600 |
| 2165 | CTAgagatagcgacacGT | H19 | ENSG00000130600 |
| 2166 | CTgccacgtcctgtAAC | H19 | ENSG00000130600 |
| 2167 | TACtaaatgaattGCGG | H19 | ENSG00000130600 |
| 2168 | TCacgcacactcgtaCT | H19 | ENSG00000130600 |
| 2169 | GCACaagagatcgagTT | HOTAIR | ENSG00000228630 |
| 2170 | GCggacagggaaatcaaCT | HOTAIR | ENSG00000228630 |
| 2171 | GTCtaggaatcagcacgAA | HOTAIR | ENSG00000228630 |
| 2172 | TCttcgacaacgcCTA | HOTAIR | ENSG00000228630 |
| 2173 | TGaacaaacgagaGCGT | HOTAIR | ENSG00000228630 |
| 2174 | GACcgctatgatcctTC | HOTAIR | ENSG00000228630 |
| 2175 | AGACtaagacggataaCG | HOTAIR | ENSG00000228630 |
| 2176 | CCactctctcatactaAAT | HOTAIR | ENSG00000228630 |
| 2177 | GTgaacaaacgagAGCG | HOTAIR | ENSG00000228630 |
| 2178 | CGGacagggaaatcaaCTA | HOTAIR | ENSG00000228630 |
| 2179 | CTGggaatgtaagAACG | HOTAIR | ENSG00000228630 |
| 2180 | AgtgcaaagtcccgtTT | HOTAIR | ENSG00000228630 |
| 2181 | CCttcaaacgctCGAA | HOTTIP | ENSG00000243766 |
| 2182 | CCTtctataaacgaccTC | HOTTIP | ENSG00000243766 |
| 2183 | GACGattctctcataTAAA | HOTTIP | ENSG00000243766 |
| 2184 | GctacactgtttgacgAT | HOTTIP | ENSG00000243766 |
| 2185 | GTCagaggcgagaaTTT | HOTTIP | ENSG00000243766 |
| 2186 | AATTcctttatccgcagAG | HULC | ENSG00000251164 |
| 2187 | CTTgtaaaggctccaatTC | HULC | ENSG00000251164 |
| 2188 | GTCgaatataatccTAG | HULC | ENSG00000251164 |
| 2189 | GTtccagattgttCGAA | HULC | ENSG00000251164 |
| 2190 | TTgtaagacatctatCATC | HULC | ENSG00000251164 |
| 2191 | TTATtgattgcgTCTT | LINC01215 | ENSG00000271856 |
| 2192 | AGCGgaagtgagtagtAA | LINC01215 | ENSG00000271856 |
| 2193 | AAggtcaggaagcacGCG | LINC-ROR | ENSG00000258609 |
| 2194 | CACtacgacacagcaGG | LINC-ROR | ENSG00000258609 |
| 2195 | CGGgacgattatttatTC | LINC-ROR | ENSG00000258609 |
| 2196 | GCaacgacgggatGTGA | LINC-ROR | ENSG00000258609 |
| 2197 | TTcgaggttatcagggTG | LINC-ROR | ENSG00000258609 |
| 2198 | CACacagcacagcCTC | MALAT1 | ENSG00000251562 |
| 2199 | ATAGacggagaacAACT | MALAT1 | ENSG00000251562 |
| 2200 | CAAAgcaaagacgcCGC | MALAT1 | ENSG00000251562 |
| 2201 | CTGataacgaagagatACC | MALAT1 | ENSG00000251562 |
| 2202 | GAgggacagtaggtataGT | MALAT1 | ENSG00000251562 |
| 2203 | GcttcagacaagattcaTG | MALAT1 | ENSG00000251562 |
| 2204 | CAgcacaactcgtcGC | MALAT1 | ENSG00000251562 |
| 2205 | TTCaccacgaactgcTG | MALAT1 | ENSG00000251562 |
| 2206 | TCACcaccaaatcgtTA | MALAT1 | ENSG00000251562 |
| 2207 | TAGAttccgtaacTTTA | MALAT1 | ENSG00000251562 |
| 2208 | CGttcttccgctcaaaTC | MALAT1 | ENSG00000251562 |
| 2209 | CTCCagtcgtttcacAA | MALAT1 | ENSG00000251562 |
| 2210 | ATTaggttctcgtGTAA | MALAT1 | ENSG00000251562 |
| 2211 | AAatcccactacgcCCA | MVIH | AK094613.1 |
| 2212 | ATaactccatcgcaACC | MVIH | AK094613.1 |
| 2213 | CAcctttactccttcGG | MVIH | AK094613.1 |
| 2214 | CAAtttgaaacgaGCTG | PCBP2-OT1 | ENSG00000282977 |
| 2215 | CAgtgtgggattaagttGA | PCBP2-OT1 | ENSG00000282977 |
| 2216 | GAAagctcgcactgtCG | PCBP2-OT1 | ENSG00000282977 |
| 2217 | ACTtcataggaacggCA | PVT1 | ENSG00000249859 |
| 2218 | AGAAtacaaacggGAGG | PVT1 | ENSG00000249859 |
| 2219 | AGtaacatacagcaCGA | PVT1 | ENSG00000249859 |
| 2220 | GCgagagacaggcTAAC | PVT1 | ENSG00000249859 |
| 2221 | TCGCtaaacaatacTCA | PVT1 | ENSG00000249859 |
| 2222 | AactgtccacgccaaCC | PVT1 | ENSG00000249859 |
| 2223 | CAtttgtcacctaacCC | PVT1 | ENSG00000249859 |
| 2224 | ACTtctcacccattcGT | PVT1 | ENSG00000249859 |
| 2225 | AAgcagacacccgttaGT | PVT1 | ENSG00000249859 |
| 2226 | CTTAaccatcccataTC | PVT1 | ENSG00000249859 |
| 2227 | TAtctccttcgtcctCA | PVT1 | ENSG00000249859 |
| 2228 | TtgttgtttcaccctCG | PVT1 | ENSG00000249859 |
| 2229 | GACaagaattatcCACG | PVT1 | ENSG00000249859 |
| 2230 | CAcgctcatatttAAGG | PVT1 | ENSG00000249859 |
| 2231 | GACGcaataccttatGTA | PVT1 | ENSG00000249859 |
| 2232 | CAACtattatactCACG | PVT1 | ENSG00000249859 |
| 2233 | CGcaacaggattCGGA | PVT1 | ENSG00000249859 |
| 2234 | CATTggagatagataCGC | PVT1 | ENSG00000249859 |
| 2235 | CAGAagcagtagtaattTG | TUG1 | ENSG00000253352 |
| 2236 | CCcatcattcaacatATTG | TUG1 | ENSG00000253352 |
| 2237 | GATAgaggatacataACG | TUG1 | ENSG00000253352 |
| 2238 | GTAatcaagtcgtCATC | TUG1 | ENSG00000253352 |
| 2239 | TAAgaataagtcggtcACA | TUG1 | ENSG00000253352 |
| 2240 | CTGTgttcggaagagTT | TUG1 | ENSG00000253352 |
| 2241 | GAggttccgcagtaGT | TUG1 | ENSG00000253352 |
| 2242 | TTaagggagtctgtcaGTG | TUG1 | ENSG00000253352 |
| 2243 | GATCtaagaataaGTCG | TUG1 | ENSG00000253352 |
| 2244 | CTtgctcagtcgttGTC | TUG1 | ENSG00000253352 |
| 2245 | AACTgtctcgcgaAGC | TUG1 | ENSG00000253352 |
| 2246 | GATCggattcagggtAC | TUG1 | ENSG00000253352 |
| 2247 | TTgtggtgtatgtgggCAA | TUG1 | ENSG00000253352 |
| 2248 | TGccgcatcgtgACAA | TUG1 | ENSG00000253352 |
| 2249 | GTgatgtgtagtttgGAAA | TUG1 | ENSG00000253352 |
| 2250 | ATGggacacgacagcatAA | UCA1 | ENSG00000214049 |
| 2251 | CccacggcagttacGG | UCA1 | ENSG00000214049 |
| 2252 | CGtatagaagacacCCA | UCA1 | ENSG00000214049 |
| 2253 | GAAGtaggataggatagTG | UCA1 | ENSG00000214049 |
| 2254 | GATTggagggtagcGAC | UCA1 | ENSG00000214049 |
| 2255 | AcctagttacccgcttGT | UFC1 | ENSG00000143222 |
| 2256 | CCcttctactaaccttcAT | UFC1 | ENSG00000143222 |
| 2257 | GCggatataaattacCTC | UFC1 | ENSG00000143222 |
| 2258 | TAGAaacacaactaaccCG | UFC1 | ENSG00000143222 |
| 2259 | TgtctacctcagtaagtTC | UFC1 | ENSG00000143222 |

Each compound listed in Table 3 are to be viewed as single embodiments.. In some preferred embodiments, the LNA units in the wings of the antisense oligonucleotide of the invention are Beta-D-Oxy LNA and the antisense oligonucleotide is anyone of SEQ ID NOs: 2149 - 2259. In some preferred embodiments, the LNA units in the wings of the antisense oligonucleotide of the invention are alpha-L-oxy-LNA and the antisense oligonucleotide is anyone of SEQ ID NOs: 2149 - 2259. In some preferred embodiments, the LNA units in the wings of the antisense oligonucleotide of the invention are beta-D-amino-LNA and the antisense oligonucleotide is anyone of SEQ ID NOs: 2149 - 2259. In some preferred embodiments, the LNA units in the wings of the antisense oligonucleotide of the invention are alpha-L-amino-LNA and the antisense oligonucleotide is anyone of SEQ ID NOs: 2149 - 2259. In some preferred embodiments, the LNA units in the wings of the antisense oligonucleotide of the invention are beta-D-thio-LNA and the antisense oligonucleotide is anyone of SEQ ID NOs: 2149 - 2259. In some preferred embodiments, the LNA units in the wings of the antisense oligonucleotide of the invention are alpha-L-thio-LNA and the antisense oligonucleotide is anyone of SEQ ID NOs: 2149 - 2259. In some preferred embodiments, the cytosine LNA units in the wings of the antisense oligonucleotide of the invention are LNA 5′-methylcytosines and the antisense oligonucleotide is anyone of SEQ ID NOs: 2149 - 2259. In some preferred embodiments, the LNA units in the wings of the antisense oligonucleotide of the invention are beta-D-ENA and the antisense oligonucleotide is anyone of SEQ ID NOs: 2149 - 2259. In some preferred embodiments, the LNA units in the wings of the antisense oligonucleotide of the invention are alpha-L-ENA and the antisense oligonucleotide is anyone of SEQ ID NOs: 2149 - 2259. In some embodiments, the nucleoside analogues in the wings are not LNA but tricyclo-DNA and the antisense oligonucleotide is anyone of SEQ ID NOs: 2149 - 2259. In some embodiments, the nucleoside analogues in the wings are not LNA but 2′Fluoro and the antisense oligonucleotide is anyone of SEQ ID NOs: 2149 - 2259. In some embodiments, the nucleoside analogues in the wings are not LNA, but 2′-O-methyl and the antisense oligonucleotide is anyone of SEQ ID NOs: 2149 - 2259. In some embodiments, the nucleoside analogues in the wings are not LNA, but 2′-MOE and antisense oligonucleotide is anyone of SEQ ID NOs: 2149 - 2259. In some embodiments, the nucleoside analogues in the wings are not LNA, but 2′cyclic ethyl (cET) and the antisense oligonucleotide is anyone of SEQ ID NOs: 2149 - 2259. In some embodiments, the nucleoside analogues in the wings are not LNA, but UNA and the antisense oligonucleotide is anyone of SEQ ID NOs: 2149 - 2259. In some embodiments, the nucleoside analogues in the wings are not LNA, but CRN and the antisense oligonucleotide is anyone of SEQ ID NOs: 2149 - 2259. In some embodiments, the nucleoside analogues in the wings are partly LNA, but mixed with another nucleotide analogue selected from the list of tricyclo-DNA, 2′Fluoro, 2′-O-methyl, 2′methoxyethyl (2′MOE), 2′cyclic ethyl (cET), UNA, and Conformationally Restricted Nucleoside (CRN) and the antisense oligonucleotide is anyone of SEQ ID NOs: 2149 - 2259.

In some embodiments, the antisense oligonucleotides of the invention comprising any one of SEQ ID NOs: 2149 - 2259, are for use in combination with another drug or treatment for cancer. In some embodiments, the antisense oligonucleotides of the invention comprising any one of SEQ ID NOs: 2149 - 2259 are for use in combination with another active ingredient. The antisense oligonucleotides of the invention may be formulated together with such other ingredient or drug, or they may be formulated separately.

### Dosages and Compositions

The antisense oligonucleotides of the invention may be used in pharmaceutical formulations and compositions, and are for use in treatment of diseases according to the invention. The compounds and compositions will be used in effective dosages, which means in dosages that are sufficient to achieve a desired effect on a disease parameter. The skilled person will without undue burden be able to determine what a reasonably effective dosage is for individual patients.

As explained initially, the antisense oligonucleotides of the invention will constitute suitable drugs with improved properties. The design of a potent and safe drug requires the fine-tuning of various parameters such as affinity/specificity, stability in biological fluids, cellular uptake, mode of action, pharmacokinetic properties and toxicity.

Accordingly, in a further aspect the antisense oligonucleotide may be used in a pharmaceutical composition comprising an oligonucleotide according to the invention and a pharmaceutically acceptable diluent, carrier or adjuvant. Preferably said carrier is saline or buffered saline.

In a still further aspect the present invention relates to an antisense oligonucleotide according to the present invention for use as a medicament.

As will be understood, dosing is dependent on severity and responsiveness of the disease state to be treated, and the course of treatment lasting from several days to several months, or until a cure is effected or a diminution of the disease state is achieved. Optimal dosing schedules can be calculated from measurements of drug accumulation in the body of the patient. Optimum dosages may vary depending on the relative potency of individual oligonucleotides. Generally it can be estimated based on EC₅₀ values found to be effective in vitro and in vivo animal models. In general, dosage is from 0.01 µg to 1 g per kg of body weight, and may be given once or more daily, weekly, monthly or yearly, or even once every 2 to 10 years or by continuous infusion for hours up to several months. The repetition rates for dosing can be estimated based on measured residence times and concentrations of the drug in bodily fluids or tissues.

Following successful treatment, it may be desirable to have the patient undergo maintenance therapy to prevent the recurrence of the disease state.

As indicated above, the invention also relates to a pharmaceutical composition, which comprises at least one oligonucleotide of the invention as an active ingredient. It should be understood that the pharmaceutical composition according to the invention optionally comprises a pharmaceutical carrier, and that the pharmaceutical composition optionally comprises further active compounds, such as in non-limiting example chemotherapeutic compounds.

The oligonucleotides of the invention can be used “as is” or in form of a variety of pharmaceutically acceptable salts. As used herein, the term “pharmaceutically acceptable salts” refers to salts that retain the desired biological activity of the herein-identified antisense oligonucleotides and exhibit minimal undesired toxicological effects. Non-limiting examples of such salts can be formed with organic amino acid and base addition salts formed with metal cations such as zinc, calcium, bismuth, barium, magnesium, aluminum, copper, cobalt, nickel, cadmium, sodium, potassium, and the like, or with a cation formed from ammonia, N,N-dibenzylethylene-diamine, D-glucosamine, tetraethylammonium, or ethylenediamine.

### Delivery

When the antisense oligonucleotides of the present invention are for use in medicine, various means for delivery may be used in order to achieve efficient targeted delivery to cells and tissues.

Targeted delivery of an antisense oligonucleotide is done depending on the target cell or tissue to reach. Such delivery may be modified by conjugation with a ligand in order to facilitate targeted delivery of the antisense oligonucleotide to target cells and tissues. In some embodiments, the antisense oligonucleotides may be formulated in saline for naked delivery.

In some embodiments, the antisense oligonucleotide of the invention is conjugated to anyone of folic acid or N-acetylgalactosamine (GalNAc). In some embodiments, the antisense oligonucleotide according to the invention is made for unconjugated delivery in a pharmaceutical composition. In some embodiments, the circRNA antisense oligonucleotide according to the invention is formulated in lipid nanoparticles for delivery.

There are several approaches for oligonucleotide delivery. One approach is to use a nanoparticle formulation, which determines the tissue distribution and the cellular interactions of the oligonucleotide. Another approach is to use a delivery vehicle to enhance the cellular uptake, in one or more embodiment the vehicle is anyone of folic acid or GalNAc. A third delivery approach is wherein the oligonucleotide is made unconjugated for delivery in a pharmaceutical composition.

The various examples of delivery may be carried out as parenteral administration. By “Parenteral administration” means administration through infusion or injection and comprises intravenous administration, subcutaneous administration, intramuscular administration, intracranial administration, intraperitoneal administration or intra-arterial administration.

The various examples of delivery may be carried out as oral or nasal administration.

The nanoparticle formulation can be a liposomal formulation and in one embodiment the anionic oligonucleotide is complexed with a cationic lipid thereby forming lipid nanoparticles. Such lipid nanoparticles are useful for treating liver diseases. The nanoparticle formulation can also be a polymeric nanoparticle (Juliano et. Al.; Survey and summary, the delivery of therapeutic oligonucleotides, Nucleic Acids Reseach, 2016).

The vehicle used in vehicle-conjugated formulation can be e.g. a lipid vehicle or a polyamine vehicle. One example of a polyamine vehicle is GalNAc - a high-affinity ligand for the hepatocyte-specific asialoglycoprotein receptor (ASGPR). GalNAc-conjugated ASOs show enhanced uptake to hepatocytes instead of non-parenchymal cells since after entry into the cells, the ASO is liberated in the liver (Prakash et. al.; Targeted delivery of antisense oligonucleotides to hepatocytes using triantennary N-acetyl galactosamine improves potency 10-fold in mice, Nucleic acids research, 2014, vol. 42, no. 13, 8796-8807). GalNAc conjugated ASOs may also show enhance potency and duration of some ASOs targeting human apolipoprotein C-III and human transthyretin (TTR). Folic acid (FA) conjugated ASOs can be used to target the folate receptor that is a cellular surface markers for many solid tumours and myeloid leukemias (Chiu et. al.; Efficient Delivery of an Antisense Oligodeoxyribonucleotide Formulated in Folate Receptor-targeted Liposomes).

In the naked delivery, the oligonucleotide is formulated into a solution comprising saline. This approach is effective in many kinds of cell types among others: primary cells, dividing and non-dividing cells (Soifer et. al.; Silencing of Gene Expression by Gymnotic Delivery of Antisense Oligonucleotides; chapter 25; Michael Kaufmann and claudia Klinger (eds.), Functional Genomics: Methods and Ptotocols).

Formulations of the pharmaceutical compositions described herein may be prepared by methods known in the art of formulation. The preparatory methods may include bringing the antisense oligonucleotide into association with a diluent or another excipient and/or one or more other ingredients, and then if desirable, packaging (e.g. shaping) the product into a desired single- or multi-dose unit. The amount of the antisense oligonucleotide depends on the delivery approach and the specific formulation. The amount of the antisense oligonucleotide will also depend on the subject to be treated (size and condition) and also depend on route of administration. An antisense oligonucleotide, a conjugate or a pharmaceutical composition of the present invention is typically administered in an effective amount.

By way of example, the composition may comprise between 0.1% and 100% (w/w) of the antisense oligonucleotide.

The pharmaceutical formulations according to the present invention may also comprise one or more of the following: a pharmaceutically acceptable excipient, e.g. one or more solvents, dispersion media, diluents, liquid vehicles, dispersion or suspension aids, isotonic agents, surface active agents, preservatives, solid binders, thickening or emulsifying agents, lubricants and the like. It is of cause important that the added excipient are pharmaceutically acceptable and suited to the particular dosage form desired. Remington’s The Science and Practice of Pharmacy, 21″Edition, A. R. Gennaro (Lippincott, Williams 8 Wilkins, Baltimore, MD, 2006; incorporated herein by reference) discloses various excipients used in formulating pharmaceutical compositions and known techniques for the preparation thereof.

The invention will be more fully understood by reference to the following examples. They should not, however, be construed as limiting the scope of the invention. All literature citations are incorporated by reference.

### Items Relating to Compounds Targeting circRNAs

1) An antisense oligonucleotide consisting of a sequence of 14-22 nucleobases in length that is a gapmer comprising a central region of 6 to 16 consecutive DNA nucleotides flanked in each end by wing regions each comprising 1 to 5 nucleotide analogues, and wherein the antisense oligonucleotide comprises 1 to 21 phosphorothioate internucleotide linkages, and wherein the oligonucleotide is complementary to an endogenous circRNA.

2) An antisense oligonucleotide consisting of a sequence of 10-22 nucleobases in length that is a mixmer which does not comprise a region of more than anyone of 2, 3, 4 or 5 consecutive DNA nucleotides, and which comprises from 3 to 22 affinity-enhancing nucleotide analogues, and wherein the antisense oligonucleotide comprises 1 to 21 phosphorothioate internucleotide linkages, and wherein the oligonucleotide is complementary to an endogenous circRNA.

3) A siRNA for inhibition of a circRNA, and wherein one strand of the siRNA has a region of 15-21 nucleotides of complementarity to a circRNA backsplice-juncion and wherein the region of complementarity overlaps the circRNA backsplice site with at least 3 nucleotides.

4) The antisense oligonucleotide according to item 1 or 2, wherein the sequence of complementarity of the antisense oligonucleotide to a circRNA, overlaps the circRNA back-splice junction by at least 3 nucleotides.

5) The antisense oligonucleotide according to anyone of items 1 - 4, wherein the circRNA is anyone of a circRNA selected from the list of ciRS-7, circFAT1, circPVT1, circHIPK3, circSRY, circSLC35E2B, circCDK11A, circUNKNOWN00000001, circARHGAP32, circSLC8A3, circHERC2, circZFAND6, circRP1-168P16.1, circAURKC, circAFTPH, circSCD, circSMC3, circSNORA23|lPO7.1, circZNF124.1, circSNX5|OVOL2, circRALY, circTFPI, circAHSG.1, circAHSG.2, circAHSG.3, circUBXN7, circAFP, circHIST1H3A, circHIST1H3C.1, circANAPC2, circRMRP|RMRP, circCENPI, circFIRRE, circMBNL3, circGPC3 and circFAT1.

6) The antisense oligonucleotide according to anyone of items 1 - 5, wherein the circRNA is anyone of a circRNA selected from the list of ciRS-7, circFAT1, circPVT1, circHIPK3, circSRY, circSLC35E2B, circCDK11A, circUNKNOWN00000001, circARHGAP32, circSLC8A3, circHERC2, circZFAND6, circRP1-168P16.1, circAURKC, circAFTPH, circSCD, circSMC3, circSNORA23|lPO7.1, circZNF124.1, circSNX5|OVOL2, circRALY, circTFPI, circAHSG.1, circAHSG.2, circAHSG.3, circUBXN7, circAFP, circHIST1H3A, circHIST1H3C.1, circANAPC2, circRMRP|RMRP, circCENPI, circFIRRE, circMBNL3, circGPC3, circPROSER2, circMALRD1, circFAM208B, circMCU, circKIF20B, circABCC2, circEIF4G2|SNORD97.1, circEIF4G2|SNORD97.2, circEIF4G2|SNORD97.3, circEIF4G2|SNORD97.4, circEIF4G2|SNORD97.5, circEIF4G2|SNORD97.6, circEIF4G2|SNORD97.7, circEIF4G2|SNORD97.8, circEIF4G2|SNORD97.9, circEIF4G2|SNORD97.10, circlGF2, circQSER1, circUNKNOWN00000002, circCHD1L, circPRUNE, circSLC27A3, circGATAD2B, circKIAA0907, circCCT3, circPLEKHM2, circVWCE, circATF6, circMALAT1.1, circMALAT1.2, circMALAT1.3, circMALAT1.4, circMALAT1.5, circMALAT1.6, circMALAT1.7, circMALAT1.8, circMALAT1.9, circMALAT1.10, circMALAT1.11, circMALAT1.12, circMALAT1.13, circUNKNOWN00000003, circMALAT1.14, circMALAT1.15, circMALAT1.16, circMALAT1.17, circMALAT1.18, circMALAT1.19, circUCK2, circSUCO, circRAB6A, circRPS3|SNORD15B.1, circRPS3|SNORD15B.2, circRPS3|SNORD15B.3, circRSF1, circABL2, circGNB1, circRPLP2|SNORA52, circPICALM.1, circPICALM.2, circSNORA23|IPO7.2, circSNORA23|IPO7.3, circCFH, circSLC41A2.1, circSLC41A2.2, circCORO1C, circElF4G3| RP11-487E1.2, circNAA25, circMED13L, circLPGAT1|RN7SL344P, circAACS, circTP53BP2, circSOX5, circDNAH14, circKDM1A| MIR3115, circTTC13, circEGLN1, circTCEA3, circTOMM20|SNORA14B, circSCCPDH, circZNF124.2, circGLS2, circR3HDM2, circDHDDS, circSNORA73A|RCC1|SNHG3.1, circSNORA73A|RCC1|SNHG3.2, circSNORA61|SNHG12, circCEP83|RBMS2P1, circFGD6, circPUM1, circTMCO3|RP11-230F18.6, circPTP4A2, circZMYM5, circN6AMT2, circRPL21|SNORA27, circGTF2F2, circZMYM4, circLINC00355, circUNKNOWN00000004, circFARP1, circDYNC1H1, circCDC42BPB, circCCNB1IP1|SNORA79|AL355075.1, circRPPH1|RPPH1.1, circRPPH1|RPPH1.2, circRPPH1|RPPH1.3, circRPPH1|RPPH1.4, circSNORD8|CHD8.1, circSNORD8|CHD8.2, circPPP1R3E, circCHMP4A|RP11-468E2.1|AL136419.6, circUNKNOWN00000005, circSEC23A, circSNORD46 | RPS8, circSAMD4A, circPCNX, circPSEN1, circFCF1, circSCARNA13|SNHG10.1, circSCARNA13|SNHG10.2, circSCARNA13|SNHG10.3, circUNKNOWN00000006, circTJP1, circRP11-632K20.7, circTTBK2, circPPIB, circUBE2Q2, circETFA, circSEC11A, circPDE8A, circDAB1|OMA1, circABHD2, circlQGAP1.1, circlQGAP1.2, circCHD2, circlGF1R, circNPRL3, circNDE1, circABCC1, circRPS2 |SNORA64, circPOLR3E, circATXN2L, circMVP, circASPHD1, circITGAL, circRP5-857K21.6.1, circRP5-857K21.6.2, circRP5-857K21.6.3, circRP5-857K21.6.4, circZNF720, circLONP2, circCHD9, circSLC7A6, circCARHSP1, circFANCA, circRAD51D|RAD51L3-RFFL, circHDAC5, circUTP18, circSRSF1, circPPM1D, circBRIP1, circPRKCA.1, circPRKCA.2, circElF4A1|SNORD10|RP11-186B7.4|SENP3-EIF4A1.1, circEIF4A1|SNORD10|RP11-186B7.4|SENP3-EIF4A1.2, circPGS1, circRPTOR, circRPL26|RP11-849F2.7, circRP11-206L10.8, circPIAS2, circTYMS, circPPP4R1, circZNF91, circWDR62, circADCK4, circARHGAP35, circNUCB1, circSNORD33|RPL13A.1, circSNORD33|RPL13A.2, circSNORD33 | RPL13A.3, circMUC16, circLZIC, circSNX5|SNORD17|OVOL2.1, circSNX5|SNORD17|OVOL2.2, circSNORA71A|SNHG17, circPLTP, circTMEM230, circCYP24A1, circZBTB46, circGART, circRAB3GAP1, circDYRK1A, circUNKNOWN00000007, circCOL18A1.1, circCOL18A1.2, circNBAS, circCH507-513H4.1.1, circCH507-513H4.1.2, circCH507-513H4.1.3, circANKAR, circGLS, circBMPR2, circRHBDD1, circATG16L1|SCARNA5, circDGKD, circPASK, circPPP6R2, circBIRC6, circPRKD3, circKIAA184|RP11-493E12.3, circRTKN, circELMOD3, circREV1, circZBTB20, circTIMMDC1, circACAD9, circPLXND1, circHDAC11, circCEP70, circRNF13.1, circRNF13.2, circGOLIM4, circElF4A2|SNORD2.1, circEIF4A2|SNORD2.2, circSDHAP1, circSETD2, circSCAP, circUSP4, circRPL29, circPHF7, circNEK4, circFLNB, circSLC25A26, circNFKB1, circFIP1L1|RP11-231C18.3, circTBC1D14, circALB.1, circALB.2, circALB.3, circNUP54, circAFF1, circSLC12A7 | MIR4635, circMAN2A1.1, circMAN2A1.2, circAFF4, circUBE2D2, circANKHD1|ANKHD1-EIF4EBP3, circMAPK9, circGPBP1, circCEP72, circRP11-98J23.2, circFAM169A, circWDR41, circRASGRF2, circRHOBTB3, circCEP85L, circARID1B.1, circARID1B.2, circTULP4|RP11-732M18.4, circTULP4, circTMEM181, circHIST1H3B, circHIST1H3C.2, circUNKNOWN00000008, circC6orf136, circHLA-C|HLA-B |XXbac-BPG248L24.10|WASF5P|XXbac-BPG248L24.13.1, circHLA-C|HLA-B|XXbac-BPG248L24.10|WASF5P|XXbac-BPG248L24.13.2, circFKBP5, circCNPY3, circSRF, circRN7SK, circFARS2, circMLIP, circZNF292, circPNRC1, circUNKNOWN00000009, circNDUFB2, circKMT2C, circESYT2, circMPP6, circHERPUD2, circOGDH, circZNF680, circKDELR2|DAGLB, circZDHHC4, circCCZ1B, circPOM121, circBAZ1B, circGTF2I, circSNORA14A, circCDK14, circCCDC132, circTRRAP|MIR3609, circCYP3A7|CYP3A7-CYP3A51P, circCCAT1.1, circCCAT1.2, circCCAT1.3, circCCAT1.4, circCCAT1.5, circCCAT1.6, circCCAT1.7, circASAP1, circPTK2.1, circPTK2.2, circSLC45A4, circADGRB1, circRBPMS, circFGFR1, circHOOK3, circASPH, circTMEM245, circUNKNOWN00000010, circHSPA5, circGLE1, circFOCAD, circNFX1, circUBAP2, circKDM4C|RP11-146B14.1, circAGTPBP1, circFAM120A.1, circFAM120A.2, circHIATL1, circPPP2R3B, circATRX, circFAT1 or circTBL1X.

7) The antisense oligonucleotide or siRNA or dsRNA according to anyone of items 1 - 6, wherein the antisense oligonucleotide or siRNA or dsRNA is at least 80%, such as at least 85%, such as at least 90 %, such as at least 100% complementary to a sequence of between 14 and 22 nucleotides in length and which is located within anyone of SEQ ID NOs: 1 - 359 and 2260.

8) The antisense oligonucleotide or siRNA or dsRNA of anyone of items 1 - 7, wherein the antisense oligonucleotide comprises in total at least three sugar-modified nucleobases that enhance the binding affinity of the antisense oligonucleotide to the circRNA.

9) The antisense oligonucleotide or siRNA or dsRNA of item 8, wherein the sugar modified nucleobase units are selected from the list of LNA (Locked nucleic acid), tricyclo-DNA, 2′-Fluoro, 2′-O-methyl, 2′methoxyethyl (2′MOE), 2′cyclic ethyl (cET), UNA, and Conformationally Restricted Nucleoside (CRN).

10) A compound according to item 9, wherein the nucleotide analogues are LNA, and selected from the list of beta-D-oxy LNA, alpha-L-oxy-LNA, beta-D-amino-LNA, alpha-L-amino-LNA, beta-D-thio-LNA, alpha-L-thio-LNA, 5′-methyl-LNA, beta-D-ENA and alpha-L-ENA.

11) A compound according to item 10, wherein the nucleosides are Beta-D-Oxy LNA.

12) A compound according to anyone of items 1 - 11, wherein the nucleoside analogues are a mixture of LNA and anyone of tricyclo-DNA, 2′-Fluoro, 2′-O-methyl, 2′-methoxyethyl (2′MOE), 2′cyclic ethyl (cET), UNA, and Conformationally Restricted Nucleoside (CRN).

13) A compound according to items 11 - 12 wherein the nucleoside analogues are a mixture of LNA and 2′-fluoro.

14) The antisense oligonucleotide according to any one of items 1-13, wherein all internucleoside linkages are phosphorothioate linkages.

15) The antisense oligonucleotide according to anyone of the preceding items, wherein the antisense oligonucleotide comprises a gap of at least 7, 8, 9, 10, 11, 12, 13 or 14 DNA units, flanked in each end by wings comprising at least one sugar-modified nucleobase.

16) The antisense oligonucleotide according to item 15, wherein the wings comprises 1, 2, 3, 4, 5, or 6 sugar modified nucleobase units, such as 2 to 5 modified nucleobase units.

17) The antisense oligonucleotide according to anyone of items 1-16, wherein the antisense oligonucleotide is anyone of SEQ ID NO’s: 360 - 2148 or anyone of SEQ ID NO’s 2285-2299.

18) The antisense oligonucleotide or siRNA according to anyone of the preceeding items, wherein the antisense oligonucleotide or siRNA is conjugated with a ligand for targeted delivery.

19) The antisense oligonucleotide or siRNA according to item 18, wherein the antisense oligonucleotide or siRNA is conjugated with folic acid or N-acetylgalactosamine (GalNAc).

20) The antisense oligonucleotide or siRNA according to anyone of items 1-17, wherein the antisense oligonucleotide or siRNA is unconjugated in a pharmaceutical composition for delivery.

21) The antisense oligonucleotide or siRNA according to anyone of items 1-17, wherein the antisense oligonucleotide or siRNA is formulated in lipid nanoparticles for delivery.

22) The antisense oligonucleotide or siRNA according to any one of the preceeding items, for use as a medicament.

23) The antisense oligonucleotide or siRNA according to item 22, wherein the antisense oligonucleotide or siRNA is for use as a medicament in the treatment of cancer.

24) The antisense oligonucleotide or siRNA according to item 23, wherein the antisense oligonucleotide or siRNA is according to items 2 - 5.

25) The antisense oligonucleotide or siRNA according to item 23 or 24, wherein the cancer is hepatocellular carcinoma.

26) A composition comprising an antisense oligonucleotide or siRNA according to anyone of the preceeding items and a carrier.

27) A composition comprising an antisense oligonucleotide or siRNA according to any one of items 1-21, for use as a pharmaceutical or in a method of treatment.

28) A composition according to items 26-27, wherein the composition comprises more than one antisense oligonucleotide or siRNA according to anyone of items 1-25.

29) A composition according to item 28, wherein the two or more antisense oligonucleotides or siRNA are selected from the list of anyone of SEQ ID NOs: 360 - 2148 or anyone of SEQ ID NO’s 2285-2299.

30) The composition according to anyone of items 26-29, wherein the antisense oligonucleotide or siRNA or composition is for treatment of hepatocellular carcinoma.

31) The composition according to item 30, wherein the cancer is selected from the list of cancers, such as hepatocellular carcinoma, breast cancer, CNS tumors, leukemias, melanoma, non-small cell lung cancer, prostate cancer or renal cancer.

32) The antisense oligonucleotide or siRNA according to anyone of items 1-25, or composition according items 26 to 31, wherein the antisense oligonucleotide or composition is for treatment of a human subject.

33) The antisense oligonucleotide or composition according to anyone of the preceding items, wherein the antisense oligonucleotide or siRNA or composition is for treatment of a cell ex vivo.

34) A method of knocking down an endogenous circRNA in a cell, by administration of an effective amount of an antisense oligonucleotide according to anyone of items 1-25, or a composition according to anyone of items 26-31 to a cell.

35) The method of item 34, wherein the cell is in a human body.

36) The method of item 35, wherein the cell is a cancer cell in a human body.

37) A method of treatment of cancer in, comprising the administration of an effective dosage of an antisense oligonucleotide or a composition according to anyone of items 1-36 to a human subject.

38) The method according to item 37, wherein the cancer is selected from the list of cancers such as hepatocellular carcinoma, breast cancer, CNS tumors, leukemias, melanoma, non-small cell lung cancer, prostate cancer or renal cancer.

39) The antisense oligonucleotides or siRNA, or compositions or methods of treatment according to any one of items 1-38, wherein the antisense oligonucleotide, or compositions or methods of treatment are for use in combination with another compound, composition or method of treatment.

40) A method of treating cancer, characterized by the following steps:
a. Isolate cancer cells from a patient.
b. Testing the presence of circRNAs in the cancer cells.
c. If the cancer cell is tested positive for a circRNA in step b, for one or more circRNAs, a composition comprising an antisense oligonucleotide or siRNA according to anyone of items 1-20 is selected, wherein the antisense oligonucleotide or siRNA is antisense to or has a region of complementarity to the circRNA that is expressed according to the test in step b.
d. The cancer is treated with the composition of step c, by administering an efficient amount of the composition to the patient having the cancer.

41) The method according to item 40, wherein the circRNA level measured in step b is any circRNA. 42) The method according to item 40 or 41, wherein the circRNA level measured in step a is anyone selected from the list of ciRS-7, circFAT1, circPVT1, circHIPK3, circSRY, circSLC35E2B, circCDK11A, circUNKNOWN00000001, circARHGAP32, circSLC8A3, circHERC2, circZFAND6, circRP1-168P16.1, circAURKC, circAFTPH, circSCD, circSMC3, circSNORA23|IP07.1, circZNF124.1, circSNX5|OVOL2, circRALY, circTFPI, circAHSG.1, circAHSG.2, circAHSG.3, circUBXN7, circAFP, circHIST1H3A, circHIST1H3C.1, circANAPC2, circRMRP|RMRP, circCENPI, circFIRRE, circMBNL3, circGPC3, circPROSER2, circMALRD1, circFAM208B, circMCU, circKIF20B, circABCC2, circElF4G2|SNORD97.1, circEIF4G2|SNORD97.2, circEIF4G2|SNORD97.3, circEIF4G2|SNORD97.4, circEIF4G2|SNORD97.5, circEIF4G2|SNORD97.6, circEIF4G2|SNORD97.7, circEIF4G2|SNORD97.8, circEIF4G2|SNORD97.9, circElF4G2|SNORD97.10, circlGF2, circQSER1, circUNKNOWN00000002, circCHD1L, circPRUNE, circSLC27A3, circGATAD2B, circKIAA0907, circCCT3, circPLEKHM2, circVWCE, circATF6, circMALAT1.1, circMALAT1.2, circMALAT1.3, circMALAT1.4, circMALAT1.5, circMALAT1.6, circMALAT1.7, circMALAT1.8, circMALAT1.9, circMALAT1.10, circMALAT1.11, circMALAT1.12, circMALAT1.13, circUNKNOWN00000003, circMALAT1.14, circMALAT1.15, circMALAT1.16, circMALAT1.17, circMALAT1.18, circMALAT1.19, circUCK2, circSUCO, circRAB6A, circRPS3|SNORD15B.1, circRPS3|SNORD15B.2, circRPS3|SNORD15B.3, circRSF1, circABL2, circGNB1, circRPLP2|SNORA52, circPICALM.1, circPICALM.2, circSNORA23|IPO7.2, circSNORA23|IPO7.3, circCFH, circSLC41A2.1, circSLC41A2.2, circCORO1C, circElF4G3|RP11-487E1.2, circNAA25, circMED13L, circLPGAT1| RN7SL344P, circAACS, circTP53BP2, circSOX5, circDNAH14, circKDM1A|MIR3115, circTTC13, circEGLN1, circTCEA3, circTOMM20|SNORA14B, circSCCPDH, circZNF124.2, circGLS2, circR3HDM2, circDHDDS, circSNORA73A|RCC1|SNHG3.1, circSNORA73A|RCC1|SNHG3.2, circSNORA61|SNHG12, circCEP83|RBMS2P1, circFGD6, circPUM1, circTMCO3|RP11-230F18.6, circPTP4A2, circZMYM5, circN6AMT2, circRPL21|SNORA27, circGTF2F2, circZMYM4, circLINC00355, circUNKNOWN00000004, circFARP1, circDYNC1H1, circCDC42BPB, circCCNB1IP1|SNORA79|AL355075.1, circRPPH1|RPPH1.1, circRPPH1|RPPH1.2, circRPPH1|RPPH1.3, circRPPH1|RPPH1.4, circSNORD8|CHD8.1, circSNORD8|CHD8.2, circPPP1R3E, circCHMP4A|RP11-468E2.1|AL136419.6, circUNKNOWN00000005, circSEC23A, circSNORD46 | RPS8, circSAMD4A, circPCNX, circPSEN1, circFCF1, circSCARNA13|SNHG10.1, circSCARNA13|SNHG10.2, circSCARNA13|SNHG10.3, circUNKNOWN00000006, circTJP1, circRP11-632K20.7, circTTBK2, circPPIB, circUBE2Q2, circETFA, circSEC11A, circPDE8A, circDAB1 |OMA1, circABHD2, circlQGAP1.1, circlQGAP1.2, circCHD2, circlGF1R, circNPRL3, circNDE1, circABCC1, circRPS2 |SNORA64, circPOLR3E, circATXN2L, circMVP, circASPHD1, circITGAL, circRP5-857K21.6.1, circRP5-857K21.6.2, circRP5-857K21.6.3, circRP5-857K21.6.4, circZNF720, circLONP2, circCHD9, circSLC7A6, circCARHSP1, circFANCA, circRAD51D|RAD51L3-RFFL, circHDAC5, circUTP18, circSRSF1, circPPM1D, circBRIP1, circPRKCA.1, circPRKCA.2, circEIF4A1|SNORD10|RP11-186B7.4|SENP3-EIF4A1.1, circEIF4A1|SNORD10|RP11-186B7.4|SENP3-EIF4A1.2, circPGS1, circRPTOR, circRPL26|RP11-849F2.7, circRP11-206L10.8, circPIAS2, circTYMS, circPPP4R1, circZNF91, circWDR62, circADCK4, circARHGAP35, circNUCB1, circSNORD33|RPL13A.1, circSNORD33|RPL13A.2, circSNORD33|RPL13A.3, circMUC16, circLZIC, circSNX5|SNORD17|OVOL2.1, circSNX5|SNORD17|OVOL2.2, circSNORA71A|SNHG17, circPLTP, circTMEM230, circCYP24A1, circZBTB46, circGART, circRAB3GAP1, circDYRK1A, circUNKNOWN00000007, circCOL18A1.1, circCOL18A1.2, circNBAS, circCH507-513H4.1.1, circCH507-513H4.1.2, circCH507-513H4.1.3, circANKAR, circGLS, circBMPR2, circRHBDD1, circATG16L1|SCARNA5, circDGKD, circPASK, circPPP6R2, circBIRC6, circPRKD3, circKIAA1841|RP11-493E12.3, circRTKN, circELMOD3, circREV1, circZBTB20, circTIMMDC1, circACAD9, circPLXND1, circHDAC11, circCEP70, circRNF13.1, circRNF13.2, circGOLIM4, circEIF4A2|SNORD2.1, circEIF4A2|SNORD2.2, circSDHAP1, circSETD2, circSCAP, circUSP4, circRPL29, circPHF7, circNEK4, circFLNB, circSLC25A26, circNFKB1, circFIP1L1|RP11-231C18.3, circTBC1D14, circALB.1, circALB.2, circALB.3, circNUP54, circAFF1, circSLC12A7|MIR4635, circMAN2A1.1, circMAN2A1.2, circAFF4, circUBE2D2, circANKHD1|ANKHD1-EIF4EBP3, circMAPK9, circGPBP1, circCEP72, circRP11-98J23.2, circFAM169A, circWDR41, circRASGRF2, circRHOBTB3, circCEP85L, circARID1B.1, circARID1B.2, circTULP4|RP11-732M18.4, circTULP4, circTMEM181, circHIST1H3B, circHIST1H3C.2, circUNKNOWN00000008, circC6orf136, circHLA-C|HLA-B|XXbac-BPG248L24.10|WASF5P|XXbac-BPG248L24.13.1, circHLA-C |HLA-B|XXbac-BPG248L24.10|WASF5P|XXbac-BPG248L24.13.2, circFKBP5, circCNPY3, circSRF, circRN7SK, circFARS2, circMLIP, circZNF292, circPNRC1, circUNKNOWN00000009, circNDUFB2, circKMT2C, circESYT2, circMPP6, circHERPUD2, circOGDH, circZNF680, circKDELR2|DAGLB, circZDHHC4, circCCZ1B, circPOM121, circBAZ1B, circGTF2I, circSNORA14A, circCDK14, circCCDC132, circTRRAP|MIR3609, circCYP3A7|CYP3A7-CYP3A51P, circCCAT1.1, circCCAT1.2, circCCAT1.3, circCCAT1.4, circCCAT1.5, circCCAT1.6, circCCAT1.7, circASAP1, circPTK2.1, circPTK2.2, circSLC45A4, circADGRB1, circRBPMS, circFGFR1, circHOOK3, circASPH, circTMEM245, circUNKNOWN00000010, circHSPA5, circGLE1, circFOCAD, circNFX1, circUBAP2, circKDM4C|RP11-146B14.1, circAGTPBP1, circFAM120A.1, circFAM120A.2, circHIATL1, circPPP2R3B, circATRX, circFAT1 or circTBL1X.

### Items Relating to Compounds Targeting IncRNAs

1) A compound comprising a gapmer antisense oligonucleotide consisting of any one of SEQ ID NOs: 2149 - 2259.

2) A compound according to item 1, wherein the nucleotide analogues of the wings are selected from the list of beta-D-oxy LNA, alpha-L-oxy-LNA, beta-D-amino-LNA, alpha-L-amino-LNA, beta-D-thio-LNA, alpha-L-thio-LNA, 5′-methyl-LNA, beta-D-ENA and alpha-L-ENA.

3) A compound according to item 2, wherein the nucleosides of the wings are Beta-D-Oxy LNA.

4) A compound according to item 1, wherein the nucleoside analogues of the wings are not LNA, but anyone of tricyclo-DNA, 2′-Fluoro, 2′-O-methyl, 2′-methoxyethyl (2′-MOE), 2′cyclic ethyl (cET), UNA, and Conformationally Restricted Nucleoside (CRN).

5) A compound according to item 1, wherein the nucleoside analogues of the wings are a mixture of LNA and anyone of tricyclo-DNA, 2′Fluoro, 2′-O-methyl, 2′-methoxyethyl (2′-MOE), 2′cyclic ethyl (cET), UNA, and Conformationally Restricted Nucleoside (CRN).

6) A compound according to item 5, wherein the nucleoside analogues of the wings are a mixture of LNA and 2′-Fluoro.

7) The compound according to anyone of items 1-6, wherein the antisense oligonucleotide is conjugated with a ligand for targeted delivery.

8) The compound according to item 7, wherein the antisense oligonucleotide is conjugated with folic acid or N-acetylgalactosamine (GalNAc).

9) The compound according to anyone of items 1-6, wherein the antisense oligonucleotide is unconjugated in a pharmaceutical composition for delivery.

10) The compound according to anyone of items 1-9, wherein the antisense oligonucleotide is formulated in lipid nanoparticles for delivery.

11) The compound according to any one of the preceding items, for use as a medicament.

12) The compound according to item 11, wherein the antisense oligonucleotide is for use as a medicament in the treatment of cancer.

13) The compound according to item 12, wherein the cancer is hepatocellular carcinoma.

14) A composition comprising a compound or an antisense oligonucleotide according to anyone of the preceding items and a carrier.

15) A composition comprising a compound or an antisense oligonucleotide according to any one of items 1-9, for use as a pharmaceutical or in a method of treatment.

16) A composition according to items 14-15, wherein the composition comprises more than one compound or antisense oligonucleotide according to anyone of items 1-13.

17) A composition according to item 16, wherein the two or more antisense oligonucleotides are selected from the list of anyone of SEQ ID NOs: 2149 - 2259.

18) The composition according to anyone of items 14-17, wherein the composition is for treatment of cancer.

19) The composition according to item 18, wherein the cancer is hepatocellular carcinoma.

20) The compound or antisense oligonucleotide according to anyone of items 1-13, or composition according items 14 to 19, wherein the compound or antisense oligonucleotide or composition is for treatment of a human subject.

21) The compound or antisense oligonucleotide or composition according to anyone of the preceding items, wherein the antisense oligonucleotide or composition is for treatment of a cell ex vivo.

22) A method of downregulating an endogenous IncRNA selected from the list of DANCR, H19, HOTAIR, HOTTIP, HULC, LINC-ROR, MALAT1, MVIH, NEAT1, PCBP2-OT1, PVT1, TUG1, UCA1, UFC1 and LINC01215 in a cell, by administration of an effective amount of a compound or antisense oligonucleotide that is complementary to the target and selected from the list of SEQ ID NOs: 2149 - 2259 and according to anyone of items 1-12, or a composition according to anyone of items 13-18 to a cell.

23) The method of item 22, wherein the cell is in a human body.

24) The method of item 23, wherein the cell is a cancer cell in a human body.

25) A method of treatment of cancer, comprising the administration of an effective dosage of a compound or antisense oligonucleotide or a composition according to anyone of items 1-21 to a human.

26) The method according to item 24, wherein the cancer is hepatocellular carcinoma.

27) The antisense oligonucleotides, or compositions or methods of treatment according to any one of items 1-26, wherein the antisense oligonucleotide, or compositions or methods of treatment are for use in combination with another compound, composition or method of treatment.

### EXAMPLES

Example 1. LNA monomer and oligonucleotide synthesis may be performed using the methodology referred to in Examples 1 and 2 of WO2007/11275. Assessment of the stability of LNA oligonucleotides in human or rat plasma may be performed using the methodology referred to in Example 4 of WO2007/112754. Treatment of cultured cells with LNA-modified antisense oligonucleotides may be performed using the methodology referred to in Example 6 of WO2007/11275.

Example 2. RNA isolation and expression analysis from cultured cells and tissues is performed using the methodology referred to in Example 10 of WO2007/112754. RNAseq-based transcriptional profiling from cultured cells and tissues is performed using the methodology referred to in (Jeck et al. 2013, RNA 19: 141-157 or Zheng et al. 2016 Nature Commun. 7: 11215).

### Example 3 General Description of the Antisense Oligonucleotide Design Workflow

Antisense oligonucleotides capable of decreasing the expression of target transcript(s) are designed as RNaseH-recruiting gapmer oligonucleotides. Gapmer oligonucleotides are designed by applying various locked nucleid acid (LNA)/DNA patterns (typically the patterns constitute a central region of DNA flanked by short LNA wings, e.g. LLLDDDDDDDDDDLLL, where L denotes LNA and D denotes DNA) to the reverse complement of target site sequences. A comprehensive list of all n-mer target sites in a transcript (n = 14-20 bases, non-limiting example) is generated and oligonucleotides that can bind to the target sites with desired specificity in the transcriptome and have desired thermodynamic and structural properties are synthesized and tested in vitro in cancer cell lines and subsequently in vivo in mouse tumor models. The ASOs of this invention, are listed in Table 2 and 3 (LNA= uppercase, DNA lowercase, complete phosphorothioate backbone), and examples demonstrating their potential in circRNA and IncRNA knockdown are described in examples 4-12 below.

### Example 4 Identification of Cancer-Associated circRNAs

RNAseq data was mapped to the human genome (hg38) using the RNAseq aligner STAR (Dobin et al. 2013, Bioinformatics 29: 15-21) with chimeric alignment detection enabled, essentially as described in the manual. Subsequent to read alignment, the chimeric reads were filtered to identify spliced reads where the donor and acceptor are on the same chromosome, same strand, and the donor is positioned downstream of the acceptor (maximum allowed distance between donor and acceptor is 100.000 bases). Donor and acceptor positions were defined as the intronic positions surrounding the bases that are covalently linked by backsplicing, and the chromosomal coordinate system used is 1-based. Each backsplice junction was uniquely identified in the hg38 genome by the chromosome name (chrName), position of the donor and acceptor (posAcceptor and posDonor), and the strand of the chromosome (strand). A unique backsplice ID (bsID) was generated from this info ([chrName]:[posAcceptor]-[posDonor] | [strand], e.g. X:140783175-140784661|+). Back-splice junctions from cancer-associated circRNAs were identified by analyzing RNAseq data from multiple myeloma patients, by searching for hepatocellular carcinoma-associated circRNAs in the circ2Traits database (http://gyanxetbeta.com/circdb/searchdis.php?trait=hepatocellular+carcinoma&but=Search), analysis of HepG2 and liver RNAseq data from the ENCODE project (https://www.encodeproject.org/), and analysis of Gene Expression Omnibus dataset with accession number GSE77661. Multiple myeloma RNAseq data was analyzed to find circRNAs showing up-regulation in sorted malignant plasma cells from multiple myeloma patients compared to plasma cells from healthy donors. The ENCODE data were analyzed to identify back-splice junctions of circRNAs that exhibited higher expression in HepG2 cells than in adolescent/adult liver samples. GSE77661 data was analyzed to find back-splice junctions of circRNAs that were upregulated in hepatocellular carcinoma compared to normal adjacent tissue. Collectively, this resulted in identification of 359 backsplice junctions (Table 1). From the list of 359 backsplice junctions, we generated a shortlist consisting of ciRS-7, circPVT1, circHIPK3, circSRY, the 10 backsplice junctions from circRNAs found to be associated with hepatocellular carcinoma in the circ2Traits database (circSLC35E2B, circCDK11A, circUNKNOWN00000001, circARHGAP32, circSLC8A3, circHERC2, circZFAND6, circRP1-168P16.1, circAURKC, and circAFTPH) and 20 backsplice junctions from circRNAs that also show fetal expression (circSCD, circSMC3, circSNORA23|IPO7.1, circZNF124.1, circSNX5|OVOL2, circRALY, circTFPI, circAHSG.1, circAHSG.2, circAHSG.3, circUBXN7, circAFP, circHIST1H3A, circHIST1H3C.1, circANAPC2, circRMRP|RMRP, circCENPI, circFIRRE, circMBNL3, and circGPC3).

### Example 5 Design of LNA-Modified Antisense Oligonucleotides for Knockdown of the ciRS-7 Circular RNA

LNA antisense oligonucleotides that can effectively knock down the ciRS-7 circRNA were designed. In this example, the target region is the sequence that is generated by back-splicing of the CDR1-AS transcript (SEQ ID: 1, see appendix), i.e. linking the end of the transcript to the start of the transcript to form a circular molecule (designated as ciRS-7). Three LNA ASOs were synthesized that cover the ciRS-7 back-splice junction site (Table 3: SEQ ID NOs: 360-362).

### Example 6. Design of LNA-Modified Antisense Oligonucleotides for Knockdown of IncRNAs In Hepatocellular Carcinoma.

Several IncRNAs, such as DANCR, H19, HOTAIR, HOTTIP, HULC, LINC-ROR, MALAT1, MVIH, NEAT1, PCBP2-OT1, PVT1, TUG1, UCA1, UFC1 and LINC01215 have been implicated in the pathogenesis of hepatocellular carcinoma (for review, see Parasramka et al., 2016, Pharmacol. Ther. 161: 67-78). LNA antisense oligonucleotides for knockdown of DANCR, H19, HOTAIR, HOTTIP, HULC, LINC-ROR, MALAT1, MVIH, NEAT1, PCBP2-OT1, PVT1, TUG1, UCA1, UFC1 and LINC01215 were designed as described in example 3. In this example, the target regions for DANCR are generated from spliced and unspliced transcripts (SEQ ID NOs 2262 and 2273, respectively), the target regions for H19 are generated from spliced and unspliced transcripts (SEQ ID NOs 2266 and 2278, respectively), the target regions for HOTAIR are generated from spliced and unspliced transcripts (SEQ ID NOs 2267 and 2280, respectively), the target regions for HOTTIP are generated from spliced and unspliced transcripts (SEQ ID NOs 2264 and 2275, respectively), the target regions for HULC are generated from spliced and unspliced transcripts (SEQ ID NOs 2263 and 2274, respectively), the target regions for LINC-ROR are generated from spliced and unspliced transcripts (SEQ ID NOs 2268 and 2281, respectively), the target regions for MALAT1 are generated from unspliced transcript (SEQ ID NO 2277), the target regions for MVIH are generated from unspliced transcript (SEQ ID NO 2284), the target regions for PCBP2-OT1 are generated from unspliced transcript (SEQ ID NO 2279), the target regions for TUG1 are generated from spliced and unspliced transcripts (SEQ ID NOs 2270 and 2283, respectively), the target regions for UCA1 are generated from spliced and unspliced transcripts (SEQ ID NOs 2269 and 2282, respectively), the target regions for UFC1 are generated from spliced and unspliced transcripts (SEQ ID NOs 2260 and 2271, respectively), and the target regions for LINC01215 are generated from spliced and unspliced transcripts (SEQ ID NOs 2261 and 2272, respectively). Two ASOs that target LINC01215 were synthesized (SEQ ID NO: 2191 - 2192).

### Example 7 Design of LNA-Modified Antisense Oligonucleotides for Knockdown of the PVT1 LincRNA

LNA antisense oligonucleotides for knockdown PVT1 lincRNA were designed. In this example, the target region is the sequence corresponding to the unspliced PVT1 transcript (SEQ ID NO: 2276) or the spliced transcript (SEQ ID NO: 2265). Two ASOs that target this target region were synthesized (SEQ ID NO: 2233-2234).

### Example 8: Cell Culture

Mammalian cancer cell lines are routinely used as models for testing the effect of the antisense oligonucleotides in vitro.

The adherent lung cancer cell line A549 (ECACC cat. no. 86012804) was purchased from Sigma and maintained in Dulbecco’s modified Eagle’s medium (Sigma cat. no. D6546) supplemented with 10% fetal calf serum (Sigma cat. no. F2442), 2 mM L-glutamine (Sigma cat. no. G7513) and penicillin/streptomycin (Sigma cat. no. P4333) in a humidified 5% CO2 incubator at 37° C. and passaged twice a week.

The adherent prostate cancer cell line PC3 (ECACC cat. no. 90112714) was purchased from Sigma and maintained in Ham’s F12K (Kaighn’s) (Life Technologies cat. no. 21127-022) supplemented with 10% fetal calf serum (Sigma cat. no. F2442), and penicillin/streptomycin (Sigma cat. no. P4333) in a humidified 5% CO2 incubator at 37° C. and passaged twice a week.

The semi-adherent multiple myeloma cell line MM.1S was a gift from Prof. K. Dybkjaer at Aalborg University and maintained in RPMI1640 medium (Sigma cat. no. R0883) supplemented with 10% fetal calf serum (Sigma cat. no. F2442), 2 mM L-glutamine (Sigma cat. no. G7513) and penicillin/streptomycin (Sigma ca. no. P4333) in a humidified 5% CO2 incubator at 37° C. and passaged twice a week.

### Example 9: Antisense-Mediated Knockdown of the PVT1 lincRNA in Cultured Cancer Cells

The knock down effect mediated by antisense oligonucleotides designed as described in example 3 can be routinely measured in vitro in cultured mammalian cell lines in a number of ways well known to a person skilled in the art. The target transcript must be expressed at a detectable level in the cell lines used, either through endogenous expression or by transient or stable transfection of the target transcript into said cell line. The level of target expression can be measured for example by quantitative PCR or Northern blot. The antisense oligonucleotide can be introduced into the cells using a lipid vehicle or via unassisted uptake.

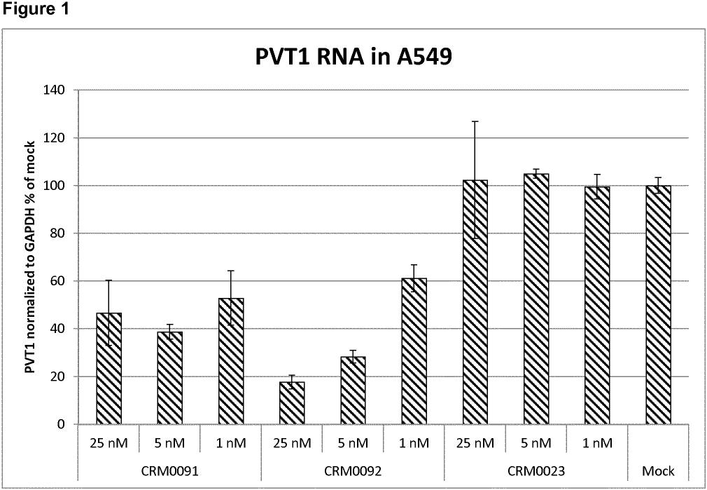

For lipid-mediated transfection of the PVT1-targeting antisense oligonucleotides listed in Table 3 (SEQ ID NOs: 2233 - 2234) A549 cells were seeded in 12-well cell culture plates the day before transfection and transfected essentially as described in Dean et al. (Journal of Biological Chemistry 1994, 269, 16416-16424) using Lipofectamine 2000 in a final concentration of 5 µl/ml Optimem I (Gibco) and antisense oligonucleotide in a concentration range of 1 nM - 25 nM final concentration. A scrambled sequence oligonucleotide and mock transfection were included as controls. 24 hours after transfection, total RNA was isolated from the cells using the RNeasy mini kit (Qiagen) according to the manufacturer’s instructions and 1 µg total RNA was reverse transcribed into cDNA using the High Capacity cDNA reverse transcription kit (Life Technologies cat. no. 4374967) according to the protocol provided by the manufacturer. PVT1 RNA levels were determined by quantitative RT-PCR using Taqman Gene Expression Master Mix (ABI cat. no. 4369542) and pre-designed PVT1 Taqman assays (IDT Hs.PT.58.24584277), furthermore the expression of GAPDH mRNA was measured (IDT Hs.PT.58.40035104) and used as an endogenous control. Quantitative PCR was carried out on a Quantstudio 6 Flex Real-Time thermocycler (ABI). An example of knockdown of the PVT1 lincRNA in A549 cells, using SEQ ID NOs 2233 and 2234, is shown in FIG. 1.

### Example 10: Antisense-Mediated Knockdown of ciRS-7 in Cultured Cancer Cells

For lipid-mediated transfection of the ciRS-7 antisense oligonucleotides listed in Table 2 (SEQ ID NOs: 360 -362), A549 cells were transfected as described in example 9.

For transfection of PC3 cells, cells were seeded in 12-well cell culture plates at a density of 125,000 cells/well the day before transfection and transfected using Lipofectamine 2000 at a final concentration of 2.5 µl/ml using the protocol described in example 9.

Levels of the ciRS-7 RNA were measured using quantitative RT-PCR as described in example 8, briefly, the total amount of ciRS-7 transcript was measured using a Taqman assay designed with convergent PCR primers specific to the RNA, while the circularized form of ciRS-7 was measured using a Taqman assay designed with divergent PCR primers specific to the ciRS-7 RNA, the expression of GAPDH mRNA was measured and used as an endogenous control as described in example 9.

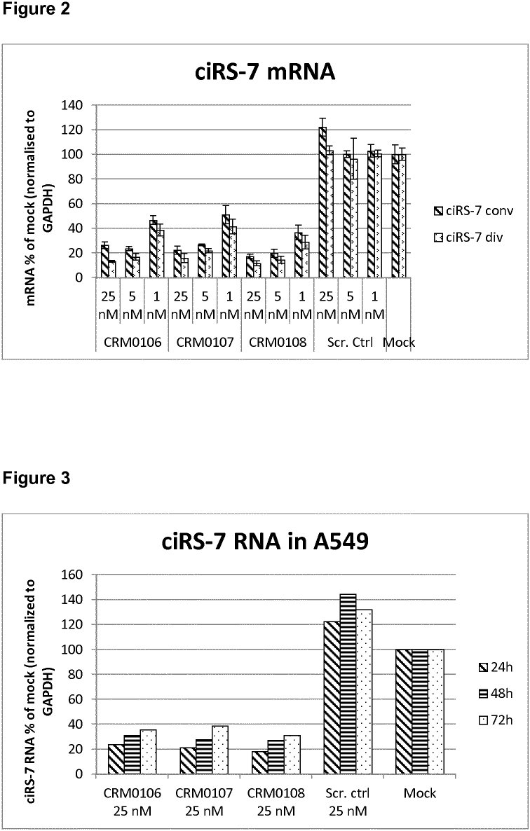

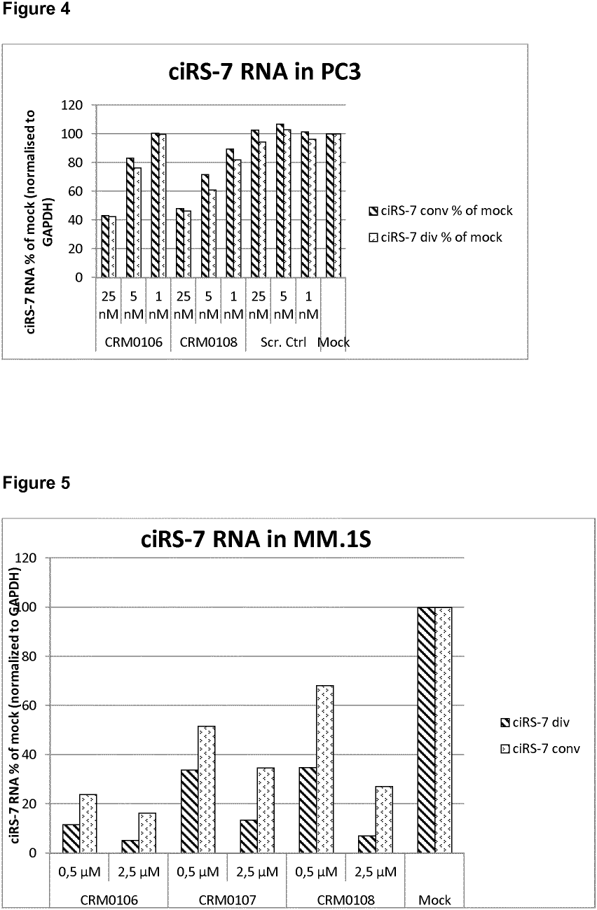

Examples of inhibition of ciRS-7 in A549 cells are shown in FIGS. 2 and 3. Examples of inhibition of ciRS-7 in PC3 cells are shown in FIG. 4.The semi-adherent multiple myeloma cell line MM.1S was a gift from Prof. K. Dybkjaer at Aalborg University and maintained in RPMI1640 medium (Sigma cat. no. R0883) supplemented with 10% fetal calf serum (Sigma cat. no. F2442), 2 mM L-glutamine (Sigma cat. no. G7513) and penicillin/streptomycin (Sigma cat. no. P4333) in a humidified 5% CO2 incubator at 37° C. and passaged twice a week. For unassisted uptake of the ciRS-7 antisense oligonucleotides listed in Table 2, MM.1S cells were seeded in 12-well cell culture plates the day before transfection at a cell density of 125.000 cells/well and transfected essentially as described in Soifer et al. (Methods Mol Biol. 2012; 815: 333-46) using antisense oligonucleotide in a final concentration range of 0.1 µM - 2.5 µM final concentration. Three to six days after transfection, total RNA was isolated from the cells using the RNeasy mini kit (Qiagen) as described in example 9 and levels of ciRS-7 RNA was measured using quantitative PCR as described above. Examples of knockdown of ciRS-7 RNA in MM.1S cells using antisense oligonucleotides CRM0106, CRM0107 and CRM0108 (SEQ ID NOs 360, 361, and 362 respectively) are shown in FIG. 5.

### Example 11: Effect of ciRS-7 Knockdown on Cancer Cell Proliferation

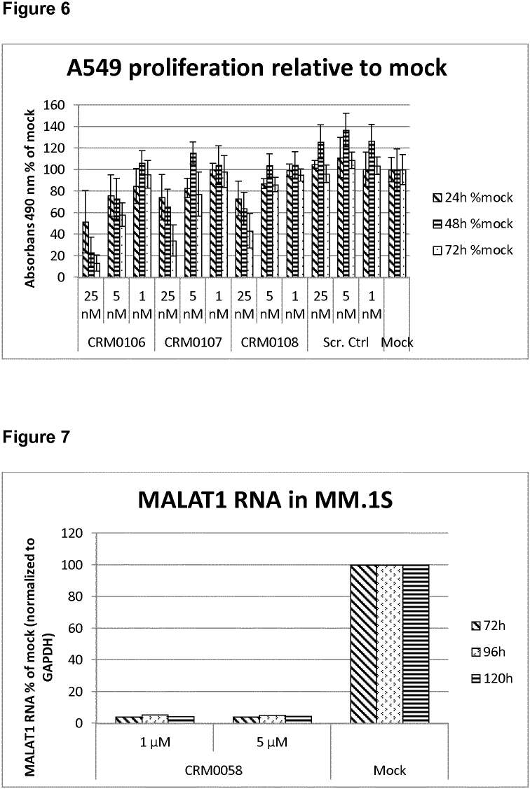

For lipid transfection, A549 cells were seeded in clear 96-well plates (NUNC) at a density of 2000 cells pr. well in complete culture medium the day before transfection. Six wells were left without cells for blank control. Lipid transfection was carried out as described in example 9 using 0.25 µl Lipofectamine 2000 in 50 µl OptiMEM pr. well and oligonucleotide concentrations ranging from 1 nM to 25 nM in 6 wells pr. concentration. After 4 hours, the cells were washed with OptiMEM, 100 µl complete culture medium was added to each well and the cells were incubated in a humidified 5% CO2 incubator at 37° C. for 24 - 72 hours. For measurement of cell proliferation, 20 µl of the CellTiter Aqueous One Solution (Promega) was added to each well and the cells were incubated for 1-4 hours in a humidified 5% CO2 incubator at 37° C. The absorbance at 490 nm was read in a Varioskan Lux plate-reader (Thermo Fisher Scientific) and the values from the blank controls were subtracted. The inhibition of proliferation was plotted relative to mock treated controls. Examples on the effect of ciRS-7 knockdown on A549 cell proliferation using antisense oligonucleotides CRM0106, CRM0107 and CRM0108 (SEQ ID NOs 360, 361, and 362 respectively) are shown in FIG. 6.

### Example 12: Antisense-Mediated Knockdown of MALAT1 lincRNA in Cultured Cancer Cells

For unassisted uptake of the MALAT1 antisense oligonucleotide listed in Table 3, MM.1S cells were seeded in 12-well cell culture plates transfected essentially as described in Soifer et al. (Methods Mol Biol. 2012; 815: 333-46) and antisense oligonucleotide was added in a concentration range of 0.1 µM -5 µM final concentration. MALAT1 antisense oligonucleotide CRM0058 (SEQ ID NO 2198) was tested against Mock. Three to six days after transfection, total RNA was isolated from the cells using the RNeasy mini kit (Qiagen) as described in example 9. MALAT1 RNA levels were determined by quantitative PCR using Taqman Gene Expression Master Mix (ABI cat. no. 4369542) and pre-designed Taqman assays for MALAT1 (IDT Hs.PT.58.3907580), furthermore the expression of GAPDH mRNA was measured (IDT Hs.PT.58.40035104) and used as an endogenous control. Quantitative PCR was carried out on a Quantstudio 6 Flex Real-Time thermocycler (ABI). Examples of knockdown of MALAT1 in MM.1S cells are shown in FIG. 7.

### Example 13: Induction of Apoptosis in Cultured Cancer Cells After MALAT1 Knockdown

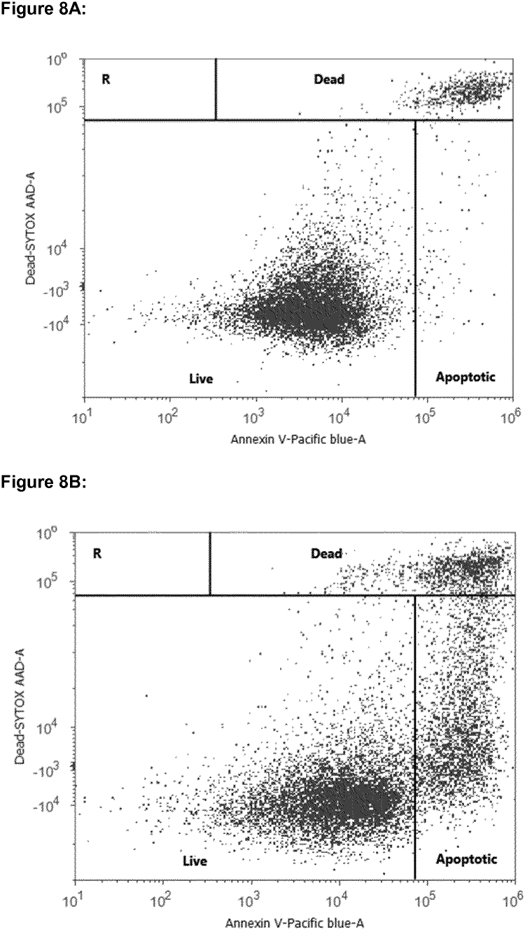

The induction of apoptosis in mammalian cells can be measured in various ways using apoptotic markers, such as the translocation of phosphatidylserine to the outer membrane, the activation of caspases, nuclear condensation and DNA fragmentation, which can all be measured by methods well known to a person skilled in the art. For assessment of apoptosis in cultured cancer cells after antisense oligonucleotide-mediated knockdown of MALAT1 (SEQ ID NO 2198), A549 cells were transfected in 12-well plates using Lipofectamine 2000 as described in example 9. Final concentration of the MALAT1 antisense oligonucleotide CRM0058 (SEQ ID NO: 2198) was 25 nM. After 24 hours, cells were harvested by trypsination, washed in cold PBS and stained using the Violet Annexin V/Dead Cell Apoptosis Kit with Pacific Blue annexin V/SYTOX AADvanced (Molecular Probes cat. No. A35136) using the manufacturer’s protocol and cells were subsequently analyzed on an Attune NxT flow cytometer (Life Technologies). Double-negative cells were considered to be live cells, cells positive for annexin V and negative for SYTOX AADvanced were considered as apoptotic cells and cells positive for SYTOX AADvanced were considered to be dead cells. The percentages of live, apoptotic and dead cells in A549 cells treated with CRM0058 (SEQ ID NO: 2198) and mock control as described above are listed in Table 4 and the corresponding dot plots are shown in FIG. 8A and FIG. 8B.

TABLE 4

The percentages of live, apoptotic and dead cells in A549 cells treated with CRM0058 (SEQ ID NO: 2198) and mock control

| Sample | Live (%) | Apoptotic (%) | Dead (%) |
|---|---|---|---|
| Mock | 92,769 | 0,893 | 6,339 |
| CRM0058 | 66,891 | 22,440 | 10,670 |

### Example 14: Cell Culture

The adherent liver adenocarcinoma cell line SK-Hep-1 (ECACC cat. no. 91091816) was in EMEM (EBSS) (Sigma cat. no. M2279) supplemented with 10% fetal calf serum (Sigma cat. no. F2442), 2 mM L-glutamine (Sigma cat. no. G7513), 1% NEAA (Sigma cat. no. M7145), 1 mM Sodium Pyruvate (NaP) (Sigma cat. no. S8636) and penicillin/streptomycin (Sigma cat. no. P4333) in a humidified 5% CO2 incubator at 37° C. and passaged twice a week.

The adherent hepatocellular carcinoma cell line Hep3B (ECACC cat. no. 86062703) was maintained in EMEM (EBSS) (Sigma cat. no. M2279) supplemented with 10% fetal calf serum (Sigma cat. no. F2442), 2 mM L-glutamine (Sigma cat. no. G7513), 1% NEAA (Sigma cat. no. M7145) and penicillin/streptomycin (Sigma cat. no. P4333) in a humidified 5% CO2 incubator at 37° C. and passaged twice a week.

The adherent hepatocellular carcinoma cell line HepG2 (ECACC cat. no. 85011430) was maintained in EMEM (EBSS) (Sigma cat. no. M2279) supplemented with 10% fetal calf serum (Sigma cat. no. F2442), 2 mM L-glutamine (Sigma cat. no. G7513), 1% NEAA (Sigma cat. no. M7145) and penicillin/streptomycin (Sigma cat. no. P4333) in a humidified 5% CO2 incubator at 37° C. and passaged twice a week.

The adherent hepatocellular carcinoma cell line Huh-7D12 (ECACC cat. no. 01042712) was maintained in Dulbecco’s modified Eagle’s medium (Sigma cat. no. D6546) supplemented with 10% fetal calf serum (Sigma cat. no. F2442), 2 mM L-glutamine (Sigma cat. no. G7513) and penicillin/streptomycin (Sigma cat. no. P4333) in a humidified 5% CO2 incubator at 37° C. and passaged twice a week.

The adherent glioblastoma cell line U87 (ECACC cat. no. 89081402) was maintained in EMEM (EBSS) (Sigma cat. no. M2279) supplemented with 10% fetal calf serum (Sigma cat. no. F2442), 2 mM L-glutamine (Sigma cat. no. G7513), 1% NEAA (Sigma cat. no. M7145), 1 mM Sodium Pyruvate (NaP) (Sigma cat. no. S8636) and penicillin/streptomycin (Sigma cat. no. P4333) in a humidified 5% CO2 incubator at 37° C. and passaged twice a week.

### Example 15: Antisense-Mediated Knockdown of ciRS-7 in Cultured Cancer Cells

For lipid-mediated transfection of the ciRS-7 antisense oligonucleotides listed in Table 2 (SEQ ID NOs: 360 - 362), cells were transfected as described in example 9.

For transfection of SK-Hep-1 cells, cells were seeded in 12-well cell culture plates at a density of 175,000 cells/well the day before transfection and transfected using Lipofectamine 2000 at a final concentration of 5 µl/ml using the protocol described in example 9.

For transfection of Hep3B cells, cells were seeded in 12-well cell culture plates at a density of 200,000 cells/well the day before transfection and transfected using Lipofectamine 2000 at a final concentration of 10 µl/ml using the protocol described in example 9.

Levels of the ciRS-7 RNA were measured using quantitative RT-PCR as described in example 8, briefly, the total amount of ciRS-7 transcript was measured using a Taqman assay designed with convergent PCR primers specific to the RNA, while ciRS-7 was measured using a Taqman assay designed with divergent PCR primers specific to the ciRS-7 RNA, the expression of GAPDH mRNA was measured and used as an endogenous control as described in example 9.

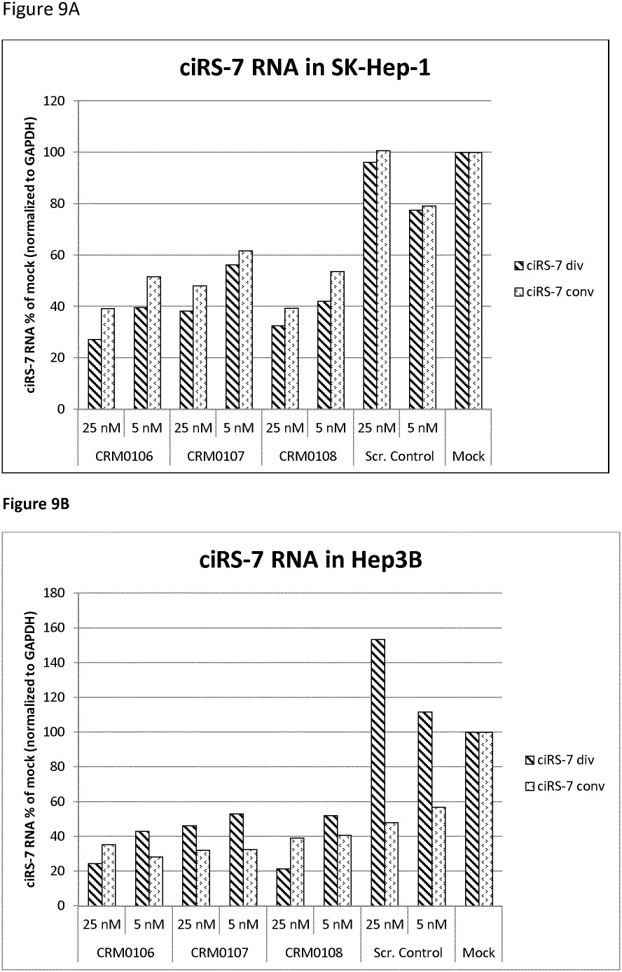

Examples of inhibition of ciRS-7 in SK-Hep-1 cells are shown in FIG. 9A. Examples of inhibition of ciRS-7 in Hep3B cells are shown in FIG. 9B.

### Example 16: Antisense-Mediated Knockdown of circRNAs in Cultured Cancercells

Antisense oligonucleotides against circRNAs identified as described in example 4 were designed as described in example 5. The antisense oligonucleotides against circRNAs are listed in Table 2.

For lipid-mediated transfection of the antisense oligonucleotides (CRM0171, CRM0167, CRM0168, CRM0169, CRM0170, CRM0172, CRM0173, CRM0174, CRM0176, CRM0177, CRM0178, CRM0179, CRM0180, CRM0181, CRM0182 and CRM0175) listed in Table 2 (SEQ ID NOs: 374, 2285, 2286, 2287, 2288, 2289, 2290, 2291, 2292, 2293, 2294, 2295, 2296, 2297, 2298 and 2299 respectively), A549 cells were transfected as described in example9.

For transfection of SK-Hep-1 cells, cells were seeded in 12-well cell culture plates at a density of 125,000 cells/well the day before transfection and transfected using Lipofectamine 2000 at a final concentration of 5 µl/ml using the protocol described in example9.

For transfection of Hep3B cells, cells were seeded in 12-well cell culture plates at a density of 200,000 cells/well the day before transfection and transfected using Lipofectamine 2000 at a final concentration of 10 µl/ml using the protocol described in example9.

Levels of the circRNAs were measured using quantitative RT-PCR as described in example 8, briefly, the amount of linear transcript was measured using a Taqman assay designed with convergent PCR primers specific to the linear RNA, while each circRNA was quantified using a Taqman assay designed with divergent PCR primers specific to the circRNA. The expression of TBP mRNA (IDT cat. no. Hs.PT.58v.39858774) was measured and used as an endogenous control.

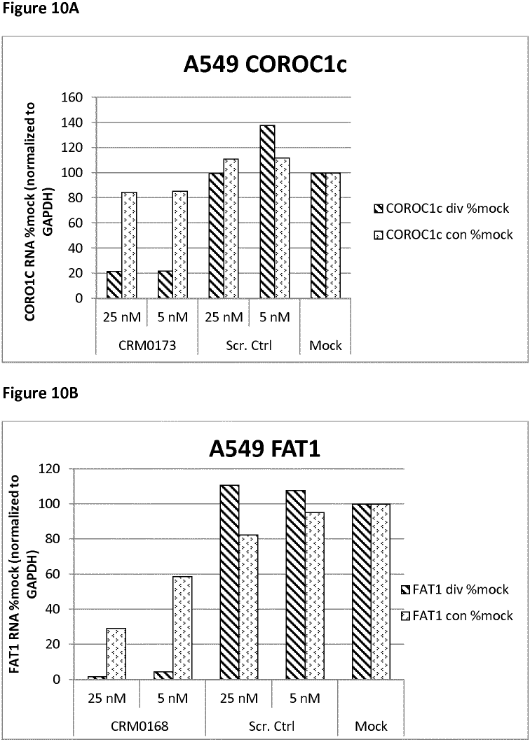

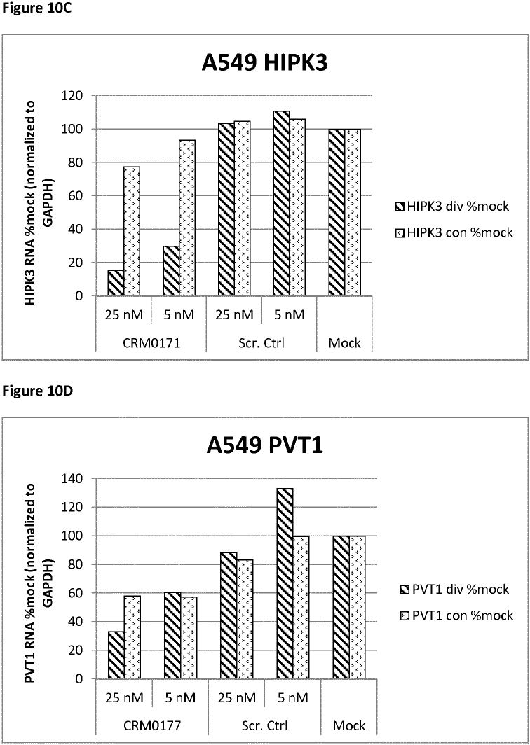

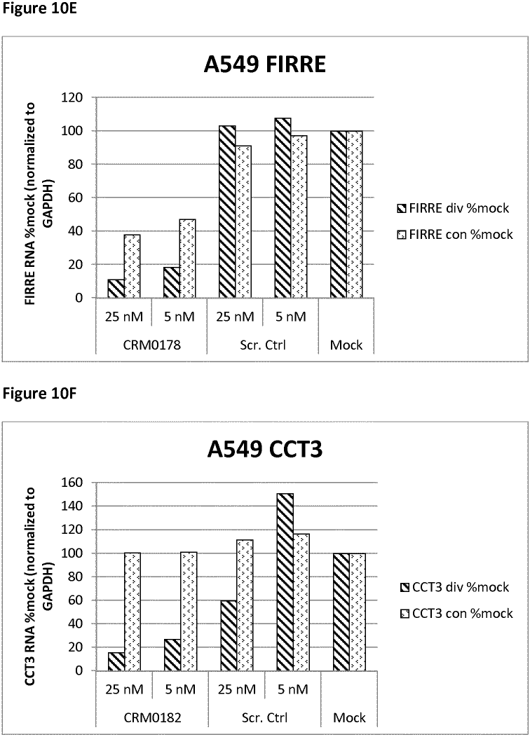

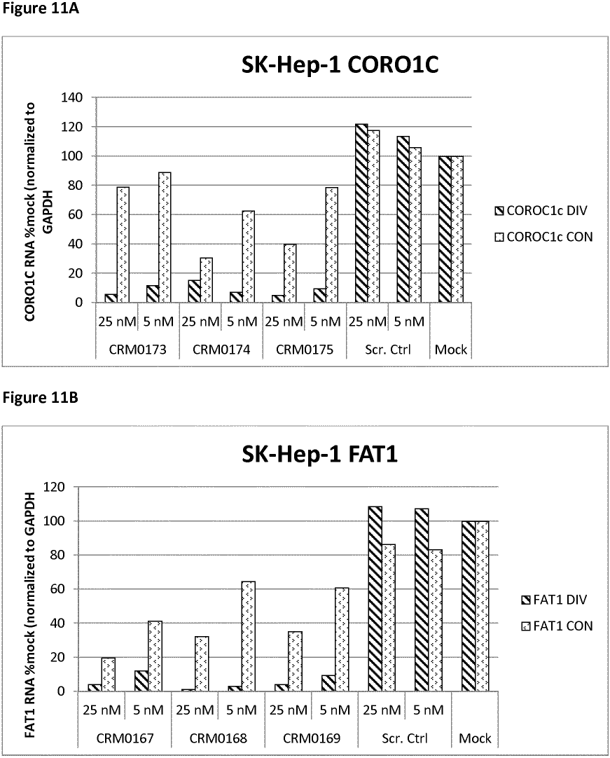

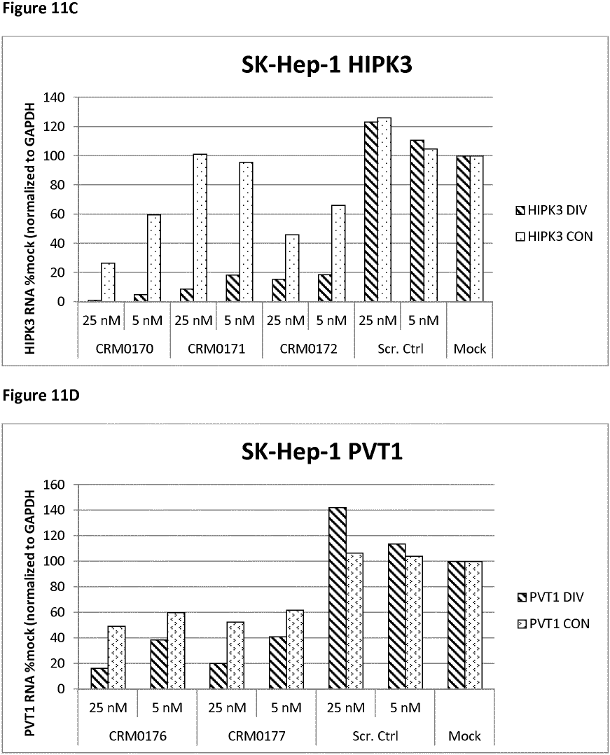

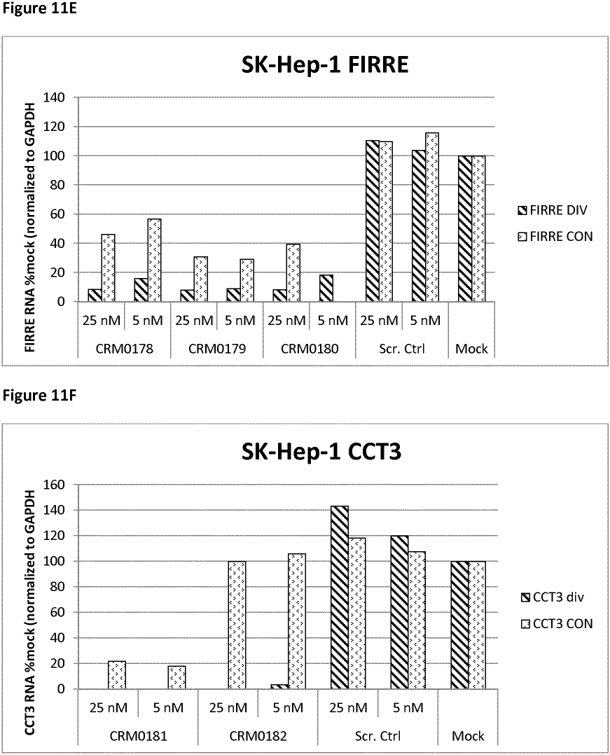

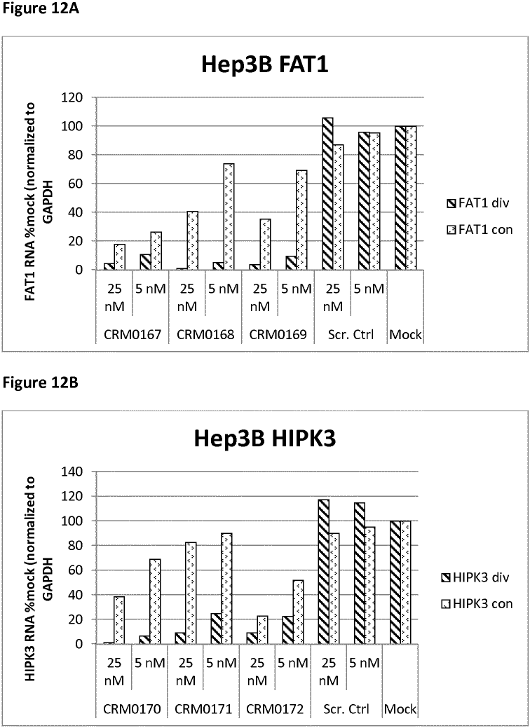

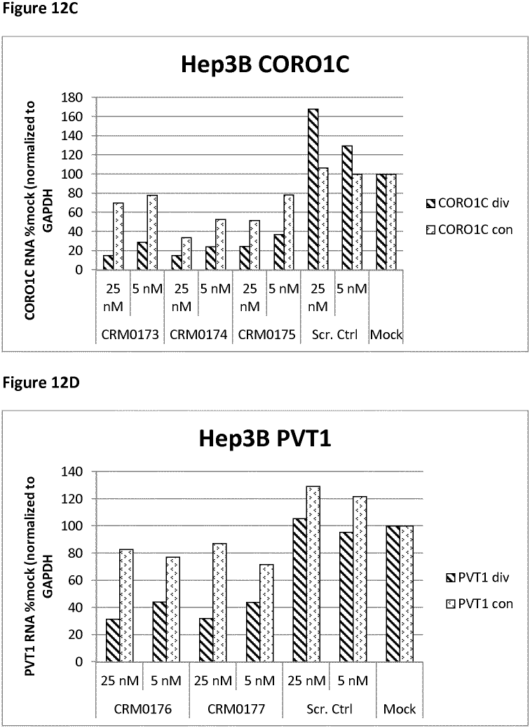

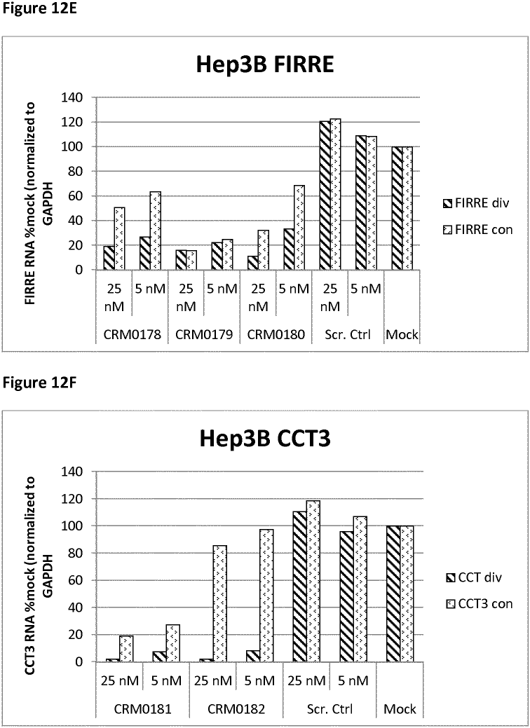

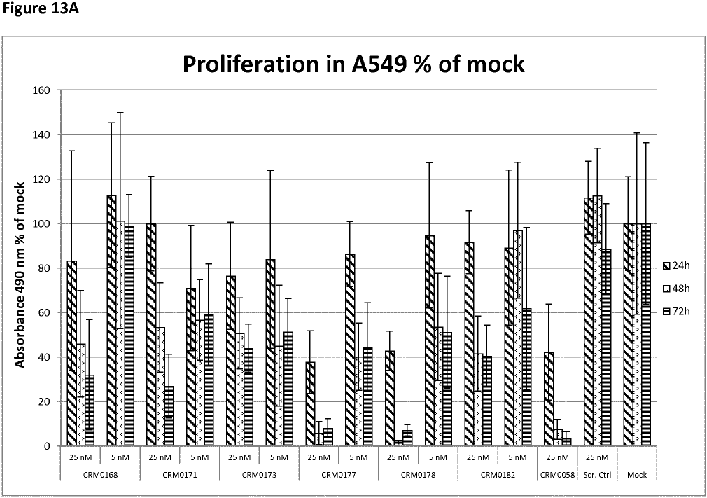

Examples of inhibition of circRNAs in A549 cells are shown in FIG. 10. Examples of inhibition of circRNAs in SK-Hep-1 cells are shown in FIG. 11. Examples of inhibition of circRNAs in Hep3B cells are shown in FIG. 12.

### Example 17: Effect of circRNA Knockdown by Antisense Oligonucleotides on Cancer Cell Proliferation

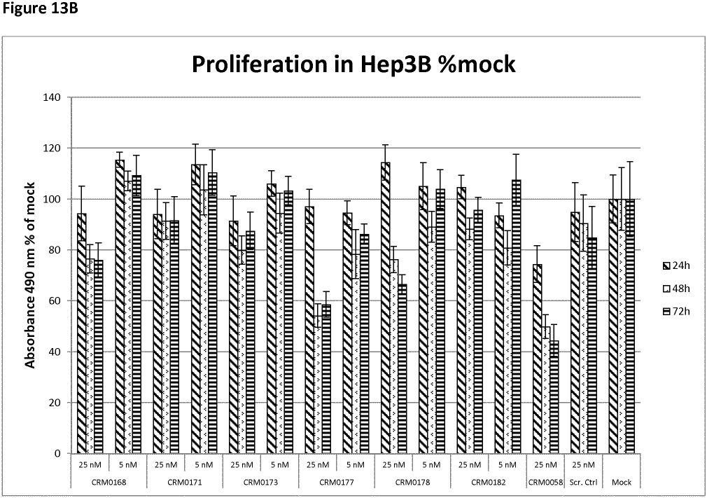

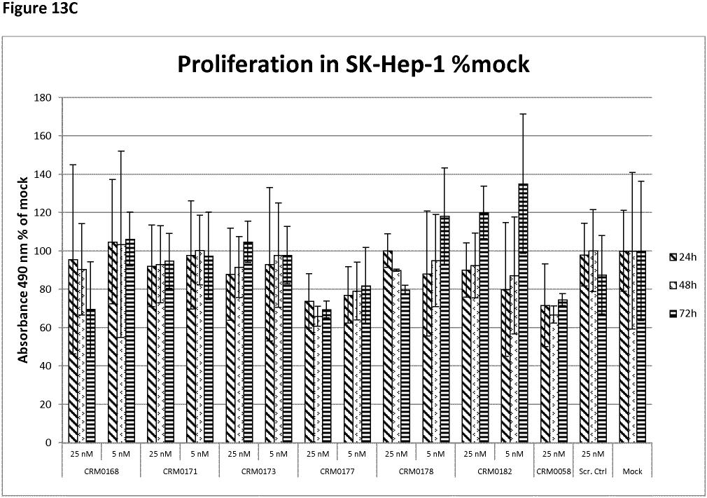

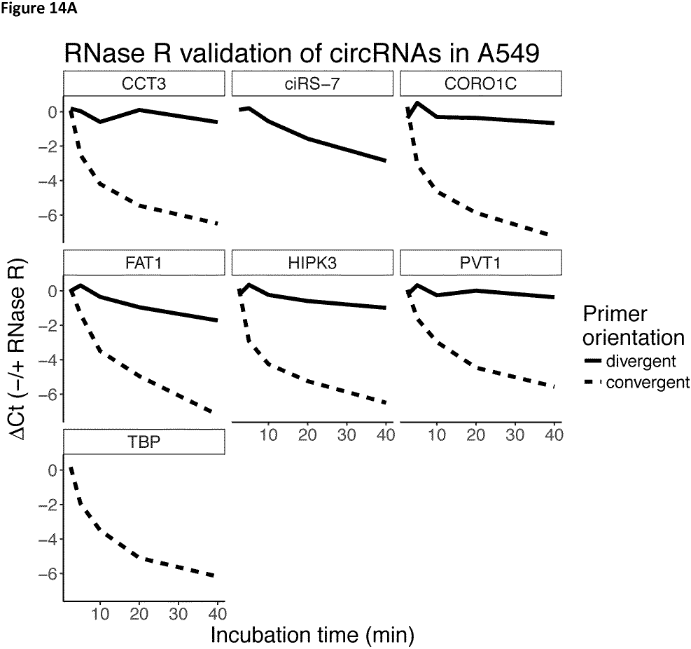

For lipid transfection in A549, cells were seeded in clear 96-well plates (NUNC) at a density of 2000 cells per well in complete culture medium the day before transfection. Six wells were left without cells for blank control. Lipid transfection was carried out as described in example 9 using 0.25 µl Lipofectamine 2000 in 50 µl OptiMEM pr. well and oligonucleotide concentrations 5 nM and 25 nM in 6 wells pr. concentration. After 4 hours, the cells were washed with OptiMEM, 100 µl complete culture medium was added to each well and the cells were incubated in a humidified 5% CO₂ incubator at 37° C. for 24 - 72 hours. For measurement of cell proliferation, 20 µl of the CellTiter Aqueous One Solution (Promega) was added to each well and the cells were incubated for 1-4 hours in a humidified 5% CO₂ incubator at 37° C. The absorbance at 490 nm was read in a Varioskan Lux plate-reader (Thermo Fisher Scientific) and the values from the blank controls were subtracted. The inhibition of proliferation was plotted relative to mock treated controls. Examples of the effect of circRNA knockdown on A549 cell proliferation using antisense oligonucleotides CRM0171, CRM0168, CRM0173, CRM0177, CRM0178, CRM0182 (SEQ ID NOs 374, 2286, 2290, 2293, 2294, and 2298 respectively) are shown in FIG. 13A.

Similarly, for lipid transfection in Hep3B cells, cells were seeded in clear 96-well plates (NUNC) at a density of 16000 cells pr. well in complete culture medium the day before transfection. Transfection and analysis was carried out as described for A549 using 0.5 µl Lipofectamine 2000 in 50 µl OptiMEM pr. well.

For lipid transfection in SK-Hep-1 cells, cells were seeded in clear 96-well plates (NUNC) at a density of 16000 cells pr. well in complete culture medium the day before transfection. Transfection and analysis was carried out as described for A549 using 0.25 µl Lipofectamine 2000 in 50 µl OptiMEM p. well.

Examples of the effect of circRNA knockdown on Hep3B cell proliferation using antisense oligonucleotides CRM0171, CRM0168, CRM0173, CRM0177, CRM0178, CRM0182 (SEQ ID NOs 374, 2286, 2290, 2293, 2294, and 2298 respectively) are shown in FIG. 13B.

Examples of the effect of circRNA knockdown on SK-Hep-1 cell proliferation using antisense oligonucleotides CRM0171, CRM0168, CRM0173, CRM0177, CRM0178, CRM0182 (SEQ ID NOs 374, 2286, 2290, 2293, 2294, and 2298 respectively) are shown in FIG. 13C.

### Example 18: RNase R Treatment of Total RNA

Circular RNAs are resistant to treatment with the 3′-5′ exoribonuclease RNase R, whereas single-stranded linear RNAs are rapidly degraded. To validate the circular nature of identified putative circRNAs, total RNA extracted from the cell lines used was treated with RNase R and circular and linear transcripts were quantified using qRT-PCR and compared to untreated controls.

Total RNA was isolated from the cells using the RNeasy mini kit (Qiagen) with the addition of a DNase I treatment step according to the manufacturer’s instructions. For the RNase R treatment, 2 µg total DNase treated RNA was incubated with 6 U RNase R (Epicentre cat. no. RNR07250) in a 10 µl reaction at 37° C. for the times indicated. Corresponding samples without enzyme added were included as controls. The reaction was stopped by transfer to ice and addition of 90 µl RNase-free H₂O and 350 µl RLT-lysis buffer and RNA was purified on the RNeasy MinElute columns (Qiagen cat. no. 74204). For the cDNA synthesis, 10 µl RNA was reverse transcribed into cDNA using the High Capacity cDNA reverse transcription kit (Life Technologies cat. no. 4374967) according to the protocol provided by the manufacturer.

Levels of the circRNAs and corresponding linear RNAs were measured using quantitative RT-PCR as described in example 14.

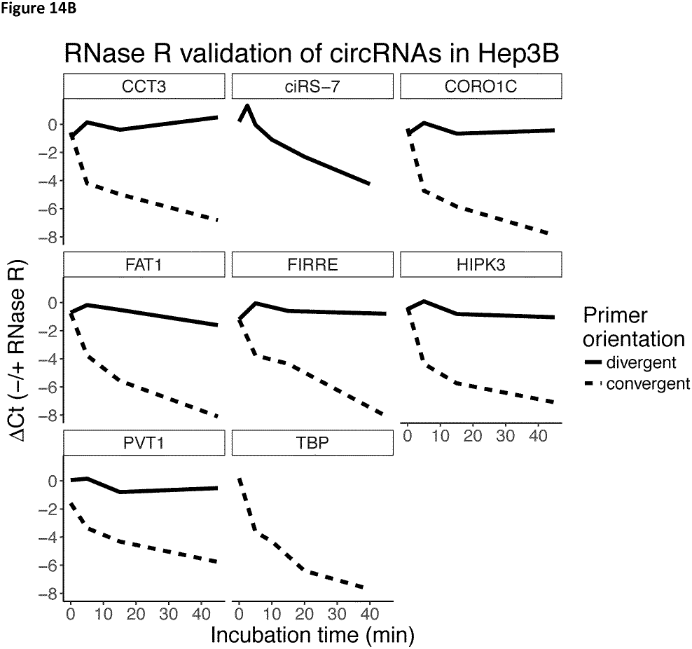

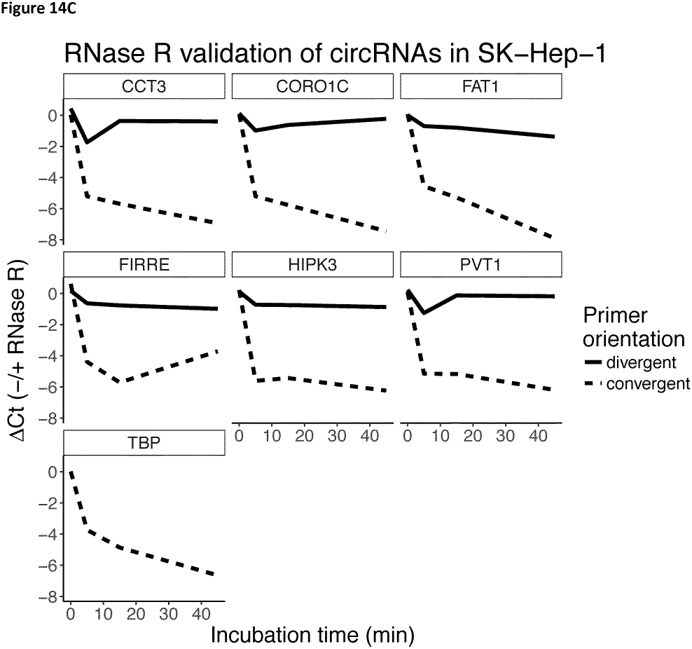

Examples of effect of RNase R on circRNAs and linear RNAs from the cell lines A549, SK-Hep-1 and Hep3B are shown in FIGS. 14 A, B and C, respectively.

### Example 19: Array Analysis of Antisense-Mediated Knockdown of ciRS-7 in Cultured Cancercells

The lung cancer cell line A549 was transfected with ciRS-7 antisense oligonucleotides CRM0106 (SEQ ID NO: 360) and CRM0108 (SEQ ID NO: 362) as described in Example 10. Cells were harvested after 48 h and 72 h and total RNA was isolated from the cells using the miRNeasy mini kit (Qiagen cat. no. 217004) according to the manufacturer’s instructions.

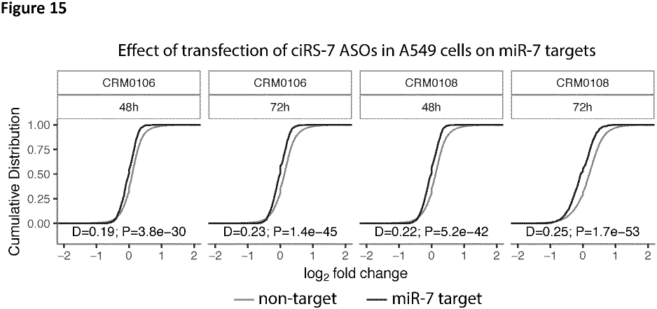

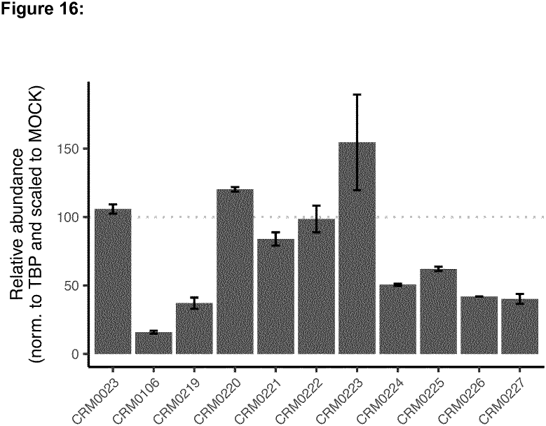

RNA was analyzed on the Affymetrix Exon array as described in Bergkvist KS et al. (BMC Immunol. 2014;15: 3. doi: 10.1186/1471-2172-15-3) and data analyses done using R and Bioconductor packages (Genome Biol. 2004; 5: R80. doi: 10.1186/ gb-2004-5-10-r80). Array data was normalized using the RMA algorithm and invariant genes removed. miR-7 target predictions were downloaded from DIANA-microT (http://diana.imis.athena-innovation.gr/DianaTools/index.php?r=microT_CDS/index). Examples of the effect of transfection of the ciRS-7 antisense oligonucleotides in A549 cells on miR-7 targets are shown in FIG. 15.

### Example 20: The Effect of Introducing Mismatches Into Gapmer Oligonucleotides on Antisense-Mediated Knockdown of ciRS7 in SK-Hep1 Cells

The knockdown effect mediated by antisense oligonucleotides harboring 2-nucleotide mismatches alongside the perfect match gapmer antisense oligonucleotide CRM0106 (SEQ ID NO: 360) targeting the backsplice site in ciRS-7 (SEQ ID NO: 1) was assessed as described in previous example 11.

The antisense oligonucleotides can be introduced into the cells using a lipid vehicle or via unassisted uptake.

For lipid-mediated transfection of the perfect match gapmer antisense oligonucleotide CRM0106 (SEQ ID NO: 360) targeting the backsplice site in ciRS-7 and the 2-nucleotide mismatched oligonucleotides (SEQ ID NOs: 2285-2293) SK-Hep1 cells were seeded in 12-well cell culture plates the day before transfection and transfected essentially as described in Dean et al. (Journal of Biological Chemistry 1994, 269, 16416-16424) using Lipofectamine 2000 in a final concentration of 5 µl/ml Optimem I (Gibco) and antisense oligonucleotide at a 5 nM final concentration. A scrambled sequence oligonucleotide and mock transfection were included as controls.

24 hours after transfection, total RNA was isolated from the cells using the RNeasy mini kit (Qiagen) according to the manufacturer’s instructions and 1 µg total RNA was reverse transcribed into cDNA using the High Capacity cDNA reverse transcription kit (Life Technologies cat. no. 4374967) according to the protocol provided by the manufacturer. ciRS-7 RNA levels were determined by quantitative RT-PCR using Taqman Gene Expression Master Mix (ABI cat. no. 4369542) and a divergent Taqman probe assay designed to specifically detect ciRS-7 (as described in example 5), furthermore the expression of TBP mRNA was measured (IDT Hs.PT.58v.39858774) and used as an endogenous control.

Quantitative PCR was carried out on a Quantstudio 6 Flex Real-Time thermocycler (ABI) An example of knockdown of ciRS-7 by perfect match gapmer antisense oligonucleotide CRM0106 (SEQ ID NO 360) compared to different mismatched gapmer antisense oligonucleotides CRM0219-227 (SEQ ID NOs 2285-2293) in cultured SK-Hep1 cells, is shown in FIG. 9.

TABLE 5

| Design of oligonucleotides for assessment of the specificity of the gapmer antisense oligonucleotide targeting the backsplice site in ciRS-7 (SEQ ID NO: 0360, 0361, 0362, 0363, 0364, 0365, 0366, 0367). Uppercase = LNA, lowercase = DNA. Nucleotides written in bold and underlined represent mismatches relative to the perfect match gapmer antisense oligonucleotide.oligoname | seqID | oligoRep |
|---|---|---|
| CRM0106 | 360 | GTgccatcggaaaccCT |
| CRM0219 | N/A | CAgccatcggaaaccCT |
| CRM0220 | N/A | GTcgcatcggaaaccCT |
| CRM0221 | N/A | GTgcgttcggaaaccCT |
| CRM0222 | N/A | GTgccaaggaaaccCT |
| CRM0223 | N/A | GTgccatcccaaaccCT |
| CRM0224 | N/A | GTgccatcggttaccCT |
| CRM0225 | N/A | GTgccatcggaatgcCT |
| CRM0226 | N/A | GTgccatcggaaacgGT |
| CRM0227 | N/A | GTgccatcggaaaccGA |

1. A compound comprising a modified antisense oligonucleotide consisting of any one of SEQ ID NOs: 2149 - 2259.

2-13. (canceled)

14. The compound according to claim 1, wherein said modified antisense oligonucleotide comprises wing regions having nucleotide analogues selected from beta-D-oxy LNA, alpha-L-oxy-LNA, beta-D-amino-LNA, alpha-L-amino-LNA, beta-D-thio-LNA, alpha-L-thio-LNA, 5′-methyl-LNA, beta-D-ENA or alpha-L-ENA.

15. The compound according to claim 14, wherein the modified antisense oligonucleotide is 100% complementary to an endogenous lncRNA.

16. The compound according to claim 14, wherein the nucleotide analogues of the wings are Beta-D-Oxy LNA.

17. The compound according to claim 1, wherein said modified antisense oligonucleotide comprises wing regions having nucleoside analogues, which are not LNA, but are selected from tricyclo-DNA, 2′-Fluoro, 2′-O-methyl, 2′-methoxyethyl (2′-MOE), 2′cyclic ethyl (cET), UNA or Conformationally Restricted Nucleoside (CRN).

18. The compound according to claim 1, wherein said modified antisense oligonucleotide comprises wing regions having nucleoside analogues, which are a mixture of LNA and anyone of tricyclo-DNA, 2′Fluoro, 2′-O-methyl, 2′-methoxyethyl (2′-MOE), 2′cyclic ethyl (cET), UNA, or Conformationally Restricted Nucleoside (CRN).

19. The compound according to claim 18, wherein the nucleoside analogues of the wing regions are a mixture of LNA and 2′-Fluoro.

20. The compound according to claim 1, wherein the modified antisense oligonucleotide is conjugated with a ligand.

21. The compound according to claim 20, wherein the modified antisense oligonucleotide is conjugated with folic acid or N-acetylgalactosamine (GalNAc).

22. The compound according to claim 1, wherein the modified antisense oligonucleotide is unconjugated in a pharmaceutical composition.

23. The compound according to claim 1, wherein the modified antisense oligonucleotide is formulated in lipid nanoparticles.

24. A method of therapy, comprising providing a subject the compound of claim 1 and a carrier.

25. The method according to claim 24, wherein the compound comprises more than one modified antisense oligonucleotide consisting of any one of SEQ ID NOs: 2149 - 2259.

26. A method of downregulating an endogenous lncRNA selected from the list of DANCR, H19, HOTAIR, HOTTIP, HULC, LINC-ROR, MALAT1, MVIH, PCBP2-OT1, PVT1, TUG1, UCA1, UFC1 or LINC01215 in a cell, comprising administering to a cell an effective amount of an antisense oligonucleotide that is complementary to DANCR, H19, HOTAIR, HOTTIP, HULC, LINC-ROR, MALAT1, MVIH, PCBP2-OT1, PVT1, TUG1, UCA1, UFC1 or LINC01215.

27. The method of claim 26, wherein the cell is in a human body.

28. The method of claim 26, wherein the cell is a cancer cell in a human body.

29. A method of inhibiting a cancer in a subject, comprising administering an effective dosage of the compound according to claim 1 to a subject in need thereof.

30. The method according to claim 29, wherein the cancer is hepatocellular carcinoma.

31. The method according to claim 30, wherein the subject is a human.

32. The method according to claim 29 further comprising administering an additional cancer therapy to said subject.
